SEQUENCE LISTING (1115 sequences)

SEQ ID NO: 1 - protein, 18 aa, Artificial
  Synthetic peptide.
VLGIAGGLDAYGELVGGN

SEQ ID NO: 2 - protein, 11 aa, Artificial
  Synthetic peptide.
LDAYGELVGGN

SEQ ID NO: 3 - protein, 15 aa, Artificial
  Synthetic peptide.
KFINGVLSQFVLERK

SEQ ID NO: 4 - protein, 20 aa, Artificial
  Synthetic peptide.
SQRIIEPVKSPQPYPGFSVS

SEQ ID NO: 5 - protein, 22 aa, Artificial
  Synthetic peptide.
FSVAACGEQRAVTFVLLIEDLI

SEQ ID NO: 6 - protein, 29 aa, Artificial
  Synthetic peptide.
WAWAESPRCVSTRSNIHALAFRVEVAALT

SEQ ID NO: 7 - protein, 12 aa, Artificial
  Synthetic peptide.
SPAGLPGDGDEA

SEQ ID NO: 8 - protein, 4 aa, Artificial
  Synthetic peptide.
RISE

SEQ ID NO: 9 - protein, 22 aa, Artificial
  Synthetic peptide.
FGNIFKGLKDVIETIVKWTAAK

SEQ ID NO: 10 - protein, 20 aa, Artificial
  Synthetic peptide.
FRSPCINNNSLQPPGVYPAR

SEQ ID NO: 11 - protein, 12 aa, Artificial
  Synthetic peptide.
ALAGLAGLISGK

SEQ ID NO: 12 - protein, 9 aa, Artificial
  Synthetic peptide.
DVILRVEAQ

SEQ ID NO: 13 - protein, 4 aa, Artificial
  Synthetic peptide.
IDMR

SEQ ID NO: 14 - protein, 8 aa, Artificial
  Synthetic peptide.
NNAIVYIS

SEQ ID NO: 15 - protein, 9 aa, Artificial
  Synthetic peptide.
YSKTLHFAD

SEQ ID NO: 16 - protein, 12 aa, Artificial
  Synthetic peptide.
PGAFRNPQMPRG

SEQ ID NO: 17 - protein, 20 aa, Artificial
  Synthetic peptide.
PALVDLSNKEAVWAVLDDHS

SEQ ID NO: 18 - protein, 30 aa, Artificial
  Synthetic peptide.
YVEEAVRAALKKEARISTEDTPVNLPSFDC

SEQ ID NO: 19 - protein, 22 aa, Artificial
  Synthetic peptide.
VPLDDGTRRPEVARNRDKDRED

SEQ ID NO: 20 - protein, 20 aa, Artificial
  Synthetic peptide.
PALVDLSNKEAVWAVLDDHS

SEQ ID NO: 21 - protein, 19 aa, Artificial
  Synthetic peptide.
EEAEEKLAEVSQAVKRLVR

SEQ ID NO: 22 - protein, 26 aa, Artificial
  Synthetic peptide.
VGLDVSVLVLFFGLQLLSVLLGAMIR

SEQ ID NO: 23 - protein, 38 aa, Artificial
  Synthetic peptide.
LTILPTTFFAIIVPILAVAFIAYSGFKIKGIVEHKDQW

SEQ ID NO: 24 - protein, 27 aa, Artificial
  Synthetic peptide.
ALFVSLEQFLVVVAKSVFALCHSGTLS

SEQ ID NO: 25 - protein, 25 aa, Artificial
  Synthetic peptide.
VSRDEAMEFIDREWTTLQPAGKSHA

SEQ ID NO: 26 - protein, 32 aa, Artificial
  Synthetic peptide.
GSVIKKRRKRMSKKKHRKMLRRTRVQRRKLGK

SEQ ID NO: 27 - protein, 27 aa, Artificial
  Synthetic peptide.
GKAKPYQVRQVLRAVDKLETRRKKGGR

SEQ ID NO: 28 - protein, 18 aa, Artificial
  Synthetic peptide.
NATGTDIGEVTLTLGRFS

SEQ ID NO: 29 - protein, 20 aa, Artificial
  Synthetic peptide.
VSFLAGWLCLGLAAWRLGNA

SEQ ID NO: 30 - protein, 37 aa, Artificial
  Synthetic peptide.
VRTLTILVIFIFNYLKSISYKLKQPFENNLAQSMISI

SEQ ID NO: 31 - protein, 27 aa, Artificial
  Synthetic peptide.
AFWLNILLTLLGYIPGIVHAVYIIAKR

SEQ ID NO: 32 - protein, 36 aa, Artificial
  Synthetic peptide.
EICLTLVFPIRGSYSEAAKFPVPIHIVEDGTVELPK

SEQ ID NO: 33 - protein, 20 aa, Artificial
  Synthetic peptide.
VYRHLRFIDGKLVEIRLERK

SEQ ID NO: 34 - protein, 22 aa, Artificial
  Synthetic peptide.
YIVGALVILAVAGLIYSMLRKA

SEQ ID NO: 35 - protein, 18 aa, Artificial
  Synthetic peptide.
VMFVLTRGRSPRPMIPAY

SEQ ID NO: 36 - protein, 19 aa, Artificial
  Synthetic peptide.
FGFCVWMYQLLAGPPGPPA

SEQ ID NO: 37 - protein, 14 aa, Artificial
  Synthetic peptide.
QRVSLWSEVEHEFR

SEQ ID NO: 38 - protein, 23 aa, Artificial
  Synthetic peptide.
KRGSKIVIAIAVVLIVLAGVWVW

SEQ ID NO: 39 - protein, 28 aa, Artificial
  Synthetic peptide.
TVLDWLSLALATGLFVYLLVALLRADRA

SEQ ID NO: 40 - protein, 18 aa, Artificial
  Synthetic peptide.
DRCLSVLSWSPPKVSPLI

SEQ ID NO: 41 - protein, 17 aa, Artificial
  Synthetic peptide.
DPALADFAAGMRAQVRT

SEQ ID NO: 42 - protein, 22 aa, Artificial
  Synthetic peptide.
WTKPSFTDLRLGFEVTLYFANR

SEQ ID NO: 43 - protein, 17 aa, Artificial
  Synthetic peptide.
FSFKQRVMFRKEVERLR

SEQ ID NO: 44 - protein, 15 aa, Artificial
  Synthetic peptide.
VIKISVPGQVQMLIP

SEQ ID NO: 45 - protein, 26 aa, Artificial
  Synthetic peptide.
KLQVHHGRATHTLLLQPPLCAPGTIR

SEQ ID NO: 46 - protein, 25 aa, Artificial
  Synthetic peptide.
SLVRIHDQQPWVTRGAFIDAARTCS

SEQ ID NO: 47 - protein, 17 aa, Artificial
  Synthetic peptide.
HSDEPIPNILFKSDSVH

SEQ ID NO: 48 - protein, 22 aa, Artificial
  Synthetic peptide.
GKPKRMPAEFIDGYGQALLAGA

SEQ ID NO: 49 - protein, 20 aa, Artificial
  Synthetic peptide.
DEYPAKLPLSDKGATEPRRH

SEQ ID NO: 50 - protein, 26 aa, Artificial
  Synthetic peptide.
SDILAEMFEKGELQTLVKDAAAKANA

SEQ ID NO: 51 - protein, 12 aa, Artificial
  Synthetic peptide.
RWVSCNPSWRIQ

SEQ ID NO: 52 - protein, 25 aa, Artificial
  Synthetic peptide.
NHKTLKEWKAKWGPEAVESWATLLG

SEQ ID NO: 53 - protein, 14 aa, Artificial
  Synthetic peptide.
LALIGAGIWMIRKG

SEQ ID NO: 54 - protein, 24 aa, Artificial
  Synthetic peptide.
RLEYRRLETQVEENPESGRRPMRG

SEQ ID NO: 55 - protein, 19 aa, Artificial
  Synthetic peptide.
CDDLHALERAGKLDALLSA

SEQ ID NO: 56 - protein, 25 aa, Artificial
  Synthetic peptide.
AVGNNLGKDNDSGHRGKKHRKHKHR

SEQ ID NO: 57 - protein, 22 aa, Artificial
  Synthetic peptide.
YLTSLGLDAAEQAQGLLTILKG

SEQ ID NO: 58 - protein, 24 aa, Artificial
  Synthetic peptide.
HATLLPAVREAISRQLLPALVPRG

SEQ ID NO: 59 - protein, 21 aa, Artificial
  Synthetic peptide.
GCKGCAQRDPCAEPEPYFRLR

SEQ ID NO: 60 - protein, 22 aa, Artificial
  Synthetic peptide.
EPLILKELVRNLFLFCYARALR

SEQ ID NO: 61 - protein, 21 aa, Artificial
  Synthetic peptide.
QTVHHIHMHVLGQRQMHWPPG

SEQ ID NO: 62 - protein, 27 aa, Artificial
  Synthetic peptide.
HARAAVGVAELPRGAAVEVELIAAVRP

SEQ ID NO: 63 - protein, 27 aa, Artificial
  Synthetic peptide.
DTDCLSRAYAQRIDELDKQYAGIDKPL

SEQ ID NO: 64 - protein, 24 aa, Artificial
  Synthetic peptide.
GQRQRLTCGRVSGCSEGPSREAAR

SEQ ID NO: 65 - protein, 11 aa, Artificial
  Synthetic peptide.
GGTKEIVYQRG

SEQ ID NO: 66 - protein, 12 aa, Artificial
  Synthetic peptide.
ILSQEADRKKLF

SEQ ID NO: 67 - protein, 15 aa, Artificial
  Synthetic peptide.
NRQAQGERAHGEQQG

SEQ ID NO: 68 - protein, 13 aa, Artificial
  Synthetic peptide.
KIDTNQWPPNKEG

SEQ ID NO: 69 - protein, 11 aa, Artificial
  Synthetic peptide.
EPTDGVACKER

SEQ ID NO: 70 - protein, 27 aa, Artificial
  Synthetic peptide.
GWWEELLHETILSKFKITKALELPIQL

SEQ ID NO: 71 - protein, 26 aa, Artificial
  Synthetic peptide.
DIDWGRKISCAAGVAYGAIDGCATTV

SEQ ID NO: 72 - protein, 23 aa, Artificial
  Synthetic peptide.
GVARGLQLGIKTRTQWGAATGAA

SEQ ID NO: 73 - protein, 17 aa, Artificial
  Synthetic peptide.
EMRLSKFFRDFILWRKK

SEQ ID NO: 74 - protein, 17 aa, Artificial
  Synthetic peptide.
EMRISRIILDFLFLRKK

SEQ ID NO: 75 - protein, 28 aa, Artificial
  Synthetic peptide.
FFKTIFVLILGALGVAAGLYIEKNYIDK

SEQ ID NO: 76 - protein, 28 aa, Artificial
  Synthetic peptide.
FGTPWSITNFWKKNFNDRPDFDSDRRRY

SEQ ID NO: 77 - protein, 13 aa, Artificial
  Synthetic peptide.
GGNLGPGFGVIIP

SEQ ID NO: 78 - protein, 19 aa, Artificial
  Synthetic peptide.
AIATGLDIVDGKFDGYLWA

SEQ ID NO: 79 - protein, 28 aa, Artificial
  Synthetic peptide.
FGVGVGIALFMAGYAIGKDLRKKFGKSC

SEQ ID NO: 80 - protein, 31 aa, Artificial
  Synthetic peptide.
QKPRKNETFIGYIQRYDIDGNGYQSLPCPQN

SEQ ID NO: 81 - protein, 32 aa, Artificial
  Synthetic peptide.
FRKKRYGLSILLWLNAFTNLVNSIHAFYMTLF

SEQ ID NO: 82 - protein, 24 aa, Artificial
  Synthetic peptide.
VMASLTWRMRAASASLPTHSRTDA

SEQ ID NO: 83 - protein, 19 aa, Artificial
  Synthetic peptide.
HRKNPVLGVGRRHRAHNVA

SEQ ID NO: 84 - protein, 13 aa, Artificial
  Synthetic peptide.
EAVGQDLVDAHHP

SEQ ID NO: 85 - protein, 23 aa, Artificial
  Synthetic peptide.
HEDDKRRGMSVEVLGFEVVQHEE

SEQ ID NO: 86 - protein, 8 aa, Artificial
  Synthetic peptide.
RNVIGQVL

SEQ ID NO: 87 - protein, 27 aa, Artificial
  Synthetic peptide.
TSVRPGAAGAAVPAGAAGAAGAGWRWP

SEQ ID NO: 88 - protein, 19 aa, Artificial
  Synthetic peptide.
GQDEGQRRAGVGEGQGVDG

SEQ ID NO: 89 - protein, 8 aa, Artificial
  Synthetic peptide.
AMRSVNQA

SEQ ID NO: 90 - protein, 18 aa, Artificial
  Synthetic peptide.
DQVAHSGDMLVQARRRDS

SEQ ID NO: 91 - protein, 27 aa, Artificial
  Synthetic peptide.
GHLLRVGGRVGGVGGVAGACAQPFGGQ

SEQ ID NO: 92 - protein, 12 aa, Artificial
  Synthetic peptide.
VAGACAQPFGGQ

SEQ ID NO: 93 - protein, 32 aa, Artificial
  Synthetic peptide.
GVAERNLDRITVAVAIIWTITIVGLGLVAKLG

SEQ ID NO: 94 - protein, 32 aa, Artificial
  Synthetic peptide.
VRSAKAVKALTAAGYTGELVNVSGGMKAWLGQ

SEQ ID NO: 95 - protein, 7 aa, Artificial
  Synthetic peptide.
MKAWLGQ

SEQ ID NO: 96 - protein, 37 aa, Artificial
  Synthetic peptide.
LDPLEPRIAPPGDRSHQGAPACHRDPLRGRSARDAER

SEQ ID NO: 97 - protein, 29 aa, Artificial
  Synthetic peptide.
RLRVGRATDLPLTSFAVGVVRNLPDAPAH

SEQ ID NO: 98 - protein, 30 aa, Artificial
  Synthetic peptide.
WKRLWPARILAGHSRRRMRWMVVWRYFAAT

SEQ ID NO: 99 - protein, 22 aa, Artificial
  Synthetic peptide.
AQFYEAIITGYALGAGQRIGQL

SEQ ID NO: 100 - protein, 24 aa, Artificial
  Synthetic peptide.
RAVAAHLQGRHHGHQVRRQRHGQR

SEQ ID NO: 101 - protein, 19 aa, Artificial
  Synthetic peptide.
GEGLPPPVLHLPPPRMSGR

SEQ ID NO: 102 - protein, 14 aa, Artificial
  Synthetic peptide.
DALRRSRSQGRRHR

SEQ ID NO: 103 - protein, 17 aa, Artificial
  Synthetic peptide.
SPVPRFTAVGGVSRGSP

SEQ ID NO: 104 - protein, 11 aa, Artificial
  Synthetic peptide.
WGPLGPERPLW

SEQ ID NO: 105 - protein, 11 aa, Artificial
  Synthetic peptide.
VTTNVRQGAGS

SEQ ID NO: 106 - protein, 14 aa, Artificial
  Synthetic peptide.
LAAKTAVCVGRAFM

SEQ ID NO: 107 - protein, 26 aa, Artificial
  Synthetic peptide.
GRLSRREEDPATSIILLRGAYRMAVF

SEQ ID NO: 108 - protein, 13 aa, Artificial
  Synthetic peptide.
SDNDGKLILGTSQ

SEQ ID NO: 109 - protein, 36 aa, Artificial
  Synthetic peptide.
HGAHQRTGQRLHHHRGRTVSGCRQNPVAGVDPDEHR

SEQ ID NO: 110 - protein, 23 aa, Artificial
  Synthetic peptide.
RQAPGPGLVTITAACSAPGSRSR

SEQ ID NO: 111 - protein, 10 aa, Artificial
  Synthetic peptide.
LLIERFSNHH

SEQ ID NO: 112 - protein, 9 aa, Artificial
  Synthetic peptide.
MILHRRRDR

SEQ ID NO: 113 - protein, 20 aa, Artificial
  Synthetic peptide.
GPGVVGPAPFSRLPAHALNL

SEQ ID NO: 114 - protein, 32 aa, Artificial
  Synthetic peptide.
TASPPAPSDQGLRTAFPATLLIALAALARISR

SEQ ID NO: 115 - protein, 39 aa, Artificial
  Synthetic peptide.
SPATQKAPTRAQPSRAPVQDCGDGRPTAAPDDVERLSPR

SEQ ID NO: 116 - protein, 20 aa, Artificial
  Synthetic peptide.
DVRDRVDLAGADLCAAHATR

SEQ ID NO: 117 - protein, 28 aa, Artificial
  Synthetic peptide.
FAKETGFGIGGAQEGWWIIADIYGPNPF

SEQ ID NO: 118 - protein, 23 aa, Artificial
  Synthetic peptide.
GAIPDPVTHRVDWEEDHQTRPSR

SEQ ID NO: 119 - protein, 29 aa, Artificial
  Synthetic peptide.
LVRRNAVAGRSDGLAGAEQLDLVRLQGVL

SEQ ID NO: 120 - protein, 9 aa, Artificial
  Synthetic peptide.
LFDERNKIA

SEQ ID NO: 121 - protein, 18 aa, Artificial
  Synthetic peptide.
DAITGGNPPLSDTDGLRP

SEQ ID NO: 122 - protein, 13 aa, Artificial
  Synthetic peptide.
QGLARPVLRRIPL

SEQ ID NO: 123 - protein, 18 aa, Artificial
  Synthetic peptide.
YDPVPKRKNKNSEGKREE

SEQ ID NO: 124 - protein, 17 aa, Artificial
  Synthetic peptide.
SGSAIRMLEIATKMLKR

SEQ ID NO: 125 - protein, 19 aa, Artificial
  Synthetic peptide.
YDKYIKYLSIQPPFIVYFI

SEQ ID NO: 126 - protein, 56 aa, Artificial
  Synthetic peptide.
QKIIDMSKFLFSLILFIMIVVIYIGKSIGGYSAIVSSIMLELDTVLYNKKIFFIYK

SEQ ID NO: 127 - protein, 9 aa, Artificial
  Synthetic peptide.
DEVWKMLGI

SEQ ID NO: 128 - protein, 39 aa, Artificial
  Synthetic peptide.
YSKKLFEYFYFIIFILIRYLIFYKIIQNKNYYINNIAYN

SEQ ID NO: 129 - protein, 35 aa, Artificial
  Synthetic peptide.
YFIKDDNEALSKDWEVIGNDLKGTIDKYGKEFKVR

SEQ ID NO: 130 - protein, 14 aa, Artificial
  Synthetic peptide.
SRLVREIKKKCRKS

SEQ ID NO: 131 - protein, 24 aa, Artificial
  Synthetic peptide.
FESLLPQATKKIVNNKGSKINKIF

SEQ ID NO: 132 - protein, 17 aa, Artificial
  Synthetic peptide.
ELLTQIRLALLYSVNEW

SEQ ID NO: 133 - protein, 55 aa, Artificial
  Synthetic peptide.
PLNFYRAVKENRLPLSEKNINDFTNIKLKVSPKLINLLQESSIFYNFSPKKRNTN

SEQ ID NO: 134 - protein, 42 aa, Artificial
  Synthetic peptide.
YPNEYCIFLENLSLEELKEIKAINGETLNLEEIINERKNLKD

SEQ ID NO: 135 - protein, 39 aa, Artificial
  Synthetic peptide.
AVAGAAVGALLGNDARSTAVGAAIGGALGAGAGELTKNK

SEQ ID NO: 136 - protein, 39 aa, Artificial
  Synthetic peptide.
IKGTIAFVGEDYVEIRVDKGVKLTFRKSAIANVINNNQQ

SEQ ID NO: 137 - protein, 28 aa, Artificial
  Synthetic peptide.
KKFIILLFILVQGLIFSATKTLSDIIAL

SEQ ID NO: 138 - protein, 16 aa, Artificial
  Synthetic peptide.
FTQGIKRIVLKRLKED

SEQ ID NO: 139 - protein, 29 aa, Artificial
  Synthetic peptide.
MPKRHYYKLEAKALQFGLPFAYSPIQLLK

SEQ ID NO: 140 - protein, 18 aa, Artificial
  Synthetic peptide.
IIELHPKSWTQDWRCSFL

SEQ ID NO: 141 - protein, 39 aa, Artificial
  Synthetic peptide.
VEAGKRNISLENIEKISKGLGISISELFKYIEEGEDKIG

SEQ ID NO: 142 - protein, 31 aa, Artificial
  Synthetic peptide.
RNSADNQTKIDKIRIDISLWDEHLNIVKQGK

SEQ ID NO: 143 - protein, 30 aa, Artificial
  Synthetic peptide.
GVENRRFYERDVSKVSMMTSEAVAPRGGSK

SEQ ID NO: 144 - protein, 22 aa, Artificial
  Synthetic peptide.
IVELDDTTILERALSMLGEANA

SEQ ID NO: 145 - protein, 23 aa, Artificial
  Synthetic peptide.
SVRAVKPIDETVARHFPGDFIVN

SEQ ID NO: 146 - protein, 32 aa, Artificial
  Synthetic peptide.
YINRRLKKAFSDADIKEAPAEFYEELRRVQYV

SEQ ID NO: 147 - protein, 23 aa, Artificial
  Synthetic peptide.
SVRAVKPIDEIVAWHFPGDFIVN

SEQ ID NO: 148 - protein, 29 aa, Artificial
  Synthetic peptide.
YVSADESAYNHIVTDDIPLADRRIEAVQQ

SEQ ID NO: 149 - protein, 9 aa, Artificial
  Synthetic peptide.
YIACPGYFY

SEQ ID NO: 150 - protein, 13 aa, Artificial
  Synthetic peptide.
YFSFLEIVGMARR

SEQ ID NO: 151 - protein, 28 aa, Artificial
  Synthetic peptide.
LKLAFGVYPFQAMSQSDTAVSERNVLWR

SEQ ID NO: 152 - protein, 29 aa, Artificial
  Synthetic peptide.
GRFQISIRGEEKSKVKVQGKGTFTDRNTT

SEQ ID NO: 153 - protein, 30 aa, Artificial
  Synthetic peptide.
RRFRKTTENREKSKNKKAVLGLSTTSTASY

SEQ ID NO: 154 - protein, 29 aa, Artificial
  Synthetic peptide.
WENKPSPLGSIKKLQGLVYRLIGYRHFWV

SEQ ID NO: 155 - protein, 30 aa, Artificial
  Synthetic peptide.
IFSLHHFALICSEMGTFAVSKRAKYKWEVL

SEQ ID NO: 156 - protein, 11 aa, Artificial
  Synthetic peptide.
AQYKYINKLLN

SEQ ID NO: 157 - protein, 30 aa, Artificial
  Synthetic peptide.
NKVLQVEVMWDGSVVGRPAGVISIKSSKKG

SEQ ID NO: 158 - protein, 28 aa, Artificial
  Synthetic peptide.
QKAKEESDRKAAVSYNGFHRVNVVSIPK

SEQ ID NO: 159 - protein, 31 aa, Artificial
  Synthetic peptide.
MENILIYIPMVLSPFGSGILLFLGKDRRYML

SEQ ID NO: 160 - protein, 29 aa, Artificial
  Synthetic peptide.
KKSHSQGKRKLKDLNSAYKIDNQLHYALR

SEQ ID NO: 161 - protein, 23 aa, Artificial
  Synthetic peptide.
CYDSFDFSIFVTFANRMKLSVGS

SEQ ID NO: 162 - protein, 17 aa, Artificial
  Synthetic peptide.
AQSAGQIKRKSKVRIHV

SEQ ID NO: 163 - protein, 35 aa, Artificial
  Synthetic peptide.
SRMSEHSPAGLVFEVGPMDKGSFIILDSYHPTVKK

SEQ ID NO: 164 - protein, 41 aa, Artificial
  Synthetic peptide.
ELHRIMSTEKIGAVTKMNFDTAPIMSILPIDIYPKEVGIGS

SEQ ID NO: 165 - protein, 31 aa, Artificial
  Synthetic peptide.
FARVRRLHQNRILTQPLTNLKYCLRQPIYSD

SEQ ID NO: 166 - protein, 30 aa, Artificial
  Synthetic peptide.
AYGKVFSMDIMLSENDKLIVLRISHHSAWH

SEQ ID NO: 167 - protein, 23 aa, Artificial
  Synthetic peptide.
SVRAVKPIDKTVARHFPGDFIVN

SEQ ID NO: 168 - protein, 13 aa, Artificial
  Synthetic peptide.
FEGLKNLLGDDII

SEQ ID NO: 169 - protein, 12 aa, Artificial
  Synthetic peptide.
LFRKEDQEHVLL

SEQ ID NO: 170 - protein, 21 aa, Artificial
  Synthetic peptide.
SGGSDTDGSSSGEPGSHSGDL

SEQ ID NO: 171 - protein, 10 aa, Artificial
  Synthetic peptide.
GEPGSHSGDL

SEQ ID NO: 172 - protein, 27 aa, Artificial
  Synthetic peptide.
PVGDIMSGFLRGANQPRFLLDHISFGS

SEQ ID NO: 173 - protein, 26 aa, Artificial
  Synthetic peptide.
GTNVPTQILGYSREERFDYEPAPEQR

SEQ ID NO: 174 - protein, 27 aa, Artificial
  Synthetic peptide.
LLASHPERLSLGVFFVYRVLHLLLENT

SEQ ID NO: 175 - protein, 16 aa, Artificial
  Synthetic peptide.
TCYPLIQRKTDRAYEA

SEQ ID NO: 176 - protein, 10 aa, Artificial
  Synthetic peptide.
VVFGGGDRLV

SEQ ID NO: 177 - protein, 7 aa, Artificial
  Synthetic peptide.
YGKESDP

SEQ ID NO: 178 - protein, 18 aa, Artificial
  Synthetic peptide.
LTASICRQWNDNSTPYQR

SEQ ID NO: 179 - protein, 27 aa, Artificial
  Synthetic peptide.
PLRSFVAEKAEHAFRVVRIADFDFGHS

SEQ ID NO: 180 - protein, 18 aa, Artificial
  Synthetic peptide.
ALLVLNLLLMQFFFGKNM

SEQ ID NO: 181 - protein, 17 aa, Artificial
  Synthetic peptide.
HYHFLLEFGFHKGDYLE

SEQ ID NO: 182 - protein, 8 aa, Artificial
  Synthetic peptide.
HRKDVYKK

SEQ ID NO: 183 - protein, 41 aa, Artificial
  Synthetic peptide.
IQIIVNAFVEKDKTGAVIEVLYASNNHEKVKAKYEELVAIS

SEQ ID NO: 184 - protein, 17 aa, Artificial
  Synthetic peptide.
ILVLLALQVELDSKFQY

SEQ ID NO: 185 - protein, 25 aa, Artificial
  Synthetic peptide.
LMIFDKHANLKYKYGNRSFGVEAIM

SEQ ID NO: 186 - protein, 17 aa, Artificial
  Synthetic peptide.
AASGFTYCASNGVWHPY

SEQ ID NO: 187 - protein, 32 aa, Artificial
  Synthetic peptide.
KPEKEKLDTNTLMKVVNKALSLFDRLLIKFGA

SEQ ID NO: 188 - protein, 41 aa, Artificial
  Synthetic peptide.
TEILNFLITVCADRENWKIKHGLSDSVLLIFFARFTGAEYW

SEQ ID NO: 189 - protein, 51 aa, Artificial
  Synthetic peptide.
MPVSKKRYMLSSAYATALGICYGQVATDEKESEITAIPDLLDYLSVEEYLL

SEQ ID NO: 190 - protein, 18 aa, Artificial
  Synthetic peptide.
RAGRIKKLSQKEAEPFEN

SEQ ID NO: 191 - protein, 42 aa, Artificial
  Synthetic peptide.
MRFKRFDRDYALSGDNVFEVLTASCDVIERNLSYREMCGLMQ

SEQ ID NO: 192 - protein, 18 aa, Artificial
  Synthetic peptide.
KRKHENVIVAEEMRVIKN

SEQ ID NO: 193 - protein, 41 aa, Artificial
  Synthetic peptide.
LCRLEKLCKQFLRQDKVVTYYLMLPYKRAIEAFYQELKERS

SEQ ID NO: 194 - protein, 37 aa, Artificial
  Synthetic peptide.
YPFCLATVDHLPEGLSVTDYERVQRLVSQFLLNKEER

SEQ ID NO: 195 - protein, 30 aa, Artificial
  Synthetic peptide.
SPLEKYGTGSMTALTFLLGCCLLVLSKKSR

SEQ ID NO: 196 - protein, 25 aa, Artificial
  Synthetic peptide.
KRKRWAILTLFLAGLGAVGIVLATF

SEQ ID NO: 197 - protein, 34 aa, Artificial
  Synthetic peptide.
VCFKDISVFLSPFRGQEVLFCGKAKHSLIYVIGT

SEQ ID NO: 198 - protein, 13 aa, Artificial
  Synthetic peptide.
FFLNVIAIRIPHF

SEQ ID NO: 199 - protein, 35 aa, Artificial
  Synthetic peptide.
MLSNVLSRSVVSPNVDIPNSMVILSPLLISISNYH

SEQ ID NO: 200 - protein, 22 aa, Artificial
  Synthetic peptide.
KLIFAALGLVFLLIGLRDSRSK

SEQ ID NO: 201 - protein, 16 aa, Artificial
  Synthetic peptide.
RNINVSATFITEKSLV

SEQ ID NO: 202 - protein, 37 aa, Artificial
  Synthetic peptide.
DIGRIIGKKGRTITAIRSIVYSVPTQGKKVRLVIDEK

SEQ ID NO: 203 - protein, 24 aa, Artificial
  Synthetic peptide.
RIEASLISAIMFSMFNAIVKFLQK

SEQ ID NO: 204 - protein, 40 aa, Artificial
  Synthetic peptide.
NQKMEINSMTSEKEKMLAGHFHNEANFAVIFKYSLFYNFF

SEQ ID NO: 205 - protein, 38 aa, Artificial
  Synthetic peptide.
RRSLGNSASFAEWIEYIRYLHYIIRVQFIHFFSKNKKI

SEQ ID NO: 206 - protein, 38 aa, Artificial
  Synthetic peptide.
KLQEKQIDRNFERVSGYSTYRAVQAAKAKEKGFISLEN

SEQ ID NO: 207 - protein, 44 aa, Artificial
  Synthetic peptide.
IFKLFEEHLLYLLDAFYYSKIFRRLKQGLYRRKEQPYTQDLFRM

SEQ ID NO: 208 - protein, 41 aa, Artificial
  Synthetic peptide.
EFLEKFKVLKQPRKANNISKNRVAMIFLTIHKSRGFLSSPY

SEQ ID NO: 209 - protein, 41 aa, Artificial
  Synthetic peptide.
TDQELEHLIVTELESKRLDFTYSKDITEFFDEAFPEYDQNY

SEQ ID NO: 210 - protein, 41 aa, Artificial
  Synthetic peptide.
DNFYLILKMEERGKSKKTSQTRGFRAFFDIIRKKIKKEDGK

SEQ ID NO: 211 - protein, 21 aa, Artificial
  Synthetic peptide.
EDPVPNHFTLRRNKKEKPSKS

SEQ ID NO: 212 - protein, 35 aa, Artificial
  Synthetic peptide.
IFNRRKFFQYFGLSKEAMVEHIQPFILDIWQIHLF

SEQ ID NO: 213 - protein, 29 aa, Artificial
  Synthetic peptide.
ADDLLNKRLTDLIMENAETVKTIDLDNSD

SEQ ID NO: 214 - protein, 40 aa, Artificial
  Synthetic peptide.
VILGNGISNIAQTLGQLPNIAWVWIYMVLIAALLEESNVC

SEQ ID NO: 215 - protein, 30 aa, Artificial
  Synthetic peptide.
KQVQNTTLIICGTVLLGILFKSYLKSQKSV

SEQ ID NO: 216 - protein, 36 aa, Artificial
  Synthetic peptide.
SENIARFAAAFENEQVVSYARWFRRSWRGSGSSSRF

SEQ ID NO: 217 - protein, 9 aa, Artificial
  Synthetic peptide.
IGGALNSCG

SEQ ID NO: 218 - protein, 52 aa, Artificial
  Synthetic peptide.
VFSVLKHTTWPTRKQSWHDFISILEYSAFFALVIFIFDKLLTLGLAELLKRF

SEQ ID NO: 219 - protein, 38 aa, Artificial
  Synthetic peptide.
LVQGDTILIENHVGTPVKDDGKDCLIIREADVLAVVND

SEQ ID NO: 220 - protein, 30 aa, Artificial
  Synthetic peptide.
MKKNLKRFYALVLGFIIGCLFVSILIFIGY

SEQ ID NO: 221 - protein, 41 aa, Artificial
  Synthetic peptide.
KTKESLTQQEKKFLKDYDRKSLHHFRDILTYCFILDKLTNK

SEQ ID NO: 222 - protein, 20 aa, Artificial
  Synthetic peptide.
KGKSLMPLLKQINQWGKLYL

SEQ ID NO: 223 - protein, 48 aa, Artificial
  Synthetic peptide.
IILAKAADLAEIERIISEDPFKINEIANYDIIEFCPTKSSKAFEKVLK

SEQ ID NO: 224 - protein, 29 aa, Artificial
  Synthetic peptide.
TINIDDKVLDYLKKINSKAITIDLIGCAS

SEQ ID NO: 225 - protein, 20 aa, Artificial
  Synthetic peptide.
EKLKKILLKLAVCGKAWYTL

SEQ ID NO: 226 - protein, 32 aa, Artificial
  Synthetic peptide.
NILYFIHDENQWEPQKAEIFRGSIKHCAWLSS

SEQ ID NO: 227 - protein, 29 aa, Artificial
  Synthetic peptide.
SFEKNKIENNLKIAQAYIYIKPKPRICQA

SEQ ID NO: 228 - protein, 12 aa, Artificial
  Synthetic peptide.
LSLPLIVLTKSI

SEQ ID NO: 229 - protein, 23 aa, Artificial
  Synthetic peptide.
FIAVSFTGNPATFKLVIGCKADN

SEQ ID NO: 230 - protein, 22 aa, Artificial
  Synthetic peptide.
LEGKFYMAEDFDKTPECFKDYV

SEQ ID NO: 231 - protein, 30 aa, Artificial
  Synthetic peptide.
GMFENLLMINFQIMNDLKIEIVVKDRICAV

SEQ ID NO: 232 - protein, 12 aa, Artificial
  Synthetic peptide.
RAGTWLVVDEIR

SEQ ID NO: 233 - protein, 26 aa, Artificial
  Synthetic peptide.
RIKEERKNRSYKFFIWRLFDEKTGFI

SEQ ID NO: 234 - protein, 26 aa, Artificial
  Synthetic peptide.
PITPKKEKCGLGTYAPKNPVFSKSRV

SEQ ID NO: 235 - protein, 16 aa, Artificial
  Synthetic peptide.
PLYVAAVEKINTAKKH

SEQ ID NO: 236 - protein, 13 aa, Artificial
  Synthetic peptide.
VHEFDIQKILQNR

SEQ ID NO: 237 - protein, 29 aa, Artificial
  Synthetic peptide.
FLIQKFLLIKTFPPYRKKYVVIVSQTGTA

SEQ ID NO: 238 - protein, 31 aa, Artificial
  Synthetic peptide.
QLAPIDKQLKAVKKIAFYESESTAAKAVTVA

SEQ ID NO: 239 - protein, 31 aa, Artificial
  Synthetic peptide.
YNEPNYKWLESYKIYKQRCEDRTGMYYTEET

SEQ ID NO: 240 - protein, 26 aa, Artificial
  Synthetic peptide.
ETTTEINAIKLHRIKQRSPQGTRRVN

SEQ ID NO: 241 - protein, 30 aa, Artificial
  Synthetic peptide.
QVLKNFSISRRYKINNPFFKILLFIQLRTL

SEQ ID NO: 242 - protein, 24 aa, Artificial
  Synthetic peptide.
ILTLLILGSIGFFILKIKLKLGRF

SEQ ID NO: 243 - protein, 18 aa, Artificial
  Synthetic peptide.
IYYMRFVNKPLEKTFFKI

SEQ ID NO: 244 - protein, 26 aa, Artificial
  Synthetic peptide.
SINSSAGIQPHCLSSSFVLRTKHCFY

SEQ ID NO: 245 - protein, 10 aa, Artificial
  Synthetic peptide.
FVLRTKHCFY

SEQ ID NO: 246 - protein, 29 aa, Artificial
  Synthetic peptide.
TNNKNKVIIKAIKFKNKDFINLDLFIYRR

SEQ ID NO: 247 - protein, 28 aa, Artificial
  Synthetic peptide.
KYEKLTKENLFIRNSGNMCVFIYFLFFG

SEQ ID NO: 248 - protein, 9 aa, Artificial
  Synthetic peptide.
ISLVFPAYT

SEQ ID NO: 249 - protein, 32 aa, Artificial
  Synthetic peptide.
LCTKLEDKQRGRIPAELFIISPIKILERNDAL

SEQ ID NO: 250 - protein, 10 aa, Artificial
  Synthetic peptide.
FQYYFSLKRV

SEQ ID NO: 251 - protein, 31 aa, Artificial
  Synthetic peptide.
FFPYYLADFYKQLKFLNEYQTKNKDKVVEFK

SEQ ID NO: 252 - protein, 29 aa, Artificial
  Synthetic peptide.
LGFFNNKADLVKADTERDNRMSSLKIKDL

SEQ ID NO: 253 - protein, 14 aa, Artificial
  Synthetic peptide.
KGYPLPFQYRLNNH

SEQ ID NO: 254 - protein, 20 aa, Artificial
  Synthetic peptide.
RWVGGEPSADIYLSAKDTKT

SEQ ID NO: 255 - protein, 15 aa, Artificial
  Synthetic peptide.
EPSADIYLSAKDTKT

SEQ ID NO: 256 - protein, 18 aa, Artificial
  Synthetic peptide.
IINQLNLILLRLMEILIL

SEQ ID NO: 257 - protein, 31 aa, Artificial
  Synthetic peptide.
DMKIIKLYIKILSFLFIKYCNKKLNSVKLKA

SEQ ID NO: 258 - protein, 18 aa, Artificial
  Synthetic peptide.
IINQLNLILLRLMEILIL

SEQ ID NO: 259 - protein, 32 aa, Artificial
  Synthetic peptide.
HVEDCFLLSNARTTAIHGRANPARGEPRTRSE

SEQ ID NO: 260 - protein, 17 aa, Artificial
  Synthetic peptide.
YDKIADGVFKIGKRGVL

SEQ ID NO: 261 - protein, 9 aa, Artificial
  Synthetic peptide.
KYKLKKIIL

SEQ ID NO: 262 - protein, 20 aa, Artificial
  Synthetic peptide.
EYSQQSFKAKPCSERGVLSP

SEQ ID NO: 263 - protein, 32 aa, Artificial
  Synthetic peptide.
RSLRLNNALTKLPKLWYNRIKEAFYAYNDYDK

SEQ ID NO: 264 - protein, 29 aa, Artificial
  Synthetic peptide.
ILNKKPKLPLWKLGKNYFRRFYVLPTFLA

SEQ ID NO: 265 - protein, 23 aa, Artificial
  Synthetic peptide.
SMLTSFLRSKNTRSLKMYKDVHF

SEQ ID NO: 266 - protein, 29 aa, Artificial
  Synthetic peptide.
PLIISKAQIKMSGDILGSCFKLFYLRPFF

SEQ ID NO: 267 - protein, 13 aa, Artificial
  Synthetic peptide.
SKLPRVLDASLKL

SEQ ID NO: 268 - protein, 15 aa, Artificial
  Synthetic peptide.
IIIILPKIYLVCKTV

SEQ ID NO: 269 - protein, 14 aa, Artificial
  Synthetic peptide.
LDYENMDCKKRIRI

SEQ ID NO: 270 - protein, 24 aa, Artificial
  Synthetic peptide.
STAGEASRRTASEASRRTAAKLRG

SEQ ID NO: 271 - protein, 26 aa, Artificial
  Synthetic peptide.
ARNALNMRDVPVDAAIIGIIDGMDEE

SEQ ID NO: 272 - protein, 25 aa, Artificial
  Synthetic peptide.
KILNEAEGKLLKVIEKNGEIDIEEI

SEQ ID NO: 273 - protein, 24 aa, Artificial
  Synthetic peptide.
NGDKKAKEELDKWDEVIKELNIQF

SEQ ID NO: 274 - protein, 34 aa, Artificial
  Synthetic peptide.
GLVIIPNLIALIILFSQVRQQTKDYFSNPKLSSR

SEQ ID NO: 275 - protein, 34 aa, Artificial
  Synthetic peptide.
EPLPLTKYDKKDTEMKKVFKEILAGKVGYEKEEE

SEQ ID NO: 276 - protein, 23 aa, Artificial
  Synthetic peptide.
TKLKKNNKLLSAKKENTLHTKDK

SEQ ID NO: 277 - protein, 9 aa, Artificial
  Synthetic peptide.
AIFDAMHNL

SEQ ID NO: 278 - protein, 5 aa, Artificial
  Synthetic peptide.
HSSHL

SEQ ID NO: 279 - protein, 6 aa, Artificial
  Synthetic peptide.
DLRKAK

SEQ ID NO: 280 - protein, 5 aa, Artificial
  Synthetic peptide.
LVRLA

SEQ ID NO: 281 - protein, 17 aa, Artificial
  Synthetic peptide.
EVYSSPTNNVAITVQNN

SEQ ID NO: 282 - protein, 32 aa, Artificial
  Synthetic peptide.
SKFELVNYASGCSCGADCKCASETECKCASKK

SEQ ID NO: 283 - protein, 28 aa, Artificial
  Synthetic peptide.
GVGIGFIMMGVVGYAVKLVHIPIRYLIV

SEQ ID NO: 284 - protein, 27 aa, Artificial
  Synthetic peptide.
HARAAVGVAELPRGAAVEVELIAAVRP

SEQ ID NO: 285 - protein, 8 aa, Artificial
  Synthetic peptide.
VVRRFQGM

SEQ ID NO: 286 - protein, 26 aa, Artificial
  Synthetic peptide.
NILFGIIGFVVAMTAAVIVTAISIAK

SEQ ID NO: 287 - protein, 30 aa, Artificial
  Synthetic peptide.
MPKARPVNHNKKKSKITIKSNFTLFYMFNP

SEQ ID NO: 288 - protein, 30 aa, Artificial
  Synthetic peptide.
MIHLTKQNTMEALHFIKQFYDMFFILNFNV

SEQ ID NO: 289 - protein, 25 aa, Artificial
  Synthetic peptide.
RFFNFEIKKSTKVDYVFAHVDLSDV

SEQ ID NO: 290 - protein, 33 aa, Artificial
  Synthetic peptide.
EILNNNQVIKELTMKYKTQFESNLGGWTARARR

SEQ ID NO: 291 - protein, 28 aa, Artificial
  Synthetic peptide.
KFQGEFTNIGQSYIVSASHMSTSLNTGK

SEQ ID NO: 292 - protein, 20 aa, Artificial
  Synthetic peptide.
VYRHLRFIDGKLVEIRLERK

SEQ ID NO: 293 - protein, 24 aa, Artificial
  Synthetic peptide.
KLRSASKKSLQEKSCGIMPEKPAG

SEQ ID NO: 294 - protein, 20 aa, Artificial
  Synthetic peptide.
FRSPCINNNSLQPPGVYPAR

SEQ ID NO: 295 - protein, 30 aa, Artificial; identical to SEQ ID NO: 18
  Synthetic peptide.

SEQ ID NO: 296 - protein, 16 aa, Artificial
  Synthetic peptide.
MTCHQAPTTTHQSNMA

SEQ ID NO: 297 - protein, 30 aa, Artificial
  Synthetic peptide.
MVILVFSLIFIFTDNYLVYQSKSIKEDVMI

SEQ ID NO: 298 - protein, 16 aa, Artificial
  Synthetic peptide.
KKIIPLITLFVVTLVG

SEQ ID NO: 299 - protein, 21 aa, Artificial
  Synthetic peptide.
DKSTQDKDIKQAKLLAQELGL

SEQ ID NO: 300 - protein, 25 aa, Artificial
  Synthetic peptide.
ISLIIFIMLFVVALFKCITNYKHQS

SEQ ID NO: 301 - protein, 10 aa, Artificial
  Synthetic peptide.
GMPQIPRLRI

SEQ ID NO: 302 - protein, 4 aa, Artificial
  Synthetic peptide.
IDMR

SEQ ID NO: 303 - protein, 26 aa, Artificial
  Synthetic peptide.
DSFDSLSPFRERGGEREDGCDAMPLP

SEQ ID NO: 304 - protein, 5 aa, Artificial
  Synthetic peptide.
NDDAQ

SEQ ID NO: 305 - protein, 32 aa, Artificial
  Synthetic peptide.
ALALLKQDLLNFEGRGRIITSTYLQFNEGCVP

SEQ ID NO: 306 - protein, 14 aa, Artificial
  Synthetic peptide.
FSLNFSKQKYVTVN

SEQ ID NO: 307 - protein, 12 aa, Artificial
  Synthetic peptide.
ALAGLAGLISGK

SEQ ID NO: 308 - protein, 9 aa, Artificial
  Synthetic peptide.
DVILRVEAQ

SEQ ID NO: 309 - protein, 12 aa, Artificial
  Synthetic peptide.
GLAAIATVFALY

SEQ ID NO: 310 - protein, 8 aa, Artificial
  Synthetic peptide.
PMNAAEPE

SEQ ID NO: 311 - protein, 7 aa, Artificial
  Synthetic peptide.
PPSSFLV

SEQ ID NO: 312 - protein, 18 aa, Artificial
  Synthetic peptide.
LIIYFSKTGNTARATRQI

SEQ ID NO: 313 - protein, 13 aa, Artificial
  Synthetic peptide.
SKKYNHILNQENR

SEQ ID NO: 314 - protein, 8 aa, Artificial
  Synthetic peptide.
NNAIVYIS

SEQ ID NO: 315 - protein, 20 aa, Artificial
  Synthetic peptide.
PALVDLSNKEAVWAVLDDHS

SEQ ID NO: 316 - protein, 11 aa, Artificial
  Synthetic peptide.
GGTKEIVYQRG

SEQ ID NO: 317 - protein, 15 aa, Artificial
  Synthetic peptide.
NRQAQGERAHGEQQG

SEQ ID NO: 318 - protein, 20 aa, Artificial
  Synthetic peptide.
AIEGVIKKGACFKLLRHEMF

SEQ ID NO: 319 - protein, 28 aa, Artificial
  Synthetic peptide.
VLPFPAIPLSRRRACVAAPRPRSRQRAS

SEQ ID NO: 320 - protein, 15 aa, Artificial
  Synthetic peptide.
RLARGRPTNLCGRRG

SEQ ID NO: 321 - protein, 19 aa, Artificial
  Synthetic peptide.
RLTSNQFLTRITPFVFAQH

SEQ ID NO: 322 - protein, 25 aa, Artificial
  Synthetic peptide.
VRQVPVDRPESRHQKPGDVPRDPRC

SEQ ID NO: 323 - protein, 32 aa, Artificial
  Synthetic peptide.
QHQCPGMRPAPADAPEVPHAARADQKRPSLRL

SEQ ID NO: 324 - protein, 32 aa, Artificial
  Synthetic peptide.
DPLVDGAARLLSIPLRHLYAALWRVGLLEVQA

SEQ ID NO: 325 - protein, 24 aa, Artificial
  Synthetic peptide.
RSPDFVDETAGQSWCAILGLNQFH

SEQ ID NO: 326 - protein, 28 aa, Artificial
  Synthetic peptide.
GLITVFAGTARILQLRRAAKKTHAAALR

SEQ ID NO: 327 - protein, 23 aa, Artificial
  Synthetic peptide.
SKRGRKRKDRRKKKANHGKRPNS

SEQ ID NO: 328 - protein, 30 aa, Artificial
  Synthetic peptide.
MNNWIIVAQLSVTVINEIIDIMKEKQKGGK

SEQ ID NO: 329 - protein, 5 aa, Artificial
  Synthetic peptide.
NDDAQ

SEQ ID NO: 330 - protein, 30 aa, Artificial
  Synthetic peptide.
MNNWIKVAQISVTVINEVIDIMKEKQNGGK

SEQ ID NO: 331 - protein, 23 aa, Artificial
  Synthetic peptide.
ARLSKAIIIAVIVVYHLDVRGLF

SEQ ID NO: 332 - protein, 30 aa, Artificial
  Synthetic peptide.
MESIFKIKLMNGICRSENMNMKKKNKGEKI

SEQ ID NO: 333 - protein, 21 aa, Artificial
  Synthetic peptide.
MGIIAGIIKFIKGLIEKFTGK

SEQ ID NO: 334 - protein, 21 aa, Artificial
  Synthetic peptide.
MGIIAGIIKVIKSLIEQFTGK

SEQ ID NO: 335 - protein, 26 aa, Artificial
  Synthetic peptide.
MIEIGSIAYLNGGSKKYNHILNQENR

SEQ ID NO: 336 - protein, 13 aa, Artificial
  Synthetic peptide.
SKKYNHILNQENR

SEQ ID NO: 337 - protein, 26 aa, Artificial
  Synthetic peptide.
MDIDVNKLLQAFVYFKSFEKLRHNNS

SEQ ID NO: 338 - protein, 30 aa, Artificial
  Synthetic peptide.
MFCYYKQHKGDNFSIEEVKNIIADNEMKVN

SEQ ID NO: 339 - protein, 24 aa, Artificial
  Synthetic peptide.
WRGPNTEAGGKSANNIVQVGGAPT

SEQ ID NO: 340 - protein, 23 aa, Artificial
  Synthetic peptide.
LIQKGLNQTFIVVIRLNNFIKKS

SEQ ID NO: 341 - protein, 8 aa, Artificial
  Synthetic peptide.
HPTDNKHN

SEQ ID NO: 342 - protein, 14 aa, Artificial
  Synthetic peptide.
SIDKRNLYNLKYYE

SEQ ID NO: 343 - protein, 13 aa, Artificial
  Synthetic peptide.
RKQYDDLSFNFLY

SEQ ID NO: 344 - protein, 5 aa, Artificial
  Synthetic peptide.
ESIIE

SEQ ID NO: 345 - protein, 9 aa, Artificial
  Synthetic peptide.
YYKTYFKEV

SEQ ID NO: 346 - protein, 30 aa, Artificial
  Synthetic peptide.
MKIILLLFLIFGFIVVVTLKSEHQLTLFSI

SEQ ID NO: 347 - protein, 14 aa, Artificial
  Synthetic peptide.
FSLNFSKQKYVTVN

SEQ ID NO: 348 - protein, 24 aa, Artificial
  Synthetic peptide.
MINELKNKNSGIMNNYVVTKESKL

SEQ ID NO: 349 - protein, 30 aa, Artificial
  Synthetic peptide.
MTKNTIISLENEKTQINDSENESSDLRKAK

SEQ ID NO: 350 - protein, 6 aa, Artificial
  Synthetic peptide.
DLRKAK

SEQ ID NO: 351 - protein, 17 aa, Artificial
  Synthetic peptide.
LLIIFRLWLELKWKNKK

SEQ ID NO: 352 - protein, 6 aa, Artificial
  Synthetic peptide.
SIHFIN

SEQ ID NO: 353 - protein, 25 aa, Artificial
  Synthetic peptide.
HNARKYLEFISQKIDGDKLTKEDSL

SEQ ID NO: 354 - protein, 25 aa, Artificial
  Synthetic peptide.
ALDCSEQSVILWYETILDKIVGVIK

SEQ ID NO: 355 - protein, 5 aa, Artificial
  Synthetic peptide.
NSTNE

SEQ ID NO: 356 - protein, 16 aa, Artificial
  Synthetic peptide.
MTCHQAPTTTHQSNMA

SEQ ID NO: 357 - protein, 30 aa, Artificial
  Synthetic peptide.
MPHHSTTSSRIVVPAHQSNMASTPNLSITP

SEQ ID NO: 358 - protein, 30 aa, Artificial
  Synthetic peptide.
MFIFKTTSKSHFHNNVKSLECIKIPINKNR

SEQ ID NO: 359 - protein, 17 aa, Artificial
  Synthetic peptide.
EPKKKHFPKMESASSEP

SEQ ID NO: 360 - protein, 6 aa, Artificial
  Synthetic peptide.
SFYESY

SEQ ID NO: 361 - protein, 21 aa, Artificial
  Synthetic peptide.
ILNRLSRIVSNEVTSLIYSLK

SEQ ID NO: 362 - protein, 30 aa, Artificial
  Synthetic peptide.
MTKKRRYDTTEFGLAHSMTAKITLHQALYK

SEQ ID NO: 363 - protein, 19 aa, Artificial
  Synthetic peptide.
MAYKDEGKETKFAVKGYKD

SEQ ID NO: 364 - protein, 9 aa, Artificial
  Synthetic peptide.
MLEEKNKSL

SEQ ID NO: 365 - protein, 30 aa, Artificial; identical to SEQ ID NO: 288
  Synthetic peptide.

SEQ ID NO: 366 - protein, 10 aa, Artificial
  Synthetic peptide.
ELLVILPGFI

SEQ ID NO: 367 - protein, 17 aa, Artificial
  Synthetic peptide.
LLLSYFRYTGALLQSLF

SEQ ID NO: 368 - protein, 22 aa, Artificial
  Synthetic peptide.
MIKNETAYQMNELLVIRSAYAK

SEQ ID NO: 369 - protein, 10 aa, Artificial
  Synthetic peptide.
KLKKYIHKPD

SEQ ID NO: 370 - protein, 10 aa, Artificial
  Synthetic peptide.
LDINDYRSTY

SEQ ID NO: 371 - protein, 13 aa, Artificial
  Synthetic peptide.
LDFYLTKHLTLML

SEQ ID NO: 372 - protein, 17 aa, Artificial
  Synthetic peptide.
NQEPSLQQDKEQKDNKG

SEQ ID NO: 373 - protein, 20 aa, Artificial
  Synthetic peptide.
LYFAFKKYQERVNQAPNIEY

SEQ ID NO: 374 - protein, 12 aa, Artificial
  Synthetic peptide.
AYYLKRREEKGK

SEQ ID NO: 375 - protein, 12 aa, Artificial
  Synthetic peptide.
SYYLKRREEKGK

SEQ ID NO: 376 - protein, 25 aa, Artificial
  Synthetic peptide.
RFFNFEIKKSTKVDYVFAHVDLSDV

SEQ ID NO: 377 - protein, 15 aa, Artificial
  Synthetic peptide.
QELINEAVNLLVKSK

SEQ ID NO: 378 - protein, 38 aa, Artificial
  Synthetic peptide.
KLFGQWGPELGSIYILPALIGSIILIAIVTLILRAMRK

SEQ ID NO: 379 - protein, 21 aa, Artificial
  Synthetic peptide.
VSISRFIGGGHVFNGNNKRNL

SEQ ID NO: 380 - protein, 12 aa, Artificial
  Synthetic peptide.
GHVFNGNNKRNL

SEQ ID NO: 381 - protein, 40 aa, Artificial
  Synthetic peptide.
AEQLFGKQKQRGVDLFLNRLTIILSILFFVLMICISYLGM

SEQ ID NO: 382 - protein, 22 aa, Artificial
  Synthetic peptide.
TMIVISIPRFEEYMKARHKKWM

SEQ ID NO: 383 - protein, 9 aa, Artificial
  Synthetic peptide.
FADQSQDNA

SEQ ID NO: 384 - protein, 5 aa, Artificial
  Synthetic peptide.
HSSHL

SEQ ID NO: 385 - protein, 24 aa, Artificial
  Synthetic peptide.
GYNSYKAVQDVKTHSEEQRVTAKK

SEQ ID NO: 386 - protein, 32 aa, Artificial
  Synthetic peptide.
MKKKRINNDILGRMIYSSSIDKRNLYNLKYYE

SEQ ID NO: 387 - protein, 26 aa, Artificial
  Synthetic peptide.
IAAIIVLVLFQKGLLQIFNWILIQLQ

SEQ ID NO: 388 - protein, 6 aa, Artificial
  Synthetic peptide.
DYYGKE

SEQ ID NO: 389 - protein, 35 aa, Artificial
  Synthetic peptide.
LEKNTRDNYFIHAIDRIYINTSKGLFPESELVAWG

SEQ ID NO: 390 - protein, 37 aa, Artificial
  Synthetic peptide.
IKGTVKAVDETTVVITVNGHGTELTFEKPAIKQVDPS

SEQ ID NO: 391 - protein, 30 aa, Artificial
  Synthetic peptide.
DLIVKVHICFVVKTASGYCYLNKREAQAAI

SEQ ID NO: 392 - protein, 37 aa, Artificial
  Synthetic peptide.
SHLINNFGLSVINPSTPICLNFSPVFNLLTVYGITCN

SEQ ID NO: 393 - protein, 21 aa, Artificial
  Synthetic peptide.
FDPVPLKKDKSASKHSHKHNH

SEQ ID NO: 394 - protein, 18 aa, Artificial
  Synthetic peptide.
SMVKSEIVDLLNGEDNDD

SEQ ID NO: 395 - protein, 30 aa, Artificial
  Synthetic peptide.
HCVIGNVVDIANLLKRRAVYRDIADVIKMR

SEQ ID NO: 396 - protein, 36 aa, Artificial
  Synthetic peptide.
CPSVTMDACALLQKFDFCNNISHFRHFFAIKQPIER

SEQ ID NO: 397 - protein, 12 aa, Artificial
  Synthetic peptide.
RDIHPIYFMTKD

SEQ ID NO: 398 - protein, 28 aa, Artificial
  Synthetic peptide.
FVNSLIMKDLSDNDMRFKYEYYNREKDT

SEQ ID NO: 399 - protein, 27 aa, Artificial
  Synthetic peptide.
LYQYELLSKEEYLKCTLIINQRRNEQK

SEQ ID NO: 400 - protein, 22 aa, Artificial
  Synthetic peptide.
QPTQGEQGTRPRRPTPMRGLLI

SEQ ID NO: 401 - protein, 13 aa, Artificial
  Synthetic peptide.
EIIAYLEGRFANA

SEQ ID NO: 402 - protein, 21 aa, Artificial
  Synthetic peptide.
DPVPERQEQACACHRTAKPGK

SEQ ID NO: 403 - protein, 10 aa, Artificial
  Synthetic peptide.
ERTAVNDLWI

SEQ ID NO: 404 - protein, 28 aa, Artificial
  Synthetic peptide.
TTRPQVAEDRQLDDALKETFPASDPISP

SEQ ID NO: 405 - protein, 38 aa, Artificial
  Synthetic peptide.
MADGQIAAIAKLHGVPVATRNIRHFQSFGVELINPWSG

SEQ ID NO: 406 - protein, 22 aa, Artificial
  Synthetic peptide.
YVVGALVILAVAGLIYSMLRKA

SEQ ID NO: 407 - protein, 34 aa, Artificial
  Synthetic peptide.
FSPEAFGIGAAGVLGSFVTGLLIGWVASLLRKAK

SEQ ID NO: 408 - protein, 30 aa, Artificial
  Synthetic peptide.
MLRYLSLFAVGLATGYAWGWIDGLAASLAV

SEQ ID NO: 409 - protein, 24 aa, Artificial
  Synthetic peptide.
GIKVVAARFEEIQFSENFDSIILA

SEQ ID NO: 410 - protein, 17 aa, Artificial
  Synthetic peptide.
MKLLARDPWVCAWNDIW

SEQ ID NO: 411 - protein, 17 aa, Artificial
  Synthetic peptide.
LQRSDEESMPRRHEKYS

SEQ ID NO: 412 - protein, 12 aa, Artificial
  Synthetic peptide.
RRAAARTKGNRR

SEQ ID NO: 413 - protein, 13 aa, Artificial
  Synthetic peptide.
RPGDGAAEQGRSR

SEQ ID NO: 414 - protein, 13 aa, Artificial
  Synthetic peptide.
GDPTAGQKPVECP

SEQ ID NO: 415 - protein, 12 aa, Artificial
  Synthetic peptide.
GKAMKRQDCSAL

SEQ ID NO: 416 - protein, 24 aa, Artificial
  Synthetic peptide.
PPARPARIPQTPTLHGASLFRQRS

SEQ ID NO: 417 - protein, 15 aa, Artificial
  Synthetic peptide.
LRGRVGRITACGYPP

SEQ ID NO: 418 - protein, 27 aa, Artificial
  Synthetic peptide.
VLGKGHDLLDVGKTALKSRVFAWLGGS

SEQ ID NO: 419 - protein, 9 aa, Artificial
  Synthetic peptide.
AVHHSLLFR

SEQ ID NO: 420 - protein, 19 aa, Artificial
  Synthetic peptide.
ALSKPAIQARTLCRRQDPP

SEQ ID NO: 421 - protein, 27 aa, Artificial
  Synthetic peptide.
FHRRVIRASEWALTTRSFSTPLRSAAR

SEQ ID NO: 422 - protein, 8 aa, Artificial
  Synthetic peptide.
VVRRFQGM

SEQ ID NO: 423 - protein, 10 aa, Artificial
  Synthetic peptide.
GIDRGCQAAR

SEQ ID NO: 424 - protein, 22 aa, Artificial
  Synthetic peptide.
LSPRPIIVSRRSRADNNNDWSR

SEQ ID NO: 425 - protein, 16 aa, Artificial
  Synthetic peptide.
RSGQPVGRPSRRAWLR

SEQ ID NO: 426 - protein, 24 aa, Artificial
  Synthetic peptide.
GIVLTGRAGLVSGACSMALGVGLG

SEQ ID NO: 427 - protein, 17 aa, Artificial
  Synthetic peptide.
GCGKRRIITKSASRDTR

SEQ ID NO: 428 - protein, 17 aa, Artificial
  Synthetic peptide.
RRPRRRRSGHGQSASAA

SEQ ID NO: 429 - protein, 25 aa, Artificial
  Synthetic peptide.
RRGCTERLRRMARRNAWDLYAEHFY

SEQ ID NO: 430 - protein, 20 aa, Artificial
  Synthetic peptide.
GKVSVLTRVPRSLGGAPANQ

SEQ ID NO: 431 - protein, 8 aa, Artificial
  Synthetic peptide.
EIQAKGTG

SEQ ID NO: 432 - protein, 20 aa, Artificial
  Synthetic peptide.
EEYPARVPLSGEDVTEARRH

SEQ ID NO: 433 - protein, 14 aa, Artificial
  Synthetic peptide.
VGYFIWKDSHSRKG

SEQ ID NO: 434 - protein, 12 aa, Artificial
  Synthetic peptide.
GILARADCSQIA

SEQ ID NO: 435 - protein, 22 aa, Artificial
  Synthetic peptide.
GIKKSKHPSTDDYVVKTTIDSL

SEQ ID NO: 436 - protein, 15 aa, Artificial
  Synthetic peptide.
GRYGDDSKERQGRAQ

SEQ ID NO: 437 - protein, 15 aa, Artificial
  Synthetic peptide.
LITAEQPATAPIAGK

SEQ ID NO: 438 - protein, 22 aa, Artificial
  Synthetic peptide.
HTAVVWLAGVSGCVALSHCEPA

SEQ ID NO: 439 - protein, 12 aa, Artificial
  Synthetic peptide.
VRLESRPADLPE

SEQ ID NO: 440 - protein, 20 aa, Artificial
  Synthetic peptide.
TMAFVEKAQLRVPVGDDLPV

SEQ ID NO: 441 - protein, 17 aa, Artificial
  Synthetic peptide.
SFHASLTKNEKPIKSTG

SEQ ID NO: 442 - protein, 18 aa, Artificial
  Synthetic peptide.
RGRALASTATTRPARRRR

SEQ ID NO: 443 - protein, 23 aa, Artificial
  Synthetic peptide.
GIRRLHSVENLNREISHRMAGLR

SEQ ID NO: 444 - protein, 26 aa, Artificial
  Synthetic peptide.
TSWLRAAERQEIGEPTKTFGEKTTSL

SEQ ID NO: 445 - protein, 19 aa, Artificial
  Synthetic peptide.
EEVSRALAGIGLGLGCRIG

SEQ ID NO: 446 - protein, 28 aa, Artificial
  Synthetic peptide.
GPVSVVASLRRGTTVQRHSQNNHNKGKP

SEQ ID NO: 447 - protein, 10 aa, Artificial
  Synthetic peptide.
SKAVSRKRSI

SEQ ID NO: 448 - protein, 20 aa, Artificial
  Synthetic peptide.
AIEGVIKKGACFKLLRHEMF

SEQ ID NO: 449 - protein, 28 aa, Artificial
  Synthetic peptide.
VLPFPAIPLSRRRACVAAPRPRSRQRAS

SEQ ID NO: 450 - protein, 15 aa, Artificial
  Synthetic peptide.
APGSAADSPRSRADD

SEQ ID NO: 451 - protein, 15 aa, Artificial
  Synthetic peptide.
RLARGRPTNLCGRRG

SEQ ID NO: 452 - protein, 9 aa, Artificial
  Synthetic peptide.
TQVTLCRTW

SEQ ID NO: 453 - protein, 21 aa, Artificial
  Synthetic peptide.
LTGVRRPWRAPWAGTSGWALR

SEQ ID NO: 454 - protein, 11 aa, Artificial
  Synthetic peptide.
AGRTAIVQGGG

SEQ ID NO: 455 - protein, 20 aa, Artificial
  Synthetic peptide.
RGGDSPARRRPGLAGPGGPG

SEQ ID NO: 456 - protein, 19 aa, Artificial
  Synthetic peptide.
RRRPAGQRPEKASQAMIAA

SEQ ID NO: 457 - protein, 19 aa, Artificial
  Synthetic peptide.
RLTSNQFLTRITPFVFAQH

SEQ ID NO: 458 - protein, 21 aa, Artificial
  Synthetic peptide.
VTSEPGIAHDIRLLPRAAAFR

SEQ ID NO: 459 - protein, 17 aa, Artificial
  Synthetic peptide.
EVYSSPTNNVAITVQNN

SEQ ID NO: 460 - protein, 13 aa, Artificial
  Synthetic peptide.
SGLGDLGFSSEAK

SEQ ID NO: 461 - protein, 15 aa, Artificial
  Synthetic peptide.
GIAPRRNEWGAVGGR

SEQ ID NO: 462 - protein, 18 aa, Artificial
  Synthetic peptide.
LPATRDKTRVPASVAGAP

SEQ ID NO: 463 - protein, 10 aa, Artificial
  Synthetic peptide.
KPGISVENRQ

SEQ ID NO: 464 - protein, 9 aa, Artificial
  Synthetic peptide.
LIADRHIRA

SEQ ID NO: 465 - protein, 20 aa, Artificial
  Synthetic peptide.
RPAQARQGPGGLIADRHIRA

SEQ ID NO: 466 - protein, 20 aa, Artificial
  Synthetic peptide.
DADKNLSLERDRFAWRVAAP

SEQ ID NO: 467 - protein, 26 aa, Artificial
  Synthetic peptide.
EIQKIAKGVSGQVYGPSRQITISKKR

SEQ ID NO: 468 - protein, 22 aa, Artificial
  Synthetic peptide.
ARTFAGRLGTRYFGGLMRSTKA

SEQ ID NO: 469 - protein, 14 aa, Artificial
  Synthetic peptide.
GNLTRSREAARATQ

SEQ ID NO: 470 - protein, 23 aa, Artificial
  Synthetic peptide.
HFILRKPLLFMIHSLKTGPLDRF

SEQ ID NO: 471 - protein, 21 aa, Artificial
  Synthetic peptide.
QFCNFAWLFLASNNAQVSALA

SEQ ID NO: 472 - protein, 11 aa, Artificial
  Synthetic peptide.
VEEDEAPPPHY

SEQ ID NO: 473 - protein, 14 aa, Artificial
  Synthetic peptide.
PPHCPPGHAKKGWC

SEQ ID NO: 474 - protein, 21 aa, Artificial
  Synthetic peptide.
MKGNKLATAHEQPVKNSAPPL

SEQ ID NO: 475 - protein, 18 aa, Artificial
  Synthetic peptide.
EMAEGSADDRLRKTPRDC

SEQ ID NO: 476 - protein, 20 aa, Artificial
  Synthetic peptide.
TTARYIRRQCHTSITPLSQG

SEQ ID NO: 477 - protein, 14 aa, Artificial
  Synthetic peptide.
CNALLRRGHPPSAL

SEQ ID NO: 478 - protein, 18 aa, Artificial
  Synthetic peptide.
GIELKSLIMAQIERWRQA

SEQ ID NO: 479 - protein, 15 aa, Artificial
  Synthetic peptide.
GCRPASLSDADPDGR

SEQ ID NO: 480 - protein, 13 aa, Artificial
  Synthetic peptide.
ALNRASLRLALGE

SEQ ID NO: 481 - protein, 9 aa, Artificial
  Synthetic peptide.
SWKCHHLAI

SEQ ID NO: 482 - protein, 23 aa, Artificial
  Synthetic peptide.
ALQKQDMNLPSVKNQLVFLKSTG

SEQ ID NO: 483 - protein, 16 aa, Artificial
  Synthetic peptide.
AGVLETPRCRGEYGAN

SEQ ID NO: 484 - protein, 24 aa, Artificial
  Synthetic peptide.
KLRSASKKSLQEKSCGIMPEKPAG

SEQ ID NO: 485 - protein, 18 aa, Artificial
  Synthetic peptide.
AAGCRDLGSLSSLVTNPS

SEQ ID NO: 486 - protein, 23 aa, Artificial
  Synthetic peptide.
DAYHCHLVRSPDAHDLSMRIGFV

SEQ ID NO: 487 - protein, 8 aa, Artificial
  Synthetic peptide.
NYAVVSHT

SEQ ID NO: 488 - protein, 12 aa, Artificial
  Synthetic peptide.
EREDGCDAMPLP

SEQ ID NO: 489 - protein, 26 aa, Artificial
  Synthetic peptide.
DSFDSLSPFRERGGEREDGCDAMPLP

SEQ ID NO: 490 - protein, 7 aa, Artificial
  Synthetic peptide.
NDSKASN

SEQ ID NO: 491 - protein, 12 aa, Artificial
  Synthetic peptide.
MTTGVDFIIEKV

SEQ ID NO: 492 - protein, 23 aa, Artificial
  Synthetic peptide.
GHLRVCWVFSASLLTPFRSATLI

SEQ ID NO: 493 - protein, 25 aa, Artificial
  Synthetic peptide.
ELKITNYNVNTVLYRYYKWGNDLCE

SEQ ID NO: 494 - protein, 20 aa, Artificial
  Synthetic peptide.
ESVDKITEALEEDGFPAKVQ

SEQ ID NO: 495 - protein, 13 aa, Artificial
  Synthetic peptide.
DWEFTHKTIPQKK

SEQ ID NO: 496 - protein, 21 aa, Artificial
  Synthetic peptide.
SETPEKPVGTFFYSIYYKIIL

SEQ ID NO: 497 - protein, 23 aa, Artificial
  Synthetic peptide.
FLALAVIAGLFKVILIYAAPYLK

SEQ ID NO: 498 - protein, 9 aa, Artificial
  Synthetic peptide.
VFDNIDINF

SEQ ID NO: 499 - protein, 6 aa, Artificial
  Synthetic peptide.
HIKETR

SEQ ID NO: 500 - protein, 16 aa, Artificial
  Synthetic peptide.
VKFCIECQTKLERKRR

SEQ ID NO: 501 - protein, 13 aa, Artificial
  Synthetic peptide.
DYFYITLSQKNTF

SEQ ID NO: 502 - protein, 16 aa, Artificial
  Synthetic peptide.
MNCASPEFKKLMELYK

SEQ ID NO: 503 - protein, 38 aa, Artificial
  Synthetic peptide.
LMFFSENMDKRDTLSGKFRYFAGSKVIKLMNWLSENGK

SEQ ID NO: 504 - protein, 11 aa, Artificial
  Synthetic peptide.
NQLGSQAFAQL

SEQ ID NO: 505 - protein, 32 aa, Artificial
  Synthetic peptide.
DPILIQIGFTRFALRKAEAEKIEIQVEEGVPA

SEQ ID NO: 506 - protein, 17 aa, Artificial
  Synthetic peptide.
EDKPTNTIQEIKPVKWQ

SEQ ID NO: 507 - protein, 38 aa, Artificial
  Synthetic peptide.
AVRDFKKSVREEDEAASLNSPRTIDAQVKTSESTSVKS

SEQ ID NO: 508 - protein, 18 aa, Artificial
  Synthetic peptide.
FDQLYALEREGKLDELLA

SEQ ID NO: 509 - protein, 27 aa, Artificial
  Synthetic peptide.
DANAMARTTIAIVYILALIALTISYSL

SEQ ID NO: 510 - protein, 8 aa, Artificial
  Synthetic peptide.
RTPYILRS

SEQ ID NO: 511 - protein, 32 aa, Artificial
  Synthetic peptide.
GIPFSKPHKRQVNYMKSDVLAYIEQNKMAHTA

SEQ ID NO: 512 - protein, 28 aa, Artificial
  Synthetic peptide.
KEIRTATVAELNAKRRLTSAEQALAEVS

SEQ ID NO: 513 - protein, 10 aa, Artificial
  Synthetic peptide.
YVKPKVGVHE

SEQ ID NO: 514 - protein, 40 aa, Artificial
  Synthetic peptide.
RNAVVVTEATFPKYEEEITNYLNRRFGEDWSLKLEKCSVA

SEQ ID NO: 515 - protein, 17 aa, Artificial
  Synthetic peptide.
PKHNVVTGVSVDLDYKP

SEQ ID NO: 516 - protein, 13 aa, Artificial
  Synthetic peptide.
RITEVPPDEHSDR

SEQ ID NO: 517 - protein, 33 aa, Artificial
  Synthetic peptide.
KLFEDPLIKSKAVENFQTTWHEQCLAKELAKNM

SEQ ID NO: 518 - protein, 27 aa, Artificial
  Synthetic peptide.
HMRTISYLLAFAKFSLFIPPKQSLKRL

SEQ ID NO: 519 - protein, 41 aa, Artificial
  Synthetic peptide.
MNDVKPVVQPKQTLKAFLVQLLSVRAGVYIKQNNQLPKTKG

SEQ ID NO: 520 - protein, 32 aa, Artificial
  Synthetic peptide.
QPDEKAEFFDPSLDKVYRHPTFYHIPDGIEHM

SEQ ID NO: 521 - protein, 8 aa, Artificial
  Synthetic peptide.
ETAASETH

SEQ ID NO: 522 - protein, 30 aa, Artificial
  Synthetic peptide.
ILSKLWFWMINSLGVVLLVSYWLLAKWGVA

SEQ ID NO: 523 - protein, 9 aa, Artificial
  Synthetic peptide.
INSRYKISF

SEQ ID NO: 524 - protein, 40 aa, Artificial
  Synthetic peptide.
MKKLVAALAVIVILTGCVYDPVNYDKIHDQEFQDHLRQNG

SEQ ID NO: 525 - protein, 6 aa, Artificial
  Synthetic peptide.
VRDDDS

SEQ ID NO: 526 - protein, 37 aa, Artificial
  Synthetic peptide.
FIYGVGFVPHFWLWKWLFSPWIAWPLMLLGYYIWFLT

SEQ ID NO: 527 - protein, 24 aa, Artificial
  Synthetic peptide.
DHKINESQHNPFRSDSNKQNVDFF

SEQ ID NO: 528 - protein, 39 aa, Artificial
  Synthetic peptide.
EYFKQVYVKNEKIYSFWICKDLSPKEAAKRAEDILVKLK

SEQ ID NO: 529 - protein, 38 aa, Artificial
  Synthetic peptide.
VWENRKKYLENEIERHNVFLKLGQEVIKGLNALASRGR

SEQ ID NO: 530 - protein, 35 aa, Artificial
  Synthetic peptide.
LPFSKIGRRVSYKKKDVLKYEQSKTVLNTAQLATV

SEQ ID NO: 531 - protein, 36 aa, Artificial
  Synthetic peptide.
DPHSEIDVTRYCQLHHFTCQTMQISEREFHYLIETQ

SEQ ID NO: 532 - protein, 7 aa, Artificial
  Synthetic peptide.
NLKKCPC

SEQ ID NO: 533 - protein, 41 aa, Artificial
  Synthetic peptide.
MKTLFFPLFLIIFVLIIQALDQSYQKKIGISKPQKHPEFMQ

SEQ ID NO: 534 - protein, 14 aa, Artificial
  Synthetic peptide.
DQEKKNKTEESTEQ

SEQ ID NO: 535 - protein, 7 aa, Artificial
  Synthetic peptide.
SDDKRTD

SEQ ID NO: 536 - protein, 15 aa, Artificial
  Synthetic peptide.
EVLLSDLRPDIFSET

SEQ ID NO: 537 - protein, 42 aa, Artificial
  Synthetic peptide.
MYLTPYAWIAVGSIFAFSVTTIKIGDQNDEKQKSHKNDVHKR

SEQ ID NO: 538 - protein, 9 aa, Artificial
  Synthetic peptide.
AAQPQTTSP

SEQ ID NO: 539 - protein, 23 aa, Artificial
  Synthetic peptide.
LVGALLIFVALIYMVLKGNADKN

SEQ ID NO: 540 - protein, 41 aa, Artificial
  Synthetic peptide.
LVSGVANTVKNTAHTVGNTAKHAGHVAADTTVKATKKQQVK

SEQ ID NO: 541 - protein, 17 aa, Artificial
  Synthetic peptide.
LDLALSTNSLNLEGFSF

SEQ ID NO: 542 - protein, 24 aa, Artificial
  Synthetic peptide.
LSLATFAKIFMTRSNWSLKRFNRL

SEQ ID NO: 543 - protein, 31 aa, Artificial
  Synthetic peptide.
SHIGFISISACLAVLLGIARLFVWTWVKFFA

SEQ ID NO: 544 - protein, 19 aa, Artificial
  Synthetic peptide.
SYNTYYNKLIHGQRTPDGM

SEQ ID NO: 545 - protein, 18 aa, Artificial
  Synthetic peptide.
QNNDTSAWCGSAHKNGNS

SEQ ID NO: 546 - protein, 30 aa, Artificial
  Synthetic peptide.
MIRIRSPTKKKLNRNSISDWKSNTSGRFFY

SEQ ID NO: 547 - protein, 21 aa, Artificial
  Synthetic peptide.
MRYITYSLIPRLLSKKVIHQQ

SEQ ID NO: 548 - protein, 14 aa, Artificial
  Synthetic peptide.
VPAKLLRVIDEIPE

SEQ ID NO: 549 - protein, 13 aa, Artificial
  Synthetic peptide.
IYQLLNIEYSEDD

SEQ ID NO: 550 - protein, 30 aa, Artificial
  Synthetic peptide.
MGRHLWNPSYFVATVSENTEEQIRKYRKNK

SEQ ID NO: 551 - protein, 10 aa, Artificial
  Synthetic peptide.
DVDGAIESEL

SEQ ID NO: 552 - protein, 19 aa, Artificial
  Synthetic peptide.
SFVSTTVRLIFEESKRYKF

SEQ ID NO: 553 - protein, 21 aa, Artificial
  Synthetic peptide.
DFLVNFLWFKGELNWGKKRYK

SEQ ID NO: 554 - protein, 32 aa, Artificial
  Synthetic peptide.
NIQVYESECGNYIFKKSDESFLIDIFDKNGTH

SEQ ID NO: 555 - protein, 28 aa, Artificial
  Synthetic peptide.
ISKGIDDIVYVINKILSIGNIFKIIKRK

SEQ ID NO: 556 - protein, 14 aa, Artificial
  Synthetic peptide.
LATKLKYEKEHKKM

SEQ ID NO: 557 - protein, 15 aa, Artificial
  Synthetic peptide.
VKDVLLELFNKIIGA

SEQ ID NO: 558 - protein, 18 aa, Artificial
  Synthetic peptide.
GIVLIGLKLIPLLANVLN

SEQ ID NO: 559 - protein, 33 aa, Artificial
  Synthetic peptide.
LVKDTSDIKNDLNNIEIVTSKNSNDIAKLKSVK

SEQ ID NO: 560 - protein, 36 aa, Artificial
  Synthetic peptide.
MREWICPSCNETHDRDINASINILKEGLRLITIQNK

SEQ ID NO: 561 - protein, 28 aa, Artificial
  Synthetic peptide.
GCILPHKKDNYNYIMSKFQDLVKITSKK

SEQ ID NO: 562 - protein, 37 aa, Artificial
  Synthetic peptide.
MKRRRCNWCGKLFYLEEKSKEAYCCKECRKKAKKVKK

SEQ ID NO: 563 - protein, 9 aa, Artificial
  Synthetic peptide.
QQYLILDRM

SEQ ID NO: 564 - protein, 22 aa, Artificial
  Synthetic peptide.
GIPGMTAAPAEENEQEENADEE

SEQ ID NO: 565 - protein, 26 aa, Artificial
  Synthetic peptide.
IDAVTKKKTTCMIRAPTKIPIAHTDN

SEQ ID NO: 566 - protein, 32 aa, Artificial
  Synthetic peptide.
YITSHKNARAIIKKFERDEILEEVITHYLNRK

SEQ ID NO: 567 - protein, 37 aa, Artificial
  Synthetic peptide.
ECLKKAIKSKALNKAFKIDVPDEVYDNLLMELEEYEK

SEQ ID NO: 568 - protein, 7 aa, Artificial
  Synthetic peptide.
LILVSDI

SEQ ID NO: 569 - protein, 41 aa, Artificial
  Synthetic peptide.
SIGSMIGMYSFRHKTKHIKFTFGIPFILFLQFLLVYFYILK

SEQ ID NO: 570 - protein, 40 aa, Artificial
  Synthetic peptide.
DSGYYALLENKEERVVWDGEVVANNIFNNLWIVVNKVKTG

SEQ ID NO: 571 - protein, 28 aa, Artificial
  Synthetic peptide.
ARESIEKSHVPVDATIVGVVDSFEVFDE

SEQ ID NO: 572 - protein, 5 aa, Artificial
  Synthetic peptide.
HFSLL

SEQ ID NO: 573 - protein, 11 aa, Artificial
  Synthetic peptide.
LTIDEKLRNHR

SEQ ID NO: 574 - protein, 38 aa, Artificial
  Synthetic peptide.
VIVGNLGAQKEKRNDTPISAKKDIMGDKTVRVRADLHH

SEQ ID NO: 575 - protein, 15 aa, Artificial
  Synthetic peptide.
NGNEKAFSEVENLVK

SEQ ID NO: 576 - protein, 12 aa, Artificial
  Synthetic peptide.
IGILFDKSVRKY

SEQ ID NO: 577 - protein, 20 aa, Artificial
  Synthetic peptide.
YMTKKLVEMAEQQMAGKSNR

SEQ ID NO: 578 - protein, 9 aa, Artificial
  Synthetic peptide.
QQYLILDRM

SEQ ID NO: 579 - protein, 23 aa, Artificial
  Synthetic peptide.
MLTSRKKRLKKIVEEQNKKDESI

SEQ ID NO: 580 - protein, 17 aa, Artificial
  Synthetic peptide.
YMTKKLVEMAERQMAGK

SEQ ID NO: 581 - protein, 17 aa, Artificial
  Synthetic peptide.
KGTSCPDQLSKAIRQSI

SEQ ID NO: 582 - protein, 15 aa, Artificial
  Synthetic peptide.
VKDVLLELFNKIIGA

SEQ ID NO: 583 - protein, 22 aa, Artificial
  Synthetic peptide.
DERLPEAKAIRNFNGSVMVLGR

SEQ ID NO: 584 - protein, 15 aa, Artificial
  Synthetic peptide.
GIFTGVTVVVSLKHC

SEQ ID NO: 585 - protein, 22 aa, Artificial
  Synthetic peptide.
ESASAAEWYNPNMNVKKAICMG

SEQ ID NO: 586 - protein, 30 aa, Artificial
  Synthetic peptide.
MPKSCHVPVLCDFFFLVIIKFLALFKTIQS

SEQ ID NO: 587 - protein, 10 aa, Artificial
  Synthetic peptide.
LAVILRAIVY

SEQ ID NO: 588 - protein, 21 aa, Artificial
  Synthetic peptide.
YLFFKGKKVAEEEATKDEVKR

SEQ ID NO: 589 - protein, 6 aa, Artificial
  Synthetic peptide.
RVKKIG

SEQ ID NO: 590 - protein, 20 aa, Artificial
  Synthetic peptide.
EKTNFKGVKRNFYKKASFFV

SEQ ID NO: 591 - protein, 19 aa, Artificial
  Synthetic peptide.
FTFSKCRASNGRGFGTLWL

SEQ ID NO: 592 - protein, 15 aa, Artificial
  Synthetic peptide.
WIAIGLLLYFSLKNQ

SEQ ID NO: 593 - protein, 21 aa, Artificial
  Synthetic peptide.
VSIKIGAIVIGMIGLMELLTE

SEQ ID NO: 594 - protein, 30 aa, Artificial
  Synthetic peptide.
MLTIIIGFIFWTMTLMLGYLIGEREGRKHE

SEQ ID NO: 595 - protein, 13 aa, Artificial
  Synthetic peptide.
RNTAHNIKWRSKN

SEQ ID NO: 596 - protein, 30 aa, Artificial
  Synthetic peptide.
MTVMEDPGSEQRNKIQSPMKGEDFSALFGR

SEQ ID NO: 597 - protein, 30 aa, Artificial
  Synthetic peptide.
MEQKVKVIFVPRSKPDNQLKTFVSAVLFKA

SEQ ID NO: 598 - protein, 12 aa, Artificial
  Synthetic peptide.
NQVTEGIRLLVE

SEQ ID NO: 599 - protein, 17 aa, Artificial
  Synthetic peptide.
NIERILKEKVWMIRCVE

SEQ ID NO: 600 - protein, 25 aa, Artificial
  Synthetic peptide.
SMLSVTVMCLMHASVAANQAMEKKV

SEQ ID NO: 601 - protein, 7 aa, Artificial
  Synthetic peptide.
LVNGIKI

SEQ ID NO: 602 - protein, 20 aa, Artificial
  Synthetic peptide.
LYKQKIQLEEELEKLKDDRQ

SEQ ID NO: 603 - protein, 19 aa, Artificial
  Synthetic peptide.
ALCSVIKAIELGIINVHLQ

SEQ ID NO: 604 - protein, 20 aa, Artificial
  Synthetic peptide.
TKTPGTFTPGTGIQKTAVPL

SEQ ID NO: 605 - protein, 6 aa, Artificial
  Synthetic peptide.
MLKQTA

SEQ ID NO: 606 - protein, 30 aa, Artificial
  Synthetic peptide.
MSEAVNLLRGARYSQRYAKNQVPYEVIIEK

SEQ ID NO: 607 - protein, 16 aa, Artificial
  Synthetic peptide.
VIFLHKESGNLKEIFY

SEQ ID NO: 608 - protein, 7 aa, Artificial
  Synthetic peptide.
TFIYNEF

SEQ ID NO: 609 - protein, 18 aa, Artificial
  Synthetic peptide.
KKQDKRIEDKYKRMKKGD

SEQ ID NO: 610 - protein, 8 aa, Artificial
  Synthetic peptide.
HFYLLFER

SEQ ID NO: 611 - protein, 20 aa, Artificial
  Synthetic peptide.
HLFFVKGMFILCQKNQINDE

SEQ ID NO: 612 - protein, 30 aa, Artificial
  Synthetic peptide.
MDSAKAQTMRTDWLAVSCLVASAYLRSMLA

SEQ ID NO: 613 - protein, 27 aa, Artificial
  Synthetic peptide.
MTVFEALMLAIAFATLIVKISNKNDKK

SEQ ID NO: 614 - protein, 21 aa, Artificial
  Synthetic peptide.
ESAKSNLNFLMQEEWALFLLL

SEQ ID NO: 615 - protein, 23 aa, Artificial
  Synthetic peptide.
VFVVLFIIYLASKLLTKLFPIKK

SEQ ID NO: 616 - protein, 16 aa, Artificial
  Synthetic peptide.
KKIIPLITLFVVTLVG

SEQ ID NO: 617 - protein, 24 aa, Artificial
  Synthetic peptide.
QGANPCQQVGFTVNDPDCRLAKTV

SEQ ID NO: 618 - protein, 21 aa, Artificial
  Synthetic peptide.
KYKCSWCKRVYTLRKDHKTAR

SEQ ID NO: 619 - protein, 12 aa, Artificial
  Synthetic peptide.
WSEIEINTKQSN

SEQ ID NO: 620 - protein, 10 aa, Artificial
  Synthetic peptide.
HISKERFEAY

SEQ ID NO: 621 - protein, 25 aa, Artificial
  Synthetic peptide.
MIKKSILKIKYYVPVLISLTLILSA

SEQ ID NO: 622 - protein, 20 aa, Artificial
  Synthetic peptide.
FTLTLITTIVAILNYKDKKK

SEQ ID NO: 623 - protein, 29 aa, Artificial
  Synthetic peptide.
GAVGIAFFAGNMKQDKRIADRQNKKSEKK

SEQ ID NO: 624 - protein, 18 aa, Artificial
  Synthetic peptide.
ITPLLDEIGKVCIDKISK

SEQ ID NO: 625 - protein, 36 aa, Artificial
  Synthetic peptide.
GLQFKEIAEEFHITTTALQQWHKDNGYPIYNKNNRK

SEQ ID NO: 626 - protein, 14 aa, Artificial
  Synthetic peptide.
VVAYVITQVGAIRF

SEQ ID NO: 627 - protein, 14 aa, Artificial
  Synthetic peptide.
DPAGCNDIVRKYCK

SEQ ID NO: 628 - protein, 14 aa, Artificial
  Synthetic peptide.
DLVQSILSEFKKSG

SEQ ID NO: 629 - protein, 10 aa, Artificial
  Synthetic peptide.
VLKEECYQKN

SEQ ID NO: 630 - protein, 16 aa, Artificial
  Synthetic peptide.
YCVPLGNMGNMNNKIW

SEQ ID NO: 631 - protein, 28 aa, Artificial
  Synthetic peptide.
LIYTILASLGVLTVLQAILGREPKAVKA

SEQ ID NO: 632 - protein, 10 aa, Artificial
  Synthetic peptide.
VEDLMEDLNA

SEQ ID NO: 633 - protein, 25 aa, Artificial
  Synthetic peptide.
ILVVLAGILLVVLSYVGISKFKMNC

SEQ ID NO: 634 - protein, 25 aa, Artificial
  Synthetic peptide.
FPIISALLGAIICIAIYSFIVNRKA

SEQ ID NO: 635 - protein, 15 aa, Artificial
  Synthetic peptide.
VIAWKFRNKFENSGV

SEQ ID NO: 636 - protein, 25 aa, Artificial
  Synthetic peptide.
YWLSRVTTGHSFAFEKPVPLSLTIK

SEQ ID NO: 637 - protein, 14 aa, Artificial
  Synthetic peptide.
FIDVLKSKINEFLN

SEQ ID NO: 638 - protein, 25 aa, Artificial
  Synthetic peptide.
LLSTEQLLKYYDGETFDGFQLPSNE

SEQ ID NO: 639 - protein, 9 aa, Artificial
  Synthetic peptide.
VLYFQATVV

SEQ ID NO: 640 - protein, 18 aa, Artificial
  Synthetic peptide.
LVRIEVDDLEEWYERNFI

SEQ ID NO: 641 - protein, 27 aa, Artificial
  Synthetic peptide.
YLEMNADYLSNMDIFDELWEKYLENNK

SEQ ID NO: 642 - protein, 20 aa, Artificial
  Synthetic peptide.
KPKNKKEKTVISYEKLLSMY

SEQ ID NO: 643 - protein, 16 aa, Artificial
  Synthetic peptide.
YCVPLGNMGNMNNKIW

SEQ ID NO: 644 - protein, 14 aa, Artificial
  Synthetic peptide.
DLVQSILSEFKKSG

SEQ ID NO: 645 - protein, 18 aa, Artificial
  Synthetic peptide.
FALELIALCRNLFIVYFP

SEQ ID NO: 646 - protein, 17 aa, Artificial
  Synthetic peptide.
WVAVAILLNIALQTQLT

SEQ ID NO: 647 - protein, 10 aa, Artificial
  Synthetic peptide.
TSGWLGQLEQ

SEQ ID NO: 648 - protein, 22 aa, Artificial
  Synthetic peptide.
TFAGSIKIGVPDLVHVTFNCKR

SEQ ID NO: 649 - protein, 7 aa, Artificial
  Synthetic peptide.
LLNKKLE

SEQ ID NO: 650 - protein, 16 aa, Artificial
  Synthetic peptide.
SKAGLYGKIERSDKRE

SEQ ID NO: 651 - protein, 6 aa, Artificial
  Synthetic peptide.
DSYFRS

SEQ ID NO: 652 - protein, 22 aa, Artificial
  Synthetic peptide.
FFLVHFYIRKRKGKVSIFLNYF

SEQ ID NO: 653 - protein, 13 aa, Artificial
  Synthetic peptide.
KHCFEITDKTDVV

SEQ ID NO: 654 - protein, 30 aa, Artificial
  Synthetic peptide.
MSRKKYENDEKSQKKLKIGRKSDVFYGIID

SEQ ID NO: 655 - protein, 21 aa, Artificial
  Synthetic peptide.
AGKKERLLSFREQFLNKNKKK

SEQ ID NO: 656 - protein, 17 aa, Artificial
  Synthetic peptide.
IAAFVTSRAFSDTVSPI

SEQ ID NO: 657 - protein, 30 aa, Artificial
  Synthetic peptide.
MMELVLKTIIGPIVVGVVLRIVDKWLNKDK

SEQ ID NO: 658 - protein, 30 aa, Artificial
  Synthetic peptide.
MLQKYTQMISVTKCIITKNKKTQENVDAYN

SEQ ID NO: 659 - protein, 20 aa, Artificial
  Synthetic peptide.
YVLEYHGLRATQDVDAFMAL

SEQ ID NO: 660 - protein, 7 aa, Artificial
  Synthetic peptide.
ENEESIF

SEQ ID NO: 661 - protein, 15 aa, Artificial
  Synthetic peptide.
AATLICVGSGIMSSL

SEQ ID NO: 662 - protein, 13 aa, Artificial
  Synthetic peptide.
AVVCGYLAYTATS

SEQ ID NO: 663 - protein, 9 aa, Artificial
  Synthetic peptide.
VAYAAICWW

SEQ ID NO: 664 - protein, 13 aa, Artificial
  Synthetic peptide.
FNGDSEFFLCIAF

SEQ ID NO: 665 - protein, 27 aa, Artificial
  Synthetic peptide.
MRKEFHNVLSSGQLLADKRPARDYNRK

SEQ ID NO: 666 - protein, 16 aa, Artificial
  Synthetic peptide.
GQLLADKRPARDYNRK

SEQ ID NO: 667 - protein, 30 aa, Artificial
  Synthetic peptide.
MSRWDGHSDKGEAPAGKPPMHGFGLNGENK

SEQ ID NO: 668 - protein, 13 aa, Artificial
  Synthetic peptide.
KKHVLVGKQEKNG

SEQ ID NO: 669 - protein, 24 aa, Artificial
  Synthetic peptide.
QPYFQNQFKKITGYTPLQYRKEKR

SEQ ID NO: 670 - protein, 25 aa, Artificial
  Synthetic peptide.
RVLVLKKFHGIMDGNRNVAVFFVGQ

SEQ ID NO: 671 - protein, 30 aa, Artificial
  Synthetic peptide.
MFIISPDLFNIAVILYILFFIHDILLLILS

SEQ ID NO: 672 - protein, 20 aa, Artificial
  Synthetic peptide.
TQVHKMARGIDPGPANGIYR

SEQ ID NO: 673 - protein, 30 aa, Artificial
  Synthetic peptide.
MQIFYIKTKIFLSFFLFLLIFSQCFYKIEE

SEQ ID NO: 674 - protein, 21 aa, Artificial
  Synthetic peptide.
KLLYFFNYFENLQQVHLLVQL

SEQ ID NO: 675 - protein, 30 aa, Artificial
  Synthetic peptide.
MAAKLWEEGKMVYASSASMTKRLKLAMSKV

SEQ ID NO: 676 - protein, 14 aa, Artificial
  Synthetic peptide.
ASMTKRLKLAMSKV

SEQ ID NO: 677 - protein, 6 aa, Artificial
  Synthetic peptide.
SGNEKV

SEQ ID NO: 678 - protein, 22 aa, Artificial
  Synthetic peptide.
IDKSRNKDQFSHIFGLYNICSG

SEQ ID NO: 679 - protein, 15 aa, Artificial
  Synthetic peptide.
SLQSQLGPCLHDQRH

SEQ ID NO: 680 - protein, 13 aa, Artificial
  Synthetic peptide.
HRNLIILQRTIFI

SEQ ID NO: 681 - protein, 30 aa, Artificial
  Synthetic peptide.
MVNYIIGSYMLYREQNNNEALRKFDITLAM

SEQ ID NO: 682 - protein, 30 aa, Artificial; identical to SEQ ID NO: 330
  Synthetic peptide.

SEQ ID NO: 683 - protein, 11 aa, Artificial
  Synthetic peptide.
IIQDIAHAFGY

SEQ ID NO: 684 - protein, 30 aa, Artificial
  Synthetic peptide.
MSVFVPVTNIFMFIMSPIFNVNLLHFKVYI

SEQ ID NO: 685 - protein, 9 aa, Artificial
  Synthetic peptide.
TCVKPRTIN

SEQ ID NO: 686 - protein, 8 aa, Artificial
  Synthetic peptide.
INKYHHIA

SEQ ID NO: 687 - protein, 25 aa, Artificial
  Synthetic peptide.
ISLIIFIMLFVVALFKCITNYKHQS

SEQ ID NO: 688 - protein, 16 aa, Artificial
  Synthetic peptide.
EKRMSFNENQSHRPLL

SEQ ID NO: 689 - protein, 30 aa, Artificial
  Synthetic peptide.
MEHVLPFQNTPPNIVIIYKDFTHLKSITFS

SEQ ID NO: 690 - protein, 30 aa, Artificial
  Synthetic peptide.
MTLAIKNCSVTKCLGFGDFVNDDSDSYFDA

SEQ ID NO: 691 - protein, 7 aa, Artificial
  Synthetic peptide.
KNKTDTL

SEQ ID NO: 692 - protein, 30 aa, Artificial; identical to SEQ ID NO: 297
  Synthetic peptide.

SEQ ID NO: 693 - protein, 10 aa, Artificial
  Synthetic peptide.
VDMVNRFLGN

SEQ ID NO: 694 - protein, 25 aa, Artificial
  Synthetic peptide.
KPVGKALEEIADGKIEPVVPKEYLG

SEQ ID NO: 695 - protein, 6 aa, Artificial
  Synthetic peptide.
VRKSDQ

SEQ ID NO: 696 - protein, 9 aa, Artificial
  Synthetic peptide.
YYKDYFKEI

SEQ ID NO: 697 - protein, 17 aa, Artificial
  Synthetic peptide.
YKVNYNNIDNHFNTLRH

SEQ ID NO: 698 - protein, 18 aa, Artificial
  Synthetic peptide.
PYSDSYATRPHWEQHRAR

SEQ ID NO: 699 - protein, 30 aa, Artificial
  Synthetic peptide.
MVGKIRGVTPRNDLLNANITGQLNLNYRLI

SEQ ID NO: 700 - protein, 30 aa, Artificial
  Synthetic peptide.
MHISHLLDEVEQTEREKAVNVLENMNGNVI

SEQ ID NO: 701 - protein, 25 aa, Artificial
  Synthetic peptide.
MAADIISTIGDLVKWIIDTVNKFKK

SEQ ID NO: 702 - protein, 30 aa, Artificial
  Synthetic peptide.
MHRNLVLVKMEPIPHIMIIANQIGIIIEKA

SEQ ID NO: 703 - protein, 30 aa, Artificial
  Synthetic peptide.
MREKVRFTQAFKLFWTNYFNFKGRSRRSEY

SEQ ID NO: 704 - protein, 14 aa, Artificial
  Synthetic peptide.
WADAQYKLCENCSE

SEQ ID NO: 705 - protein, 12 aa, Artificial
  Synthetic peptide.
HKNKLNIPHIKS

SEQ ID NO: 706 - protein, 20 aa, Artificial
  Synthetic peptide.
HLFILKSHLKPFPPFRYTYD

SEQ ID NO: 707 - protein, 12 aa, Artificial
  Synthetic peptide.
AYILKRREEKNK

SEQ ID NO: 708 - protein, 30 aa, Artificial
  Synthetic peptide.
MVEILVNTAISVYIVALYTQWLSTRDNLKA

SEQ ID NO: 709 - protein, 36 aa, Artificial
  Synthetic peptide.
DELYEIMDKVIEEFNKDIEQNNNNGNNEDLTENKIN

SEQ ID NO: 710 - protein, 17 aa, Artificial
  Synthetic peptide.
LVGYVRTSGTVRSYKIN

SEQ ID NO: 711 - protein, 12 aa, Artificial
  Synthetic peptide.
EDNKDKKDKKDK

SEQ ID NO: 712 - protein, 12 aa, Artificial
  Synthetic peptide.
HKKDIRKQVFKN

SEQ ID NO: 713 - protein, 9 aa, Artificial
  Synthetic peptide.
MQKEGEEDY

SEQ ID NO: 714 - protein, 30 aa, Artificial
  Synthetic peptide.
MYKAIAVLAMTIMAFFIFVYPFFIVGLILG

SEQ ID NO: 715 - protein, 18 aa, Artificial
  Synthetic peptide.
YPNEQGHHKNNLKNIIIE

SEQ ID NO: 716 - protein, 14 aa, Artificial
  Synthetic peptide.
KVDRVSTTITEKIK

SEQ ID NO: 717 - protein, 13 aa, Artificial
  Synthetic peptide.
RLILVSGNATVQK

SEQ ID NO: 718 - protein, 23 aa, Artificial
  Synthetic peptide.
IHQYSSKPDIVGQEAKTVQQINS

SEQ ID NO: 719 - protein, 17 aa, Artificial
  Synthetic peptide.
IQIDAASFYSISKSTIK

SEQ ID NO: 720 - protein, 18 aa, Artificial
  Synthetic peptide.
PGAFFFCRGRGCWCGIGW

SEQ ID NO: 721 - protein, 24 aa, Artificial
  Synthetic peptide.
FTEPLRPLQAKGQIISIKPSTSSS

SEQ ID NO: 722 - protein, 9 aa, Artificial
  Synthetic peptide.
KGIYKKRTY

SEQ ID NO: 723 - protein, 16 aa, Artificial
  Synthetic peptide.
EVTKRLVALAQQQLRG

SEQ ID NO: 724 - protein, 22 aa, Artificial
  Synthetic peptide.
LVLRICTDLFTFIKWTIKQRKS

SEQ ID NO: 725 - protein, 30 aa, Artificial
  Synthetic peptide.
MSEEEEVSEKVYNYLRRNEFFEVRKEEFSA

SEQ ID NO: 726 - protein, 24 aa, Artificial
  Synthetic peptide.
VYSFLYVLVIVRKLLSMKKRIERL

SEQ ID NO: 727 - protein, 30 aa, Artificial
  Synthetic peptide.
MGIFKEEKIKFIDCKGEEVILKIKIKDIKK

SEQ ID NO: 728 - protein, 24 aa, Artificial
  Synthetic peptide.
GSTAHKSPIGSTNNQWGMKKTPTD

SEQ ID NO: 729 - protein, 19 aa, Artificial
  Synthetic peptide.
NKGKQMQDQTGKQPIVDNG

SEQ ID NO: 730 - protein, 19 aa, Artificial
  Synthetic peptide.
VVTLKDIVAVIEDQGYDVQ

SEQ ID NO: 731 - protein, 15 aa, Artificial
  Synthetic peptide.
ILSVELSTKTSASGS

SEQ ID NO: 732 - protein, 10 aa, Artificial
  Synthetic peptide.
GYTKDPGTGI

SEQ ID NO: 733 - protein, 24 aa, Artificial
  Synthetic peptide.
SGRGFALIVVLFILLIIVGAACIR

SEQ ID NO: 734 - protein, 13 aa, Artificial
  Synthetic peptide.
LALSIANLFKKKA

SEQ ID NO: 735 - protein, 14 aa, Artificial
  Synthetic peptide.
VSTFGKVVKVVDEK

SEQ ID NO: 736 - protein, 16 aa, Artificial
  Synthetic peptide.
EAKVQAKGEQIACNNY

SEQ ID NO: 737 - protein, 17 aa, Artificial
  Synthetic peptide.
WYLYKKQSNQNDRGIPK

SEQ ID NO: 738 - protein, 21 aa, Artificial
  Synthetic peptide.
VMQSLYVKPPLILVTKLAQQN

SEQ ID NO: 739 - protein, 16 aa, Artificial
  Synthetic peptide.
SFMPEIQKNTIPTQMK

SEQ ID NO: 740 - protein, 16 aa, Artificial
  Synthetic peptide.
SNGVGLGVGIGSGIRF

SEQ ID NO: 741 - protein, 24 aa, Artificial
  Synthetic peptide.
QRFYKLFYHIDLTNEQALKLFQVK

SEQ ID NO: 742 - protein, 21 aa, Artificial
  Synthetic peptide.
DKSTQDKDIKQAKLLAQELGL

SEQ ID NO: 743 - protein, 14 aa, Artificial
  Synthetic peptide.
VKPTMTASLISTVC

SEQ ID NO: 744 - protein, 21 aa, Artificial
  Synthetic peptide.
SFYSKYSRYIDNLAGAIFLFF

SEQ ID NO: 745 - protein, 14 aa, Artificial
  Synthetic peptide.
YLVYSGVLATAAAF

SEQ ID NO: 746 - protein, 39 aa, Artificial
  Synthetic peptide.
LGLTAGVAYAAQPTNQPTNQPTNQPTNQPTNQPTNQPRW

SEQ ID NO: 747 - protein, 15 aa, Artificial
  Synthetic peptide.
CGKLLEQKNFFLKTR

SEQ ID NO: 748 - protein, 20 aa, Artificial
  Synthetic peptide.
FELVDWLETNLGKILKSKSA

SEQ ID NO: 749 - protein, 30 aa, Artificial
  Synthetic peptide.
ASKQASKQASKQASKQASKQASRSLKNHLL

SEQ ID NO: 750 - protein, 24 aa, Artificial
  Synthetic peptide.
PDAPRTCYHKPILAALSRIVVTDR

SEQ ID NO: 751 - protein, 15 aa, Artificial
  Synthetic peptide.
VLLLFIFQPFQKQLL

SEQ ID NO: 752 - protein, 32 aa, Artificial
  Synthetic peptide.
GSVIKKRRKRMAKKKHRKLLKKTRIQRRRAGK

SEQ ID NO: 753 - protein, 17 aa, Artificial
  Synthetic peptide.
LVDVVVLIRRHLPKSCS

SEQ ID NO: 754 - protein, 13 aa, Artificial
  Synthetic peptide.
LSEMERRRLRKRA

SEQ ID NO: 755 - protein, 13 aa, Artificial
  Synthetic peptide.
LANDYYKKTKKSW

SEQ ID NO: 756 - protein, 27 aa, Artificial
  Synthetic peptide.
SIILTKKKRRKIPLSIDSQIYKYTFKQ

SEQ ID NO: 757 - protein, 19 aa, Artificial
  Synthetic peptide.
KSILILIKVIFIGQTTIIL

SEQ ID NO: 758 - protein, 23 aa, Artificial
  Synthetic peptide.
KKDNPSLNDQDKNAVLNLLALAK

SEQ ID NO: 759 - protein, 26 aa, Artificial
  Synthetic peptide.
NILFGIIGFVVAMTAAVIVTAISIAK

SEQ ID NO: 760 - protein, 17 aa, Artificial
  Synthetic peptide.
FGEKQMRSWWKVHWFHP

SEQ ID NO: 761 - protein, 16 aa, Artificial
  Synthetic peptide.
RESKLIAMADMIRRRI

SEQ ID NO: 762 - protein, 12 aa, Artificial
  Synthetic peptide.
PIIAPTIKTQIQ

SEQ ID NO: 763 - protein, 18 aa, Artificial
  Synthetic peptide.
WSRVPGHSDTGWKVWHRW

SEQ ID NO: 764 - protein, 26 aa, Artificial
  Synthetic peptide.
ARPIADLIHFNSTTVTASGDVYYGPG

SEQ ID NO: 765 - protein, 16 aa, Artificial
  Synthetic peptide.
TGIGPIARPIEHGLDS

SEQ ID NO: 766 - protein, 25 aa, Artificial
  Synthetic peptide.
STENGWQEFESYADVGVDPRRYVPL

SEQ ID NO: 767 - protein, 27 aa, Artificial
  Synthetic peptide.
QVKEKRREIELQFRDAEKKLEASVQAE

SEQ ID NO: 768 - protein, 24 aa, Artificial
  Synthetic peptide.
ELDKADAALGPAKNLAPLDVINRS

SEQ ID NO: 769 - protein, 26 aa, Artificial
  Synthetic peptide.
LTIVGNALQQKNQKLLLNQKKITSLG

SEQ ID NO: 770 - protein, 25 aa, Artificial
  Synthetic peptide.
AKNFLTRTAEEIGEQAVREGNINGP

SEQ ID NO: 771 - protein, 28 aa, Artificial
  Synthetic peptide.
EAYMRFLDREMEGLTAAYNVKLFTEAIS

SEQ ID NO: 772 - protein, 19 aa, Artificial
  Synthetic peptide.
SLQIRMNTLTAAKASIEAA

SEQ ID NO: 773 - protein, 22 aa, Artificial
  Synthetic peptide.
AANKAREQAAAEAKRKAEEQAR

SEQ ID NO: 774 - protein, 12 aa, Artificial
  Synthetic peptide.
ADAPPPLIVRYS

SEQ ID NO: 775 - protein, 25 aa, Artificial
  Synthetic peptide.
SRPGKPGGVSIDVSRDRQDILSNYP

SEQ ID NO: 776 - protein, 20 aa, Artificial
  Synthetic peptide.
FGNPFRGFTLAMEADFKKRK

SEQ ID NO: 777 - protein, 20 aa, Artificial
  Synthetic peptide.
TPEQWLERSTVVVTGLLNRK

SEQ ID NO: 778 - protein, 25 aa, Artificial
  Synthetic peptide.
RPELDNELDVVQNSASLDKLQASYN

SEQ ID NO: 779 - protein, 25 aa, Artificial
  Synthetic peptide.
TIILNDQINSLQERLNKLNAETDRR

SEQ ID NO: 780 - protein, 24 aa, Artificial
  Synthetic peptide.
EAQQVTQQLGADFNAITTPTATKV

SEQ ID NO: 781 - protein, 25 aa, Artificial
  Synthetic peptide.
QQRVKAVDASLSQVSTQVSGAVASA

SEQ ID NO: 782 - protein, 14 aa, Artificial
  Synthetic peptide.
TQAVQVKTAQAQQQ

SEQ ID NO: 783 - protein, 24 aa, Artificial
  Synthetic peptide.
KSKISEYTEKEFLEFVEDIYTNNK

SEQ ID NO: 784 - protein, 25 aa, Artificial
  Synthetic peptide.
KKFPTEESHIQAVLEFKKLTEHPSG

SEQ ID NO: 785 - protein, 13 aa, Artificial
  Synthetic peptide.
WRASKGLPGFKAG

SEQ ID NO: 786 - protein, 23 aa, Artificial
  Synthetic peptide.
EKKLIVKLIDSIGKSHEEIVGAG

SEQ ID NO: 787 - protein, 25 aa, Artificial
  Synthetic peptide.
LNFRAENKILEKIHISLIDTVEGSA

SEQ ID NO: 788 - protein, 25 aa, Artificial
  Synthetic peptide.
AYSGELPEPLVRKMSKEQVRSVMGK

SEQ ID NO: 789 - protein, 25 aa, Artificial
  Synthetic peptide.
PFETRESFRVPVIGILGGWDYFMHP

SEQ ID NO: 790 - protein, 28 aa, Artificial
  Synthetic peptide.
QKANLRIGFTYTSDSNVCNLTFALLGSK

SEQ ID NO: 791 - protein, 30 aa, Artificial
  Synthetic peptide.
MILVCAAVIWGRVLFILKFPIYFSIRLAFL

SEQ ID NO: 792 - protein, 33 aa, Artificial; identical to SEQ ID NO: 290
  Synthetic peptide.

SEQ ID NO: 793 - protein, 7 aa, Artificial
  Synthetic peptide.
WTARARR

SEQ ID NO: 794 - protein, 28 aa, Artificial
  Synthetic peptide.
KFQGEFTNIGQSYIVSASHMSTSLNTGK

SEQ ID NO: 795 - protein, 15 aa, Artificial
  Synthetic peptide.
SYIKNLSNQKFLIAF

SEQ ID NO: 796 - protein, 16 aa, Artificial
  Synthetic peptide.
DYNHLLNVVQDWVNTN

SEQ ID NO: 797 - protein, 12 aa, Artificial
  Synthetic peptide.
FFNQANYFFKEF

SEQ ID NO: 798 - protein, 35 aa, Artificial
  Synthetic peptide.
ASGKYQSYLLNVYVDSKKDRLDIFDKLKAKAKFVL

SEQ ID NO: 799 - protein, 12 aa, Artificial
  Synthetic peptide.
ESVEAIKAKAIK

SEQ ID NO: 800 - protein, 36 aa, Artificial
  Synthetic peptide.
APLRIDEIRNSNVIDEVLDCAPKKQEHFFVVPKIIE

SEQ ID NO: 801 - protein, 10 aa, Artificial
  Synthetic peptide.
YYQAKLFPLL

SEQ ID NO: 802 - protein, 18 aa, Artificial
  Synthetic peptide.
IMKNYKYFKLFIVKYALF

SEQ ID NO: 803 - protein, 26 aa, Artificial
  Synthetic peptide.
MEISTLKKEKLHVKDELSQYLANYKK

SEQ ID NO: 804 - protein, 6 aa, Artificial
  Synthetic peptide.
IVSAIV

SEQ ID NO: 805 - protein, 19 aa, Artificial
  Synthetic peptide.
LQNKIYELLYIKERSKLCS

SEQ ID NO: 806 - protein, 24 aa, Artificial
  Synthetic peptide.
SKMWDKILTILILILELIRELIKL

SEQ ID NO: 807 - protein, 19 aa, Artificial
  Synthetic peptide.
DEIKVSDEEIEKFIKENNL

SEQ ID NO: 808 - protein, 23 aa, Artificial
  Synthetic peptide.
MKFMLEVRNKAISAYKEITRTQI

SEQ ID NO: 809 - protein, 9 aa, Artificial
  Synthetic peptide.
LFEIFKPKH

SEQ ID NO: 810 - protein, 41 aa, Artificial
  Synthetic peptide.
TKKIELKRFVDAFVKKSYENYILERELKKLIKAINEELPTK

SEQ ID NO: 811 - protein, 9 aa, Artificial
  Synthetic peptide.
YRVTVKALE

SEQ ID NO: 812 - protein, 16 aa, Artificial
  Synthetic peptide.
LEKEKKEYIEKLFKTK

SEQ ID NO: 813 - protein, 30 aa, Artificial
  Synthetic peptide.
IDKLKKMNLQKLSYEVRISQDGKSIYARIK

SEQ ID NO: 814 - protein, 7 aa, Artificial
  Synthetic peptide.
LMEQVEV

SEQ ID NO: 815 - protein, 11 aa, Artificial
  Synthetic peptide.
HYRWNTQWWKY

SEQ ID NO: 816 - protein, 19 aa, Artificial
  Synthetic peptide.
YIESDPRKFDYIFGAIRDH

SEQ ID NO: 817 - protein, 23 aa, Artificial
  Synthetic peptide.
TEIKLDNNEYLVLNLDDILGILK

SEQ ID NO: 818 - protein, 19 aa, Artificial
  Synthetic peptide.
VFLKLKTSKIDLASIIFYP

SEQ ID NO: 819 - protein, 35 aa, Artificial
  Synthetic peptide.
GTTLKYGLERQLKIDIHPEITIINLNGGADEFAKL

SEQ ID NO: 820 - protein, 7 aa, Artificial
  Synthetic peptide.
ADEFAKL

SEQ ID NO: 821 - protein, 6 aa, Artificial
  Synthetic peptide.
GLDIYA

SEQ ID NO: 822 - protein, 25 aa, Artificial
  Synthetic peptide.
FLNRFIFYIFTVKTKSALIKNLFLD

SEQ ID NO: 823 - protein, 10 aa, Artificial
  Synthetic peptide.
IVFVVTKEKK

SEQ ID NO: 824 - protein, 8 aa, Artificial
  Synthetic peptide.
PMNAAEPE

SEQ ID NO: 825 - protein, 18 aa, Artificial
  Synthetic peptide.
WSRVPGHSDTGWKVWHRW

SEQ ID NO: 826 - protein, 7 aa, Artificial
  Synthetic peptide.
PPSSFLV

SEQ ID NO: 827 - protein, 19 aa, Artificial
  Synthetic peptide.
TREDVFSVRLINNIVNKQA

SEQ ID NO: 828 - protein, 21 aa, Artificial
  Synthetic peptide.
VLFAVYLGALDWLFSWLTQKM

SEQ ID NO: 829 - protein, 23 aa, Artificial
  Synthetic peptide.
SDSTNNARTRKKARDVTTKDIDK

SEQ ID NO: 830 - protein, 12 aa, Artificial
  Synthetic peptide.
KYDFDDFEPEEA

SEQ ID NO: 831 - protein, 13 aa, Artificial
  Synthetic peptide.
INDLLSYFTLHEK

SEQ ID NO: 832 - protein, 12 aa, Artificial
  Synthetic peptide.
GLAAIATVFALY

SEQ ID NO: 833 - protein, 9 aa, Artificial
  Synthetic peptide.
IPATPIIHS

SEQ ID NO: 834 - protein, 18 aa, Artificial
  Synthetic peptide.
LIIYFSKTGNTARATRQI

SEQ ID NO: 835 - protein, 24 aa, Artificial
  Synthetic peptide.
TTIQGVASLEKHGFRYTIIYPTRI

SEQ ID NO: 836 - protein, 30 aa, Artificial; identical to SEQ ID NO: 287
  Synthetic peptide.

SEQ ID NO: 837 - protein, 30 aa, Artificial
  Synthetic peptide.
MNAHGHSLIFQKMIVHAFAFFSKQKNYLYF

SEQ ID NO: 838 - protein, 5 aa, Artificial
  Synthetic peptide.
LVRLA

SEQ ID NO: 839 - protein, 26 aa, Artificial
  Synthetic peptide.
SRIKQDARSVRKYDRIGIFFYSFKSA

SEQ ID NO: 840 - protein, 6 aa, Artificial
  Synthetic peptide.
TFILPK

SEQ ID NO: 841 - protein, 17 aa, Artificial
  Synthetic peptide.
QATQIKSWIDRLLVSED

SEQ ID NO: 842 - protein, 8 aa, Artificial
  Synthetic peptide.
MGDINRNF

SEQ ID NO: 843 - protein, 20 aa, Artificial
  Synthetic peptide.
SWKCHHLAIGGSWKCHHLAI

SEQ ID NO: 844 - protein, 27 aa, Artificial
  Synthetic peptide.
FTTPMIGIPAGLLGGSYYLKRREEKGK

SEQ ID NO: 845 - protein, 5 aa, Artificial
  Synthetic peptide.
VRCRL

SEQ ID NO: 846 - protein, 14 aa, Artificial
  Synthetic peptide.
TSGLIIGENGLNGL

SEQ ID NO: 847 - protein, 7 aa, Artificial
  Synthetic peptide.
SNSVQQG

SEQ ID NO: 848 - protein, 10 aa, Artificial
  Synthetic peptide.
APASPGRRPG

SEQ ID NO: 849 - protein, 14 aa, Artificial
  Synthetic peptide.
GTFLGQKCAAATAS

SEQ ID NO: 850 - protein, 9 aa, Artificial
  Synthetic peptide.
ARRYPAAGS

SEQ ID NO: 851 - protein, 23 aa, Artificial
  Synthetic peptide.
CPRYPFVDVGPAGPWRARWRVGS

SEQ ID NO: 852 - protein, 24 aa, Artificial
  Synthetic peptide.
IRSDQPGRQSRSSPRWPTGAGRHR

SEQ ID NO: 853 - protein, 11 aa, Artificial
  Synthetic peptide.
PRWPTGAGRHR

SEQ ID NO: 854 - protein, 18 aa, Artificial
  Synthetic peptide.
FLAPARPDLQAQRQALAQ

SEQ ID NO: 855 - protein, 16 aa, Artificial
  Synthetic peptide.
QSVHPLPAETPVADVI

SEQ ID NO: 856 - protein, 9 aa, Artificial
  Synthetic peptide.
LSGRLAGRR

SEQ ID NO: 857 - protein, 17 aa, Artificial
  Synthetic peptide.
DAPCFDDQFGDLKCQMC

SEQ ID NO: 858 - protein, 14 aa, Artificial
  Synthetic peptide.
RGMFVPFHDVDCVQ

SEQ ID NO: 859 - protein, 25 aa, Artificial
  Synthetic peptide.
YVANYTITQFGRDFDDRLAVAIHFA

SEQ ID NO: 860 - protein, 21 aa, Artificial
  Synthetic peptide.
PTTPPPTTPPEIPTGGTVIST

SEQ ID NO: 861 - protein, 5 aa, Artificial
  Synthetic peptide.
TVIST

SEQ ID NO: 862 - protein, 16 aa, Artificial
  Synthetic peptide.
TDPQATAAPRRRTSPR

SEQ ID NO: 863 - protein, 23 aa, Artificial
  Synthetic peptide.
PDEDIRRRAILPPAGPCRPMSPE

SEQ ID NO: 864 - protein, 21 aa, Artificial
  Synthetic peptide.
GKQSRAHGPVASRREFRRKSG

SEQ ID NO: 865 - protein, 8 aa, Artificial
  Synthetic peptide.
ATLIPRKA

SEQ ID NO: 866 - protein, 14 aa, Artificial
  Synthetic peptide.
DQLCVEYPARVSTG

SEQ ID NO: 867 - protein, 15 aa, Artificial
  Synthetic peptide.
VLRVATAVGEVPTGL

SEQ ID NO: 868 - protein, 8 aa, Artificial
  Synthetic peptide.
PNRRSRPR

SEQ ID NO: 869 - protein, 24 aa, Artificial
  Synthetic peptide.
PAHQRLRIDQRLVADRDMVQDYES

SEQ ID NO: 870 - protein, 24 aa, Artificial
  Synthetic peptide.
TNAESMALAFRGRVHMSVNIAGLT

SEQ ID NO: 871 - protein, 24 aa, Artificial
  Synthetic peptide.
RADRIESYPADGDRVITLWRNPYR

SEQ ID NO: 872 - protein, 11 aa, Artificial
  Synthetic peptide.
TVIVAPMHSGV

SEQ ID NO: 873 - protein, 9 aa, Artificial
  Synthetic peptide.
TVSAFRTVH

SEQ ID NO: 874 - protein, 6 aa, Artificial
  Synthetic peptide.
VRRLRM

SEQ ID NO: 875 - protein, 12 aa, Artificial
  Synthetic peptide.
DGCDSEPALTYR

SEQ ID NO: 876 - protein, 24 aa, Artificial
  Synthetic peptide.
EIIPISPTRRCEMHTMSSAEYRGL

SEQ ID NO: 877 - protein, 6 aa, Artificial
  Synthetic peptide.
AEYRGL

SEQ ID NO: 878 - protein, 8 aa, Artificial
  Synthetic peptide.
TCRGAGMH

SEQ ID NO: 879 - protein, 19 aa, Artificial
  Synthetic peptide.
RDRRWTRRDMYDWLESARV

SEQ ID NO: 880 - protein, 8 aa, Artificial
  Synthetic peptide.
CRARFIRR

SEQ ID NO: 881 - protein, 9 aa, Artificial
  Synthetic peptide.
ADPHPTTGI

SEQ ID NO: 882 - protein, 23 aa, Artificial
  Synthetic peptide.
TALTTVGVSGARLITYCVGVEDI

SEQ ID NO: 883 - protein, 12 aa, Artificial
  Synthetic peptide.
RRGKSEQGLSRR

SEQ ID NO: 884 - protein, 5 aa, Artificial
  Synthetic peptide.
LWPVA

SEQ ID NO: 885 - protein, 17 aa, Artificial
  Synthetic peptide.
RKLSLASGFALWRRSLV

SEQ ID NO: 886 - protein, 8 aa, Artificial
  Synthetic peptide.
PTLWLACL

SEQ ID NO: 887 - protein, 22 aa, Artificial
  Synthetic peptide.
LAVLMGYIGYRGWSGKRHINRQ

SEQ ID NO: 888 - protein, 20 aa, Artificial
  Synthetic peptide.
AKRVLSLAVAPHRRQPVQGT

SEQ ID NO: 889 - protein, 10 aa, Artificial
  Synthetic peptide.
ARNHAVIPAG

SEQ ID NO: 890 - protein, 5 aa, Artificial
  Synthetic peptide.
SAPSG

SEQ ID NO: 891 - protein, 30 aa, Artificial
  Synthetic peptide.
MIPLAGDPVSSHRTVEFGVLGTYLVSGGSL

SEQ ID NO: 892 - protein, 19 aa, Artificial
  Synthetic peptide.
HRTVEFGVLGTYLVSGGSL

SEQ ID NO: 893 - protein, 21 aa, Artificial
  Synthetic peptide.
GVAREDPLEPDPLAPIIDDSR

SEQ ID NO: 894 - protein, 5 aa, Artificial
  Synthetic peptide.
PDPAR

SEQ ID NO: 895 - protein, 15 aa, Artificial
  Synthetic peptide.
DLIRPLYSMSAPSVA

SEQ ID NO: 896 - protein, 19 aa, Artificial
  Synthetic peptide.
ALSVMLGNIPLVVPNANQL

SEQ ID NO: 897 - protein, 14 aa, Artificial
  Synthetic peptide.
IRSGISAAYARPLR

SEQ ID NO: 898 - protein, 7 aa, Artificial
  Synthetic peptide.
RADARAK

SEQ ID NO: 899 - protein, 15 aa, Artificial
  Synthetic peptide.
SSGRAGVKCRRPTGR

SEQ ID NO: 900 - protein, 13 aa, Artificial
  Synthetic peptide.
GRAGVKCRRPTGR

SEQ ID NO: 901 - protein, 8 aa, Artificial
  Synthetic peptide.
LNWPFTGR

SEQ ID NO: 902 - protein, 9 aa, Artificial
  Synthetic peptide.
PRGAQSGHG

SEQ ID NO: 903 - protein, 9 aa, Artificial
  Synthetic peptide.
LSGRLAGRR

SEQ ID NO: 904 - protein, 30 aa, Artificial
  Synthetic peptide.
MTTVDNIVGLVIAVALMAFLFAALLFPEKF

SEQ ID NO: 905 - protein, 8 aa, Artificial
  Synthetic peptide.
APAARAAL

SEQ ID NO: 906 - protein, 9 aa, Artificial
  Synthetic peptide.
GEEEGTVAD

SEQ ID NO: 907 - protein, 20 aa, Artificial
  Synthetic peptide.
LGYGAWIGCGLGLNGFHRID

SEQ ID NO: 908 - protein, 19 aa, Artificial
  Synthetic peptide.
IDPESIVTTNNKQDNVDEQ

SEQ ID NO: 909 - protein, 8 aa, Artificial
  Synthetic peptide.
NKKHSPMD

SEQ ID NO: 910 - protein, 13 aa, Artificial
  Synthetic peptide.
KTAGPTGTIYKTN

SEQ ID NO: 911 - protein, 19 aa, Artificial
  Synthetic peptide.
QIYRHVHKVQAKSANLRLY

SEQ ID NO: 912 - protein, 13 aa, Artificial
  Synthetic peptide.
FVVTQRMLRMYKK

SEQ ID NO: 913 - protein, 21 aa, Artificial
  Synthetic peptide.
HGENHHHKSDEKDNDSSEKKD

SEQ ID NO: 914 - protein, 23 aa, Artificial
  Synthetic peptide.
PQSEVTFENIYAPKANGGGLYGI

SEQ ID NO: 915 - protein, 6 aa, Artificial
  Synthetic peptide.
SLDMGK

SEQ ID NO: 916 - protein, 14 aa, Artificial
  Synthetic peptide.
CYRFLTPKRPTRIS

SEQ ID NO: 917 - protein, 19 aa, Artificial
  Synthetic peptide.
AYARCRHDYPFTLGQMQTH

SEQ ID NO: 918 - protein, 21 aa, Artificial
  Synthetic peptide.
AIGQEQDRREYYYYSGYPYYY

SEQ ID NO: 919 - protein, 22 aa, Artificial
  Synthetic peptide.
RHKLIRLPLSESVFCFLNNPKI

SEQ ID NO: 920 - protein, 20 aa, Artificial
  Synthetic peptide.
DRPSQTTHHTLSSSRITGPS

SEQ ID NO: 921 - protein, 18 aa, Artificial
  Synthetic peptide.
VISRQMGSEAVLELFIIM

SEQ ID NO: 922 - protein, 10 aa, Artificial
  Synthetic peptide.
YDPLFPNDKN

SEQ ID NO: 923 - protein, 19 aa, Artificial
  Synthetic peptide.
KSSGSSASASSTAGGSSSK

SEQ ID NO: 924 - protein, 17 aa, Artificial
  Synthetic peptide.
KSGATSAASGAKSGASS

SEQ ID NO: 925 - protein, 20 aa, Artificial
  Synthetic peptide.
AKREDTVAAQIGANILNLIQ

SEQ ID NO: 926 - protein, 25 aa, Artificial
  Synthetic peptide.
LGVGTFVGKVLIKNQQKQKSKKKAQ

SEQ ID NO: 927 - protein, 38 aa, Artificial
  Synthetic peptide.
ANSQNSLFSNRSSFKSIFDKKSNITTNATTPNSNIIIN

SEQ ID NO: 928 - protein, 11 aa, Artificial
  Synthetic peptide.
FLGNSQYFTRK

SEQ ID NO: 929 - protein, 40 aa, Artificial
  Synthetic peptide.
FQGFFDVAVNKWWEEHNKAKLWKNVKGKFLEGEGEEEDDE

SEQ ID NO: 930 - protein, 33 aa, Artificial
  Synthetic peptide.
GVNKWWEEHNKAKLWKNVKGKFLEGEGEEEDDE

SEQ ID NO: 931 - protein, 10 aa, Artificial
  Synthetic peptide.
AESSPAKTTA

SEQ ID NO: 932 - protein, 10 aa, Artificial
  Synthetic peptide.
AESSPAQETT

SEQ ID NO: 933 - protein, 30 aa, Artificial
  Synthetic peptide.
LHVIRPRPELSELKFPITKILKVNKQGLKK

SEQ ID NO: 934 - protein, 7 aa, Artificial
  Synthetic peptide.
DALLRLA

SEQ ID NO: 935 - protein, 11 aa, Artificial
  Synthetic peptide.
PQAISSVQQNA

SEQ ID NO: 936 - protein, 11 aa, Artificial
  Synthetic peptide.
PEIIKIVSGLL

SEQ ID NO: 937 - protein, 21 aa, Artificial
  Synthetic peptide.
DHITLDDYEIHDGFNFELYYG

SEQ ID NO: 938 - protein, 32 aa, Artificial; identical to SEQ ID NO: 282
  Synthetic peptide.

SEQ ID NO: 939 - protein, 19 aa, Artificial
  Synthetic peptide.
PAPAPSAPAPAPEQPEQPA

SEQ ID NO: 940 - protein, 43 aa, Artificial
  Synthetic peptide.
GIWMARNYFHRSSIRKVYVESDKEYERVHPMQKIQYEGNYKSQ

SEQ ID NO: 941 - protein, 9 aa, Artificial
  Synthetic peptide.
GYFEPGKRD

SEQ ID NO: 942 - protein, 32 aa, Artificial
  Synthetic peptide.
YLYWEVEHKPIIAKRDAYYAQLRKQKEIEEGA

SEQ ID NO: 943 - protein, 17 aa, Artificial
  Synthetic peptide.
DAYYAQLRKQKEIEEGA

SEQ ID NO: 944 - protein, 15 aa, Artificial
  Synthetic peptide.
DGKQGEPVALKPTDN

SEQ ID NO: 945 - protein, 12 aa, Artificial
  Synthetic peptide.
GFRGGKRGGARG

SEQ ID NO: 946 - protein, 28 aa, Artificial
  Synthetic peptide.
GVGIGFIMMGVVGYAVKLVHIPIRYLIV

SEQ ID NO: 947 - protein, 6 aa, Artificial
  Synthetic peptide.
TKESSS

SEQ ID NO: 948 - protein, 6 aa, Artificial
  Synthetic peptide.
TLKESK

SEQ ID NO: 949 - protein, 29 aa, Artificial
  Synthetic peptide.
VSILLYLSATIILPNVLRLLVARAIIVRV

SEQ ID NO: 950 - protein, 20 aa, Artificial
  Synthetic peptide.
PGADGKLAEASAAIARLVRS

SEQ ID NO: 951 - protein, 10 aa, Artificial
  Synthetic peptide.
MNLILTAHGT

SEQ ID NO: 952 - protein, 42 aa, Artificial
  Synthetic peptide.
IYGDFFNFYLCDISLKVNGLQPGGPVRTVKLFGQPTGRCTPQ

SEQ ID NO: 953 - protein, 40 aa, Artificial
  Synthetic peptide.
AVYDALVALAAAEHRAELATRDARAKDTYEKIGVHVVVAA

SEQ ID NO: 954 - protein, 15 aa, Artificial
  Synthetic peptide.
PLVVVNHRRAERSRG

SEQ ID NO: 955 - protein, 29 aa, Artificial
  Synthetic peptide.
TGPRRGIDLTSNRALSEVLDEGLELNSRK

SEQ ID NO: 956 - protein, 37 aa, Artificial
  Synthetic peptide.
FTSEVRGVFTYRVNKAGLITNMRGYWNLDMMTFGNQE

SEQ ID NO: 957 - protein, 32 aa, Artificial
  Synthetic peptide.
MAMTTVDNIVGLVIAVALMAFLFAALLFPEKF

SEQ ID NO: 958 - protein, 22 aa, Artificial
  Synthetic peptide.
MRPQHSPAGKAFVVKKITHEQS

SEQ ID NO: 959 - protein, 14 aa, Artificial
  Synthetic peptide.
LSERERRRLKRGII

SEQ ID NO: 960 - protein, 34 aa, Artificial
  Synthetic peptide.
MTERQRRALLKQHPEVVSWSDYLEKRKRRTGTAG

SEQ ID NO: 961 - protein, 28 aa, Artificial
  Synthetic peptide.
GLITVFAGTARILQLRRAAKKTHAAALR

SEQ ID NO: 962 - protein, 9 aa, Artificial
  Synthetic peptide.
PRGAQSGHG

SEQ ID NO: 963 - protein, 13 aa, Artificial
  Synthetic peptide.
PAGPDHLDQRDHR

SEQ ID NO: 964 - protein, 16 aa, Artificial
  Synthetic peptide.
IFLTTQNTDYSEHNAA

SEQ ID NO: 965 - protein, 10 aa, Artificial
  Synthetic peptide.
ALHASGIQAI

SEQ ID NO: 966 - protein, 21 aa, Artificial
  Synthetic peptide.
YTQXNNASAYAMLLTNKDTVP

SEQ ID NO: 967 - protein, 9 aa, Artificial
  Synthetic peptide.
NLYFENQGN

SEQ ID NO: 968 - protein, 20 aa, Artificial
  Synthetic peptide.
ALHKSGIQVIADWVPDQIYN

SEQ ID NO: 969 - protein, 20 aa, Artificial
  Synthetic peptide.
YTQSNIPTAYALMLSNKDSI

SEQ ID NO: 970 - protein, 10 aa, Artificial
  Synthetic peptide.
WYYFDNNGYM

SEQ ID NO: 971 - protein, 20 aa, Artificial
  Synthetic peptide.
ALHSKGIKVMADWVPDQMYA

SEQ ID NO: 972 - protein, 21 aa, Artificial
  Synthetic peptide.
YTHYNTALSYALLLTNKSSVP

SEQ ID NO: 973 - protein, 10 aa, Artificial
  Synthetic peptide.
WYYFDNNGYM

SEQ ID NO: 974 - protein, 22 aa, Artificial
  Synthetic peptide.
FCSVDHDVITIAADHVKQGAEA

SEQ ID NO: 975 - protein, 27 aa, Artificial
  Synthetic peptide.
AQPRRTWLVNFGEVPSPGLTNDGMPDH

SEQ ID NO: 976 - protein, 21 aa, Artificial
  Synthetic peptide.
HPMPITVRSRKPGPLTAPSEH

SEQ ID NO: 977 - protein, 26 aa, Artificial
  Synthetic peptide.
FREGMGWPLSNEGSPTAPLPKHRNQV

SEQ ID NO: 978 - protein, 13 aa, Artificial
  Synthetic peptide.
QGLARPVLRRIPL

SEQ ID NO: 979 - protein, 7 aa, Artificial
  Synthetic peptide.
SRFRNGV

SEQ ID NO: 980 - protein, 25 aa, Artificial
  Synthetic peptide.
YNLSIYIYFLHTITIAGLITLPFII

SEQ ID NO: 981 - protein, 27 aa, Artificial
  Synthetic peptide.
YFWWYWVQDCIPYKNNEVWLELSNNMK

SEQ ID NO: 982 - protein, 36 aa, Artificial
  Synthetic peptide.
FETGFGDGYYMSLWGLNEKDEVCKVVIPFINPELID

SEQ ID NO: 983 - protein, 33 aa, Artificial
  Synthetic peptide.
TLNYKKMFFSVIFLLGLNYLICNSPLFFKQIEF

SEQ ID NO: 984 - protein, 24 aa, Artificial
  Synthetic peptide.
PLARATEVVATLFIICSLLLYLTR

SEQ ID NO: 985 - protein, 7 aa, Artificial
  Synthetic peptide.
SHFRKGD

SEQ ID NO: 986 - protein, 24 aa, Artificial
  Synthetic peptide.
DEEALEMGANLYAQFAIDFLNSKK

SEQ ID NO: 987 - protein, 40 aa, Artificial
  Synthetic peptide.
DEERYSDSYFLKEKVFYLILALFLILFHQKYLYFLEIITI

SEQ ID NO: 988 - protein, 7 aa, Artificial
  Synthetic peptide.
LNLFASI

SEQ ID NO: 989 - protein, 28 aa, Artificial
  Synthetic peptide.
NALMLREMQLAKNIKVEVTDVLSNKKYC

SEQ ID NO: 990 - protein, 7 aa, Artificial
  Synthetic peptide.
QVIVKIL

SEQ ID NO: 991 - protein, 41 aa, Artificial
  Synthetic peptide.
KKMFSLIRKVNWIFFILFIVLDLTNVFPLIRTILFAILSRQ

SEQ ID NO: 992 - protein, 26 aa, Artificial
  Synthetic peptide.
KALVISVFAIVFSIIFVKFFYWRDKK

SEQ ID NO: 993 - protein, 7 aa, Artificial
  Synthetic peptide.
INIPGLF

SEQ ID NO: 994 - protein, 24 aa, Artificial
  Synthetic peptide.
FFSVIFLFGLNYLICNSPLFNILR

SEQ ID NO: 995 - protein, 25 aa, Artificial
  Synthetic peptide.
KKFKIFVIINWFYHKYIILNFEENF

SEQ ID NO: 996 - protein, 30 aa, Artificial
  Synthetic peptide.
ELFFTILSDCNELFLLHLLQQPLFYIKKGK

SEQ ID NO: 997 - protein, 17 aa, Artificial
  Synthetic peptide.
DIANNILNSVSERLIIA

SEQ ID NO: 998 - protein, 31 aa, Artificial
  Synthetic peptide.
ASNTPRFVRLTLFNFYSKIWNVTHLFLFNNL

SEQ ID NO: 999 - protein, 7 aa, Artificial
  Synthetic peptide.
EKLGTMV

SEQ ID NO: 1000 - protein, 35 aa, Artificial
  Synthetic peptide.
LLALNMNEDTYYFELFFIFDNQNKKWLIFDLKERG

SEQ ID NO: 1001 - protein, 36 aa, Artificial
  Synthetic peptide.
PETKGKVSAFVFGIVVANVIAVVYILYMLREIGIIQ

SEQ ID NO: 1002 - protein, 33 aa, Artificial
  Synthetic peptide.
ASLSTMTFKVMELKELIILLCGLTMLMIQTEFV

SEQ ID NO: 1003 - protein, 27 aa, Artificial
  Synthetic peptide.
QWIVAKREIRMHIYCHISVIHVIIFFG

SEQ ID NO: 1004 - protein, 29 aa, Artificial
  Synthetic peptide.
NELMKYPATLTATATTPGIKYSHLCSVCL

SEQ ID NO: 1005 - protein, 36 aa, Artificial
  Synthetic peptide.
KNTHAYLRVLRLSSLILSYQASVYPLFAYLCQQKDY

SEQ ID NO: 1006 - protein, 22 aa, Artificial
  Synthetic peptide.
LILSYQASVYPLFAYLCQQKDY

SEQ ID NO: 1007 - protein, 25 aa, Artificial
  Synthetic peptide.
QRMYWFKRGFETGDFSAGDTFAELK

SEQ ID NO: 1008 - protein, 27 aa, Artificial
  Synthetic peptide.
LLASHPERLSLGVFFVYRVLHLLLENT

SEQ ID NO: 1009 - protein, 28 aa, Artificial
  Synthetic peptide.
DFPPLSFFRRRFHAYTAPIDNFFGANPF

SEQ ID NO: 1010 - protein, 10 aa, Artificial
  Synthetic peptide.
VVFGGGDRLV

SEQ ID NO: 1011 - protein, 7 aa, Artificial
  Synthetic peptide.
YGKESDP

SEQ ID NO: 1012 - protein, 17 aa, Artificial
  Synthetic peptide.
AASGFTYCASNGVWHPY

SEQ ID NO: 1013 - protein, 40 aa, Artificial
  Synthetic peptide.
TVEELDKAFTWGAAAALAIGVIAINVGLAAGYCYNNNDVF

SEQ ID NO: 1014 - protein, 33 aa, Artificial
  Synthetic peptide.
KMRAGQVVFIYKLILVLLFYVLQKLFDLKKGCF

SEQ ID NO: 1015 - protein, 41 aa, Artificial
  Synthetic peptide.
NTNDLLQAFELMGLGMAGVFIVLGILYIVAELLIKIFPVNN

SEQ ID NO: 1016 - protein, 11 aa, Artificial
  Synthetic peptide.
AEIQPHCLSVL

SEQ ID NO: 1017 - protein, 13 aa, Artificial
  Synthetic peptide.
FFPSYYSIIITYF

SEQ ID NO: 1018 - protein, 31 aa, Artificial
  Synthetic peptide.
KNMLKRRMKQKRLFDEEDRLRVLSKYTKSYY

SEQ ID NO: 1019 - protein, 30 aa, Artificial
  Synthetic peptide.
KKEKLLTAIRLQHRAEIRGYFTIFFLFFRI

SEQ ID NO: 1020 - protein, 32 aa, Artificial
  Synthetic peptide.
FTIIELKKQKIKHGENNKKTAHPLNEPFCARA

SEQ ID NO: 1021 - protein, 30 aa, Artificial
  Synthetic peptide.
GNVHPESDFHNLIQFIKTFLYFTIFFKYFL

SEQ ID NO: 1022 - protein, 29 aa, Artificial
  Synthetic peptide.
HPFLTGTGCPLFLIFRLFFVKAYFSFTVF

SEQ ID NO: 1023 - protein, 32 aa, Artificial; identical to SEQ ID NO: 305
  Synthetic peptide.

SEQ ID NO: 1024 - protein, 31 aa, Artificial
  Synthetic peptide.
VLLNIFRTLLEFFSPSNAPGAEDVPLPDTQA

SEQ ID NO: 1025 - protein, 34 aa, Artificial
  Synthetic peptide.
VVAGVVLLTALAVGSKRKEKKQIKEIQRLLAATR

SEQ ID NO: 1026 - protein, 12 aa, Artificial
  Synthetic peptide.
IENLERGARRPP

SEQ ID NO: 1027 - protein, 10 aa, Artificial
  Synthetic peptide.
GMPQIPRLRI

SEQ ID NO: 1028 - protein, 26 aa, Artificial
  Synthetic peptide.
MAEDERRALKRRTNRGRTRTRKRITV

SEQ ID NO: 1029 - protein, 24 aa, Artificial
  Synthetic peptide.
TELKYNGEEYLLLTQRDILAVIEK

SEQ ID NO: 1030 - protein, 32 aa, Artificial
  Synthetic peptide.
TSDTQSQSPWLFDNADIVNIYPVQLMHSSDND

SEQ ID NO: 1031 - protein, 16 aa, Artificial
  Synthetic peptide.
TFFRLFNRSFTQALGK

SEQ ID NO: 1032 - protein, 15 aa, Artificial
  Synthetic peptide.
KFINGVLSQFVLERK

SEQ ID NO: 1033 - protein, 9 aa, Artificial
  Synthetic peptide.
FIDSFIRSF

SEQ ID NO: 1034 - protein, 16 aa, Artificial
  Synthetic peptide.
TFFRLFNRSFTQALGK

SEQ ID NO: 1035 - protein, 9 aa, Artificial
  Synthetic peptide.
FIKHFIHRF

SEQ ID NO: 1036 - protein, 12 aa, Artificial
  Synthetic peptide.
KKHRKHRKHRKH

SEQ ID NO: 1037 - protein, 17 aa, Artificial
  Synthetic peptide.
GLGRVIGRLIKQIIWRR

SEQ ID NO: 1038 - protein, 19 aa, Artificial
  Synthetic peptide.
VYRKRKSILKIYAKLKGWH

SEQ ID NO: 1039 - protein, 20 aa, Artificial
  Synthetic peptide.
NYRLVNAIFSKIFKKKFIKF

SEQ ID NO: 1040 - protein, 11 aa, Artificial
  Synthetic peptide.
KILKFLFKKVF

SEQ ID NO: 1041 - protein, 11 aa, Artificial
  Synthetic peptide.
FIRKFLKKWLL

SEQ ID NO: 1042 - protein, 11 aa, Artificial
  Synthetic peptide.
KLFKFLRKHLL

SEQ ID NO: 1043 - protein, 11 aa, Artificial
  Synthetic peptide.
KILKFLFKQVF

SEQ ID NO: 1044 - protein, 11 aa, Artificial
  Synthetic peptide.
KILKKLFKFVF

SEQ ID NO: 1045 - protein, 11 aa, Artificial
  Synthetic peptide.
GILKKLFTKVF

SEQ ID NO: 1046 - protein, 9 aa, Artificial
  Synthetic peptide.
LRKFLHKLF

SEQ ID NO: 1047 - protein, 9 aa, Artificial
  Synthetic peptide.
LRKNLRWLF

SEQ ID NO: 1048 - protein, 11 aa, Artificial
  Synthetic peptide.
FIRKFLQKLHL

SEQ ID NO: 1049 - protein, 11 aa, Artificial
  Synthetic peptide.
FTRKFLKFLHL

SEQ ID NO: 1050 - protein, 12 aa, Artificial
  Synthetic peptide.
KKFKKFKVLKIL

SEQ ID NO: 1051 - protein, 12 aa, Artificial
  Synthetic peptide.
LLKLLKLKKLKF

SEQ ID NO: 1052 - protein, 14 aa, Artificial
  Synthetic peptide.
FLKFLKKFFKKLKY

SEQ ID NO: 1053 - protein, 16 aa, Artificial
  Synthetic peptide.
GWLKMFKKIIGKFGKF

SEQ ID NO: 1054 - protein, 16 aa, Artificial
  Synthetic peptide.
GIFKKFVKILYKVQKL

SEQ ID NO: 1055 - protein, 24 aa, Artificial
  Synthetic peptide.
GRLVLEITADEVKALGEALANAKI

SEQ ID NO: 1056 - protein, 21 aa, Artificial
  Synthetic peptide.
YIQFHLNQQPRPKVKKIKIFL

SEQ ID NO: 1057 - protein, 32 aa, Artificial; identical to SEQ ID NO: 752
  Synthetic peptide.

SEQ ID NO: 1058 - protein, 30 aa, Artificial
  Synthetic peptide.
MRFGSLALVAYDSAIKHSWPRPSSVRRLRM

SEQ ID NO: 1059 - protein, 39 aa, Artificial
  Synthetic peptide.
FESKILNASKELDKEKKVNTALSFNSHQDFAKAYQNGKI

SEQ ID NO: 1060 - protein, 18 aa, Artificial
  Synthetic peptide.
WSRVPGHSDTGWKVWHRW

SEQ ID NO: 1061 - protein, 21 aa, Artificial
  Synthetic peptide.
MGIIAGIIKFIKGLIEKFTGK

SEQ ID NO: 1062 - protein, 16 aa, Artificial
  Synthetic peptide.
RESKLIAMADMIRRRI

SEQ ID NO: 1063 - protein, 24 aa, Artificial
  Synthetic peptide.
LSLATFAKIFMTRSNWSLKRFNRL

SEQ ID NO: 1064 - protein, 30 aa, Artificial; identical to SEQ ID NO: 546
  Synthetic peptide.

SEQ ID NO: 1065 - protein, 37 aa, Artificial; identical to SEQ ID NO: 562
  Synthetic peptide.

SEQ ID NO: 1066 - protein, 28 aa, Artificial
  Synthetic peptide.
VLPFPAIPLSRRRACVAAPRPRSRQRAS

SEQ ID NO: 1067 - protein, 30 aa, Artificial
  Synthetic peptide.
KNKKQTDILEKVKEILDKKKKTKSVGQKLY

SEQ ID NO: 1068 - protein, 15 aa, Artificial
  Synthetic peptide.
SLQSQLGPCLHDQRH

SEQ ID NO: 1069 - protein, 28 aa, Artificial
  Synthetic peptide.
KFQGEFTNIGQSYIVSASHMSTSLNTGK

SEQ ID NO: 1070 - protein, 41 aa, Artificial; identical to SEQ ID NO: 810
  Synthetic peptide.

SEQ ID NO: 1071 - protein, 20 aa, Artificial
  Synthetic peptide.
KFSDQIDKGQDALKDKLGDL

SEQ ID NO: 1072 - protein, 13 aa, Artificial
  Synthetic peptide.
LSEMERRRLRKRA

SEQ ID NO: 1073 - protein, 25 aa, Artificial
  Synthetic peptide.
RRGCTERLRRMARRNAWDLYAEHFY

SEQ ID NO: 1074 - protein, 26 aa, Artificial
  Synthetic peptide.
SKFKVLRKIIIKEYKGELMLSIQKQR

SEQ ID NO: 1075 - protein, 20 aa, Artificial
  Synthetic peptide.
FELVDWLETNLGKILKSKSA

SEQ ID NO: 1076 - protein, 22 aa, Artificial
  Synthetic peptide.
LVLRICTDLFTFIKWTIKQRKS

SEQ ID NO: 1077 - protein, 24 aa, Artificial
  Synthetic peptide.
VYSFLYVLVIVRKLLSMKKRIERL

SEQ ID NO: 1078 - protein, 18 aa, Artificial
  Synthetic peptide.
GIVLIGLKLIPLLANVLR

SEQ ID NO: 1079 - protein, 21 aa, Artificial
  Synthetic peptide.
VMQSLYVKPPLILVTKLAQQN

SEQ ID NO: 1080 - protein, 16 aa, Artificial
  Synthetic peptide.
SFMPEIQKNTIPTQMK

SEQ ID NO: 1081 - protein, 39 aa, Artificial; identical to SEQ ID NO: 746
  Synthetic peptide.

SEQ ID NO: 1082 - protein, 15 aa, Artificial
  Synthetic peptide.
CGKLLEQKNFFLKTR

SEQ ID NO: 1083 - protein, 30 aa, Artificial; identical to SEQ ID NO: 749
  Synthetic peptide.

SEQ ID NO: 1084 - protein, 24 aa, Artificial
  Synthetic peptide.
PDAPRTCYHKPILAALSRIVVTDR

SEQ ID NO: 1085 - protein, 8 aa, Artificial
  Synthetic peptide.
NYAVVSHT

SEQ ID NO: 1086 - protein, 23 aa, Artificial
  Synthetic peptide.
FQKPFTGEEVEDFQDDDEIPTII

SEQ ID NO: 1087 - protein, 16 aa, Artificial
  Synthetic peptide.
KNLRIIRKGIHIIKKY

SEQ ID NO: 1088 - protein, 11 aa, Artificial
  Synthetic peptide.
KLFKFLRKHLL

SEQ ID NO: 1089 - protein, 14 aa, Artificial
  Synthetic peptide.
FLKFLKKFFKKLKY

SEQ ID NO: 1090 - protein, 16 aa, Artificial
  Synthetic peptide.
KNLRIIRKGIHIIKKY

SEQ ID NO: 1091 - protein, 18 aa, Artificial
  Synthetic peptide.
KNLRRIIRKGIHIIKKYG

SEQ ID NO: 1092 - protein, 5 aa, Artificial
  Synthetic peptide linker.
PSGSP

SEQ ID NO: 1093 - protein, 5 aa, Artificial
  Synthetic peptide linker.
ASASA

SEQ ID NO: 1094 - protein, 15 aa, Artificial
  Synthetic peptide linker.
GGGGSGGGGSGGGGS

SEQ ID NO: 1095 - protein, 6 aa, Artificial
  Synthetic peptide linker.
GGSGGS

SEQ ID NO: 1096 - protein, 4 aa, Artificial
  Synthetic peptide linker.
GGGG

SEQ ID NO: 1097 - protein, 5 aa, Artificial
  Synthetic peptide linker.
PSPSP

SEQ ID NO: 1098 - protein, 5 aa, Artificial
  Synthetic peptide linker.
ASASA

SEQ ID NO: 1099 - protein, 5 aa, Artificial
  Synthetic peptide linker.
PSPSP

SEQ ID NO: 1100 - protein, 4 aa, Artificial
  Synthetic peptide linker.
KKKK

SEQ ID NO: 1101 - protein, 4 aa, Artificial
  Synthetic peptide linker.
RRRR

SEQ ID NO: 1102 - protein, 5 aa, Artificial
  Synthetic peptide linker.
GGGGS

SEQ ID NO: 1103 - protein, 10 aa, Artificial
  Synthetic peptide linker.
GGGGSGGGGS

SEQ ID NO: 1104 - protein, 15 aa, Artificial
  Synthetic peptide linker.
GGGGSGGGGSGGGGS

SEQ ID NO: 1105 - protein, 20 aa, Artificial
  Synthetic peptide linker.
GGGGSGGGGSGGGGSGGGGS

SEQ ID NO: 1106 - protein, 25 aa, Artificial
  Synthetic peptide linker.
GGGGSGGGGSGGGGSGGGGSGGGGS

SEQ ID NO: 1107 - protein, 30 aa, Artificial
  Synthetic peptide linker.
GGGGSGGGGSGGGGSGGGGSGGGGSGGGGS

SEQ ID NO: 1108 - protein, 16 aa, Artificial
  Synthetic peptide.
TFFRLFNRSFTQALGK

SEQ ID NO: 1109 - protein, 16 aa, Artificial
  Synthetic peptide.
KNLRIIRKGIHIIKKY

SEQ ID NO: 1110 - protein, 35 aa, Artificial
  Synthetic peptide.
TFFRLFNRSFTQALGKGGGKNLRIIRKGIHIIKKY

SEQ ID NO: 1111 - protein, 29 aa, Artificial
  Synthetic peptide.
KFINGVLSQFVLERKPYPKLFKFLRKHLL

SEQ ID NO: 1112 - protein, 23 aa, Artificial
  Synthetic peptide.
FIDSFIRSFGGGKLFKFLRKHLL

SEQ ID NO: 1113 - protein, 33 aa, Artificial
  Synthetic peptide.
TFFRLFNRSFTQALGKGGGFLKFLKKFFKKLKY

SEQ ID NO: 1114 - protein, 28 aa, Artificial
  Synthetic peptide.
FIKHFIHRFGGGKNLRIIRKGIHIIKKY

SEQ ID NO: 1115 - protein, 36 aa, Artificial
  Synthetic peptide.
KKHRKHRKHRKHGGSGGSKNLRRIIRKGIHIIKKYG

(19) United States
(12) Patent Application Publication
(10) Pub. No.: US 2012/0003661 A1
(43) Pub. Date: Jan. 5, 2012

(54) METHODS AND DEVICES FOR THE SELECTIVE DETECTION OF MICROORGANISMS

(75) Inventors: Randal H. Eckert, Redondo Beach, CA (US); Chris Kaplan, Los Angeles, CA (US); Jian He, Los Angeles, CA (US); Daniel K. Yarbrough, Los Angeles, CA (US); Maxwell Anderson, Sequim, WA (US); Jee-Hyun Sim, Garden Grove, CA (US)

(73) Assignee: C3 JIAN, INC., INGLEWOOD, CA (US)

(21) Appl. No.: 13/170,045

(22) Filed: Jun. 27, 2011

Related U.S. Application Data
(60) Provisional application No. 61/446,910, filed on Feb. 25, 2011.
(60) Provisional application No. 61/361,463, filed on Jul. 5, 2010.

(51) Int. Cl. C12Q 1/68; C12M 1/34; C12Q 1/66; C12Q 1/40; C12Q 1/32; G01N 27/26; C12Q 1/42; C12Q 1/26; C12Q 1/04; C12Q 1/34; C12Q 1/44; C12Q 1/06; G01N 33/569; C12Q 1/48

(52) U.S. Cl. 435/6.15; 435/7.31; 435/7.32; 435/7.37; 435/7.33; 435/7.34; 435/287.1; 435/8; 435/22; 435/26; 435/15; 435/21; 435/25; 435/34; 435/18; 435/19; 435/39; 205/781; 204/403.1

(57) ABSTRACT

Methods and devices are provided for the rapid and specific detection of target microorganisms, cells, and the like. In one embodiment, the methods involve contacting a target microorganism (e.g., in a sample) with a permeabilization reagent that selectively permeabilizes or lyses the microorganism; contacting the selectively permeabilized microorganism with a detection reagent that is taken into the selectively permeabilized organism or that contacts metabolites or enzymes released by the selectively permeabilized microorganism, where the detection reagent produces a signal in the presence of said metabolites or enzymes; and detecting a signal produced by the detection reagent in the presence of the metabolites or enzymes wherein the strength of the signal indicates the presence and/or amount of the target microorganism in the sample.

### CROSS-REFERENCE TO RELATED APPLICATIONS

This application claims benefit of and priority to U.S. Ser. No. 61/446,910, filed on Feb. 25, 2011 and to U.S. Ser. No. 61/361,463, filed on Jul. 5, 2010, both of which are incorporated herein by reference in their entirety for all purposes.

### STATEMENT OF GOVERNMENTAL SUPPORT

[Not Applicable]

### FIELD OF THE INVENTION

The present invention relates to field of assays and diagnostics. In particular assays methods and devices are provided for the rapid and specific detection of target microorganisms, cells, and the like.

### BACKGROUND OF THE INVENTION

Various bacteria are responsible for numerous human diseases. For example, Escherichia coli can cause several intestinal and extra-intestinal infections such as urinary tract infections, meningitis, peritonitis, mastitis, septicemia and Gram-negative pneumonia. Bacterial infections from Mycoplasma pneumoniae, may lead to tracheobronchitis, primary atypical pneumonia, contribute to the onset and exacerbation of asthma, and other respiratory disorders. Infections from Mycoplasma genitalium may lead to urogenital disease. Bacterial infections, such as these noted above, are the cause of millions of hospitalizations and thousands of deaths each year. Other infections impact the food and agriculture industries. For example, Mycoplasma gallisepticum (MG) causes severe chronic respiratory disease in chickens and turkeys resulting in hundreds of millions of dollars in annual losses to the poultry industry in the US alone.

Classical microbiological methods are still the most commonly used techniques for identifying and quantifying specific bacterial pathogens. These methods are generally easy to perform, do not require expensive supplies or laboratory facilities, and offer high levels of selectivity; however, they are slow. Classical microbiological methods, however, are hindered by the requirement to first grow or cultivate pure cultures of the targeted organism, which can take many hours to days. This time constraint severely limits the ability to provide a rapid and ideal response to the presence of virulent strains of microorganisms. The extensive time it takes to identify microorganisms using standard methods is a serious problem resulting in significant human morbidity and increased economic costs.

Molecular biology techniques are quickly gaining acceptance as valuable alternatives to standard microbiological tests. Serological methods have been widely employed to evaluate a host of matrices for targeted microorganisms (see, e.g., Kingsbury and Falkow (1985) Rapid Detection And Identification of Infectious Agents, Academic Press, Inc., New York). These tests focus on using antibodies to first trap and then separate targeted organisms from other constituents in complicated biological mixtures. Once isolated, the captured organism can be concentrated and detected by a variety of different techniques that do not require cultivating the biological analyte. One such approach, termed “immunomagnetic separation” (IMS), involves immobilizing antibodies to spherical, micro-sized magnetic or paramagnetic beads and using these beads to trap targeted microorganisms from liquid media. The beads are easily manipulated under the influence of a magnetic field facilitating the retrieval and concentration of targeted organisms.

Detection methods previously used with IMS include, for example, ELISA (Kofitsyo et al. (1995) Int. J. Food Microbiol., 27: 11-25), dot blot assays (Skjerve et al. (1990) Appl. & Env. Microbiol., 3478-3481), electrochemiluminescence (Yu and Bruno (1996) Appl. & Env. Microbiol., 587-592), and flow cytometry (Pyle et al. (1999) Appl. & Env. Microbiol., 1966-1972). Although these tests provide satisfactory results, they are laborious to perform and give binary responses (yes/no) that are highly susceptible to false-positive results due to cross-reactivity with non-target analytes. While these approaches can offer faster results than do traditional microbiology methods, they do not typically achieve the sensitivity levels that substrate-based assays do, are more expensive, and typically require more highly trained technicians than do classical substrate-based methods.

Other molecular biology techniques that have received a great deal of attention recently are Polymerase Chain Reaction (PCR) methods. PCR detection of specific microorganisms in a sample involves extraction of the genetic material (RNA and/or DNA) in a sample, amplification of a target genetic sequence specific to the microorganism of interest, and then detection of the amplified genetic material. PCR techniques offer high selectivity owing to the uniqueness of the detected genetic material, high sensitivity because of the substantial amplification of the target genetic material, and rapid results owing to the potentially fast amplification process. However, PCR instruments and reagents are quite expensive and highly trained technicians are needed to perform the tests. In addition, numerous steps are involved that increase the chance of errors.

### SUMMARY OF THE INVENTION

In various embodiments methods and devices are provided for the rapid and specific detection of target microorganisms, cells, and the like. In one embodiment, the methods involve contacting a target microorganism (e.g., in a sample) with a selective permeabilization reagent that selectively permeabilizes or lyses the target microorganism; contacting the selectively permeabilized or lysed microorganism with a detection reagent that is taken into the selectively permeabilized organism or that contacts metabolites or enzymes released by the selectively permeabilized microorganism, where the detection reagent produces a signal in the presence of said metabolites or enzymes; and detecting a signal produced by the detection reagent in the presence of the metabolites or enzymes wherein the strength of the signal indicates the presence or amount of the target microorganism.

In another illustrative embodiment, the method involves contacting the target microorganism with a permeabilization reagent that selectively permeabilizes the target microorganism; contacting the selectively permeabilized target microorganism with a cell-impermeant label; and detecting said label in the microorganism (cell) where the presence or amount of said label associated with a microorganism indicates the presence or amount of the target microorganism.

In certain embodiments the methods provide methods of detecting or quantifying a target microorganism in a sample are provided. The methods typically involve contacting the target microorganism with a selective permeabilization reagent that selectively permeabilizes or lyses the microorganism; contacting the selectively permeabilized microorganism with a detection reagent that is taken into the selectively permeabilized organism or that contacts metabolites or enzymes released by the selectively permeabilized microorganism, where the detection reagent produces a signal in the presence of the metabolites or enzymes; and detecting a signal produced by the detection reagent in the presence of the metabolites or enzymes where the strength of the signal indicates the presence and/or amount of the target microorganism in the sample. In certain embodiments the metabolites or enzymes comprise a metabolite or enzyme selected from the group consisting of ATP, DNA, RNA, calcium, beta-galactosidase (beta-gal), beta-glucuronidase, alcohol dehydrogenase or other NAD oxidoreductase, a transferase, an alkaline phosphatase or other hydrolase, a lyase, an isomerase, an oxidase, a gyrase, a DNA nuclease (DNases), and 1RNA nuclease (RNase), and a restriction enzyme. In certain embodiments the metabolites or enzymes comprise ATP. In certain embodiments the detection reagent comprises a luciferase and the signal comprises a luminescence signal. In certain embodiments the detection reagent comprises a target responsive electrochemical aptamer switch (TREAS) for ATP detection and the signal comprises an electrochemical signal. In certain embodiments the detection reagent comprises a molecular beacon (MB)-like DNA for the detection of ATP and the signal comprises a fluorescent signal. In certain embodiments the detection reagent comprises an enzyme substrate (e.g., beta-galactosidase (beta-gal), beta-glucuronidase, alcohol dehydrogenase or other NAD oxidoreductases, transferases, alkaline phosphatases or other hydrolases, lyases, isomerases, oxidases, gyrases, a DNA nuclease (DNases), and 1RNA nuclease (RNase), a restriction enzyme, and the like) and the detecting comprises detecting a reaction between the released enzyme and the enzyme substrate. In certain embodiments the substrate is selected from the group consisting of coumarin-4-acetic acid 7-O-caprylate, coumarin-4-acetic acid 7-O-beta-D-glucuronide, and coumarin-4-acetic acid 7-O-beta-D-galactopyranoside. In certain embodiments the detection reagent comprises an enzyme (e.g., an enzyme that uses NAD, NADP, or FAD as a cofactor) and a substrate for that enzyme and the detecting comprises detecting the reaction between the enzyme and the substrate in the presence of a cofactor or a coenzyme that is released from the microorganism. In various embodiments the enzyme substrate and/or the enzyme is provided on and/or in a solid support. In certain embodiments the substrate comprises glucose or another substrate for glucose oxidase, and glucose dehydrogenase. In certain embodiments the detecting comprises detecting the reduction of one or more coenzymes selected from the group consisting of NAD, NADP, and FAD. In certain embodiments the substrate comprises hexokinase, a hexose, glucose-6-phosphate dehydrogenase, and NAD. In certain embodiments the detecting comprises detecting released ATP by detecting the reduction of the NAD to NADH. In certain embodiments the substrate comprises glucose-6-phosphate dehydrogenase. In certain embodiments the detecting comprises detecting released NAD by detecting the reduction of the NAD to NADH. In various embodiments the detection of the reduction of NAD NADP, or FAD comprises detection of a colorimetric reagent that changes color when oxidized or reduced. In certain embodiments the detection of the reduction of NAD NADP or FAD comprises electrochemical detection of a reagent that is oxidized or reduced. In certain embodiments the substrate comprises a test strip compatible with a glucometer readout device. In certain embodiments the test strip comprises a calibration code. In various embodiments the contacting the target microorganism with a selective permeabilization reagent occurs on and/or in the substrate (support). In certain embodiments the contacting the target microorganism with a selective permeabilization reagent occurs in a sample collection device before application to the substrate.

In certain embodiments methods of detecting or quantifying a target microorganism in a sample are provided where the methods involve contacting the target microorganism with a permeabilization reagent that selectively permeabilizes the microorganism; contacting the selectively permeabilized microorganism with a cell-impermeant label; and detecting the label in the cell where the presence or amount of the label associated with a microorganism indicates the presence or amount of the target microorganism in the sample. In certain embodiments the detecting comprises a method selected from the group consisting of microscopy, flow cytometry, solid phase cytometry, luminometry, and spectroscopy. In certain embodiments the impermeant label comprises a label selected from the group consisting of propidium iodide, SYTOX Green, SYBR®-14, YoYo®-1, YO-PRO™-1, BO-PRO-1, PO-PRO-1, YO-PRO-1, TO-PRO-1, TO-PRO-3, BO-PRO-3, YO-PRO-3, TO-PRO-#, POPO-1, BOBO-1, YOYO-1, TOTO-1, POPO-3, BOBO-2, YOYO-3, TOTO-3, ethidium homodimers-1, ethidium homodimers-2, ethidium bromide, ethidium monoazide, and Trypan blue. In certain embodiments the detecting comprises a method selected from the group consisting of microscopy, flow cytometry, solid phase cytometry. In various embodiments the methods further involve concentrating the microorganisms before detecting the label.

In certain embodiments of any one of the foregoing methods, the permeabilization reagent comprises a reagent that disrupts or permeabilizes a microorganism or cell (e.g., an antimicrobial peptide) attached to a targeting peptide or antibody that preferentially or specifically binds to the target microorganism. In certain embodiments the targeting peptide preferentially or specifically binds to a target microorganism selected from the group consisting of Acinetobacter baumannii, Actinomyces naeslundii, Aspergillus niger, Bacteroides fragilis, Bacillus subtilis, Candida albicans, Clostridium difficile, Corynebacterium jeikeium, Campylobacter jejuni, Escherichia coli, Enterococcus faecalis, Fusobacterium nucleatum, Lactobacillus acidophilus, Legionella pneumophila, Micrococcus luteus, Mycobacterium smegmatis, Malassezia furfur, Methicillin-resistant Staphylococcus aureus (MRSA), Myxococcus xanthus, Pseudomonas aeruginosa, Porphyromonas gingivalis, Progeussmirabilis, S. epidermidis, Streptococcus mutans, Streptococcus pneumoniae, Treponema denticola, and Trichophyton rubrum. In certain embodiments the targeting peptide is a targeting peptide selected from the targeting peptides listed in Table 2. In certain embodiments the targeting peptide is attached directly or indirectly (e.g. via a linker) to an antimicrobial peptide. In certain embodiments the antimicrobial peptide is an antimicrobial peptide selected from the antimicrobial peptides listed in Table 4. In various embodiments the target microorganism is S. mutans, and the targeting peptide attached to an antimicrobial peptide comprises an amino acid sequence selected from the group consisting of TFFRLFNRSFTQALGKGGGKNLRIIRKGIHIIKKY (C16G2, SEQ ID NO:1117), KFINGVLSQFVLERKPYPKLFKFLRKHLL (1845L621, SEQ ID NO:1118), FIDSFIRSFGGGKLFKFLRKHLL (b43BD2.21, (SEQ ID NO:1119), TFFRLFNRSFTQALGKGGGFLKFLKKFFKKLKY (C16AF5, (SEQ ID NO:1120), and FIKHFIHRFGGGKNLRIIRKGIHIIKKY (2_{—}1G2, (SEQ ID NO:1121). In certain embodiments the targeting peptide attached to an antimicrobial peptide comprises an amino acid sequence selected from the group consisting of KKHRKHRKHRKH GGSGGS KNLRRIIRKGIHIIKKYG (G10KHc, (SEQ ID NO:1122). In various embodiments of any one of the foregoing methods the method is performed in a well of a multi-well plate. In certain embodiments different wells of the multi-well plate contain permeabilization reagents that selectively permeabilize different microorganisms. In any one of the foregoing embodiments, the sample comprises a sample from saliva, plaque, urine, feces, cerebrospinal fluid, blood, vaginal secretions, soil, a surface swab, an agricultural product, a meat product, a poultry product, and a fish product.

In various embodiments a diagnostic test device is provided. The device typically comprises a substrate test strip comprising a selective permeabilization reagent; an enzyme substrate; and a detection reagent that detects a change in oxidation state of a coenzyme. In certain embodiments the substrate comprises glucose or another substrate for glucose oxidase, and glucose dehydrogenase. In certain embodiments substrate comprises one or more coenzymes selected from the group consisting of NAD and FAD. In certain embodiments the substrate comprises hexokinase, a hexose, glucose-6-phosphate dehydrogenase, and NAD. In certain embodiments the substrate comprises glucose-6-phosphate dehydrogenase. In certain embodiments the detection reagent comprises a colorimetric reagent that changes color when oxidized or reduced. In certain embodiments the detection reagent that is detectable using an electrochemical detection device. In certain embodiments substrate comprises a test strip compatible with a glucometer readout device. In certain embodiments the test strip comprises a calibration code.

In certain embodiments a diagnostic test unit is provided. The test unit typically comprises a swab member carried by a housing base defining a sample chamber a housing cap comprising a first reagent chamber where the housing cap interfits with the housing base to cooperatively form a capped sample chamber with the swab disposed therein and a break-off nib, channel, or port that communicates between the first reagent chamber and the sample chamber; and a permeabilization reagent that selectively permeabilizes or lyses a target microorganism where the permeabilization reagent is disposed within the first reagent chamber or within the sample chamber. In certain embodiments the first reaction chamber further contains a detection reagent. In certain embodiments the housing cap or the housing base comprises a second reagent chamber containing a detection reagent. In certain embodiments the sample chamber contains a detection reagent. In certain embodiments the detection reagent comprises an enzyme substrate or a luciferase. In certain embodiments permeabilization reagent comprises a reagent that disrupts or permeabilizes a microorganism attached to a targeting peptide that preferentially or specifically binds to the target microorganism. In certain embodiments the targeting peptide preferentially or specifically binds to a target microorganism selected from the group consisting of Acinetobacter baumannii, Actinomyces naeslundii, Aspergillus niger, Bacteroides fragilis, Bacillus subtilis, Candida albicans, Clostridium difficile, Corynebacterium jeikeium, Campylobacter jejuni, Escherichia coli, Enterococcus faecalis, Fusobacterium nucleatum, Lactobacillus acidophilus, Legionella pneumophila, Micrococcus luteus, Mycobacterium smegmatis, Malassezia furfur, Methicillin-resistant Staphylococcus aureus (MRSA), Myxococcus xanthus, Pseudomonas aeruginosa, Porphyromonas gingivalis, Progeussmirabilis, S. epidermidis, Streptococcus mutans, Streptococcus pneumoniae, Treponema denticola, and Trichophyton rubrum. In certain embodiments targeting peptide is a targeting peptide selected from the targeting peptides listed in Table 2. In certain embodiments the targeting peptide is attached directly or indirectly to an antimicrobial peptide. In certain embodiments the antimicrobial peptide is an antimicrobial peptide selected from the antimicrobial peptides listed in Table 4. In certain embodiments the target microorganism is S. mutans, and the targeting peptide attached to an antimicrobial peptide comprises an amino acid sequence selected from the group consisting of TFFRLFNRSFTQALGKGGGKNLRIIRKGIHIIKKY (C16G2, SEQ ID NO:1129), KFINGVLSQFVLERKPYPKLFKFLRKHLL (1845L621, SEQ ID NO:1130), FIDSFIRSFGGGKLFKFLRKHLL (b43BD2.21, (SEQ ID NO:1131), TFFRLFNRSFTQALGKGGGFLKFLKKFFKKLKY (C16AF5, (SEQ ID NO:1132), and FIKHFIHRFGGGKNLRIIRKGIHIIKKY (2_{—}1G2, (SEQ ID NO:1133). In certain embodiments the targeting peptide attached to an antimicrobial peptide comprises an amino acid sequence selected from the group consisting of KKHRKHRKHRKH GGSGGS KNLRRIIRKGIHIIKKYG (G10KHc, (SEQ ID NO:1134).

In certain embodiments in any of the foregoing methods, diagnostic devices, or diagnostic test units, the selective permeabilization reagent comprises one of the targeting peptides selected from Table 2 attached to an antimicrobial peptide (AMP). Such “STAMPS” include, for example, 1T-3 attached to an AMP, 1T-4 attached to an AMP, 1T-6 attached to an AMP, 1T-7 attached to an AMP, 1T-8 attached to an AMP, 1T-9 attached to an AMP, 1T-10 attached to an AMP, 1T-11 attached to an AMP, 1T-12 attached to an AMP, 1T-13 attached to an AMP, 1T-14 attached to an AMP, 1T-15 attached to an AMP, 1T-16 attached to an AMP, 1T-17 attached to an AMP, 1T-18 attached to an AMP, 1T-19 attached to an AMP, 1T-20 attached to an AMP, 1T-21 attached to an AMP, 1T-22 attached to an AMP, 1T-23 attached to an AMP, 1T-24 attached to an AMP, 1T-25 attached to an AMP, 1T-26 attached to an AMP, 1T-27 attached to an AMP, 1T-28 attached to an AMP, 1T-29 attached to an AMP, 1T-30 attached to an AMP, 1T-31 attached to an AMP, 1T-32 attached to an AMP, 1T-33 attached to an AMP, 1T-34 attached to an AMP, 1T-35 attached to an AMP, 1T-36 attached to an AMP, 1T-37 attached to an AMP, 1T-38 attached to an AMP, 1T-39 attached to an AMP, 1T-40 attached to an AMP, 1T-41 attached to an AMP, 1T-42 attached to an AMP, 1T-43 attached to an AMP, 1T-44 attached to an AMP, 1T-45 attached to an AMP, 1T-46 attached to an AMP, 1T-47 attached to an AMP, 1T-48 attached to an AMP, 1T-49 attached to an AMP, 1T-50 attached to an AMP, 1T-51 attached to an AMP, 1T-52 attached to an AMP, 1T-53 attached to an AMP, 1T-54 attached to an AMP, 1T-55 attached to an AMP, 1T-56 attached to an AMP, 1T-57 attached to an AMP, 1T-58 attached to an AMP, 1T-59 attached to an AMP, 1T-60 attached to an AMP, 1T-61 attached to an AMP, 1T-62 attached to an AMP, 1T-63 attached to an AMP, 1T-64 attached to an AMP, 1T-65 attached to an AMP, 1T-66 attached to an AMP, 1T-67 attached to an AMP, 1T-68 attached to an AMP, 1T-69 attached to an AMP, 1T-70 attached to an AMP, 1T-71 attached to an AMP, 1T-72 attached to an AMP, 1T-73 attached to an AMP, 1T-74 attached to an AMP, 1T-75 attached to an AMP, 1T-76 attached to an AMP, 1T-77 attached to an AMP, 1T-78 attached to an AMP, 1T-79 attached to an AMP, 1T-80 attached to an AMP, 1T-81 attached to an AMP, 1T-82 attached to an AMP, 1T-83 attached to an AMP, 1T-84 attached to an AMP, 1T-85 attached to an AMP, 1T-86 attached to an AMP, 1T-87 attached to an AMP, 1T-89 attached to an AMP, 1T-90 attached to an AMP, 1T-91 attached to an AMP, 1T-92 attached to an AMP, 1T-93 attached to an AMP, 1T-94 attached to an AMP, 1T-95 attached to an AMP, 1T-96 attached to an AMP, 1T-97 attached to an AMP, 1T-98 attached to an AMP, 1T-99 attached to an AMP, 1T-100 attached to an AMP, 1T-101 attached to an AMP, 1T-102 attached to an AMP, 1T-103 attached to an AMP, 1T-104 attached to an AMP, 1T-105 attached to an AMP, 1T-106 attached to an AMP, 1T-107 attached to an AMP, 1T-108 attached to an AMP, 1T-109 attached to an AMP, 1T-110 attached to an AMP, 1T-111 attached to an AMP, 1T-112 attached to an AMP, 1T-113 attached to an AMP, 1T-114 attached to an AMP, 1T-115 attached to an AMP, 1T-116 attached to an AMP, 1T-117 attached to an AMP, 1T-118 attached to an AMP, 1T-119 attached to an AMP, 1T-120 attached to an AMP, 1T-121 attached to an AMP, 1T-122 attached to an AMP, 1T-123 attached to an AMP, 1T-124 attached to an AMP, 1T-125 attached to an AMP, 1T-126 attached to an AMP, 1T-127 attached to an AMP, 1T-128 attached to an AMP, 1T-129 attached to an AMP, 1T-130 attached to an AMP, 1T-131 attached to an AMP, 1T-132 attached to an AMP, 1T-133 attached to an AMP, 1T-134 attached to an AMP, 1T-135 attached to an AMP, 1T-136 attached to an AMP, 1T-137 attached to an AMP, 1T-138 attached to an AMP, 1T-139 attached to an AMP, 1T-140 attached to an AMP, 1T-141 attached to an AMP, 1T-142 attached to an AMP, 1T-143 attached to an AMP, 1T-144 attached to an AMP, 1T-145 attached to an AMP, 1T-146 attached to an AMP, 1T-147 attached to an AMP, 1T-148 attached to an AMP, 1T-149 attached to an AMP, 1T-150 attached to an AMP, 1T-151 attached to an AMP, 1T-152 attached to an AMP, 1T-153 attached to an AMP, 1T-154 attached to an AMP, 1T-155 attached to an AMP, 1T-156 attached to an AMP, 1T-157 attached to an AMP, 1T-158 attached to an AMP, 1T-159 attached to an AMP, 1T-160 attached to an AMP, 1T-161 attached to an AMP, 1T-162 attached to an AMP, 1T-163 attached to an AMP, 1T-164 attached to an AMP, 1T-165 attached to an AMP, 1T-166 attached to an AMP, 1T-167 attached to an AMP, 1T-168 attached to an AMP, 1T-169 attached to an AMP, 1T-170 attached to an AMP, 1T-171 attached to an AMP, 1T-172 attached to an AMP, 1T-173 attached to an AMP, 1T-174 attached to an AMP, 1T-175 attached to an AMP, 1T-176 attached to an AMP, 1T-177 attached to an AMP, 1T-178 attached to an AMP, 1T-179 attached to an AMP, 1T-180 attached to an AMP, 1T-181 attached to an AMP, 1T-182 attached to an AMP, 1T-183 attached to an AMP, 1T-184 attached to an AMP, 1T-185 attached to an AMP, 1T-188 attached to an AMP, 1T-190 attached to an AMP, 1T-192 attached to an AMP, 1T-193 attached to an AMP, 1T-195 attached to an AMP, 1T-196 attached to an AMP, 1T-197 attached to an AMP, 1T-198 attached to an AMP, 1T-199 attached to an AMP, 1T-200 attached to an AMP, 1T-201 attached to an AMP, 1T-202 attached to an AMP, 1T-203 attached to an AMP, 1T-207 attached to an AMP, 1T-208 attached to an AMP, 1T-215 attached to an AMP, 1T-216 attached to an AMP, 1T-217 attached to an AMP, 1T-218 attached to an AMP, 1T-219 attached to an AMP, 1T-221 attached to an AMP, 1T-222 attached to an AMP, 1T-223 attached to an AMP, 1T-225 attached to an AMP, 1T-226 attached to an AMP, 1T-228 attached to an AMP, 1T-230 attached to an AMP, 1T-233 attached to an AMP, 1T-234 attached to an AMP, 1T-237 attached to an AMP, 1T-238 attached to an AMP, 1T-239 attached to an AMP, 1T-240 attached to an AMP, 1T-242 attached to an AMP, 1T-243 attached to an AMP, 1T-248 attached to an AMP, 1T-249 attached to an AMP, 1T-250 attached to an AMP, 1T-252 attached to an AMP, 1T-253 attached to an AMP, 1T-256 attached to an AMP, 1T-257 attached to an AMP, 1T-258 attached to an AMP, 1T-259 attached to an AMP, 1T-260 attached to an AMP, 1T-261 attached to an AMP, 1T-262 attached to an AMP, 1T-263 attached to an AMP, 1T-264 attached to an AMP, 1T-265 attached to an AMP, 1T-266 attached to an AMP, 1T-267 attached to an AMP, 1T-268 attached to an AMP, 1T-269 attached to an AMP, 1T-270 attached to an AMP, 1T-271 attached to an AMP, 1T-272 attached to an AMP, 1T-273 attached to an AMP, 1T-274 attached to an AMP, 1T-275 attached to an AMP, 1T-276 attached to an AMP, 1T-277 attached to an AMP, 1T-278 attached to an AMP, 1T-279 attached to an AMP, 1T-280 attached to an AMP, 1T-281 attached to an AMP, 1T-282 attached to an AMP, 1T-283 attached to an AMP, 1T-284 attached to an AMP, 1T-285 attached to an AMP, 1T-286 attached to an AMP, 1T-287 attached to an AMP, 1T-288 attached to an AMP, 1T-289 attached to an AMP, 1T-290 attached to an AMP, 1T-291 attached to an AMP, 1T-292 attached to an AMP, 1T-293 attached to an AMP, 1T-294 attached to an AMP, 1T-295 attached to an AMP, 1T-296 attached to an AMP, 1T-297 attached to an AMP, 1T-298 attached to an AMP, 1T-299 attached to an AMP, 1T-300 attached to an AMP, 1T-301 attached to an AMP, 1T-302 attached to an AMP, 1T-303 attached to an AMP, 1T-304 attached to an AMP, TT-305 attached to an AMP, TT-306 attached to an AMP, TT-307 attached to an AMP, TT-308 attached to an AMP, TT-309 attached to an AMP, TT-310 attached to an AMP, TT-311 attached to an AMP, PF-060 attached to an AMP, PF-024 attached to an AMP, PF-636 attached to an AMP, PF-178 attached to an AMP, PF-761 attached to an AMP, PF-770 attached to an AMP, 1T-65 attached to an AMP, PF-141 attached to an AMP, PF-543 attached to an AMP, PF-634 attached to an AMP, PF-040 attached to an AMP, PF-051 attached to an AMP, PF-580 attached to an AMP, PF-583 attached to an AMP, 1T-36 attached to an AMP, PF-206 attached to an AMP, 1T-13 attached to an AMP, 1T-21 attached to an AMP, PF-030 attached to an AMP, PF-463 attached to an AMP, PF-380 attached to an AMP, PF-515 attached to an AMP, PF-458 attached to an AMP, PF-S018 attached to an AMP, 1T-16 attached to an AMP, PF-211 attached to an AMP, PF-002 attached to an AMP, PF-S003 attached to an AMP, PF-021 attached to an AMP, 1T-14 attached to an AMP, 1T-15 attached to an AMP, PF-629 attached to an AMP, PF-617 attached to an AMP, PF-621 attached to an AMP, PF-631 attached to an AMP, PF-C009 attached to an AMP, 1T-17 attached to an AMP, 1T-20 attached to an AMP, 1T-68 attached to an AMP, 1T-70 attached to an AMP, PF-167 attached to an AMP, PF-168 attached to an AMP, PF-170 attached to an AMP, PF-176 attached to an AMP, attached to an AMP, Rv2369c attached to an AMP, PF2-038 attached to an AMP, Rv2561 attached to an AMP, PF2-040 attached to an AMP, Rv1535 attached to an AMP, PF2-033 attached to an AMP, Rv2660c attached to an AMP, PF2-044 attached to an AMP, Rv3760 attached to an AMP, PF2-059 attached to an AMP, PF-S024 attached to an AMP, PF-001 attached to an AMP, PF-002 attached to an AMP, PF-003 attached to an AMP, PF-004 attached to an AMP, PF-005 attached to an AMP, PF-006 attached to an AMP, PF-007 attached to an AMP, PF-008 attached to an AMP, PF-009 attached to an AMP, PF-010 attached to an AMP, PF-011 attached to an AMP, PF-012 attached to an AMP, PF-013 attached to an AMP, PF-014 attached to an AMP, PF-015 attached to an AMP, PF-016 attached to an AMP, PF-017 attached to an AMP, PF-018 attached to an AMP, PF-020 attached to an AMP, PF-021 attached to an AMP, PF-022 attached to an AMP, PF-023 attached to an AMP, PF-024 attached to an AMP, PF-025 attached to an AMP, PF-026 attached to an AMP, PF-027 attached to an AMP, PF-028 attached to an AMP, PF-029 attached to an AMP, PF-030 attached to an AMP, PF-031 attached to an AMP, PF-033 attached to an AMP, PF-034 attached to an AMP, PF-035 attached to an AMP, PF-036 attached to an AMP, PF-037 attached to an AMP, PF-038 attached to an AMP, PF-039 attached to an AMP, PF-040 attached to an AMP, PF-041 attached to an AMP, PF-042 attached to an AMP, PF-043 attached to an AMP, PF-044 attached to an AMP, PF-045 attached to an AMP, PF-046 attached to an AMP, PF-047 attached to an AMP, PF-048 attached to an AMP, PF-049 attached to an AMP, PF-050 attached to an AMP, PF-051 attached to an AMP, PF-052 attached to an AMP, PF-053 attached to an AMP, PF-054 attached to an AMP, PF-055 attached to an AMP, PF-056 attached to an AMP, PF-057 attached to an AMP, PF-058 attached to an AMP, PF-060 attached to an AMP, PF-061 attached to an AMP, PF-062 attached to an AMP, PF-063 attached to an AMP, PF-064 attached to an AMP, PF-065 attached to an AMP, PF-066 attached to an AMP, PF-067 attached to an AMP, PF-068 attached to an AMP, PF-069 attached to an AMP, PF-070 attached to an AMP, PF-071 attached to an AMP, PF-073 attached to an AMP, PF-074 attached to an AMP, PF-075 attached to an AMP, PF-076 attached to an AMP, PF-097 attached to an AMP, PF-099 attached to an AMP, PF-101 attached to an AMP, PF-104 attached to an AMP, PF-123 attached to an AMP, PF-124 attached to an AMP, PF-125 attached to an AMP, PF-126 attached to an AMP, PF-127 attached to an AMP, PF-128 attached to an AMP, PF-129 attached to an AMP, PF-130 attached to an AMP, PF-131 attached to an AMP, PF-132 attached to an AMP, PF-133 attached to an AMP, PF-134 attached to an AMP, PF-135 attached to an AMP, PF-136 attached to an AMP, PF-137 attached to an AMP, PF-138 attached to an AMP, PF-139 attached to an AMP, PF-140 attached to an AMP, PF-141 attached to an AMP, PF-142 attached to an AMP, PF-143 attached to an AMP, PF-144 attached to an AMP, PF-145 attached to an AMP, PF-146 attached to an AMP, PF-147 attached to an AMP, PF-148 attached to an AMP, PF-149 attached to an AMP, PF-150 attached to an AMP, PF-151 attached to an AMP, PF-152 attached to an AMP, PF-153 attached to an AMP, PF-154 attached to an AMP, PF-155 attached to an AMP, PF-156 attached to an AMP, PF-157 attached to an AMP, PF-158 attached to an AMP, PF-159 attached to an AMP, PF-160 attached to an AMP, PF-161 attached to an AMP, PF-162 attached to an AMP, PF-163 attached to an AMP, PF-164 attached to an AMP, PF-165 attached to an AMP, PF-166 attached to an AMP, PF-167 attached to an AMP, PF-168 attached to an AMP, PF-169 attached to an AMP, PF-170 attached to an AMP, PF-171 attached to an AMP, PF-172 attached to an AMP, PF-173 attached to an AMP, PF-174 attached to an AMP, PF-175 attached to an AMP, PF-176 attached to an AMP, PF-177 attached to an AMP, PF-178 attached to an AMP, PF-180 attached to an AMP, PF-181 attached to an AMP, PF-182 attached to an AMP, PF-183 attached to an AMP, PF-184 attached to an AMP, PF-185 attached to an AMP, PF-186 attached to an AMP, PF-187 attached to an AMP, PF-188 attached to an AMP, PF-189 attached to an AMP, PF-190 attached to an AMP, PF-191 attached to an AMP, PF-192 attached to an AMP, PF-193 attached to an AMP, PF-194 attached to an AMP, PF-195 attached to an AMP, PF-196 attached to an AMP, PF-197 attached to an AMP, PF-200 attached to an AMP, PF-201 attached to an AMP, PF-202 attached to an AMP, PF-203 attached to an AMP, PF-204 attached to an AMP, PF-205 attached to an AMP, PF-206 attached to an AMP, PF-207 attached to an AMP, PF-208 attached to an AMP, PF-209 attached to an AMP, PF-210 attached to an AMP, PF-211 attached to an AMP, PF-212 attached to an AMP, PF-213 attached to an AMP, PF-214 attached to an AMP, PF-215 attached to an AMP, PF-216 attached to an AMP, PF-217 attached to an AMP, PF-218 attached to an AMP, PF-219 attached to an AMP, PF-221 attached to an AMP, PF-222 attached to an AMP, PF-223 attached to an AMP, PF-224 attached to an AMP, PF-225 attached to an AMP, PF-226 attached to an AMP, PF-228 attached to an AMP, PF-229 attached to an AMP, PF-230 attached to an AMP, PF-231 attached to an AMP, PF-232 attached to an AMP, PF-233 attached to an AMP, PF-234 attached to an AMP, PF-235 attached to an AMP, PF-236 attached to an AMP, PF-237 attached to an AMP, PF-238 attached to an AMP, PF-239 attached to an AMP, PF-240 attached to an AMP, PF-242 attached to an AMP, PF-244 attached to an AMP, PF-245 attached to an AMP, PF-246 attached to an AMP, PF-247 attached to an AMP, PF-248 attached to an AMP, PF-249 attached to an AMP, PF-252 attached to an AMP, PF-253 attached to an AMP, PF-254 attached to an AMP, PF-255 attached to an AMP, PF-256 attached to an AMP, PF-257 attached to an AMP, PF-259 attached to an AMP, PF-262 attached to an AMP, PF-263 attached to an AMP, PF-265 attached to an AMP, PF-266 attached to an AMP, PF-267 attached to an AMP, PF-268 attached to an AMP, PF-270 attached to an AMP, PF-271 attached to an AMP, PF-273 attached to an AMP, PF-275 attached to an AMP, PF-276 attached to an AMP, PF-278 attached to an AMP, PF-279 attached to an AMP, PF-281 attached to an AMP, PF-282 attached to an AMP, PF-283 attached to an AMP, PF-284 attached to an AMP, PF-285 attached to an AMP, PF-288 attached to an AMP, PF-289 attached to an AMP, PF-291 attached to an AMP, PF-292 attached to an AMP, PF-294 attached to an AMP, PF-295 attached to an AMP, PF-297 attached to an AMP, PF-299 attached to an AMP, PF-300 attached to an AMP, PF-301 attached to an AMP, PF-304 attached to an AMP, PF-305 attached to an AMP, PF-306 attached to an AMP, PF-307 attached to an AMP, PF-308 attached to an AMP, PF-309 attached to an AMP, PF-311 attached to an AMP, PF-313 attached to an AMP, PF-314 attached to an AMP, PF-317 attached to an AMP, PF-319 attached to an AMP, PF-320 attached to an AMP, PF-323 attached to an AMP, PF-324 attached to an AMP, PF-325 attached to an AMP, PF-326 attached to an AMP, PF-328 attached to an AMP, PF-329 attached to an AMP, PF-333 attached to an AMP, PF-334 attached to an AMP, PF-336 attached to an AMP, PF-337 attached to an AMP, PF-338 attached to an AMP, PF-340 attached to an AMP, PF-344 attached to an AMP, PF-347 attached to an AMP, PF-348 attached to an AMP, PF-349 attached to an AMP, PF-350 attached to an AMP, PF-351 attached to an AMP, PF-352 attached to an AMP, PF-353 attached to an AMP, PF-354 attached to an AMP, PF-355 attached to an AMP, PF-356 attached to an AMP, PF-357 attached to an AMP, PF-358 attached to an AMP, PF-359 attached to an AMP, PF-360 attached to an AMP, PF-361 attached to an AMP, PF-362 attached to an AMP, PF-363 attached to an AMP, PF-364 attached to an AMP, PF-365 attached to an AMP, PF-366 attached to an AMP, PF-367 attached to an AMP, PF-368 attached to an AMP, PF-369 attached to an AMP, PF-370 attached to an AMP, PF-371 attached to an AMP, PF-372 attached to an AMP, PF-373 attached to an AMP, PF-374 attached to an AMP, PF-375 attached to an AMP, PF-376 attached to an AMP, PF-378 attached to an AMP, PF-379 attached to an AMP, PF-380 attached to an AMP, PF-381 attached to an AMP, PF-382 attached to an AMP, PF-383 attached to an AMP, PF-384 attached to an AMP, PF-385 attached to an AMP, PF-386 attached to an AMP, PF-387 attached to an AMP, PF-388 attached to an AMP, PF-389 attached to an AMP, PF-390 attached to an AMP, PF-392 attached to an AMP, PF-393 attached to an AMP, PF-394 attached to an AMP, PF-395 attached to an AMP, PF-396 attached to an AMP, PF-397 attached to an AMP, PF-398 attached to an AMP, PF-399 attached to an AMP, PF-400 attached to an AMP, PF-401 attached to an AMP, PF-402 attached to an AMP, PF-403 attached to an AMP, PF-404 attached to an AMP, PF-405 attached to an AMP, PF-406 attached to an AMP, PF-407 attached to an AMP, PF-408 attached to an AMP, PF-409 attached to an AMP, PF-410 attached to an AMP, PF-411 attached to an AMP, PF-412 attached to an AMP, PF-413 attached to an AMP, PF-414 attached to an AMP, PF-416 attached to an AMP, PF-417 attached to an AMP, PF-418 attached to an AMP, PF-421 attached to an AMP, PF-422 attached to an AMP, PF-423 attached to an AMP, PF-424 attached to an AMP, PF-425 attached to an AMP, PF-426 attached to an AMP, PF-427 attached to an AMP, PF-428 attached to an AMP, PF-429 attached to an AMP, PF-430 attached to an AMP, PF-431 attached to an AMP, PF-432 attached to an AMP, PF-433 attached to an AMP, PF-434 attached to an AMP, PF-435 attached to an AMP, PF-436 attached to an AMP, PF-438 attached to an AMP, PF-439 attached to an AMP, PF-440 attached to an AMP, PF-441 attached to an AMP, PF-442 attached to an AMP, PF-443 attached to an AMP, PF-444 attached to an AMP, PF-445 attached to an AMP, PF-446 attached to an AMP, PF-447 attached to an AMP, PF-448 attached to an AMP, PF-450 attached to an AMP, PF-451 attached to an AMP, PF-452 attached to an AMP, PF-453 attached to an AMP, PF-454 attached to an AMP, PF-456 attached to an AMP, PF-457 attached to an AMP, PF-458 attached to an AMP, PF-459 attached to an AMP, PF-460 attached to an AMP, PF-461 attached to an AMP, PF-462 attached to an AMP, PF-463 attached to an AMP, PF-464 attached to an AMP, PF-465 attached to an AMP, PF-466 attached to an AMP, PF-467 attached to an AMP, PF-469 attached to an AMP, PF-470 attached to an AMP, PF-471 attached to an AMP, PF-472 attached to an AMP, PF-473 attached to an AMP, PF-474 attached to an AMP, PF-475 attached to an AMP, PF-476 attached to an AMP, PF-477 attached to an AMP, PF-478 attached to an AMP, PF-479 attached to an AMP, PF-480 attached to an AMP, PF-481 attached to an AMP, PF-482 attached to an AMP, PF-483 attached to an AMP, PF-484 attached to an AMP, PF-486 attached to an AMP, PF-487 attached to an AMP, PF-488 attached to an AMP, PF-489 attached to an AMP, PF-490 attached to an AMP, PF-491 attached to an AMP, PF-492 attached to an AMP, PF-493 attached to an AMP, PF-494 attached to an AMP, PF-495 attached to an AMP, PF-496 attached to an AMP, PF-497 attached to an AMP, PF-498 attached to an AMP, PF-499 attached to an AMP, PF-500 attached to an AMP, PF-501 attached to an AMP, PF-502 attached to an AMP, PF-503 attached to an AMP, PF-504 attached to an AMP, PF-505 attached to an AMP, PF-506 attached to an AMP, PF-507 attached to an AMP, PF-508 attached to an AMP, PF-509 attached to an AMP, PF-510 attached to an AMP, PF-511 attached to an AMP, PF-512 attached to an AMP, PF-513 attached to an AMP, PF-514 attached to an AMP, PF-515 attached to an AMP, PF-517 attached to an AMP, PF-518 attached to an AMP, PF-519 attached to an AMP, PF-520 attached to an AMP, PF-521 attached to an AMP, PF-522 attached to an AMP, PF-523 attached to an AMP, PF-524 attached to an AMP, PF-526 attached to an AMP, PF-527 attached to an AMP, PF-528 attached to an AMP, PF-529 attached to an AMP, PF-537 attached to an AMP, PF-539 attached to an AMP, PF-540 attached to an AMP, PF-542 attached to an AMP, PF-543 attached to an AMP, PF-544 attached to an AMP, PF-545 attached to an AMP, PF-546 attached to an AMP, PF-547 attached to an AMP, PF-548 attached to an AMP, PF-549 attached to an AMP, PF-550 attached to an AMP, PF-551 attached to an AMP, PF-552 attached to an AMP, PF-553 attached to an AMP, PF-554 attached to an AMP, PF-555 attached to an AMP, PF-556 attached to an AMP, PF-557 attached to an AMP, PF-558 attached to an AMP, PF-559 attached to an AMP, PF-560 attached to an AMP, PF-562 attached to an AMP, PF-563 attached to an AMP, PF-564 attached to an AMP, PF-566 attached to an AMP, PF-567 attached to an AMP, PF-568 attached to an AMP, PF-569 attached to an AMP, PF-570 attached to an AMP, PF-572 attached to an AMP, PF-573 attached to an AMP, PF-575 attached to an AMP, PF-576 attached to an AMP, PF-577 attached to an AMP, PF-578 attached to an AMP, PF-579 attached to an AMP, PF-580 attached to an AMP, PF-581 attached to an AMP, PF-583 attached to an AMP, PF-584 attached to an AMP, PF-585 attached to an AMP, PF-586 attached to an AMP, PF-587 attached to an AMP, PF-588 attached to an AMP, PF-589 attached to an AMP, PF-590 attached to an AMP, PF-592 attached to an AMP, PF-593 attached to an AMP, PF-594 attached to an AMP, PF-595 attached to an AMP, PF-596 attached to an AMP, PF-597 attached to an AMP, PF-598 attached to an AMP, PF-599 attached to an AMP, PF-600 attached to an AMP, PF-601 attached to an AMP, PF-602 attached to an AMP, PF-603 attached to an AMP, PF-604 attached to an AMP, PF-605 attached to an AMP, PF-607 attached to an AMP, PF-609 attached to an AMP, PF-610 attached to an AMP, PF-612 attached to an AMP, PF-613 attached to an AMP, PF-614 attached to an AMP, PF-615 attached to an AMP, PF-616 attached to an AMP, PF-617 attached to an AMP, PF-619 attached to an AMP, PF-621 attached to an AMP, PF-622 attached to an AMP, PF-623 attached to an AMP, PF-625 attached to an AMP, PF-626 attached to an AMP, PF-627 attached to an AMP, PF-629 attached to an AMP, PF-630 attached to an AMP, PF-631 attached to an AMP, PF-632 attached to an AMP, PF-634 attached to an AMP, PF-635 attached to an AMP, PF-636 attached to an AMP, PF-637 attached to an AMP, PF-638 attached to an AMP, PF-639 attached to an AMP, PF-640 attached to an AMP, PF-641 attached to an AMP, PF-642 attached to an AMP, PF-643 attached to an AMP, PF-644 attached to an AMP, PF-645 attached to an AMP, PF-646 attached to an AMP, PF-647 attached to an AMP, PF-648 attached to an AMP, PF-649 attached to an AMP, PF-650 attached to an AMP, PF-651 attached to an AMP, PF-652 attached to an AMP, PF-653 attached to an AMP, PF-654 attached to an AMP, PF-655 attached to an AMP, PF-656 attached to an AMP, PF-657 attached to an AMP, PF-658 attached to an AMP, PF-659 attached to an AMP, PF-660 attached to an AMP, PF-661 attached to an AMP, PF-662 attached to an AMP, PF-663 attached to an AMP, PF-664 attached to an AMP, PF-665 attached to an AMP, PF-666 attached to an AMP, PF-667 attached to an AMP, PF-668 attached to an AMP, PF-669 attached to an AMP, PF670 attached to an AMP, PF-671 attached to an AMP, PF-673 attached to an AMP, PF-674 attached to an AMP, PF-675 attached to an AMP, PF-676 attached to an AMP, PF-677 attached to an AMP, PF-678 attached to an AMP, PF-679 attached to an AMP, PF-680 attached to an AMP, PF-681 attached to an AMP, PF-682 attached to an AMP, PF-683 attached to an AMP, PF-684 attached to an AMP, PF-685 attached to an AMP, PF-686 attached to an AMP, PF-687 attached to an AMP, PF-688 attached to an AMP, PF-689 attached to an AMP, PF-690 attached to an AMP, PF-691 attached to an AMP, PF-692 attached to an AMP, PF-693 attached to an AMP, PF-694 attached to an AMP, PF-695 attached to an AMP, PF-696 attached to an AMP, PF-697 attached to an AMP, PF-698 attached to an AMP, PF-699 attached to an AMP, PF-700 attached to an AMP, PF-701 attached to an AMP, PF-702 attached to an AMP, PF-703 attached to an AMP, PF-704 attached to an AMP, PF-705 attached to an AMP, PF-706 attached to an AMP, PF-707 attached to an AMP, PF-709 attached to an AMP, PF-711 attached to an AMP, PF-712 attached to an AMP, PF-714 attached to an AMP, PF-716 attached to an AMP, PF-717 attached to an AMP, PF-720 attached to an AMP, PF-724 attached to an AMP, PF-727 attached to an AMP, PF-728 attached to an AMP, PF-731 attached to an AMP, PF-732 attached to an AMP, PF-733 attached to an AMP, PF-735 attached to an AMP, PF-737 attached to an AMP, PF-738 attached to an AMP, PF-741 attached to an AMP, PF-744 attached to an AMP, PF-745 attached to an AMP, PF-746 attached to an AMP, PF-748 attached to an AMP, PF-749 attached to an AMP, PF-750 attached to an AMP, PF-751 attached to an AMP, PF-752 attached to an AMP, PF-756 attached to an AMP, PF-757 attached to an AMP, PF-758 attached to an AMP, PF-760 attached to an AMP, PF-761 attached to an AMP, PF-762 attached to an AMP, PF-763 attached to an AMP, PF-764 attached to an AMP, PF-765 attached to an AMP, PF-766 attached to an AMP, PF-767 attached to an AMP, PF-768 attached to an AMP, PF-770 attached to an AMP, PF-772 attached to an AMP, PF-773 attached to an AMP, PF-776 attached to an AMP, PF-777 attached to an AMP, PF-778 attached to an AMP, PF-779 attached to an AMP, PF-780 attached to an AMP, PF-781 attached to an AMP, PF-782 attached to an AMP, PF-783 attached to an AMP, PF-784 attached to an AMP, PF-785 attached to an AMP, PF-786 attached to an AMP, PF-787 attached to an AMP, PF-788 attached to an AMP, PF-789 attached to an AMP, PF-790 attached to an AMP, PF-791 attached to an AMP, PF-792 attached to an AMP, PF-793 attached to an AMP, PF-794 attached to an AMP, PF-795 attached to an AMP, PF-796 attached to an AMP, PF-797 attached to an AMP, PF-798 attached to an AMP, PF-799 attached to an AMP, PF-800 attached to an AMP, PF-C003 attached to an AMP, PF-C008 attached to an AMP, PF-C034 attached to an AMP, PF-C045 attached to an AMP, PF-C050 attached to an AMP, PF-C052 attached to an AMP, PF-C055 attached to an AMP, PF-C057 attached to an AMP, PF-C058 attached to an AMP, PF-C061 attached to an AMP, PF-C062 attached to an AMP, PF-C063 attached to an AMP, PF-C064 attached to an AMP, PF-C065 attached to an AMP, PF-C068 attached to an AMP, PF-C069 attached to an AMP, PF-C071 attached to an AMP, PF-C072 attached to an AMP, PF-C075 attached to an AMP, PF-C0080 attached to an AMP, PF-C0084 attached to an AMP, PF-C0085 attached to an AMP, PF-C0086 attached to an AMP, PF-C088 attached to an AMP, PF-C091 attached to an AMP, PF-C093 attached to an AMP, PF-C095 attached to an AMP, PF-C098 attached to an AMP, PF-C120 attached to an AMP, PF-C131 attached to an AMP, PF-C134 attached to an AMP, PF-C 135 attached to an AMP, PF-C 136 attached to an AMP, PF-C 137 attached to an AMP, PF-C 139 attached to an AMP, PF-C 142 attached to an AMP, PF-C 143 attached to an AMP, PF-C145 attached to an AMP, PF-C160 attached to an AMP, PF-C180 attached to an AMP, PF-C181 attached to an AMP, PF-C194 attached to an AMP, PF-C259 attached to an AMP, PF-C271 attached to an AMP, PF-C273 attached to an AMP, PF-C281 attached to an AMP, PF-C285 attached to an AMP, PF-C290 attached to an AMP, PF-C291 attached to an AMP, PF-S003 attached to an AMP, PF-S004 attached to an AMP, PF-S007 attached to an AMP, PF-S015 attached to an AMP, PF-S018 attached to an AMP, PF-S023 attached to an AMP, PF-S026 attached to an AMP, or PF-S029 attached to an AMP. In certain illustrative embodiments the STAMP comprises any one of the foregoing targeting peptides attached (directly or through a linker (e.g., one of the linkers shown in Table 5)) to an one of the AMPs shown in Table 4 (e.g., one of K-1, K-2, K-7, K-8, K-9, K-10, K-11, K-12, K-13, K-14, K-15, K-16, K-17, K-18, K-19, K-20, K-21, K-22, 1T-88, PF-531, PF-527, PF-672, PF-606, PF-547, PF-C06, PF-545, PF-278, PF-283, PF-307, PF-168, PF-538, PF-448, PF-583, PF-600, PF-525, PF-529, PF-148, PF-530, PF-522, PF-497, PF-499, PF-322, PF-511, PF-512, PF-520, PF-521, PF-523, PF-524, PF-209, PF-437, V, W, X, Y, or Z).

In certain embodiments in any of the foregoing methods, diagnostic devices, or diagnostic test units the selective permeabilization reagent comprises one of the antimicrobial peptides selected from Table 4 attached to a targeting peptide forming a STAMP. In certain embodiments the targeting peptide is a peptide found in Table 2. Thus in various embodiments, the permeabilization reagent comprises a STAMP such as K-1 attached to one of the peptides 1T-3 through PF-Z in Table 2, K-2 attached to one of the peptides 1T-3 through PF-Z in Table 2, K-7 attached to one of the peptides 1T-3 through PF-Z in Table 2, K-8 attached to one of the peptides 1T-3 through PF-Z in Table 2, K-9 attached to one of the peptides 1T-3 through PF-Z in Table 2, K-10 attached to one of the peptides 1T-3 through PF-Z in Table 2, K-11 attached to one of the peptides 1T-3 through PF-Z in Table 2, K-12 attached to one of the peptides 1T-3 through PF-Z in Table 2, K-13 attached to one of the peptides 1T-3 through PF-Z in Table 2, K-14 attached to one of the peptides 1T-3 through PF-Z in Table 2, K-15 attached to one of the peptides 1T-3 through PF-Z in Table 2, K-16 attached to one of the peptides 1T-3 through PF-Z in Table 2, K-17 attached to one of the peptides 1T-3 through PF-Z in Table 2, K-18 attached to one of the peptides 1T-3 through PF-Z in Table 2, K-19 attached to one of the peptides 1T-3 through PF-Z in Table 2, K-20 attached to one of the peptides 1T-3 through PF-Z in Table 2, K-21 attached to one of the peptides 1T-3 through PF-Z in Table 2, K-22 attached to one of the peptides 1T-3 through PF-Z in Table 2, 1T-88 attached to one of the peptides 1T-3 through PF-Z in Table 2, PF-531 attached to one of the peptides 1T-3 through PF-Z in Table 2, PF-527 attached to one of the peptides 1T-3 through PF-Z in Table 2, PF-672 attached to one of the peptides 1T-3 through PF-Z in Table 2, PF-606 attached to one of the peptides 1T-3 through PF-Z in Table 2, PF-547 attached to one of the peptides 1T-3 through PF-Z in Table 2, PF-606 attached to one of the peptides 1T-3 through PF-Z in Table 2, PF-545 attached to one of the peptides 1T-3 through PF-Z in Table 2, PF-278 attached to one of the peptides 1T-3 through PF-Z in Table 2, PF-283 attached to one of the peptides 1T-3 through PF-Z in Table 2, PF-307 attached to one of the peptides 1T-3 through PF-Z in Table 2, PF-168 attached to one of the peptides 1T-3 through PF-Z in Table 2, PF-538 attached to one of the peptides 1T-3 through PF-Z in Table 2, PF-448 attached to one of the peptides 1T-3 through PF-Z in Table 2, PF-583 attached to one of the peptides 1T-3 through PF-Z in Table 2, PF-600 attached to one of the peptides 1T-3 through PF-Z in Table 2, PF-525 attached to one of the peptides 1T-3 through PF-Z in Table 2, PF-529 attached to one of the peptides 1T-3 through PF-Z in Table 2, PF-148 attached to one of the peptides 1T-3 through PF-Z in Table 2, PF-530 attached to one of the peptides 1T-3 through PF-Z in Table 2, PF-522 attached to one of the peptides 1T-3 through PF-Z in Table 2, PF-497 attached to one of the peptides 1T-3 through PF-Z in Table 2, PF-499 attached to one of the peptides 1T-3 through PF-Z in Table 2, PF-322 attached to one of the peptides 1T-3 through PF-Z in Table 2, PF-511 attached to one of the peptides 1T-3 through PF-Z in Table 2, PF-512 attached to one of the peptides 1T-3 through PF-Z in Table 2, PF-520 attached to one of the peptides 1T-3 through PF-Z in Table 2, PF-521 attached to one of the peptides 1T-3 through PF-Z in Table 2, PF-523 attached to one of the peptides 1T-3 through PF-Z in Table 2, PF-524 attached to one of the peptides 1T-3 through PF-Z in Table 2, PF-209 attached to one of the peptides 1T-3 through PF-Z in Table 2, PF-437 attached to one of the peptides 1T-3 through PF-Z in Table 2, V attached to one of the peptides 1T-3 through PF-Z in Table 2, W attached to one of the peptides 1T-3 through PF-Z in Table 2, X attached to one of the peptides 1T-3 through PF-Z in Table 2, Y attached to one of the peptides 1T-3 through PF-Z in Table 2, or Z attached to one of the peptides 1T-3 through PF-Z in Table 2.

In certain embodiments, the selective permeabilization reagent is not or does not comprise a biological organism. In certain embodiments the selective permeabilization reagent is not a bacteriophage (phage).

### DEFINITIONS

The terms “selectively permeabilize” or “selectively lyse” refers to increasing the permeability of the membrane (and/or where present a cell wall) of a target microorganism (or target cell) while having no or a substantially reduced effect on other target microorganisms (or target cell(s)) that may be present in the sample. In certain embodiments, a target microorganism or cell is selectively permeabilized by a reagent when contact of the reagent permits entry of at least 1.2×, preferably at least 1.5×, or 2×, more preferably at least 3×, 5×, or 10× the amount of the reagent into the target microorganism or cell as compared to the amount of the reagent that enters other microorganisms or cells in the same sample.

A “selective permeabilization reagent” refers to a reagent that selectively permeabilizes or selectively lyses a particular target microorganism or a particular group of target microorganisms (e.g., gram−bacteria, gram+bacteria, etc.).

The term “detection reagent” refers to a reagent or combination of reagents that can be used to detect the presence or quantity of a metabolite, enzyme, ionic species or other cellular component.

An “impermeant label” refers to a label that is unable to pass through or substantially unable to pass through a semipermeable membrane (e.g., a cell membrane), and/or where present a cell wall. The impermeant label thereby distinguishes a permeabilized or lysed cell from an unaltered (intact) cell.

A sample refers to target and substance or collection of substances in which or from which it is desired to ascertain the presence and/or quantity of one or more target microorganisms and/or cells. Illustrative samples, include, but are not limited to, samples of water, soil, crops and vegetation, meats, fish, and poultry, milk and cheese, and various biological samples derived from human or non-human organisms. In certain embodiments the sample comprises isolated cells, a mixed cellular community, or a clinical sample. Clinical sample materials include, but are not limited to blood or blood fractions, cerebrospinal fluid, urine, saliva, mucus, tissue samples, and the like.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies, including, but are not limited to, Fab′₂, IgG, IgM, IgA, scFv, dAb, nanobodies, unibodies, and diabodies.

In certain embodiments antibodies and fragments of the present invention can be bispecific. Bispecific antibodies or fragments can be of several configurations. For example, bispecific antibodies may resemble single antibodies (or antibody fragments) but have two different antigen binding sites (variable regions). In various embodiments bispecific antibodies can be produced by chemical techniques (Kranz et al. (1981) Proc. Natl. Acad. Sci., USA, 78: 5807), by “polydoma” techniques (see, e.g., U.S. Pat. No. 4,474,893), or by recombinant DNA techniques. In certain embodiments bispecific antibodies of the present invention can have binding specificities for at least two different epitopes, at least one of which is an epitope of a microbial organism. The microbial binding antibodies and fragments can also be heteroantibodies. Heteroantibodies are two or more antibodies, or antibody binding fragments (e.g., Fab) linked together, each antibody or fragment having a different specificity.

The term “STAMP” refers to Specifically Targeted Anti-Microbial Peptides. In various embodiments, a STAMP comprises one or more peptide targeting moieties attached to one or more antimicrobial moieties (e.g., antimicrobial peptides (AMPs)). An MH-STAMP is a STAMP bearing two or more targeting domains (i.e., a multi-headed STAMP).

### BRIEF DESCRIPTION OF THE DRAWINGS

FIGS. 1A and 1B schematically illustrate two embodiments of the methods described herein. FIG. 1A illustrates that in contrast to a typical lysis reagent (e.g., a detergent) a selectively permeabilization reagent permeabilizes the target microorganism (e.g., S. mutans) without substantially permeabilizing other microorganisms in the sample. This permits metabolites, enzymes, or other cellular components to exit the microorganism where they are detected indicating the presence and/or the amount of the target microorganism. FIG. 1B illustrates an assay where the selective permeabilization reagent permits entry of an impermeant label into the target microorganism

FIG. 2 illustrates one method of performing an assay described herein. As illustrated therein, in step A, a sample (e.g., saliva) is collected on a collection device (e.g., a swab). In step B, the sample is incubated with a selective permeabilization reagent (e.g., a STAMP). In step C, a detection reagent is added to the mix and the reaction is optionally agitated. In step D, the reaction mixture is inserted into a test reader, and in step E, the results are read.

FIG. 3 shows that assays described herein are capable of quantitatively detecting as little as 10⁴ cells/ml of cultured S. mutans grown in the lab.

FIG. 4 shows that assays described herein are capable of quantifying S. mutans spiked in a fresh unfiltered saliva sample.

FIG. 5 demonstrates targeted permeabilization of spiked S. mutans in fresh saliva samples.

FIG. 6 shows a schematic diagram of one illustrative diagnostic test unit.

### DETAILED DESCRIPTION

Novel methods and devices for the detection and/or quantification of microorganisms are provided herein. The methods are rapid, do not require significant instrumentation, and show high specificity and selectivity.

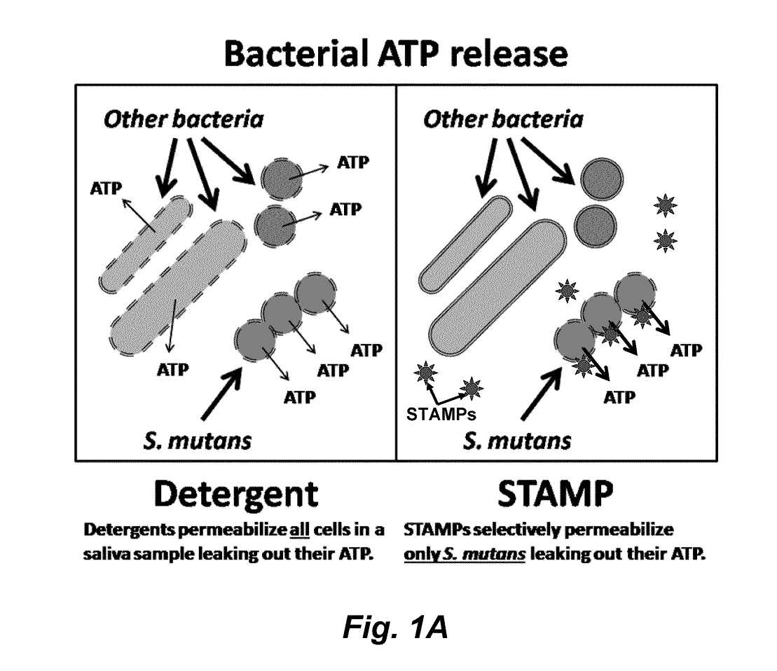

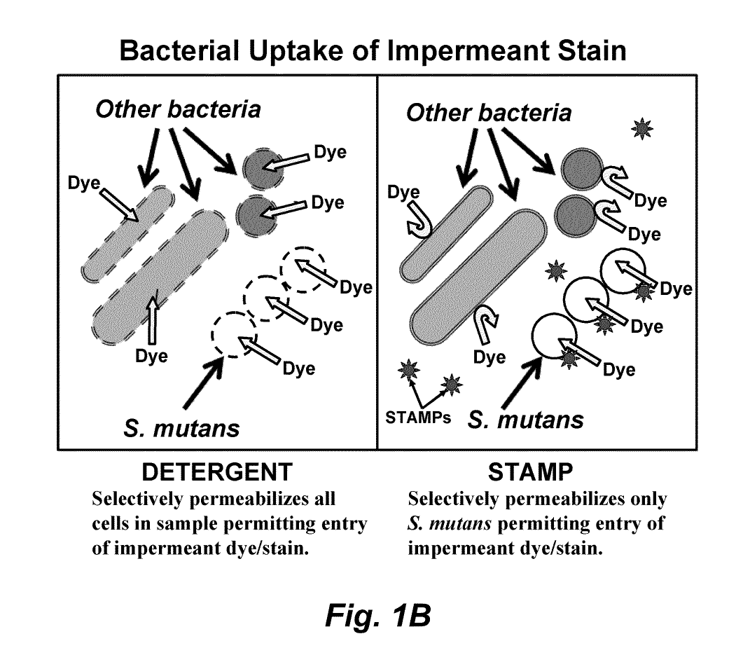

In certain embodiments the methods involve contacting a target microorganism (or a sample containing one or more target microorganisms) with a permeabilization reagent that selectively permeabilizes or lyses the target microorganism. The selective permeabilization releases enzymes or metabolites from the target microorganism where they can be contacted with one or more detection reagents that produce signal(s) upon contact/reaction with the enzyme or metabolite (see, e.g., FIG. 1). The magnitude of the signal provides an indication of the presence and/or amount of target microorganism present. Because the permeabilization reagent is selective for the target microorganism, the assay provides a signal that predominantly represents the presence or quantity of the target microorganism even in the presence of other microorganisms.

It will be appreciated, that in certain embodiments, the selective permeabilization permits entrance of the detection reagent(s) into the target microorganism where they react with metabolites or substrates and the reaction provides an indication of the presence and/or amount of the target microorganism.

Similarly, in another illustrative embodiment, the methods involve contacting the target microorganism (e.g., in a biological sample) with a permeabilization reagent that selectively permeabilizes the microorganism. The microorganism is contacted with a cell-impermeant label (e.g., a cell impermeant fluorescent dye (e.g., propidium iodide, SYTOX Green, etc.), a colorimetric dye (e.g. Trypan blue, etc.)) and, because the microorganisms is selectively permeabilized by the permeabilization reagent, the label enters the microorganism. Because the permeabilization reagent is selective for the target microorganism, other microorganisms that may be present are not permeabilized and internalize little or no label (see, e.g., FIG. 1B). The label is then detected in the microorganism where the presence or amount of said label associated with the microorganism indicates the presence or amount of the target microorganism in said sample. Additionally, a concentration step (filter, centrifugation, other) allows increased sensitivity of the method.

In certain embodiments microorganisms/cells permeabilized and stained with fluorescent or colorimetric dyes can be filtered (single pore size filter, serial filters, etc.) to remove debris, concentrate and capture bacteria/cells on the filter surface. Bacteria/cells can be quantitated by measuring the fluorescent or color intensity using a measuring device or by visual observation. Additionally bacteria/cells captured on the filter surface can be imaged via microscopy, solid-phase cytometry or other method.

The methods described herein can be performed to detect essentially any microorganism. Such microorganisms include, but are not limited to bacteria, yeasts, fungi, molds, viruses, algae, protozoa, and the like. In certain embodiments, the methods can be used to detect and/or quantify Gram-negative bacteria (e.g., Acinetobacter baumannii, Escherichia coli, Fusobacterium nucleatum, Pseudomonas aeruginosa, Porphyromonas gingivalis, and the like), Gram-positive bacteria (e.g., Actinomyces naeslundii, Bacillus subtilis, Clostridium difficile, Enterococcus faecalis, Staphylococcus aureus (and MRSA), S. epidermidis, Streptococcus mutans, Streptococcus pneumoniae, and the like), yeast or fungi (e.g., Aspergillus niger, Candida albicans, Malassezia furfur, Trichophyton rubrum, and the like) (see, e.g., Table 1 identifying illustrative target microorganisms and associated pathologies).

TABLE 1
Illustrative target microorganisms and associated pathology.

| | |
|---|---|
| Acinetobacter baumannii | Pathogenic gram-negative bacillus that is naturally sensitive |
| (A. baumannii) | to relatively few antibiotics. |
| Actinomyces naeslundii | Gram positive rod shaped bacteria that occupy the oral |
| (A. naeslundii) | cavity and are implicated in periodontal disease and root caries. |
| Aspergillus niger | A fungal infection that often causes a black mold to appear |
| (A. niger) | on some fruit and vegetables but may also infect humans |
| | through inhalation of fungal spores. |
| Bacteroides fragilis | Gram positive bacilli that are opportunistic human |
| (B. fragilis) | pathogens, causing infections of the peritoneal cavity, |
| | gastrointestinal surgery, and appendicitis via abscess |
| | formation, inhibiting phagocytosis. Resistant to a wide |
| | variety of antibiotics --β-lactams, aminoglycosides, and |
| | recently many species have acquired resistance to |
| | erythromycin and tetracycline. |
| Bacillus subtilis | Gram-positive, catalase-positive bacterium. |
| (B. subtilis) | |
| Candida albicans | Causal agent of opportunistic oral and genital fungal |
| (C. albicans) | infections in humans. |
| Clostridium difficile | A gram-positive, anaerobic, spore-forming bacillus that is |
| (C. difficile) | responsible for the development of antibiotic-associated |
| | diarrhea and colitis. |
| Corynebacterium jeikeium | Gram positive, opportunistic pathogen primarily of |
| (C. jeikeium) | immunocompromised (neutropenic) patients. Highly |
| | resistant to antibiotics |
| Campylobacter jejuni | Gram negative cause of human gastroenteritis/food |
| (C. jejuni) | poisoning. |
| Escherichia coli | Gram negative rod-shaped bacterium commonly found in the |
| (E. coli) | lower intestine of warm-blooded organisms. Certain strains |
| | cause serious food poisoning in humans. |
| Enterococcus faecalis | Gram-positive commensal bacterium |
| (E. faecalis) | |
| Fusobacterium nucleatum | Gram negative schizomycetes bacterium often seen in |
| (F. nucleatum) | necrotic tissue and implicated, but not conclusively, with |
| | other organisms in the causation and perpetuation of |
| | periodontal disease. |
| Lactobacillus acidophilus | Gram-positive commensal bacterium. |
| (L. acidophilus) | |
| Legionella pneumophila | Gram negative bacterium that is the causative agent of |
| (L. pneumophila) | legionellosis or Legionnaires' disease. |
| (Micrococcus luteus) | Gram positive, spherical, saprotrophic bacterium found in |
| M. luteus | soil, dust, water and air, and as part of the normal flora of the |
| | mammalian skin. The bacterium also colonizes the human |
| | mouth, mucosae, oropharynx and upper respiratory tract. |
| | Considered an emerging nosocomial pathogen in |
| | immunocompromised patients. |
| Mycobacterium smegmatis | Gram-variable (acid-fast) soil-dwelling organism utilized as |
| (M. smegmatis) | a proxy for Mycobacterium tuberculosis during research and |
| | development. |
| Malassezia furfur | Yeast-cutaneous pathogen. |
| (M. furfur) | |
| Methicillin-resistant | Any strain of Staphylococcus aureus bacteria (gram positive) |
| Staphylococcus aureus | that is resistant to a one or more members of a large group of |
| (MRSA) | antibiotics called the beta-lactams. Responsible for skin and |
| | systemic infections. |
| Myxococcus xanthus | Gram negative cells that form biofilms and display primitive |
| (M. xanthus) | social motility and fruiting body organization. |
| Pseudomonas aeruginosa | Gram-negative rod. Frequent opportunistic pathogen and |
| P. aeruginosa | infects burn wounds. Causes ear infections in children. |
| | Infects the lungs of cystic fibrosis patients. |
| Porphyromonas gingivalis | Non-motile, gram-negative, rod-shaped, anaerobic |
| (P. gingivalis) | pathogenic bacterium (periodontal disease) |
| Proteus mirabilis | Gram-negative, facultatively anaerobic bacterium. Causes |
| (P. mirabilis) | 90% of all ‘Proteus’ infections in humans. |
| Staphylococcus epidermidis | Gram-positive, coagulase-negative cocci. Nosocomial |
| (S. epidermidis) | pathogen associated with infection (biofilm) of implanted |
| | medical device. |
| Streptococcus mutans | Gram-positive, facultatively anaerobic bacterium commonly |
| (S. mutans) | found in the human oral cavity and is a significant |
| | contributor to tooth decay |
| Streptococcus pneumoniae | Gram-positive, alpha-hemolytic, bile soluble aerotolerant |
| (S. pneumoniae) | anaerobe. Causal agent for streptococcal pneumonia. |
| Treponema denticola | Gram-negative oral spirochete associated with the incidence |
| (T. denticola) | and severity of human periodontal disease and localized |
| | osteitis following tooth extraction (dry socket) |
| Trichophyton rubrum | Most common cause of athlete's foot, jock itch and |
| (T. rubrum) | ringworm. |

The methods described herein are not only useful to detect pathogens in biological samples derived from animals or humans, but can also be used to detect contaminants in foods/agricultural products, to detect environmental contaminants in, for example, soil or water, to detect contaminants in clean/sterile environments (e.g., hospitals, operating rooms), to detect contaminants of devices (e.g., surgical devices, etc.), and the like.

By way of example, Campylobacter jejuni is a common contaminant of poultry. Similarly, Clostridium botulinum is a common food toxin. Escherichia coli is a common toxin found in ground beef, raw milk, chicken, vegetables, and fruit. Salmonella typhimurium is typically found in meats, poultry, eggs or milk products. Shigella is often found as a contaminant of salads (potato, chicken, seafood, vegetable), raw vegetables, milk and other dairy products, and meat products especially poultry. Staphylococcus aureus is typically found in custard or cream-filled baked goods, ham, poultry, eggs, potato salad, cream sauces, sandwich fillings. Vibrio cholera, the causal agent of cholera can be transmitted by water or food. Vibrio vulnificus is a free-living ocean bacterium that can cause food borne illnesses from contaminated seafood and is especially dangerous in the warm weather months when eating shellfish that are undercooked or raw. Water contamination is usually due to the presence of three bacteria, E. coli, Clostridium perfringens, and enterococci, the bacteria normally found in the feces of people and many animals. The methods described herein can easily be used to screen foods, processing plants, and equipment for these various pathogens.

Similarly, the methods can be used to detect certain parasites. Such parasites include, but are not limited to Entamoeba histolytica, Giardia duodenalis, Cryptosporidium parvum, Cyclospora cayetanensis, Toxoplasma gondii, Trichinella spiralis, Taenia saginatajsolium, and Taenia saginata.

In certain embodiments the methods can be used to distinguish particular strains of microorganism.

The methods described herein are not limited to the detection of microorganisms. It will be recognized that such methods can be used to detect particular cells (using a targeting moiety that binds the target cell type), tissues comprising such cells, and the like.

The methods described herein are not limited to the detection/quantification of a single class (e.g., gram+/gram−), genus/species/strain of microorganism at a time. Using various formats, multiple microorganisms can be detected/quantified at a time. For example, the assays can be set up in a multi-well plate (e.g., 6, 24, 96, 384, 1536 well microtiter plates) where different wells contain different permeabilization reagents selective for different microorganisms and thereby permit detection of different target microorganisms. Similarly different permeabilization reagents can be provided in different regions of an array. In various embodiments, flow through systems can be used where different regions in a channel or tube can introduce a sample to different permeabilization reagents selective for different microorganisms and thereby permit sequential screening for different target microorganisms.

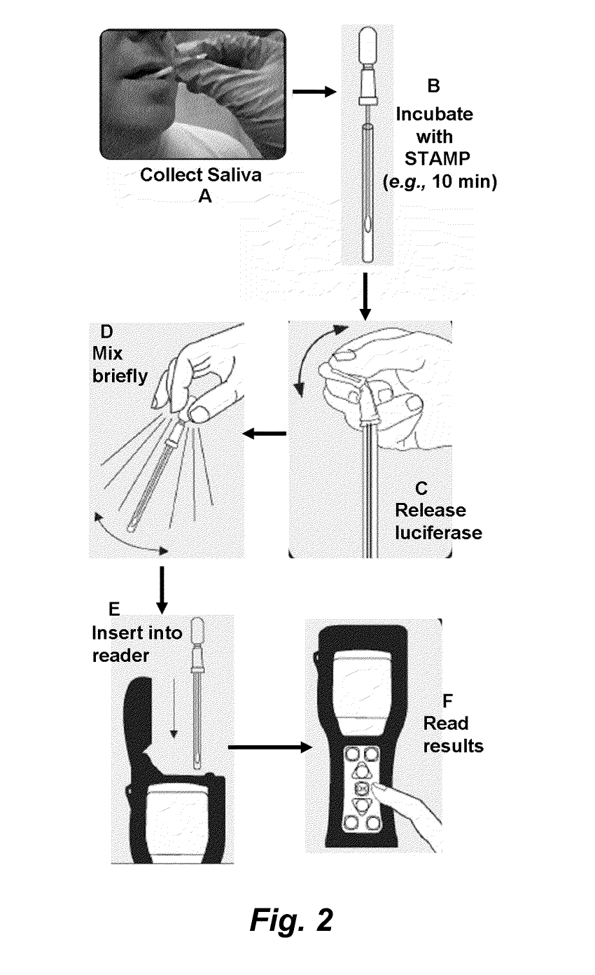

In one illustrative embodiment (schematically shown in FIG. 2), a saliva sample is collected (using for example, a swab). The saliva sample is deposited in a reaction chamber with a selective permeabilizing reagent (e.g., a STAMP) and the in another control reaction chamber (e.g., without a selective permeabilizing reagent). A luciferase reagent is added to the samples and they are mixed. Then light from the samples is measured to determine the presence of a targeted, permeabilized microorganism (e.g. S. mutans).

In a dental office, for example, the procedure could be carried out as follows: 1) Upon entering the exam room the dental assistant unpackages the S. mutans diagnostic containing a control and test reaction; 2) The patient holds the saliva collector in his mouth for 10 second allowing it to absorb saliva and bacteria present in the oral cavity; 3) The collector is removed, e.g., by the dental assistant and incubated for e.g., 10 minutes; 4) After incubation the luciferase reagent is added to the collected saliva and luminescence is measured in a handheld luminometer as prompted by the device to determine the presence and/or quantity of S. mutans.

The foregoing uses, target microorganisms, and embodiments are illustrative and not limiting. Using the teachings provided herein, other variants on the assays and uses thereof will be available to one of skill in the art.

### Selective Permeabilization Reagents.

In various embodiments selectivity/specificity of the assays described herein is provided (at least in part) by the use of a selective permeabilization reagent that selectively permeabilizes or lyses the target microorganism.

Various permeabilization reagents can be used to selectively permeabilize or lyse the target microorganism. In certain embodiments the permeabilzation reagent comprises a reagent that is intrinsically selective for a particular (e.g., genus, species, strain, etc.) target microorganism. Such reagents include, for example, certain antimicrobial peptides (AMPs).

In certain embodiments, however, selectivity can be conferred by providing a targeting moiety (e.g., a target specific peptide, a target specific antibody, a target specific receptor ligand, etc.) attached to a moiety that permeabilizes or lyses a microorganism. The targeting moiety is selected to specifically or preferentially bind to the target microorganism thereby selectively delivering the permeabilizing moiety to the target microorganism. Suitable targeting moieties are described below.

Targeting Moieties.

In certain embodiments, the targeting moiety comprises one or more targeting peptides that bind particular bacteria, fungi, and/or yeasts, and/or algae, and/or viruses, and/or cells, and/or that bind particular groups of bacteria, and/or groups of fungi, and/or groups of yeasts, and/or groups of algae.

In certain embodiments the targeting peptides include peptides comprising or consisting of one or more of the amino acid sequences shown in Table 2 (SEQ ID NOs:1-1030). In various embodiments the peptides include peptides comprising or consisting of the retro, inverso, retro-inverso, and/or beta form of one or more of the amino acid sequences shown in Table 2. Also contemplated are circular permutations of these sequences as well as peptides comprising or consisting of the retro, inverso, retro-inverso, and/or beta form of such circular permutations. Also contemplated are targeting peptides comprising one, two, three four, or five conservative substitutions of these amino acid sequences.

TABLE 2
Illustrative list of novel targeting peptides.

| | | | |
|---|---|---|---|
| | | | SEQ ID |
| ID | Target(s) | Targeting Peptide Sequence | NO |

| | | | |
|---|---|---|---|
| 1T-3 | S. mutans, S. gordonii | VLGIAGGLDAYGELVGGN | 1 |
| 1T-4 | S. mutans, S. gordonii, S. sanguinis, | LDAYGELVGGN | 2 |
| | S. oralis, V. atypica, | | |
| | L. casei | | |
| 1T-6 | S. mutans | KFINGVLSQFVLERK | 3 |
| 1T-7 | M. xanthus | SQRIIEPVKSPQPYPGFSVS | 4 |
| 1T-8 | M. xanthus | FSVAACGEQRAVTFVLLIE | 5 |
| | | DLI | |
| 1T-9 | M. xanthus | WAWAESPRCVSTRSNIHA | 6 |
| | | LAFRVEVAALT | |
| 1T-10 | M. xanthus | SPAGLPGDGDEA | 7 |
| 1T-11 | S. mutans, S. epidermidis, P. aeruginosa | RISE | 8 |
| 1T-12 | C. xerosis, C. striatum, S. epidermidis, | FGNIFKGLKDVIETIVKWT | 9 |
| | S. mutans | AAK | |
| 1T-13 | S. aureus, S. epidermidis, P. aeruginosa | FRSPCINNNSLQPPGVYPAR | 10 |
| 1T-14 | S. mutans, S. aureus, S. epidermidis, | ALAGLAGLISGK | 11 |
| | C. xerosis | | |
| 1T-15 | S. mutans | DVILRVEAQ | 12 |
| 1T-16 | P. aeruginosa | IDMR | 13 |
| 1T-17 | S. mutans | NNAIVYIS | 14 |
| 1T-18 | S. aureus, S. epidermidis, C. striatum, | YSKTLHFAD | 15 |
| | P. aeruginosa | | |
| 1T-19 | S. aureus, S. epidermidis, P. aeruginosa | PGAFRNPQMPRG | 16 |
| 1T-20 | S. mutans, P. aeruginosa | PALVDLSNKEAVWAVLD | 17 |
| | | DHS | |
| 1T-21 | S. mutans, P. aeruginosa | YVEEAVRAALKKEARIST | 18 |
| | | EDTPVNLPSFDC | |
| 1T-22 | S. epidermidis, P. aeruginosa | VPLDDGTRRPEVARNRDK | 19 |
| | | DRED | |
| 1T-23 | S. mutans, P. aeruginosa | PALVDLSNKEAVWAVLD | 20 |
| | | DHS | |
| 1T-24 | P. aeruginosa | EEAEEKLAEVSQAVKRLVR | 21 |
| 1T-25 | S. aureus, S. epidermidis, C. xerosis, | VGLDVSVLVLFFGLQLLS | 22 |
| | C. striatum, P. aeruginosa | VLLGAMIR | |
| 1T-26 | S. mutans, S. aureus, S. epidermidis, | LTILPTTFFAIIVPILAVAFI | 23 |
| | C. jeikeium, C. xerosis, | AYSGFKIKGIVEHKDQW | |
| | C. striatum, P. aeruginosa | | |
| 1T-27 | S. mutans, S. aureus, S. epidermidis, | ALFVSLEQFLVVVAKSVF | 24 |
| | C. jeikeium, C. xerosis, | ALCHSGTLS | |
| | C. striatum, P. aeruginosa | | |
| 1T-28 | P. aeruginosa | VSRDEAMEFIDREWTTLQ | 25 |
| | | PAGKSHA | |
| 1T-29 | S. mutans, S. aureus, S. epidermidis, | GSVIKKRRKRMSKKKHRK | 26 |
| | C. jeikeium, C. xerosis, | MLRRTRVQRRKLGK | |
| | C. striatum, P. aeruginosa | | |
| 1T-30 | S. aureus, S. epidermidis, C. xerosis, | GKAKPYQVRQVLRAVDK | 27 |
| | C. striatum, P. aeruginosa | LETRRKKGGR | |
| 1T-31 | S. mutans, P. aeruginosa | NATGTDIGEVTLTLGRFS | 28 |
| 1T-32 | S. mutans | VSFLAGWLCLGLAAWRL | 29 |
| | | GNA | |
| 1T-33 | S. aureus, S. epidermidis, C. jeikeium, | VRTLTILVIFIFNYLKSISY | 30 |
| | C. xerosis, C. striatum, | KLKQPFENNLAQSMISI | |
| | P. aeruginosa | | |
| 1T-34 | S. aureus, S. epidermidis, C. jeikeium, | AFWLNILLTLLGYIPGIVH | 31 |
| | C. xerosis, C. striatum, | AVYIIAKR | |
| | P. aeruginosa | | |
| 1T-35 | P. aeruginosa | EICLTLVFPIRGSYSEAAKF | 32 |
| | | PVPIHIVEDGTVELPK | |
| 1T-36 | S. aureus, S. epidermidis, C. jeikeium, | VYRHLRFIDGKLVEIRLERK | 33 |
| | C. xerosis, C. striatum, | | |
| | P. aeruginosa | | |
| 1T-37 | S. mutans, S. aereus, S. epidermidis, | YIVGALVILAVAGLIYSML | 34 |
| | C. jeikeium, C. xerosis, | RKA | |
| | C. striatum, P. aeruginosa | | |
| 1T-38 | S. mutans, S. aereus, S. epidermidis, | VMFVLTRGRSPRPMIPAY | 35 |
| | C. jeikeium, C. xerosis, | | |
| | C. striatum, P. aeruginosa | | |
| 1T-39 | S. mutans, P. aeruginosa | FGFCVWMYQLLAGPPGPPA | 36 |
| 1T-40 | S. mutans, P. aeruginosa | QRVSLWSEVEHEFR | 37 |
| 1T-41 | S. mutans, S. aureus, S. epidermidis, | KRGSKIVIAIAVVLIVLAG | 38 |
| | C. jeikeium, C. striatum, | VWVW | |
| | P. aeruginosa | | |
| 1T-42 | S. aureus, S. epidermidis, C. xerosis, | TVLDWLSLALATGLFVYL | 39 |
| | C. striatum, P. aeruginosa | LVALLRADRA | |
| 1T-43 | C. jeikium, P. aeruginosa | DRCLSVLSWSPPKVSPLI | 40 |
| 1T-44 | S. mutans, S. aureus, S. epidermidis, | DPALADFAAGMRAQVRT | 41 |
| | C. jeikeium, C. striatum, | | |
| | P. aeruginosa | | |
| 1T-45 | S. aureus, S. epidermidis, C. striatum, | WTKPSFTDLRLGFEVTLY | 42 |
| | P. aeruginosa | FANR | |
| 1T-46 | S. aureus, S. epidermidis, C. jeikeium, | FSFKQRVMFRKEVERLR | 43 |
| | C. xerosis, C. striatum, | | |
| | P. aeruginosa | | |
| 1T-47 | S. mutans, S. epidermidis, P. aeruginosa | VIKISVPGQVQMLIP | 44 |
| 1T-48 | S. aureus, S. epidermidis, C. jeikeium, | KLQVHHGRATHTLLLQPP | 45 |
| | C. xerosis, C. striatum, | LCAPGTIR | |
| | P. aeruginosa | | |
| 1T-49 | S. aureus, S. epidermidis, C. jeikeium, | SLVRIHDQQPWVTRGAFI | 46 |
| | P. aeruginosa | DAARTCS | |
| 1T-50 | P. aeruginosa | HSDEPIPNILFKSDSVH | 47 |
| 1T-51 | S. aureus, P. aeruginosa | GKPKRMPAEFIDGYGQAL | 48 |
| | | LAGA | |
| 1T-52 | S. aureus, C. xerosis, P. aeruginosa | DEYPAKLPLSDKGATEPR | 49 |
| | | RH | |
| 1T-53 | P. aeruginosa | SDILAEMFEKGELQTLVK | 50 |
| | | DAAAKANA | |
| 1T-54 | S. epidermidis, C. xerosis, C. striatum, | RWVSCNPSWRIQ | 51 |
| | P. aeruginosa | | |
| 1T-55 | C. xerosis, P. aeruginosa | NHKTLKEWKAKWGPEAV | 52 |
| | | ESWATLLG | |
| 1T-56 | C. xerosis, P. aeruginosa | LALIGAGIWMIRKG | 53 |
| 1T-57 | P. aeruginosa | RLEYRRLETQVEENPESG | 54 |
| | | RRPMRG | |
| 1T-58 | P. aeruginosa | CDDLHALERAGKLDALLSA | 55 |
| 1T-59 | S. aureus, S. epidermidis, P. aeruginosa | AVGNNLGKDNDSGHRGK | 56 |
| | | KHRKHKHR | |
| 1T-60 | S. aureus, S. epidermidis, C. jeikeium, | YLTSLGLDAAEQAQGLLT | 57 |
| | C. striatum, P. aeruginosa | ILKG | |
| 1T-61 | P. aeruginosa | HATLLPAVREAISRQLLPA | 58 |
| | | LVPRG | |
| 1T-62 | S. epidermidis, P. aeruginosa | GCKGCAQRDPCAEPEPYF | 59 |
| | | RLR | |
| 1T-63 | S. aureus, S. epidermidis, C. jeikeium, | EPLILKELVRNLFLFCYAR | 60 |
| | C. xerosis, C. striatum, | ALR | |
| | P. aeruginosa | | |
| 1T-64 | S. aureus, S. epidermidis, C. jeikeium, | QTVHHIHMHVLGQRQMH | 61 |
| | C. xerosis, C. striatum, | WPPG | |
| | P. aeruginosa | | |
| 1T-65 | S. mutans, S. aureus, S. epidermidis, | HARAAVGVAELPRGAAV | 62 |
| | C. jeikeium, C. xerosis, | EVELIAAVRP | |
| | C. striatum, P. aeruginosa | | |
| 1T-66 | S. mutans, S. aureus, S. epidermidis, | DTDCLSRAYAQRIDELDK | 63 |
| | C. jeikeium, C. xerosis, | QYAGIDKPL | |
| | C. striatum, P. aeruginosa | | |
| 1T-67 | S. aureus, S. epidermidis, C. jeikeium, | GQRQRLTCGRVSGCSEGP | 64 |
| | C. xerosis, C. striatum, | SREAAR | |
| | P. aeruginosa | | |
| 1T-68 | S. mutans, S. aureus, C. jeikeium, | GGTKEIVYQRG | 65 |
| | C. xerosis, C. striatum, | | |
| | P. aeruginosa | | |
| 1T-69 | S. mutans, P. aeruginosa | ILSQEADRKKLF | 66 |
| 1T-70 | S. aureus, C. jeikeium, P. aeruginosa | NRQAQGERAHGEQQG | 67 |
| 1T-71 | P. aeruginosa | KIDTNQWPPNKEG | 68 |
| 1T-72 | P. aeruginosa | EPTDGVACKER | 69 |
| 1T-73 | S. pneumoniae | GWWEELLHETILSKFKITK | 70 |
| | | ALELPIQL | |
| 1T-74 | S. pneumoniae | DIDWGRKISCAAGVAYGA | 71 |
| | | IDGCATTV | |
| 1T-75 | S. pneumoniae | GVARGLQLGIKTRTQWG | 72 |
| | | AATGAA | |
| 1T-76 | S. pneumoniae | EMRLSKFFRDFILWRKK | 73 |
| 1T-77 | S. pneumoniae | EMRISRIILDFLFLRKK | 74 |
| 1T-78 | S. pneumoniae | FFKTIFVLILGALGVAAGL | 75 |
| | | YIEKNYIDK | |
| 1T-79 | S. pneumoniae | FGTPWSITNFWKKNFNDR | 76 |
| | | PDFDSDRRRY | |
| 1T-80 | S. pneumoniae | GGNLGPGFGVIIP | 77 |
| 1T-81 | S. pneumoniae | AIATGLDIVDGKFDGYLWA | 78 |
| 1T-82 | S. pneumoniae | FGVGVGIALFMAGYAIGK | 79 |
| | | DLRKKFGKSC | |
| 1T-83 | S. pneumoniae | QKPRKNETFIGYIQRYDID | 80 |
| | | GNGYQSLPCPQN | |
| 1T-84 | S. pneumoniae | FRKKRYGLSILLWLNAFT | 81 |
| | | NLVNSIHAFYMTLF | |
| 1T-85 | A. naeslundii, F. nucleatum, P. gingivalis, | VMASLTWRMRAASASLP | 82 |
| | S. epidermidis, S. gordonii, | THSRTDA | |
| | S. mitis, S. mutans, S. oralis, | | |
| | S. salivarious, S. sanguinis | | |
| 1T-86 | S. mitis, S. oralis, S. salivarious | HRKNPVLGVGRRHRAHN | 83 |
| | | VA | |
| 1T-87 | S. mitis, S. mutans, S. oralis | EAVGQDLVDAHHP | 84 |
| 1T-89 | S. mitis, S. mutans | HEDDKRRGMSVEVLGFE | 85 |
| | | VVQHEE | |
| 1T-90 | S. gordonii, S. mitis, S. mutans, | RNVIGQVL | 86 |
| | S. oralis, S. sanguinis | | |
| 1T-91 | S. mitis, S. mutans, S. oralis, S. sanguinis | TSVRPGAAGAAVPAGAA | 87 |
| | | GAAGAGWRWP | |
| 1T-92 | S. mitis, S. mutans | GQDEGQRRAGVGEGQGV | 88 |
| | | DG | |
| 1T-93 | S. epidermidis, S. gordonii, S. mitis, | AMRSVNQA | 89 |
| | S. mutans, S. oralis, S. sanguinis | | |
| 1T-94 | S. mitis, S. mutans, S. oralis | DQVAHSGDMLVQARRRDS | 90 |
| 1T-95 | S. gordonii, S. mitis, S. mutans, | GHLLRVGGRVGGVGGVA | 91 |
| | S. oralis, S. sanguinis | GACAQPFGGQ | |
| 1T-96 | S. gordonii, S. mitis, S. mutans, | VAGACAQPFGGQ | 92 |
| | S. oralis, S. sanguinis | | |
| 1T-97 | A. naeslundii, F. nucleatum, P. gingivalis, | GVAERNLDRITVAVAIIWT | 93 |
| | S. epidermidis, S. gordonii, | ITIVGLGLVAKLG | |
| | S. mitis, S. mutans, S. oralis, | | |
| | S. salivarious, S. sanguinis | | |
| 1T-98 | A. naeslundii, F. nucleatum, P. gingivalis, | VRSAKAVKALTAAGYTG | 94 |
| | S. epidermidis, S. gordonii, | ELVNVSGGMKAWLGQ | |
| | S. mitis, S. mutans, S. oralis, | | |
| | S. salivarious, S. sanguinis | | |
| 1T-99 | S. gordonii, S. mitis, S. mutans, | MKAWLGQ | 95 |
| | S. oralis, S. sanguinis | | |
| 1T-100 | S. gordonii, S. mitis, S. mutans | LDPLEPRIAPPGDRSHQGA | 96 |
| | | PACHRDPLRGRSARDAER | |
| 1T-101 | A. naeslundii, P. gingivalis, S. epidermidis, | RLRVGRATDLPLTSFAVG | 97 |
| | S. gordonii, S. mitis, | VVRNLPDAPAH | |
| | S. mutans, S. oralis, S. sanguinis | | |
| 1T-102 | A. naeslundii, F. nucleatum, P. gingivalis, | WKRLWPARILAGHSRRR | 98 |
| | S. epidermidis, S. gordonii, | MRWMVVWRYFAAT | |
| | S. mitis, S. mutans, S. oralis, | | |
| | S. salivarious, S. sanguinis | | |
| 1T-103 | A. naeslundii, F. nucleatum, P. gingivalis, | AQFYEAIITGYALGAGQRI | 99 |
| | S. epidermidis, S. gordonii, | GQL | |
| | S. mitis, S. mutans, S. oralis, | | |
| | S. sanguinis | | |
| 1T-104 | S. mitis | RAVAAHLQGRHHGHQVR | 100 |
| | | RQRHGQR | |
| 1T-105 | S. epidermidis, S. gordonii, S. mitis, | GEGLPPPVLHLPPPRMSGR | 101 |
| | S. mutans, S. oralis | | |
| 1T-106 | S. gordonii, S. mitis, S. mutans, | DALRRSRSQGRRHR | 102 |
| | S. oralis, S. salivarious | | |
| 1T-107 | A. naeslundii, S. epidermidis, S. gordonii, | SPVPRFTAVGGVSRGSP | 103 |
| | S. mitis, S. mutans, S. oralis, | | |
| | S. salivarious, S. sanguinis | | |
| 1T-108 | S. gordonii, S. mitis, S. mutans, | WGPLGPERPLW | 104 |
| | S. oralis, S. salivarious, S. sanguinis | | |
| 1T-109 | A. naeslundii, S. epidermidis, S. gordonii, | VTTNVRQGAGS | 105 |
| | S. mitis, S. mutans, S. oralis, | | |
| | S. salivarious, S. sanguinis | | |
| 1T-110 | A. naeslundii, P. gingivalis, S. epidermidis, | LAAKTAVCVGRAFM | 106 |
| | S. gordonii, S. mitis, | | |
| | S. mutans, S. oralis, S. sanguinis | | |
| 1T-111 | A. naeslundii, F. nucleatum, P. gingivalis, | GRLSRREEDPATSIILLRG | 107 |
| | S. epidermidis, S. gordonii, | AYRMAVF | |
| | S. mitis, S. mutans, S. oralis, | | |
| | S. salivarious, S. sanguinis | | |
| 1T-112 | S. gordonii | SDNDGKLILGTSQ | 108 |
| 1T-113 | S. mitis | HGAHQRTGQRLHHHRGR | 109 |
| | | TVSGCRQNPVAGVDPDEHR | |
| 1T-114 | A. naeslundii, P. gingivalis, S. epidermidis, | RQAPGPGLVTITAACSAPG | 110 |
| | S. gordonii, S. mitis, | SRSR | |
| | S. mutans, S. oralis, S. sanguinis | | |
| 1T-115 | A. naeslundii, F. nucleatum, P. gingivalis, | LLIERFSNHH | 111 |
| | S. epidermidis, S. gordonii, | | |
| | S. mitis, S. mutans, S. oralis, | | |
| | S. salivarious, S. sanguinis | | |
| 1T-116 | A. naeslundii, P. gingivalis, S. epidermidis, | MILHRRRDR | 112 |
| | S. gordonii, S. mitis, | | |
| | S. mutans, S. oralis, S. salivarious, | | |
| | S. sanguinis | | |
| 1T-117 | S. mutans | GPGVVGPAPFSRLPAHAL | 113 |
| | | NL | |
| 1T-118 | A. naeslundii, F. nucleatum, P. gingivalis, | TASPPAPSDQGLRTAFPAT | 114 |
| | S. epidermidis, S. gordonii, | LLIALAALARISR | |
| | S. mitis, S. mutans, S. oralis, | | |
| | S. salivarious, S. sanguinis | | |
| 1T-119 | S. gordonii, S. mitis, S. mutans, | SPATQKAPTRAQPSRAPV | 115 |
| | S. oralis | QDCGDGRPTAAPDDVERL | |
| | | SPR | |
| 1T-120 | A. naeslundii, F. nucleatum, P. gingivalis, | DVRDRVDLAGADLCAAH | 116 |
| | S. epidermidis, S. gordonii, | ATR | |
| | S. mitis, S. mutans, S. oralis, | | |
| | S. salivarious, S. sanguinis | | |
| 1T-121 | S. gordonii, S. mitis, S. mutans, | FAKETGFGIGGAQEGWWI | 117 |
| | S. oralis, S. salivarious, S. sanguinis | IADIYGPNPF | |
| 1T-122 | S. mitis | GAIPDPVTHRVDWEEDHQ | 118 |
| | | TRPSR | |
| 1T-123 | S. gordonii | LVRRNAVAGRSDGLAGA | 119 |
| | | EQLDLVRLQGVL | |
| 1T-124 | S. mitis, S. mutans, S. oralis | LFDERNKIA | 120 |
| 1T-125 | S. epidermidis, S. gordonii, S. mutans, | DAITGGNPPLSDTDGLRP | 121 |
| | S. oralis | | |
| 1T-126 | S. gordonii, S. mitis, S. mutans | QGLARPVLRRIPL | 122 |
| 1T-127 | A. naeslundii, F. nucleatum, P. gingivalis, | YDPVPKRKNKNSEGKREE | 123 |
| | T. denticola, S. gordonii, | | |
| | S. mitis, S. mutans, S. oralis, | | |
| | S. salivarious, S. sanguinis | | |
| 1T-128 | A. naeslundii, P. gingivalis, S. epidermidis, | SGSAIRMLEIATKMLKR | 124 |
| | S. gordonii, S. mitis, | | |
| | S. mutans, S. oralis, S. salivarious, | | |
| | S. sanguinis | | |
| 1T-129 | A. naeslundii, P. gingivalis, S. epidermidis, | YDKYIKYLSIQPPFIVYFI | 125 |
| | S. gordonii, S. mitis, | | |
| | S. mutans, S. oralis, S. salivarious, | | |
| | S. sanguinis | | |
| 1T-130 | A. naeslundii, F. nucleatum, P. gingivalis, | QKIIDMSKFLFSLILFIMIV | 126 |
| | S. epidermidis, S. gordonii, | VIYIGKSIGGYSAIVSSIML | |
| | S. mitis, S. mutans, S. oralis, | ELDTVLYNKKIFFIYK | |
| | S. salivarious, S. sanguinis | | |
| 1T-131 | A. naeslundii, F. nucleatum, P. gingivalis, | DEVWKMLGI | 127 |
| | T. denticola, S. gordonii, | | |
| | S. mitis, S. mutans, S. oralis, | | |
| | S. salivarious, S. sanguinis | | |
| 1T-132 | A. naeslundii, F. nucleatum, P. gingivalis, | YSKKLFEYFYFIIFILIRYLI | 128 |
| | S. epidermidis, S. gordonii, | FYKIIQNKNYYINNIAYN | |
| | S. mitis, S. mutans, S. oralis, | | |
| | S. salivarious, S. sanguinis | | |
| 1T-133 | A. naeslundii, P. gingivalis, S. epidermidis, | YFIKDDNEALSKDWEVIG | 129 |
| | S. gordonii, S. mitis, | NDLKGTIDKYGKEFKVR | |
| | S. mutans, S. oralis, S. salivarious, | | |
| | S. sanguinis | | |
| 1T-134 | A. naeslundii, F. nucleatum, P. gingivalis, | SRLVREIKKKCRKS | 130 |
| | S. epidermidis, S. gordonii, | | |
| | S. mitis, S. mutans, S. oralis, | | |
| | S. salivarious, S. sanguinis | | |
| 1T-135 | A. naeslundii, P. gingivalis, S. epidermidis, | FESLLPQATKKIVNNKGSK | 131 |
| | S. gordonii, S. mitis, | INKIF | |
| | S. mutans, S. oralis, S. salivarious, | | |
| | S. sanguinis | | |
| 1T-136 | A. naeslundii, F. nucleatum, P. gingivalis, | ELLTQIRLALLYSVNEW | 132 |
| | S. epidermidis, S. gordonii, | | |
| | S. mitis, S. mutans, S. oralis, | | |
| | S. salivarious, S. sanguinis | | |
| 1T-137 | A. naeslundii, F. nucleatum, P. gingivalis, | PLNFYRAVKENRLPLSEK | 133 |
| | S. epidermidis, S. gordonii, | NINDFTNIKLKVSPKLINLL | |
| | S. mitis, S. mutans, S. oralis, | QESSIFYNFSPKKRNTN | |
| | S. salivarious, S. sanguinis | | |
| 1T-138 | A. naeslundii, F. nucleatum, P. gingivalis, | YPNEYCIFLENLSLEELKEI | 134 |
| | S. epidermidis, S. gordonii, | KAINGETLNLEEIINERKN | |
| | S. mitis, S. mutans, S. oralis, | LKD | |
| | S. salivarious, S. sanguinis | | |
| 1T-139 | A. naeslundii, S. gordonii, S. mitis, | AVAGAAVGALLGNDARS | 135 |
| | S. mutans, S. oralis | TAVGAAIGGALGAGAGEL | |
| | | TKNK | |
| 1T-140 | A. naeslundii, F. nucleatum, P. gingivalis, | IKGTIAFVGEDYVEIRVDK | 136 |
| | S. epidermidis, S. gordonii, | GVKLTFRKSAIANVINNN | |
| | S. mitis, S. mutans, S. oralis, | QQ | |
| | S. salivarious, S. sanguinis | | |
| 1T-141 | F. nucleatum, P. gingivalis, S. epidermidis, | KKFIILLFILVQGLIFSATK | 137 |
| | S. gordonii, S. mitis, | TLSDIIAL | |
| | S. mutans, S. oralis, S. sanguinis | | |
| 1T-142 | A. naeslundii, F. nucleatum, P. gingivalis, | FTQGIKRIVLKRLKED | 138 |
| | T. denticola, S. gordonii, | | |
| | S. mitis, S. mutans, S. oralis, | | |
| | S. salivarious, S. sanguinis | | |
| 1T-143 | A. naeslundii, F. nucleatum, P. gingivalis, | MPKRHYYKLEAKALQFG | 139 |
| | S. epidermidis, S. gordonii, | LPFAYSPIQLLK | |
| | S. mitis, S. mutans, S. oralis, | | |
| | S. salivarious, S. sanguinis | | |
| 1T-144 | A. naeslundii, F. nucleatum, P. gingivalis, | IIELHPKSWTQDWRCSFL | 140 |
| | T. denticola, S. gordonii, | | |
| | S. mitis, S. mutans, S. oralis, | | |
| | S. salivarious, S. sanguinis | | |
| 1T-145 | S. mitis, S. mutans, S. oralis | VEAGKRNISLENIEKISKG | 141 |
| | | LGISISELFKYIEEGEDKIG | |
| 1T-146 | A. naeslundii, F. nucleatum, P. gingivalis, | RNSADNQTKIDKIRIDISL | 142 |
| | T. denticola, S. gordonii, | WDEHLNIVKQGK | |
| | S. mitis, S. mutans, S. oralis, | | |
| | S. salivarious, S. sanguinis | | |
| 1T-147 | A. naeslundii, F. nucleatum, P. gingivalis, | GVENRRFYERDVSKVSM | 143 |
| | T. denticola, S. gordonii, | MTSEAVAPRGGSK | |
| | S. mitis, S. mutans, S. oralis, | | |
| | S. salivarious, S. sanguinis | | |
| 1T-148 | A. naeslundii, F. nucleatum, P. gingivalis, | IVELDDTTILERALSMLGE | 144 |
| | T. denticola, S. gordonii, | ANA | |
| | S. mitis, S. mutans, S. oralis, | | |
| | S. salivarious, S. sanguinis | | |
| 1T-149 | A. naeslundii, F. nucleatum, P. gingivalis, | SVRAVKPIDETVARHFPG | 145 |
| | T. denticola, S. gordonii, | DFIVN | |
| | S. mitis, S. mutans, S. oralis, | | |
| | S. salivarious, S. sanguinis | | |
| 1T-150 | A. naeslundii, F. nucleatum, P. gingivalis, | YINRRLKKAFSDADIKEAP | 146 |
| | T. denticola, S. gordonii, | AEFYEELRRVQYV | |
| | S. mitis, S. mutans, S. oralis, | | |
| | S. salivarious, S. sanguinis | | |
| 1T-151 | A. naeslundii, F. nucleatum, P. gingivalis, | SVRAVKPIDEIVAWHFPG | 147 |
| | T. denticola, S. gordonii, | DFIVN | |
| | S. mitis, S. mutans, S. oralis, | | |
| | S. salivarious, S. sanguinis | | |
| 1T-152 | A. naeslundii, F. nucleatum, P. gingivalis, | YVSADESAYNHIVTDDIPL | 148 |
| | S. epidermidis, S. gordonii, | ADRRIEAVQQ | |
| | S. mitis, S. mutans, S. oralis, | | |
| | S. salivarious, S. sanguinis | | |
| 1T-153 | A. naeslundii, F. nucleatum, P. gingivalis, | YIACPGYFY | 149 |
| | S. epidermidis, S. gordonii, | | |
| | S. mitis, S. mutans, S. oralis, | | |
| | S. salivarious, S. sanguinis | | |
| 1T-154 | P. gingivalis | YFSFLEIVGMARR | 150 |
| 1T-155 | A. naeslundii, F. nucleatum, P. gingivalis, | LKLAFGVYPFQAMSQSDT | 151 |
| | S. epidermidis, S. gordonii, | AVSERNVLWR | |
| | S. mitis, S. mutans, S. oralis, | | |
| | S. salivarious, S. sanguinis | | |
| 1T-156 | A. naeslundii, F. nucleatum, P. gingivalis, | GRFQISIRGEEKSKVKVQG | 152 |
| | T. denticola, S. gordonii, | KGTFTDRNTT | |
| | S. mitis, S. mutans, S. oralis, | | |
| | S. salivarious, S. sanguinis | | |
| 1T-157 | A. naeslundii, F. nucleatum, P. gingivalis, | RRFRKTTENREKSKNKKA | 153 |
| | T. denticola, S. gordonii, | VLGLSTTSTASY | |
| | S. mitis, S. mutans, S. oralis, | | |
| | S. salivarious, S. sanguinis | | |
| 1T-158 | A. naeslundii, F. nucleatum, P. gingivalis, | WENKPSPLGSIKKLQGLV | 154 |
| | S. epidermidis, S. gordonii, | YRLIGYRHFWV | |
| | S. mitis, S. mutans, S. oralis, | | |
| | S. salivarious, S. sanguinis | | |
| 1T-159 | P. gingivalis | IFSLHHFALICSEMGTFAV | 155 |
| | | SKRAKYKWEVL | |
| 1T-160 | A. naeslundii, F. nucleatum, P. gingivalis, | AQYKYINKLLN | 156 |
| | T. denticola, S. gordonii, | | |
| | S. mitis, S. mutans, S. oralis, | | |
| | S. salivarious, S. sanguinis | | |
| 1T-161 | A. naeslundii, F. nucleatum, P. gingivalis, | NKVLQVEVMWDGSVVGR | 157 |
| | S. epidermidis, S. gordonii, | PAGVISIKSSKKG | |
| | S. mitis, S. mutans, S. oralis, | | |
| | S. salivarious, S. sanguinis | | |
| 1T-162 | A. naeslundii, F. nucleatum, P. gingivalis, | QKAKEESDRKAAVSYNGF | 158 |
| | T. denticola, S. gordonii, | HRVNVVSIPK | |
| | S. mitis, S. mutans, S. oralis, | | |
| | S. salivarious, S. sanguinis | | |
| 1T-163 | A. naeslundii, F. nucleatum, P. gingivalis, | MENILIYIPMVLSPFGSGIL | 159 |
| | S. epidermidis, S. gordonii, | LFLGKDRRYML | |
| | S. mitis, S. mutans, S. oralis, | | |
| | S. salivarious, S. sanguinis | | |
| 1T-164 | A. naeslundii, F. nucleatum, P. gingivalis, | KKSHSQGKRKLKDLNSAY | 160 |
| | S. epidermidis, S. gordonii, | KIDNQLHYALR | |
| | S. mitis, S. mutans, S. oralis, | | |
| | S. salivarious, S. sanguinis | | |
| 1T-165 | A. naeslundii, F. nucleatum, P. gingivalis, | CYDSFDFSIFVTFANRMKL | 161 |
| | S. epidermidis, S. gordonii, | SVGS | |
| | S. mitis, S. mutans, S. oralis, | | |
| | S. salivarious, S. sanguinis | | |
| 1T-166 | A. naeslundii, F. nucleatum, P. gingivalis, | AQSAGQIKRKSKVRIHV | 162 |
| | S. epidermidis, S. gordonii, | | |
| | S. mitis, S. mutans, S. oralis, | | |
| | S. salivarious, S. sanguinis | | |
| 1T-167 | A. naeslundii, F. nucleatum, P. gingivalis, | SRMSEHSPAGLVFEVGPM | 163 |
| | S. epidermidis, S. gordonii, | DKGSFIILDSYHPTVKK | |
| | S. mitis, S. mutans, S. oralis, | | |
| | S. salivarious, S. sanguinis | | |
| 1T-168 | A. naeslundii, F. nucleatum, P. gingivalis, | ELHRIMSTEKIGAVTKMN | 164 |
| | S. epidermidis, S. gordonii, | FDTAPIMSILPIDIYPKEVGI | |
| | S. mitis, S. mutans, S. oralis, | GS | |
| | S. salivarious, S. sanguinis | | |
| 1T-169 | A. naeslundii, F. nucleatum, P. gingivalis, | FARVRRLHQNRILTQPLTN | 165 |
| | S. epidermidis, S. gordonii, | LKYCLRQPIYSD | |
| | S. mitis, S. mutans, S. oralis, | | |
| | S. salivarious, S. sanguinis | | |
| 1T-170 | P. gingivalis | AYGKVFSMDIMLSENDKL | 166 |
| | | IVLRISHHSAWH | |
| 1T-171 | A. naeslundii, F. nucleatum, P. gingivalis, | SVRAVKPIDKTVARHFPG | 167 |
| | S. epidermidis, S. gordonii, | DFIVN | |
| | S. mitis, S. mutans, S. oralis, | | |
| | S. salivarious, S. sanguinis | | |
| 1T-172 | A. naeslundii, F. nucleatum, P. gingivalis, | FEGLKNLLGDDII | 168 |
| | S. epidermidis, S. gordonii, | | |
| | S. mitis, S. mutans, S. oralis, | | |
| | S. salivarious, S. sanguinis | | |
| 1T-173 | A. naeslundii, F. nucleatum, P. gingivalis, | LFRKEDQEHVLL | 169 |
| | S. gordonii, S. mitis, | | |
| | S. mutans, S. oralis, S. salivarious, | | |
| | S. sanguinis | | |
| 1T-174 | A. naeslundii, F. nucleatum, P. gingivalis, | SGGSDTDGSSSGEPGSHSG | 170 |
| | T. denticola, S. gordonii, | DL | |
| | S. mitis, S. mutans, S. oralis, | | |
| | S. salivarious, S. sanguinis | | |
| 1T-175 | A. naeslundii, F. nucleatum, P. gingivalis, | GEPGSHSGDL | 171 |
| | S. epidermidis, S. gordonii, | | |
| | S. mitis, S. mutans, S. oralis, | | |
| | S. salivarious, S. sanguinis | | |
| 1T-176 | A. naeslundii, P. gingivalis, S. epidermidis, | PVGDIMSGFLRGANQPRF | 172 |
| | S. gordonii, S. mitis, | LLDHISFGS | |
| | S. mutans, S. oralis, S. salivarious, | | |
| | S. sanguinis | | |
| 1T-177 | P. gingivalis, S. gordonii, S. mitis, | GTNVPTQILGYSREERFDY | 173 |
| | S. mutans, S. oralis, S. salivarious, | EPAPEQR | |
| | S. sanguinis | | |
| 1T-178 | A. naeslundii, F. nucleatum, P. gingivalis, | LLASHPERLSLGVFFVYRV | 174 |
| | S. epidermidis, S. gordonii, | LHLLLENT | |
| | S. mitis, S. mutans, S. oralis, | | |
| | S. salivarious, S. sanguinis | | |
| 1T-179 | A. naeslundii, F. nucleatum, P. gingivalis, | TCYPLIQRKTDRAYEA | 175 |
| | T. denticola, S. gordonii, | | |
| | S. mitis, S. mutans, S. oralis, | | |
| | S. salivarious, S. sanguinis | | |
| 1T-180 | A. naeslundii, F. nucleatum, P. gingivalis, | VVFGGGDRLV | 176 |
| | T. denticola, S. gordonii, | | |
| | S. mitis, S. mutans, S. oralis, | | |
| | S. salivarious, S. sanguinis | | |
| 1T-181 | A. naeslundii, F. nucleatum, P. gingivalis, | YGKESDP | 177 |
| | T. denticola, S. gordonii, | | |
| | S. mitis, S. mutans, S. oralis, | | |
| | S. salivarious, S. sanguinis | | |
| 1T-182 | A. naeslundii, F. nucleatum, P. gingivalis, | LTASICRQWNDNSTPYQR | 178 |
| | T. denticola, S. gordonii, | | |
| | S. mitis, S. mutans, S. oralis, | | |
| | S. salivarious, S. sanguinis | | |
| 1T-183 | A. naeslundii, F. nucleatum, P. gingivalis, | PLRSFVAEKAEHAFRVVRI | 179 |
| | S. epidermidis, S. gordonii, | ADFDFGHS | |
| | S. mitis, S. mutans, S. oralis, | | |
| | S. salivarious, S. sanguinis | | |
| 1T-184 | A. naeslundii, F. nucleatum, P. gingivalis, | ALLVLNLLLMQFFFGKNM | 180 |
| | T. denticola, S. gordonii, | | |
| | S. mitis, S. mutans, S. oralis, | | |
| | S. salivarious, S. sanguinis | | |
| 1T-185 | A. naeslundii, F. nucleatum, P. gingivalis, | HYHFLLEFGFHKGDYLE | 181 |
| | T. denticola, S. gordonii, | | |
| | S. mitis, S. mutans, S. oralis, | | |
| | S. salivarious, S. sanguinis | | |
| 1T-188 | S. sanguinis | HRKDVYKK | 182 |
| 1T-190 | A. naeslundii, F. nucleatum, P. gingivalis, | IQIIVNAFVEKDKTGAVIE | 183 |
| | S. epidermidis, S. gordonii, | VLYASNNHEKVKAKYEE | |
| | S. mitis, S. mutans, S. oralis, | LVAIS | |
| | S. salivarious, S. sanguinis | | |
| 1T-192 | S. sanguinis | ILVLLALQVELDSKFQY | 184 |
| 1T-193 | S. sanguinis | LMIFDKHANLKYKYGNRS | 185 |
| | | FGVEAIM | |
| 1T-195 | S. mutans | AASGFTYCASNGVWHPY | 186 |
| 1T-196 | F. nucleatum, S. sanguinis | KPEKEKLDTNTLMKVVN | 187 |
| | | KALSLFDRLLIKFGA | |
| 1T-197 | A. naeslundii, F. nucleatum, P. gingivalis, | TEILNFLITVCADRENWKI | 188 |
| | S. epidermidis, S. gordonii, | KHGLSDSVLLIFFARFTGA | |
| | S. mitis, S. mutans, S. oralis, | EYW | |
| | S. salivarious, S. sanguinis | | |
| 1T-198 | P. gingivalis, S. epidermidis, S. gordonii, | MPVSKKRYMLSSAYATA | 189 |
| | S. mitis, S. mutans, S. oralis, | LGICYGQVATDEKESEITA | |
| | S. sanguinis | IPDLLDYLSVEEYLL | |
| 1T-199 | S. sanguinis | RAGRIKKLSQKEAEPFEN | 190 |
| 1T-200 | A. naeslundii, F. nucleatum, S. epidermidis, | MRFKRFDRDYALSGDNV | 191 |
| | S. gordonii, S. mitis, | FEVLTASCDVIERNLSYRE | |
| | S. mutans, S. oralis, S. salivarious, | MCGLMQ | |
| | S. sanguinis | | |
| 1T-201 | S. sanguinis | KRKHENVIVAEEMRVIKN | 192 |
| 1T-202 | A. naeslundii, F. nucleatum, P. gingivalis, | LCRLEKLCKQFLRQDKVV | 193 |
| | S. epidermidis, S. gordonii, | TYYLMLPYKRAIEAFYQE | |
| | S. mitis, S. mutans, S. oralis, | LKERS | |
| | S. salivarious, S. sanguinis | | |
| 1T-203 | A. naeslundii, F. nucleatum, P. gingivalis, | YPFCLATVDHLPEGLSVT | 194 |
| | S. epidermidis, S. gordonii, | DYERVQRLVSQFLLNKEER | |
| | S. mitis, S. mutans, S. oralis, | | |
| | S. salivarious, S. sanguinis | | |
| 1T-207 | F. nucleatum, S. sanguinis | SPLEKYGTGSMTALTFLL | 195 |
| | | GCCLLVLSKKSR | |
| 1T-208 | Unanalyzed | KRKRWAILTLFLAGLGAV | 196 |
| | | GIVLATF | |
| 1T-215 | S. sanguinis | VCFKDISVFLSPFRGQEVL | 197 |
| | | FCGKAKHSLIYVIGT | |
| 1T-216 | S. sanguinis | FFLNVIAIRIPHF | 198 |
| 1T-217 | F. nucleatum, S. sanguinis | MLSNVLSRSVVSPNVDIP | 199 |
| | | NSMVILSPLLISISNYH | |
| 1T-218 | F. nucleatum, S. sanguinis | KLIFAALGLVFLLIGLRDS | 200 |
| | | RSK | |
| 1T-219 | S. sanguinis | RNINVSATFITEKSLV | 201 |
| 1T-221 | A. naeslundii, F. nucleatum, P. gingivalis, | DIGRIIGKKGRTITAIRSIV | 202 |
| | S. epidermidis, S. gordonii, | YSVPTQGKKVRLVIDEK | |
| | S. mitis, S. mutans, S. oralis, | | |
| | S. salivarious, S. sanguinis | | |
| 1T-222 | F. nucleatum, S. sanguinis | RIEASLISAIMFSMFNAIVK | 203 |
| | | FLQK | |
| 1T-223 | A. naeslundii, F. nucleatum, P. gingivalis, | NQKMEINSMTSEKEKMLA | 204 |
| | S. epidermidis, S. gordonii, | GHFHNEANFAVIFKYSLF | |
| | S. mitis, S. mutans, S. oralis, | YNFF | |
| | S. salivarious, S. sanguinis | | |
| 1T-225 | A. naeslundii, F. nucleatum, P. gingivalis, | RRSLGNSASFAEWIEYIRY | 205 |
| | S. epidermidis, S. gordonii, | LHYIIRVQFIHFFSKNKKI | |
| | S. mitis, S. mutans, S. oralis, | | |
| | S. salivarious, S. sanguinis | | |
| 1T-226 | A. naeslundii, F. nucleatum, S. epidermidis, | KLQEKQIDRNFERVSGYS | 206 |
| | S. gordonii, S. mitis, | TYRAVQAAKAKEKGFISL | |
| | S. mutans, S. oralis, S. salivarious, | EN | |
| | S. sanguinis | | |
| 1T-228 | A. naeslundii, F. nucleatum, P. gingivalis, | IFKLFEEHLLYLLDAFYYS | 207 |
| | S. epidermidis, S. gordonii, | KIFRRLKQGLYRRKEQPY | |
| | S. mitis, S. mutans, S. oralis, | TQDLFRM | |
| | S. salivarious, S. sanguinis | | |
| 1T-230 | A. naeslundii, F. nucleatum, P. gingivalis, | EFLEKFKVLKQPRKANNIS | 208 |
| | S. epidermidis, S. gordonii, | KNRVAMIFLTIHKSRGFLS | |
| | S. mitis, S. mutans, S. oralis, | SPY | |
| | S. salivarious, S. sanguinis | | |
| 1T-233 | A. naeslundii, P. gingivalis, S. epidermidis, | TDQELEHLIVTELESKRLD | 209 |
| | S. gordonii, S. mitis, | FTYSKDITEFFDEAFPEYD | |
| | S. mutans, S. oralis, S. salivarious, | QNY | |
| | S. sanguinis | | |
| 1T-234 | A. naeslundii, F. nucleatum, P. gingivalis, | DNFYLILKMEERGKSKKT | 210 |
| | S. epidermidis, S. gordonii, | SQTRGFRAFFDIIRKKIKKE | |
| | S. mitis, S. mutans, S. oralis, | DGK | |
| | S. salivarious, S. sanguinis | | |
| 1T-237 | S. sanguinis | EDPVPNHFTLRRNKKEKP | 211 |
| | | SKS | |
| 1T-238 | A. naeslundii, F. nucleatum, P. gingivalis, | IFNRRKFFQYFGLSKEAM | 212 |
| | S. epidermidis, S. gordonii, | VEHIQPFILDIWQIHLF | |
| | S. mitis, S. mutans, S. oralis, | | |
| | S. salivarious, S. sanguinis | | |
| 1T-239 | A. naeslundii, S. gordonii, S. mitis, | ADDLLNKRLTDLIMENAE | 213 |
| | S. mutans, S. oralis, S. sanguinis | TVKTIDLDNSD | |
| 1T-240 | A. naeslundii, F. nucleatum, P. gingivalis, | VILGNGISNIAQTLGQLPNI | 214 |
| | S. epidermidis, S. gordonii, | AWVWIYMVLIAALLEESN | |
| | S. mitis, S. mutans, S. oralis, | VC | |
| | S. salivarious, S. sanguinis | | |
| 1T-242 | F. nucleatum, S. sanguinis | KQVQNTTLIICGTVLLGIL | 215 |
| | | FKSYLKSQKSV | |
| 1T-243 | A. naeslundii, P. gingivalis, S. epidermidis, | SENIARFAAAFENEQVVS | 216 |
| | S. gordonii, S. mitis, | YARWFRRSWRGSGSSSRF | |
| | S. mutans, S. oralis, S. salivarious, | | |
| | S. sanguinis | | |
| 1T-248 | S. sanguinis | IGGALNSCG | 217 |
| 1T-249 | F. nucleatum, S. sanguinis | VFSVLKHTTWPTRKQSW | 218 |
| | | HDFISILEYSAFFALVIFIFD | |
| | | KLLTLGLAELLKRF | |
| 1T-250 | S. mitis, S. mutans, S. oralis | LVQGDTILIENHVGTPVKD | 219 |
| | | DGKDCLIIREADVLAVVND | |
| 1T-252 | F. nucleatum, S. sanguinis | MKKNLKRFYALVLGFIIG | 220 |
| | | CLFVSILIFIGY | |
| 1T-253 | A. naeslundii, F. nucleatum, P. gingivalis, | KTKESLTQQEKKFLKDYD | 221 |
| | S. epidermidis, S. gordonii, | RKSLHHFRDILTYCFILDK | |
| | S. mitis, S. mutans, S. oralis, | LTNK | |
| | S. salivarious, S. sanguinis | | |
| 1T-256 | S. sanguinis | KGKSLMPLLKQINQWGKL | 222 |
| | | YL | |
| 1T-257 | A. naeslundii, F. nucleatum, P. gingivalis, | IILAKAADLAEIERIISEDPF | 223 |
| | S. epidermidis, S. gordonii, | KINEIANYDIIEFCPTKSSK | |
| | S. mitis, S. mutans, S. oralis, | AFEKVLK | |
| | S. salivarious, S. sanguinis | | |
| 1T-258 | A. naeslundii, F. nucleatum, P. gingivalis, | TINIDDKVLDYLKKINSKA | 224 |
| | T. denticola, S. mitis, | ITIDLIGCAS | |
| | S. mutans, S. oralis | | |
| 1T-259 | F. nucleatum, P. gingivalis, T. denticola, | EKLKKILLKLAVCGKAWY | 225 |
| | S. mitis, S. mutans, S. oralis, | TL | |
| | S. sanguinis | | |
| 1T-260 | A. naeslundii, P. gingivalis, S. epidermidis, | NILYFIHDENQWEPQKAEI | 226 |
| | S. gordonii, S. mitis, | FRGSIKHCAWLSS | |
| | S. mutans, S. oralis, S. sanguinis | | |
| 1T-261 | F. nucleatum, S. mutans, S. oralis, | SFEKNKIENNLKIAQAYIYI | 227 |
| | S. sanguinis | KPKPRICQA | |
| 1T-262 | A. naeslundii, F. nucleatum, P. gingivalis, | LSLPLIVLTKSI | 228 |
| | S. epidermidis, S. gordonii, | | |
| | S. mitis, S. mutans, S. oralis, | | |
| | S. salivarious, S. sanguinis | | |
| 1T-263 | A. naeslundii, F. nucleatum, P. gingivalis, | FIAVSFTGNPATFKLVIGC | 229 |
| | S. epidermidis, S. gordonii, | KADN | |
| | S. mitis, S. oralis, S. salivarious, | | |
| | S. sanguinis | | |
| 1T-264 | S. sanguinis | LEGKFYMAEDFDKTPECF | 230 |
| | | KDYV | |
| 1T-265 | A. naeslundii, F. nucleatum, P. gingivalis, | GMFENLLMINFQIMNDLK | 231 |
| | S. epidermidis, S. gordonii, | IEIVVKDRICAV | |
| | S. mitis, S. mutans, S. oralis, | | |
| | S. salivarious, S. sanguinis | | |
| 1T-266 | S. sanguinis | RAGTWLVVDEIR | 232 |
| 1T-267 | A. naeslundii, F. nucleatum, P. gingivalis, | RIKEERKNRSYKFFIWRLF | 233 |
| | T. denticola, S. mitis, | DEKTGFI | |
| | S. mutans, S. oralis, S. sanguinis | | |
| 1T-268 | F. nucleatum, S. mutans, S. oralis, | PITPKKEKCGLGTYAPKNP | 234 |
| | S. sanguinis | VFSKSRV | |
| 1T-269 | F. nucleatum, S. mutans, S. oralis, | PLYVAAVEKINTAKKH | 235 |
| | S. sanguinis | | |
| 1T-270 | F. nucleatum, S. mutans, S. oralis, | VHEFDIQKILQNR | 236 |
| | S. sanguinis | | |
| 1T-271 | A. naeslundii, F. nucleatum, P. gingivalis, | FLIQKFLLIKTFPPYRKKY | 237 |
| | S. epidermidis, S. gordonii, | VVIVSQTGTA | |
| | S. mitis, S. mutans, S. oralis, | | |
| | S. salivarious, S. sanguinis | | |
| 1T-272 | F. nucleatum, S. mutans, S. oralis, | QLAPIDKQLKAVKKIAFY | 238 |
| | S. sanguinis | ESESTAAKAVTVA | |
| 1T-273 | F. nucleatum, P. gingivalis, T. denticola, | YNEPNYKWLESYKIYKQR | 239 |
| | S. mitis, S. mutans, S. oralis | CEDRTGMYYTEET | |
| 1T-274 | F. nucleatum, S. mutans, S. oralis, | ETTTEINAIKLHRIKQRSPQ | 240 |
| | S. sanguinis | GTRRVN | |
| 1T-275 | A. naeslundii, F. nucleatum, P. gingivalis, | QVLKNFSISRRYKINNPFF | 241 |
| | T. denticola, S. epidermidis, | KILLFIQLRTL | |
| | S. gordonii, S. mitis, | | |
| | S. mutans, S. oralis, S. salivarious, | | |
| | S. sanguinis | | |
| 1T-276 | A. naeslundii, F. nucleatum, P. gingivalis, | ILTLLILGSIGFFILKIKLKL | 242 |
| | S. epidermidis, S. gordonii, | GRF | |
| | gordonii, S. mitis, S. mutans, S. oralis, | | |
| | S. sanguinis | | |
| 1T-277 | A. naeslundii, F. nucleatum, P. gingivalis, | IYYMRFVNKPLEKTFFKI | 243 |
| | T. denticola, S. gordonii, | | |
| | S. mitis, S. mutans, S. oralis, | | |
| | S. salivarious, S. sanguinis | | |
| 1T-278 | A. naeslundii, F. nucleatum, P. gingivalis, | SINSSAGIQPHCLSSSFVLR | 244 |
| | S. gordonii, S. mitis, | TKHCFY | |
| | S. mutans, S. oralis, S. salivarious, | | |
| | S. sanguinis | | |
| 1T-279 | A. naeslundii, F. nucleatum, P. gingivalis, | FVLRTKHCFY | 245 |
| | S. gordonii, S. mitis, | | |
| | S. mutans, S. oralis, S. salivarious, | | |
| | S. sanguinis | | |
| 1T-280 | A. naeslundii, F. nucleatum, P. gingivalis, | TNNKNKVIIKAIKFKNKDF | 246 |
| | T. denticola, S. gordonii, | INLDLFIYRR | |
| | S. mitis, S. mutans, S. oralis, | | |
| | S. salivarious, S. sanguinis | | |
| 1T-281 | A. naeslundii, F. nucleatum, P. gingivalis, | KYEKLTKENLFIRNSGNM | 247 |
| | S. epidermidis, S. gordonii, | CVFIYFLFFG | |
| | S. mitis, S. mutans, S. oralis, | | |
| | S. salivarious, S. sanguinis | | |
| 1T-282 | F. nucleatum, P. gingivalis, S. gordonii, | ISLVFPAYT | 248 |
| | S. mitis, S. mutans, S. oralis, | | |
| | S. salivarious, S. sanguinis | | |
| 1T-283 | A. naeslundii, F. nucleatum, P. gingivalis, | LCTKLEDKQRGRIPAELFII | 249 |
| | T. denticola, S. epidermidis, | SPIKILERNDAL | |
| | S. gordonii, S. mitis, | | |
| | S. mutans, S. oralis, S. salivarious, | | |
| | S. sanguinis | | |
| 1T-284 | A. naeslundii, F. nucleatum, P. gingivalis, | FQYYFSLKRV | 250 |
| | S. gordonii, S. mitis, | | |
| | S. mutans, S. oralis, S. salivarious, | | |
| | S. sanguinis | | |
| 1T-285 | A. naeslundii, F. nucleatum, P. gingivalis, | FFPYYLADFYKQLKFLNE | 251 |
| | S. gordonii, S. mitis, | YQTKNKDKVVEFK | |
| | S. mutans, S. oralis, S. salivarious, | | |
| | S. sanguinis | | |
| 1T-286 | S. sanguinis | LGFFNNKADLVKADTERD | 252 |
| | | NRMSSLKIKDL | |
| 1T-287 | P. gingivalis, T. denticola, S. gordonii, | KGYPLPFQYRLNNH | 253 |
| | S. mitis, S. mutans, S. oralis, | | |
| | S. salivarious, S. sanguinis | | |
| 1T-288 | F. nucleatum, S. gordonii, S. salivarious, | RWVGGEPSADIYLSAKDT | 254 |
| | S. sanguinis | KT | |
| 1T-289 | F. nucleatum, P. gingivalis, S. gordonii, | EPSADIYLSAKDTKT | 255 |
| | S. mitis, S. mutans, S. oralis, | | |
| | S. sanguinis | | |
| 1T-290 | A. naeslundii, F. nucleatum, P. gingivalis, | IINQLNLILLRLMEILIL | 256 |
| | S. gordonii, S. mitis, | | |
| | S. mutans, S. oralis, S. salivarious, | | |
| | S. sanguinis | | |
| 1T-291 | A. naeslundii, F. nucleatum, P. gingivalis, | DMKIIKLYIKILSFLFIKYC | 257 |
| | T. denticola, S. mitis, | NKKLNSVKLKA | |
| | S. mutans, S. oralis | | |
| 1T-292 | A. naeslundii, F. nucleatum, P. gingivalis, | IINQLNLILLRLMEILIL | 258 |
| | S. epidermidis, S. gordonii, | | |
| | S. mitis, S. mutans, S. oralis, | | |
| | S. salivarious, S. sanguinis | | |
| 1T-293 | A. naeslundii, F. nucleatum, P. gingivalis, | HVEDCFLLSNARTTAIHG | 259 |
| | S. epidermidis, S. gordonii, | RANPARGEPRTRSE | |
| | S. mitis, S. mutans, S. oralis, | | |
| | S. salivarious, S. sanguinis | | |
| 1T-294 | T. denticola | YDKIADGVFKIGKRGVL | 260 |
| 1T-295 | S. mitis, S. salivarious, S. sanguinis | KYKLKKIIL | 261 |
| 1T-296 | A. naeslundii, F. nucleatum, P. gingivalis, | EYSQQSFKAKPCSERGVL | 262 |
| | S. gordonii, S. mitis, | SP | |
| | S. mutans, S. oralis, S. salivarious, | | |
| | S. sanguinis | | |
| 1T-297 | A. naeslundii, F. nucleatum, T. denticola, | RSLRLNNALTKLPKLWYN | 263 |
| | S. mitis, S. mutans, S. oralis | RIKEAFYAYNDYDK | |
| 1T-298 | A. naeslundii, F. nucleatum, P. gingivalis, | ILNKKPKLPLWKLGKNYF | 264 |
| | T. denticola, S. gordonii, | RRFYVLPTFLA | |
| | S. mitis, S. mutans, S. oralis, | | |
| | S. salivarious, S. sanguinis | | |
| 1T-299 | A. naeslundii, F. nucleatum, S. epidermidis, | SMLTSFLRSKNTRSLKMY | 265 |
| | S. gordonii, S. mitis, | KDVHF | |
| | S. mutans, S. oralis, S. salivarious, | | |
| | S. sanguinis | | |
| 1T-300 | A. naeslundii, F. nucleatum, P. gingivalis, | PLIISKAQIKMSGDILGSCF | 266 |
| | S. epidermidis, S. gordonii, | KLFYLRPFF | |
| | S. mitis, S. mutans, S. oralis, | | |
| | S. salivarious, S. sanguinis | | |
| 1T-301 | F. nucleatum, S. gordonii, S. sanguinis | SKLPRVLDASLKL | 267 |
| 1T-302 | A. naeslundii, P. gingivalis, S. epidermidis, | IIIILPKIYLVCKTV | 268 |
| | S. gordonii, S. mitis, | | |
| | S. mutans, S. oralis, S. salivarious, | | |
| | S. sanguinis | | |
| 1T-303 | A. naeslundii, F. nucleatum, P. gingivalis, | LDYENMDCKKRIRI | 269 |
| | S. gordonii, S. mitis, | | |
| | S. mutans, S. oralis, S. salivarious, | | |
| | S. sanguinis | | |
| 1T-304 | P. gingivalis | STAGEASRRTASEASRRT | 270 |
| | | AAKLRG | |
| TT-305 | F. nucleatum | ARNALNMRDVPVDAAIIG | 271 |
| | | IIDGMDEE | |
| TT-306 | F. nucleatum | KILNEAEGKLLKVIEKNGE | 272 |
| | | IDIEEI | |
| TT-307 | F. nucleatum | NGDKKAKEELDKWDEVI | 273 |
| | | KELNIQF | |
| TT-308 | F. nucleatum | GLVIIPNLIALIILFSQVRQQ | 274 |
| | | TKDYFSNPKLSSR | |
| TT-309 | F. nucleatum | EPLPLTKYDKKDTEMKKV | 275 |
| | | FKEILAGKVGYEKEEE | |
| TT-310 | F. nucleatum, | TKLKKNNKLLSAKKENTL | 276 |
| | | HTKDK | |
| TT-311 | S. mutans, S. sobrinus | AIFDAMHNL | 277 |
| PF-060 | C. albicans hyphae | HSSHL | 278 |
| PF-024 | C. albicans hyphae | DLRKAK | 279 |
| PF-636 | C. albicans hyphae | LVRLA | 280 |
| PF-178 | C. albicans hyphae | EVYSSPTNNVAITVQNN | 281 |
| PF-761 | C. albicans hyphae | SKFELVNYASGCSCGADC | 282 |
| | | KCASETECKCASKK | |
| PF-770 | C. albicans hyphae | GVGIGFIMMGVVGYAVK | 283 |
| | | LVHIPIRYLIV | |
| 1T-65 | C. albicans hyphae | HARAAVGVAELPRGAAV | 284 |
| | | EVELIAAVRP | |
| PF-141 | C. albicans hyphae | VVRRFQGM | 285 |
| PF-543 | C. albicans hyphae | NILFGIIGFVVAMTAAVIV | 286 |
| | | TAISIAK | |
| PF-634 | C. albicans hyphae | MPKARPVNHNKKKSKITI | 287 |
| | | KSNFTLFYMFNP | |
| PF-040 | C. albicans hyphae | MIHLTKQNTMEALHFIKQ | 288 |
| | | FYDMFFILNFNV | |
| PF-051 | C. albicans hyphae | RFFNFEIKKSTKVDYVFAH | 289 |
| | | VDLSDV | |
| PF-580 | C. albicans hyphae | EILNNNQVIKELTMKYKT | 290 |
| | | QFESNLGGWTARARR | |
| PF-583 | C. albicans hyphae | KFQGEFTNIGQSYIVSASH | 291 |
| | | MSTSLNTGK | |
| 1T-36 | C. albicans hyphae | VYRHLRFIDGKLVEIRLERK | 292 |
| PF-206 | C. albicans hyphae | KLRSASKKSLQEKSCGIMP | 293 |
| | | EKPAG | |
| 1T-13 | yeast and hyphal forms | FRSPCINNNSLQPPGVYPAR | 294 |
| 1T-21 | yeast and hyphal forms | YVEEAVRAALKKEARIST | 295 |
| | | EDTPVNLPSFDC | |
| PF-030 | yeast and hyphal forms | MTCHQAPTTTHQSNMA | 296 |
| PF-463 | yeast and hyphal forms | MVILVFSLIFIFTDNYLVY | 297 |
| | | QSKSIKEDVMI | |
| PF-380 | yeast and hyphal forms | KKIIPLITLFVVTLVG | 298 |
| PF-515 | yeast and hyphal forms | DKSTQDKDIKQAKLLAQE | 299 |
| | | LGL-NH2 | |
| PF-458 | yeast and hyphal forms | ISLIIFIMLFVVALFKCITNY | 300 |
| | | KHQS | |
| PF-S018 | yeast and hyphal forms | GMPQIPRLRI | 301 |
| 1T-16 | yeast and hyphal forms | IDMR | 302 |
| PF-211 | yeast and hyphal forms | DSFDSLSPFRERGGEREDG | 303 |
| | | CDAMPLP | |
| PF-002 | yeast and hyphal forms | NDDAQ | 304 |
| PF-S003 | yeast and hyphal forms | ALALLKQDLLNFEGRGRII | 305 |
| | | TSTYLQFNEGCVP | |
| PF-021 | yeast and hyphal forms | FSLNFSKQKYVTVN | 306 |
| 1T-14 | yeast and hyphal forms | ALAGLAGLISGK | 307 |
| 1T-15 | yeast and hyphal forms | DVILRVEAQ | 308 |
| PF-629 | yeast and hyphal forms | GLAAIATVFALY | 309 |
| PF-617 | yeast and hyphal forms | PMNAAEPE | 310 |
| PF-621 | yeast and hyphal forms | PPSSFLV | 311 |
| PF-631 | yeast and hyphal forms | LIIYFSKTGNTARATRQI | 312 |
| PF-009 | yeast and hyphal forms | SKKYNHILNQENR | 313 |
| 1T-17 | yeast and hyphal forms | NNAIVYIS | 314 |
| 1T-20 | yeast and hyphal forms | PALVDLSNKEAVWAVLD | 315 |
| | | DHS | |
| 1T-68 | yeast cells | GGTKEIVYQRG | 316 |
| 1T-70 | yeast cells | NRQAQGERAHGEQQG | 317 |
| PF-167 | yeast cells | AIEGVIKKGACFKLLRHE | 318 |
| | | MF | |
| PF-168 | yeast cells | VLPFPAIPLSRRRACVAAP | 319 |
| | | RPRSRQRAS | |
| PF-170 | yeast cells | RLARGRPTNLCGRRG | 320 |
| PF-176 | yeast cells | RLTSNQFLTRITPFVFAQH | 321 |
| Rv2369c | C. albicans | VRQVPVDRPESRHQKPGD | 322 |
| PF2-038 | | VPRDPRC | |
| Rv2561 | C. albicans | QHQCPGMRPAPADAPEVP | 323 |
| PF2-040 | | HAARADQKRPSLRL | |
| Rv1535 | C. albicans | DPLVDGAARLLSIPLRHLY | 324 |
| PF2-033 | | AALWRVGLLEVQA | |
| Rv2660c | C. albicans | RSPDFVDETAGQSWCAIL | 325 |
| PF2-044 | | GLNQFH | |
| Rv3760 | C. albicans | GLITVFAGTARILQLRRAA | 326 |
| PF2-059 | | KKTHAAALR | |
| PF-S024 | Corynebacteria spp. | SKRGRKRKDRRKKKANH | 327 |
| | | GKRPNS | |
| PF-001 | S. epidermidis, M. luteus, P. mirabilis, | MNNWIIVAQLSVTVINEII | 328 |
| | E. coli, P. aeruginosa, | DIMKEKQKGGK | |
| | C. albicans, MRSA, E. faecalis, | | |
| | C. jeikeium | | |
| PF-002 | S. epidermidis, P. mirabilis, C. albicans, | NDDAQ | 329 |
| | C. jeikeium, C. jejuni | | |
| PF-003 | S. epidermidis, M. luteus, P. mirabilis, | MNNWIKVAQISVTVINEVI | 330 |
| | C. albicans, MRSA, C. jeikeium | DIMKEKQNGGK | |
| PF-004 | S. epidermidis, B. subtilis, B. fragilis, | ARLSKAIIIAVIVVYHLDV | 331 |
| | E. coli, P. aeruginosa, C. albicans, | RGLF | |
| | S. pneumoniae, E. faecalis, | | |
| | C. jeikeium | | |
| PF-005 | S. epidermidis, E. coli, MRSA, S. pneumoniae, | MESIFKIKLMNGICRSENM | 332 |
| | E. faecalis | NMKKKNKGEKI | |
| PF-006 | S. epidermidis, M. luteus, E. coli, | MGIIAGIIKFIKGLIEKFTGK | 333 |
| | P. aeruginosa, MRSA, E. faecalis, | | |
| | C. jeikeium, C. jejuni | | |
| PF-007 | S. epidermidis, M. luteus, E. coli, | MGIIAGIIKVIKSLIEQFTGK | 334 |
| | P. aeruginosa, C. albicans, | | |
| | MRSA, S. pneumoniae, E. faecalis, | | |
| | C. jeikeium | | |
| PF-008 | S. epidermidis, M. luteus, MRSA, | MIEIGSIAYLNGGSKKYNH | 335 |
| | C. jejuni | ILNQENR | |
| PF-009 | M. luteus, P. mirabilis, C. albicans | SKKYNHILNQENR | 336 |
| PF-010 | S. epidermidis, M. luteus, E. coli, | MDIDVNKLLQAFVYFKSF | 337 |
| | C. albicans | EKLRHNNS | |
| PF-011 | M. luteus, E. coli, P. aeruginosa, | MFCYYKQHKGDNFSIEEV | 338 |
| | S. pneumoniae, C. jeikeium | KNIIADNEMKVN | |
| PF-012 | S. epidermidis, M. luteus, P. mirabilis, | WRGPNTEAGGKSANNIVQ | 339 |
| | E. coli, P. aeruginosa, | VGGAPT | |
| | MRSA, S. pneumoniae, C. jeikeium, | | |
| | C. jejuni | | |
| PF-013 | M. luteus, P. mirabilis, E. coli, P. aeruginosa, | LIQKGLNQTFIVVIRLNNFI | 340 |
| | MRSA, S. pneumoniae, | KKS | |
| | C. jeikeium, C. jejuni | | |
| PF-014 | E. coli, C. jeikeium | HPTDNKHN | 341 |
| PF-015 | E. faecalis, C. jeikeium | SIDKRNLYNLKYYE | 342 |
| PF-016 | S. epidermidis, E. faecalis, C. jeikeium | RKQYDDLSFNFLY | 343 |
| PF-017 | E. coli | ESIIE | 344 |
| PF-018 | E. coli, C. jeikeium | YYKTYFKEV | 345 |
| PF-020 | S. epidermidis, M. luteus, C. albicans, | MKIILLLFLIFGFIVVVTLK | 346 |
| | MRSA, S. pneumoniae, | SEHQLTLFSI | |
| | E. faecalis | | |
| PF-021 | S. epidermidis, M. luteus, P. mirabilis, | FSLNFSKQKYVTVN | 347 |
| | E. coli, C. albicans, E. faecalis, | | |
| | C. jeikeium | | |
| PF-022 | M. luteus, P. mirabilis, E. coli, P. aeruginosa, | MINELKNKNSGIMNNYVV | 348 |
| | C. albicans, MRSA, | TKESKL | |
| | S. pneumoniae, E. faecalis, C. jeikeium, | | |
| | C. jejuni | | |
| PF-023 | M. luteus, C. jeikeium | MTKNTIISLENEKTQINDS | 349 |
| | | ENESSDLRKAK | |
| PF-024 | M. luteus, C. albicans, MRSA, E. faecalis, | DLRKAK | 350 |
| | C. jeikeium | | |
| PF-025 | S. epidermidis, M. luteus, P. mirabilis, | LLIIFRLWLELKWKNKK | 351 |
| | E. coli, P. aeruginosa, | | |
| | MRSA, E. faecalis, C. jejuni | | |
| PF-026 | S. epidermidis, M. luteus, P. mirabilis, | SIHFIN | 352 |
| | C. albicans, MRSA, E. faecalis, | | |
| | C. jeikeium | | |
| PF-027 | M. luteus, MRSA, E. faecalis, C. jejuni | HNARKYLEFISQKIDGDKL | 353 |
| | | TKEDSL | |
| PF-028 | S. epidermidis, M. luteus, MRSA | ALDCSEQSVILWYETILDK | 354 |
| | | IVGVIK | |
| PF-029 | S. epidermidis, M. luteus, C. albicans, | NSTNE | 355 |
| | C. jejuni | | |
| PF-030 | S. epidermidis, M. luteus, P. mirabilis, | MTCHQAPTTTHQSNMA | 356 |
| | E. coli, P. aeruginosa, | | |
| | C. albicans, MRSA, S. pneumoniae, | | |
| | E. faecalis, C. jeikeium, | | |
| | C. jejuni | | |
| PF-031 | M. luteus, C. albicans | MPHHSTTSSRIVVPAHQS | 357 |
| | | NMASTPNLSITP | |
| PF-033 | S. epidermidis, M. luteus, E. coli, | MFIFKTTSKSHFHNNVKSL | 358 |
| | P. aeruginosa, C. albicans, | ECIKIPINKNR | |
| | MRSA, S. pneumoniae | | |
| PF-034 | M. luteus | EPKKKHFPKMESASSEP | 359 |
| PF-035 | S. epidermidis, M. luteus, E. coli, | SFYESY | 360 |
| | C. albicans, MRSA, C. jeikeium, | | |
| | C. jejuni | | |
| PF-036 | S. epidermidis, M. luteus, P. mirabilis, | ILNRLSRIVSNEVTSLIYSLK | 361 |
| | E. coli, C. albicans, | | |
| | MRSA, S. pneumoniae, C. jejuni | | |
| PF-037 | S. epidermidis, M. luteus, P. aeruginosa, | MTKKRRYDTTEFGLAHS | 362 |
| | C. albicans, MRSA, | MTAKITLHQALYK | |
| | S. pneumoniae, E. faecalis, C. jeikeium | | |
| PF-038 | M. luteus | MAYKDEGKETKFAVKGY | 363 |
| | | KD | |
| PF-039 | P. mirabilis, C. jeikeium | MLEEKNKSL | 364 |
| PF-040 | S. epidermidis, M. luteus, P. mirabilis, | MIHLTKQNTMEALHFIKQ | 365 |
| | E. coli, P. aeruginosa, | FYDMFFILNFNV | |
| | C. albicans, MRSA, S. pneumoniae, | | |
| | E. faecalis, C. jeikeium, | | |
| | C. jejuni | | |
| PF-041 | MRSA | ELLVILPGFI | 366 |
| PF-042 | S. epidermidis, M. luteus, P. mirabilis, | LLLSYFRYTGALLQSLF | 367 |
| | E. coli, P. aeruginosa, | | |
| | C. albicans, MRSA, S. pneumoniae, | | |
| | E. faecalis, C. jeikeium | | |
| PF-043 | M. luteus, C. jejuni | MIKNETAYQMNELLVIRS | 368 |
| | | AYAK | |
| PF-044 | S. epidermidis, M. luteus, MRSA, | KLKKYIHKPD | 369 |
| | C. jeikeium | | |
| PF-045 | S. epidermidis, E. coli, E. faecalis, | LDINDYRSTY | 370 |
| | C. jejuni | | |
| PF-046 | E. coli, E. faecalis, C. jeikeium | LDFYLTKHLTLML | 371 |
| PF-047 | S. mutans | NQEPSLQQDKEQKDNKG | 372 |
| PF-048 | S. epidermidis, M. luteus, E. coli, | LYFAFKKYQERVNQAPNI | 373 |
| | MRSA, C. jeikeium, C. jejuni | EY | |
| PF-049 | S. epidermidis, MRSA, C. jeikeium, | AYYLKRREEKGK | 374 |
| | C. jejuni | | |
| PF-050 | S. epidermidis, M. luteus, E. coli, | SYYLKRREEKGK | 375 |
| | C. jeikeium | | |
| PF-051 | S. epidermidis, M. luteus, P. aeruginosa, | RFFNFEIKKSTKVDYVFAH | 376 |
| | C. albicans, MRSA, | VDLSDV | |
| | S. pneumoniae, E. faecalis | | |
| PF-052 | S. epidermidis, M. luteus, E. coli, | QELINEAVNLLVKSK | 377 |
| | MRSA, E. faecalis, C. jeikeium, | | |
| | C. jejuni | | |
| PF-053 | S. epidermidis, M. luteus, E. coli, | KLFGQWGPELGSIYILPAL | 378 |
| | P. aeruginosa, C. albicans, | IGSIILIAIVTLILRAMRK | |
| | MRSA, S. pneumoniae, E. faecalis | | |
| PF-054 | S. epidermidis, E. coli | VSISRFIGGGHVFNGNNKR | 379 |
| | | NL | |
| PF-055 | S. mutans | GHVFNGNNKRNL | 380 |
| PF-056 | S. epidermidis, M. luteus, P. mirabilis, | AEQLFGKQKQRGVDLFLN | 381 |
| | E. coli, P. aeruginosa, | RLTIILSILFFVLMICISYLGM | |
| | C. albicans, MRSA, S. pneumoniae, | | |
| | E. faecalis, C. jeikeium | | |
| PF-057 | S. epidermidis, M. luteus, P. mirabilis, | TMIVISIPRFEEYMKARHK | 382 |
| | E. coli, P. aeruginosa, | KWM | |
| | C. albicans, MRSA, S. pneumoniae, | | |
| | E. faecalis, C. jeikeium, | | |
| | C. jejuni | | |
| PF-058 | S. epidermidis, M. luteus, E. coli, | FADQSQDNA | 383 |
| | C. albicans, MRSA, C. jeikeium, | | |
| | C. jejuni | | |
| PF-060 | E. coli, C. albicans, C. jeikeium | HSSHL | 384 |
| PF-061 | S. epidermidis, S. pneumoniae | GYNSYKAVQDVKTHSEE | 385 |
| | | QRVTAKK | |
| PF-062 | S. epidermidis, M. luteus, E. coli, | MKKKRINNDILGRMIYSSS | 386 |
| | P. aeruginosa, MRSA, S. pneumoniae, | IDKRNLYNLKYYE | |
| | E. faecalis, C. jeikeium | | |
| PF-063 | S. epidermidis, M. luteus, E. coli, | IAAIIVLVLFQKGLLQIFN | 387 |
| | P. aeruginosa, MRSA, S. pneumoniae, | WILIQLQ | |
| | E. faecalis, C. jeikeium, | | |
| | C. jejuni | | |
| PF-064 | E. coli | DYYGKE | 388 |
| PF-065 | M. luteus, E. coli, P. aeruginosa, | LEKNTRDNYFIHAIDRIYI | 389 |
| | C. albicans, MRSA, S. pneumoniae, | NTSKGLFPESELVAWG | |
| | C. jeikeium, C. jejuni | | |
| PF-066 | M. luteus, E. coli, C. jeikeium | IKGTVKAVDETTVVITVN | 390 |
| | | GHGTELTFEKPAIKQVDPS | |
| PF-067 | S. epidermidis, M. luteus, P. mirabilis, | DLIVKVHICFVVKTASGY | 391 |
| | E. coli, P. aeruginosa, | CYLNKREAQAAI | |
| | C. albicans, MRSA, S. pneumoniae, | | |
| | E. faecalis, C. jeikeium, | | |
| | C. jejuni | | |
| PF-068 | S. epidermidis, M. luteus, P. mirabilis, | SHLINNFGLSVINPSTPICL | 392 |
| | E. coli, P. aeruginosa, | NFSPVFNLLTVYGITCN | |
| | C. albicans, MRSA, S. pneumoniae, | | |
| | E. faecalis, C. jeikeium | | |
| PF-069 | E. faecalis, C. jejuni | FDPVPLKKDKSASKHSHK | 393 |
| | | HNH | |
| PF-070 | S. epidermidis, C. jejuni | SMVKSEIVDLLNGEDNDD | 394 |
| PF-071 | S. epidermidis, E. coli, P. aeruginosa, | HCVIGNVVDIANLLKRRA | 395 |
| | C. albicans, MRSA, | VYRDIADVIKMR | |
| | S. pneumoniae, E. faecalis, C. jeikeium, | | |
| | C. jejuni | | |
| PF-073 | S. epidermidis, M. luteus, P. aeruginosa, | CPSVTMDACALLQKFDFC | 396 |
| | C. albicans, MRSA, | NNISHFRHFFAIKQPIER | |
| | S. pneumoniae, E. faecalis | | |
| PF-074 | S. epidermidis, M. luteus, MRSA | RDIHPIYFMTKD | 397 |
| PF-075 | M. luteus, E. coli, P. aeruginosa, | FVNSLIMKDLSDNDMRFK | 398 |
| | MRSA, C. jeikeium | YEYYNREKDT | |
| PF-076 | S. epidermidis, M. luteus, E. coli, | LYQYELLSKEEYLKCTLII | 399 |
| | P. aeruginosa, C. albicans, | NQRRNEQK | |
| | MRSA, S. pneumoniae, E. faecalis, | | |
| | C. jeikeium, C. jejuni | | |
| PF-097 | C. jeikeium | QPTQGEQGTRPRRPTPMR | 400 |
| | | GLLI | |
| PF-099 | S. epidermidis, M. luteus, E. coli, | EIIAYLEGRFANA | 401 |
| | C. jeikeium | | |
| PF-101 | S. mutans | DPVPERQEQACACHRTAK | 402 |
| | | PGK | |
| PF-104 | MRSA, C. jeikeium | ERTAVNDLWI | 403 |
| PF-123 | M. luteus, E. coli | TTRPQVAEDRQLDDALKE | 404 |
| | | TFPASDPISP | |
| PF-124 | S. epidermidis, M. luteus, P. mirabilis, | MADGQIAAIAKLHGVPVA | 405 |
| | E. coli, P. aeruginosa, | TRNIRHFQSFGVELINPWSG | |
| | C. albicans, MRSA, E. faecalis, | | |
| | C. jejuni | | |
| PF-125 | S. epidermidis, M. luteus, P. mirabilis, | YVVGALVILAVAGLIYSM | 406 |
| | E. coli, P. aeruginosa, | LRKA | |
| | C. albicans, MRSA, S. pneumoniae, | | |
| | E. faecalis, C. jejuni | | |
| PF-126 | S. epidermidis, M. luteus, P. mirabilis, | FSPEAFGIGAAGVLGSFVT | 407 |
| | E. coli, P. aeruginosa, | GLLIGWVASLLRKAK | |
| | C. albicans, MRSA, S. pneumoniae, | | |
| | E. faecalis, C. jeikeium, | | |
| | C. jejuni | | |
| PF-127 | S. epidermidis, M. luteus, E. coli, | MLRYLSLFAVGLATGYA | 408 |
| | P. aeruginosa, C. albicans, | WGWIDGLAASLAV | |
| | MRSA, S. pneumoniae, E. faecalis | | |
| PF-128 | M. luteus, P. aeruginosa, E. faecalis | GIKVVAARFEEIQFSENFD | 409 |
| | | SIILA | |
| PF-129 | S. epidermidis, MRSA, E. faecalis, | MKLLARDPWVCAWNDIW | 410 |
| | C. jeikeium, C. jejuni | | |
| PF-130 | E. faecalis, C. jeikeium, C. jejuni | LQRSDEESMPRRHEKYS | 411 |
| PF-131 | S. epidermidis, E. coli, MRSA, C. jeikeium | RRAAARTKGNRR | 412 |
| PF-132 | S. epidermidis, C. jeikeium | RPGDGAAEQGRSR | 413 |
| PF-133 | S. epidermidis, C. jeikeium, C. jejuni, | GDPTAGQKPVECP | 414 |
| | M. smegmatis | | |
| PF-134 | S. epidermidis, C. jeikeium | GKAMKRQDCSAL | 415 |
| PF-135 | S. epidermidis, M. luteus, E. coli, | PPARPARIPQTPTLHGASL | 416 |
| | P. aeruginosa, MRSA, C. jeikeium, | FRQRS | |
| | M. smegmatis | | |
| PF-136 | S. epidermidis, M. luteus, P. mirabilis, | LRGRVGRITACGYPP | 417 |
| | E. coli, MRSA, E. faecalis, | | |
| | C. jeikeium, C. jejuni, M. smegmatis | | |
| PF-137 | S. epidermidis, P. mirabilis, S. pneumoniae, | VLGKGHDLLDVGKTALK | 418 |
| | C. jeikeium, C. jejuni | SRVFAWLGGS | |
| PF-138 | S. epidermidis, M. luteus, P. mirabilis, | AVHHSLLFR | 419 |
| | E. coli, C. albicans, | | |
| | MRSA, C. jeikeium, C. jejuni | | |
| PF-139 | S. epidermidis, M. luteus, P. mirabilis, | ALSKPAIQARTLCRRQDPP | 420 |
| | E. coli, P. aeruginosa, | | |
| | C. albicans, S. pneumoniae, E. faecalis, | | |
| | C. jeikeium, C. jejuni | | |
| PF-140 | S. epidermidis, M. luteus, P. mirabilis, | FHRRVIRASEWALTTRSFS | 421 |
| | E. coli, P. aeruginosa, | TPLRSAAR | |
| | C. albicans, MRSA, S. pneumoniae, | | |
| | E. faecalis, C. jeikeium, | | |
| | C. jejuni, M. smegmatis | | |
| PF-141 | S. epidermidis, M. luteus, C. albicans, | VVRRFQGM | 422 |
| | MRSA, C. jeikeium | | |
| PF-142 | S. mutans | GIDRGCQAAR | 423 |
| PF-143 | S. epidermidis, MRSA, C. jeikeium | LSPRPIIVSRRSRADNNND | 424 |
| | | WSR | |
| PF-144 | S. epidermidis, M. luteus, E. coli, | RSGQPVGRPSRRAWLR | 425 |
| | C. albicans, MRSA, S. pneumoniae, | | |
| | E. faecalis, C. jeikeium | | |
| PF-145 | S. epidermidis, M. luteus, P. mirabilis, | GIVLTGRAGLVSGACSMA | 426 |
| | E. coli, P. aeruginosa, | LGVGLG | |
| | C. albicans, MRSA, S. pneumoniae, | | |
| | E. faecalis, C. jeikeium, | | |
| | C. jejuni, M. smegmatis | | |
| PF-146 | S. epidermidis, M. luteus, P. aeruginosa, | GCGKRRIITKSASRDTR | 427 |
| | C. albicans, MRSA, | | |
| | C. jeikeium | | |
| PF-147 | S. epidermidis, M. luteus, MRSA | RRPRRRRSGHGQSASAA | 428 |
| PF-148 | S. epidermidis, M. luteus, P. mirabilis, | RRGCTERLRRMARRNAW | 429 |
| | E. coli, P. aeruginosa, | DLYAEHFY | |
| | C. albicans, MRSA, S. pneumoniae, | | |
| | E. faecalis, C. jeikeium, | | |
| | C. jejuni, M. smegmatis | | |
| PF-149 | S. epidermidis, M. luteus, E. coli, | GKVSVLTRVPRSLGGAPA | 430 |
| | MRSA, C. jeikeium | NQ | |
| PF-150 | S. epidermidis, MRSA | EIQAKGTG | 431 |
| PF-151 | S. epidermidis, MRSA, E. faecalis, | EEYPARVPLSGEDVTEAR | 432 |
| | C. jeikeium | RH | |
| PF-152 | S. epidermidis, C. albicans, | VGYFIWKDSHSRKG | 433 |
| | MRSA, E. faecalis, C. jeikeium | | |
| PF-153 | M. luteus, P. mirabilis, E. coli, | GILARADCSQIA | 434 |
| | MRSA | | |
| PF-154 | S. mutans | GIKKSKHPSTDDYVVKTTI | 435 |
| | | DSL | |
| PF-155 | C. jeikeium | GRYGDDSKERQGRAQ | 436 |
| PF-156 | S. epidermidis, C. jeikeium | LITAEQPATAPIAGK | 437 |
| PF-157 | S. epidermidis, M. luteus, P. mirabilis, | HTAVVWLAGVSGCVALS | 438 |
| | E. coli, P. aeruginosa, | HCEPA | |
| | C. albicans, MRSA, S. pneumoniae, | | |
| | E. faecalis, C. jeikeium, | | |
| | C. jejuni | | |
| PF-158 | S. epidermidis | VRLESRPADLPE | 439 |
| PF-159 | S. epidermidis | TMAFVEKAQLRVPVGDD | 440 |
| | | LPV | |
| PF-160 | S. epidermidis | SFHASLTKNEKPIKSTG | 441 |
| PF-161 | S. epidermidis, M. luteus, E. coli, | RGRALASTATTRPARRRR | 442 |
| | C. jejuni | | |
| PF-162 | S. epidermidis, MRSA | GIRRLHSVENLNREISHRM | 443 |
| | | AGLR | |
| PF-163 | S. epidermidis | TSWLRAAERQEIGEPTKTF | 444 |
| | | GEKTTSL | |
| PF-164 | S. epidermidis, M. luteus, E. coli, | EEVSRALAGIGLGLGCRIG | 445 |
| | C. jeikeium | | |
| PF-165 | MRSA, C. jejuni | GPVSVVASLRRGTTVQRH | 446 |
| | | SQNNHNKGKP | |
| PF-166 | E. coli, C. jeikeium | SKAVSRKRSI | 447 |
| PF-167 | S. epidermidis, E. coli, C. albicans, | AIEGVIKKGACFKLLRHE | 448 |
| | MRSA, C. jeikeium, C. jejuni | MF | |
| PF-168 | S. epidermidis, M. luteus, E. coli, | VLPFPAIPLSRRRACVAAP | 449 |
| | C. albicans, MRSA, C. jeikeium, | RPRSRQRAS | |
| | C. jejuni | | |
| PF-169 | S. epidermidis, E. coli, C. albicans, | APGSAADSPRSRADD | 450 |
| | E. faecalis, C. jeikeium | | |
| PF-170 | S. epidermidis, M. luteus, P. mirabilis, | RLARGRPTNLCGRRG | 451 |
| | E. coli, P. aeruginosa, | | |
| | C. albicans, MRSA, S. pneumoniae, | | |
| | E. faecalis, C. jejuni | | |
| PF-171 | S. epidermidis, E. coli, P. aeruginosa, | TQVTLCRTW | 452 |
| | S. pneumoniae | | |
| PF-172 | S. epidermidis, M. luteus, E. coli, | LTGVRRPWRAPWAGTSG | 453 |
| | P. aeruginosa, MRSA, E. faecalis, | WALR | |
| | C. jejuni | | |
| PF-173 | S. epidermidis, M. luteus, P. mirabilis, | AGRTAIVQGGG | 454 |
| | E. coli, P. aeruginosa, | | |
| | C. albicans, C. jeikeium, C. jejuni | | |
| PF-174 | S. epidermidis, P. aeruginosa, C. jeikeium | RGGDSPARRRPGLAGPGG | 455 |
| | | PG | |
| PF-175 | S. epidermidis, E. faecalis | RRRPAGQRPEKASQAMIAA | 456 |
| PF-176 | S. epidermidis, M. luteus, P. mirabilis, | RLTSNQFLTRITPFVFAQH | 457 |
| | E. coli, C. albicans, | | |
| | MRSA, E. faecalis, C. jeikeium | | |
| PF-177 | M. luteus, MRSA, E. faecalis, C. jeikeium | VTSEPGIAHDIRLLPRAAA | 458 |
| | | FR | |
| PF-178 | S. epidermidis, M. luteus, B. subtilis, | EVYSSPTNNVAITVQNN | 459 |
| | P. mirabilis, E. coli, P. aeruginosa, | | |
| | C. albicans, MRSA, | | |
| | S. pneumoniae, E. faecalis, C. jeikeium | | |
| PF-180 | S. epidermidis, M. luteus, P. aeruginosa, | SGLGDLGFSSEAK | 460 |
| | C. albicans, MRSA, | | |
| | E. faecalis, C. jejuni, M. smegmatis | | |
| PF-181 | S. epidermidis, M. luteus, E. coli, | GIAPRRNEWGAVGGR | 461 |
| | MRSA, E. faecalis, C. jeikeium | | |
| PF-182 | S. epidermidis, M. luteus, E. coli, | LPATRDKTRVPASVAGAP | 462 |
| | E. faecalis, C. jeikeium | | |
| PF-183 | S. epidermidis, M. luteus, E. coli, | KPGISVENRQ | 463 |
| | C. albicans, MRSA, E. faecalis, | | |
| | C. jeikeium | | |
| PF-184 | S. epidermidis, M. luteus, E. coli, | LIADRHIRA | 464 |
| | P. aeruginosa, C. albicans, | | |
| | MRSA, C. jeikeium | | |
| PF-185 | E. coli, P. aeruginosa | RPAQARQGPGGLIADRHI | 465 |
| | | RA | |
| PF-186 | S. epidermidis, M. luteus, E. coli, | DADKNLSLERDRFAWRV | 466 |
| | P. aeruginosa, MRSA, C. jeikeium | AAP | |
| PF-187 | S. epidermidis, M. luteus, E. coli, | EIQKIAKGVSGQVYGPSR | 467 |
| | MRSA | QITISKKR | |
| PF-188 | S. epidermidis, M. luteus, E. coli, | ARTFAGRLGTRYFGGLMR | 468 |
| | C. albicans, MRSA, E. faecalis | STKA | |
| PF-189 | S. epidermidis, M. luteus, C. albicans, | GNLTRSREAARATQ | 469 |
| | MRSA, E. faecalis, C. jejuni | | |
| PF-190 | S. epidermidis, M. luteus, P. mirabilis, | HFILRKPLLFMIHSLKTGP | 470 |
| | E. coli, P. aeruginosa, | LDRF | |
| | C. albicans, MRSA, S. pneumoniae, | | |
| | E. faecalis, C. jeikeium | | |
| PF-191 | E. coli, P. aeruginosa, C. jejuni | QFCNFAWLFLASNNAQVS | 471 |
| | | ALA | |
| PF-192 | S. epidermidis, M. luteus, P. aeruginosa, | VEEDEAPPPHY | 472 |
| | C. albicans, E. faecalis, | | |
| | C. jeikeium | | |
| PF-193 | S. epidermidis, M. luteus, E. coli, | PPHCPPGHAKKGWC | 473 |
| | MRSA, E. faecalis, C. jejuni | | |
| PF-194 | C. jeikeium | MKGNKLATAHEQPVKNS | 474 |
| | | APPL | |
| PF-195 | S. epidermidis, M. luteus, E. faecalis, | EMAEGSADDRLRKTPRDC | 475 |
| | C. jeikeium | | |
| PF-196 | S. epidermidis, M. luteus, P. mirabilis, | TTARYIRRQCHTSITPLSQG | 476 |
| | E. coli, P. aeruginosa, | | |
| | C. albicans, MRSA, S. pneumoniae, | | |
| | E. faecalis, C. jejuni | | |
| PF-197 | S. epidermidis, M. luteus, C. albicans, | CNALLRRGHPPSAL | 477 |
| | E. faecalis, C. jejuni | | |
| PF-200 | S. epidermidis, M. luteus, MRSA, | GIELKSLIMAQIERWRQA | 478 |
| | E. faecalis, C. jeikeium | | |
| PF-201 | S. epidermidis, M. luteus, E. coli, | GCRPASLSDADPDGR | 479 |
| | C. albicans, E. faecalis, C. jeikeium, | | |
| | C. jejuni | | |
| PF-202 | S. epidermidis, M. luteus, E. coli, | ALNRASLRLALGE | 480 |
| | MRSA, E. faecalis, C. jeikeium, | | |
| | C. jejuni | | |
| PF-203 | S. epidermidis, M. luteus, P. mirabilis, | SWKCHHLAI | 481 |
| | E. coli, P. aeruginosa, | | |
| | C. albicans, MRSA, S. pneumoniae, | | |
| | E. faecalis, C. jejuni | | |
| PF-204 | S. epidermidis, P. mirabilis, E. coli, | ALQKQDMNLPSVKNQLV | 482 |
| | P. aeruginosa, C. albicans, | FLKSTG | |
| | C. jejuni | | |
| PF-205 | S. epidermidis, M. luteus, E. coli, | AGVLETPRCRGEYGAN | 483 |
| | P. aeruginosa, C. albicans, | | |
| | MRSA, E. faecalis, C. jeikeium, | | |
| | C. jejuni | | |
| PF-206 | M. luteus, C. albicans, C. jeikeium, | KLRSASKKSLQEKSCGIMP | 484 |
| | C. jejuni | EKPAG | |
| PF-207 | M. luteus, C. jeikeium | AAGCRDLGSLSSLVTNPS | 485 |
| PF-208 | S. epidermidis, C. albicans, | DAYHCHLVRSPDAHDLS | 486 |
| | MRSA, S. pneumoniae, E. faecalis, | MRIGFV | |
| | C. jeikeium, C. jejuni | | |
| PF-209 | C. albicans, C. jeikeium, C. jejuni | NYAVVSHT | 487 |
| PF-210 | S. epidermidis, P. aeruginosa, C. albicans, | EREDGCDAMPLP | 488 |
| | MRSA, E. faecalis, C. jeikeium, | | |
| | C. jejuni | | |
| PF-211 | S. epidermidis, M. luteus, E. coli, | DSFDSLSPFRERGGEREDG | 489 |
| | P. aeruginosa, C. albicans, S. pneumoniae, | CDAMPLP | |
| | E. faecalis, C. jeikeium, | | |
| | C. jejuni, M. smegmatis | | |
| PF-212 | M. luteus, P. aeruginosa | NDSKASN | 490 |
| PF-213 | S. epidermidis | MTTGVDFIIEKV | 491 |
| PF-214 | S. mutans, S. epidermidis, M. luteus, | GHLRVCWVFSASLLTPFR | 492 |
| | E. coli, P. aeruginosa, A. baumannii | SATLI | |
| PF-215 | S. epidermidis, M. luteus, P. aeruginosa, | ELKITNYNVNTVLYRYYK | 493 |
| | A. baumannii | WGNDLCE | |
| PF-216 | S. mutans, E. coli | ESVDKITEALEEDGFPAKVQ | 494 |
| PF-217 | S. mutans | DWEFTHKTIPQKK | 495 |
| PF-218 | S. epidermidis, M. luteus, P. aeruginosa, | SETPEKPVGTFFYSIYYKIIL | 496 |
| | A. baumannii | | |
| PF-219 | S. epidermidis, M. luteus, P. aeruginosa, | FLALAVIAGLFKVILIYAA | 497 |
| | A. baumannii | PYLK | |
| PF-221 | S. epidermidis, M. luteus, P. aeruginosa | VFDNIDINF | 498 |
| PF-222 | S. epidermidis | HIKETR | 499 |
| PF-223 | S. epidermidis, M. luteus, A. baumannii | VKFCIECQTKLERKRR | 500 |
| PF-224 | S. epidermidis, P. aeruginosa, A. baumannii | DYFYITLSQKNTF | 501 |
| PF-225 | S. epidermidis | MNCASPEFKKLMELYK | 502 |
| PF-226 | A. baumannii | LMFFSENMDKRDTLSGKF | 503 |
| | | RYFAGSKVIKLMNWLSEN | |
| | | GK | |
| PF-228 | S. mutans | NQLGSQAFAQL | 504 |
| PF-229 | S. epidermidis, M. luteus, P. aeruginosa, | DPILIQIGFTRFALRKAEAE | 505 |
| | A. baumannii | KIEIQVEEGVPA | |
| PF-230 | S. mutans | EDKPTNTIQEIKPVKWQ | 506 |
| PF-231 | S. mutans | AVRDFKKSVREEDEAASL | 507 |
| | | NSPRTIDAQVKTSESTSVKS | |
| PF-232 | S. epidermidis, M. luteus | FDQLYALEREGKLDELLA | 508 |
| PF-233 | S. epidermidis, M. luteus, P. aeruginosa, | DANAMARTTIAIVYILALI | 509 |
| | A. baumannii | ALTISYSL | |
| PF-234 | S. epidermidis, M. luteus | RTPYILRS | 510 |
| PF-235 | S. epidermidis, M. luteus | GIPFSKPHKRQVNYMKSD | 511 |
| | | VLAYIEQNKMAHTA | |
| PF-236 | S. mutans, S. epidermidis, E. coli, | KEIRTATVAELNAKRRLTS | 512 |
| | C. albicans, S. pneumoniae, E. faecalis | AEQALAEVS | |
| PF-237 | S. epidermidis | YVKPKVGVHE | 513 |
| PF-238 | S. mutans, S. epidermidis, E. coli, | RNAVVVTEATFPKYEEEIT | 514 |
| | P. aeruginosa, C. albicans, | NYLNRRFGEDWSLKLEKC | |
| | MRSA, S. pneumoniae, E. faecalis | SVA | |
| PF-239 | S. mutans, E. coli | PKHNVVTGVSVDLDYKP | 515 |
| PF-240 | S. mutans, E. coli | RITEVPPDEHSDR | 516 |
| PF-242 | S. mutans, E. coli | KLFEDPLIKSKAVENFQTT | 517 |
| | | WHEQCLAKELAKNM | |
| PF-244 | S. epidermidis, M. luteus, P. aeruginosa, | HMRTISYLLAFAKFSLFIPP | 518 |
| | A. baumannii | KQSLKRL | |
| PF-245 | S. epidermidis, M. luteus, P. aeruginosa, | MNDVKPVVQPKQTLKAF | 519 |
| | A. baumannii | LVQLLSVRAGVYIKQNNQ | |
| | | LPKTKG | |
| PF-246 | S. mutans | QPDEKAEFFDPSLDKVYR | 520 |
| | | HPTFYHIPDGIEHM | |
| PF-247 | S. epidermidis | ETAASETH | 521 |
| PF-248 | S. mutans, S. epidermidis, M. luteus, | ILSKLWFWMINSLGVVLL | 522 |
| | E. coli, P. aeruginosa, A. baumannii | VSYWLLAKWGVA | |
| PF-249 | S. epidermidis, M. luteus | INSRYKISF | 523 |
| PF-252 | S. mutans | MKKLVAALAVIVILTGCV | 524 |
| | | YDPVNYDKIHDQEFQDHL | |
| | | RQNG | |
| PF-253 | S. epidermidis, M. luteus | VRDDDS | 525 |
| PF-254 | S. epidermidis, M. luteus, P. aeruginosa, | FIYGVGFVPHFWLWKWLF | 526 |
| | A. baumannii | SPWIAWPLMLLGYYIWFLT | |
| PF-255 | P. aeruginosa | DHKINESQHNPFRSDSNK | 527 |
| | | QNVDFF | |
| PF-256 | S. epidermidis | EYFKQVYVKNEKIYSFWI | 528 |
| | | CKDLSPKEAAKRAEDILV | |
| | | KLK | |
| PF-257 | S. epidermidis | VWENRKKYLENEIERHNV | 529 |
| | | FLKLGQEVIKGLNALASR | |
| | | GR | |
| PF-259 | S. epidermidis, P. aeruginosa, A. baumannii | LPFSKIGRRVSYKKKDVL | 530 |
| | | KYEQSKTVLNTAQLATV | |
| PF-262 | S. mutans, S. epidermidis, M. luteus, | DPHSEIDVTRYCQLHHFTC | 531 |
| | E. coli, P. aeruginosa, A. baumannii | QTMQISEREFHYLIETQ | |
| PF-263 | S. epidermidis, M. luteus, A. baumannii | NLKKCPC | 532 |
| PF-265 | S. epidermidis, M. luteus, A. baumannii | MKTLFFPLFLIIFVLIIQAL | 533 |
| | | DQSYQKKIGISKPQKHPEF | |
| | | MQ | |
| PF-266 | S. mutans | DQEKKNKTEESTEQ | 534 |
| PF-267 | M. luteus | SDDKRTD | 535 |
| PF-268 | S. mutans | EVLLSDLRPDIFSET | 536 |
| PF-270 | S. epidermidis, M. luteus, P. aeruginosa | MYLTPYAWIAVGSIFAFS | 537 |
| | | VTTIKIGDQNDEKQKSHK | |
| | | NDVHKR | |
| PF-271 | S. epidermidis, M. luteus, P. aeruginosa, | AAQPQTTSP | 538 |
| | A. baumannii | | |
| PF-273 | S. epidermidis, M. luteus, P. aeruginosa, | LVGALLIFVALIYMVLKG | 539 |
| | A. baumannii | NADKN | |
| PF-275 | S. mutans | LVSGVANTVKNTAHTVG | 540 |
| | | NTAKHAGHVAADTTVKA | |
| | | TKKQQVK | |
| PF-276 | S. epidermidis | LDLALSTNSLNLEGFSF | 541 |
| PF-278 | M. luteus, A. baumannii | LSLATFAKIFMTRSNWSL | 542 |
| | | KRFNRL | |
| PF-279 | S. mutans, S. epidermidis, M. luteus, | SHIGFISISACLAVLLGIAR | 543 |
| | E. coli, P. aeruginosa, A. baumannii | LFVWTWVKFFA | |
| PF-281 | S. mutans, E. coli | SYNTYYNKLIHGQRTPDGM | 544 |
| PF-282 | S. mutans | QNNDTSAWCGSAHKNGNS | 545 |
| PF-283 | B. subtilis, B. fragilis, C. difficile | MIRIRSPTKKKLNRNSISD | 546 |
| | | WKSNTSGRFFY | |
| PF-284 | C. difficile | MRYITYSLIPRLLSKKVIH | 547 |
| | | QQ | |
| PF-285 | S. mutans | VPAKLLRVIDEIPE | 548 |
| PF-288 | S. mutans, E. coli | IYQLLNIEYSEDD | 549 |
| PF-289 | C. difficile | MGRHLWNPSYFVATVSE | 550 |
| | | NTEEQIRKYRKNK | |
| PF-291 | S. mutans, E. coli | DVDGAIESEL | 551 |
| PF-292 | S. epidermidis, B. subtilis, B. fragilis | SFVSTTVRLIFEESKRYKF | 552 |
| PF-294 | S. epidermidis, C. difficile | DFLVNFLWFKGELNWGK | 553 |
| | | KRYK | |
| PF-295 | C. difficile | NIQVYESECGNYIFKKSDE | 554 |
| | | SFLIDIFDKNGTH | |
| PF-297 | S. epidermidis, B. subtilis, B. fragilis | ISKGIDDIVYVINKILSIGNI | 555 |
| | | FKIIKRK | |
| PF-299 | B. subtilis | LATKLKYEKEHKKM | 556 |
| PF-300 | B. subtilis, C. difficile | VKDVLLELFNKIIGA | 557 |
| PF-301 | C. difficile | GIVLIGLKLIPLLANVLN | 558 |
| PF-304 | S. mutans | LVKDTSDIKNDLNNIEIVT | 559 |
| | | SKNSNDIAKLKSVK | |
| PF-305 | C. difficile | MREWICPSCNETHDRDIN | 560 |
| | | ASINILKEGLRLITIQNK | |
| PF-306 | C. difficile | GCILPHKKDNYNYIMSKF | 561 |
| | | QDLVKITSKK | |
| PF-307 | S. epidermidis, B. subtilis, B. fragilis, | MKRRRCNWCGKLFYLEE | 562 |
| | C. difficile | KSKEAYCCKECRKKAKK | |
| | | VKK | |
| PF-308 | C. difficile | QQYLILDRM | 563 |
| PF-309 | S. mutans, E. coli | GIPGMTAAPAEENEQEEN | 564 |
| | | ADEE | |
| PF-311 | C. difficile | IDAVTKKKTTCMIRAPTKI | 565 |
| | | PIAHTDN | |
| PF-313 | S. epidermidis, C. difficile | YITSHKNARAIIKKFERDEI | 566 |
| | | LEEVITHYLNRK | |
| PF-314 | S. mutans | ECLKKAIKSKALNKAFKID | 567 |
| | | VPDEVYDNLLMELEEYEK | |
| PF-317 | S. mutans | LILVSDI | 568 |
| PF-319 | S. epidermidis, B. subtilis, C. difficile | SIGSMIGMYSFRHKTKHIK | 569 |
| | | FTFGIPFILFLQFLLVYFYILK | |
| PF-320 | S. mutans, E. coli | DSGYYALLENKEERVVW | 570 |
| | | DGEVVANNIFNNLWIVVN | |
| | | KVKTG | |
| PF-323 | S. mutans | ARESIEKSHVPVDATIVGV | 571 |
| | | VDSFEVFDE | |
| PF-324 | C. difficile | HFSLL | 572 |
| PF-325 | S. mutans, E. coli | LTIDEKLRNHR | 573 |
| PF-326 | S. mutans, E. coli | VIVGNLGAQKEKRNDTPIS | 574 |
| | | AKKDIMGDKTVRVRADL | |
| | | HH | |
| PF-328 | S. mutans | NGNEKAFSEVENLVK | 575 |
| PF-329 | S. epidermidis | IGILFDKSVRKY | 576 |
| PF-333 | S. mutans | YMTKKLVEMAEQQMAG | 577 |
| | | KSNR | |
| PF-334 | S. epidermidis, C. difficile | QQYLILDRM | 578 |
| PF-336 | S. mutans, E. coli | MLTSRKKRLKKIVEEQNK | 579 |
| | | KDESI | |
| PF-337 | S. epidermidis | YMTKKLVEMAERQMAGK | 580 |
| PF-338 | S. mutans | KGTSCPDQLSKAIRQSI | 581 |
| PF-340 | S. mutans, E. coli | VKDVLLELFNKIIGA | 582 |
| PF-344 | B. subtilis, C. jejuni | DERLPEAKAIRNFNGSVM | 583 |
| | | VLGR | |
| PF-347 | S. epidermidis, B. subtilis, B. fragilis, | GIFTGVTVVVSLKHC | 584 |
| | E. coli, P. aeruginosa, C. albicans, | | |
| | MRSA, S. pneumoniae, | | |
| | E. faecalis | | |
| PF-348 | B. subtilis, E. coli, P. aeruginosa, | ESASAAEWYNPNMNVKK | 585 |
| | C. albicans, E. faecalis, C. jejuni | AICMG | |
| PF-349 | S. epidermidis, B. subtilis, B. fragilis, | MPKSCHVPVLCDFFFLVII | 586 |
| | E. coli, P. aeruginosa, C. albicans, | KFLALFKTIQS | |
| | MRSA, S. pneumoniae, | | |
| | E. faecalis, C. jeikeium | | |
| PF-350 | S. epidermidis, E. coli, E. faecalis, | LAVILRAIVY | 587 |
| | C. jeikeium, C. jejuni | | |
| PF-351 | S. mutans | YLFFKGKKVAEEEATKDE | 588 |
| | | VKR | |
| PF-352 | C. jeikeium | RVKKIG | 589 |
| PF-353 | S. epidermidis, M. luteus, B. subtilis, | EKTNFKGVKRNFYKKASF | 590 |
| | E. coli, C. albicans, S. pneumoniae, | FV | |
| | E. faecalis, C. jeikeium, | | |
| | C. jejuni | | |
| PF-354 | S. epidermidis, B. subtilis, E. coli, | FTFSKCRASNGRGFGTLWL | 591 |
| | P. aeruginosa, C. albicans, | | |
| | MRSA, S. pneumoniae, E. faecalis, | | |
| | C. jeikeium, C. jejuni | | |
| PF-355 | S. epidermidis, B. subtilis, B. fragilis, | WIAIGLLLYFSLKNQ | 592 |
| | E. coli, P. aeruginosa, C. albicans, | | |
| | MRSA, S. pneumoniae, | | |
| | E. faecalis, C. jeikeium | | |
| PF-356 | S. epidermidis, B. subtilis, B. fragilis, | VSIKIGAIVIGMIGLMELLTE | 593 |
| | E. coli, P. aeruginosa, C. albicans, | | |
| | MRSA, S. pneumoniae, | | |
| | E. faecalis, C. jeikeium | | |
| PF-357 | S. epidermidis, M. luteus, P. mirabilis, | MLTIIIGFIFWTMTLMLGY | 594 |
| | E. coli, P. aeruginosa, | LIGEREGRKHE | |
| | C. albicans, MRSA, S. pneumoniae, | | |
| | E. faecalis, C. jeikeium | | |
| PF-358 | S. epidermidis, B. subtilis, E. coli, | RNTAHNIKWRSKN | 595 |
| | C. albicans, MRSA, S. pneumoniae, | | |
| | E. faecalis, C. jeikeium, | | |
| | C. jejuni | | |
| PF-359 | S. epidermidis, B. fragilis, P. aeruginosa, | MTVMEDPGSEQRNKIQSP | 596 |
| | C. albicans, MRSA, | MKGEDFSALFGR | |
| | E. faecalis, C. jeikeium | | |
| PF-360 | S. epidermidis, B. subtilis, E. coli, | MEQKVKVIFVPRSKPDNQ | 597 |
| | P. aeruginosa, C. albicans, E. faecalis, | LKTFVSAVLFKA | |
| | C. jeikeium, C. jejuni | | |
| PF-361 | S. epidermidis, E. coli, E. faecalis, | NQVTEGIRLLVE | 598 |
| | C. jejuni | | |
| PF-362 | S. epidermidis, E. coli, P. aeruginosa, | NIERILKEKVWMIRCVE | 599 |
| | C. albicans, E. faecalis, | | |
| | C. jejuni | | |
| PF-363 | B. subtilis, E. coli, P. aeruginosa, | SMLSVTVMCLMHASVAA | 600 |
| | C. albicans, S. pneumoniae, E. faecalis | NQAMEKKV | |
| PF-364 | S. epidermidis, B. fragilis, P. aeruginosa, | LVNGIKI | 601 |
| | C. jeikeium, C. jejuni | | |
| PF-365 | S. epidermidis, B. subtilis, B. fragilis, | LYKQKIQLEEELEKLKDD | 602 |
| | P. aeruginosa, C. albicans | RQ | |
| PF-366 | S. epidermidis, M. luteus, B. fragilis, | ALCSVIKAIELGIINVHLQ | 603 |
| | P. mirabilis, E. coli, P. aeruginosa, | | |
| | C. albicans, MRSA, | | |
| | S. pneumoniae, E. faecalis, C. jeikeium, | | |
| | C. jejuni | | |
| PF-367 | B. subtilis | TKTPGTFTPGTGIQKTAVPL | 604 |
| PF-368 | C. jeikeium, C. jejuni | MLKQTA | 605 |
| PF-369 | B. subtilis, B. fragilis, E. coli, P. aeruginosa, | MSEAVNLLRGARYSQRY | 606 |
| | C. albicans, S. pneumoniae, | AKNQVPYEVIIEK | |
| | C. jeikeium, C. jejuni | | |
| PF-370 | S. epidermidis, E. coli, P. aeruginosa, | VIFLHKESGNLKEIFY | 607 |
| | E. faecalis, C. jejuni | | |
| PF-371 | S. epidermidis, B. fragilis, C. jejuni | TFIYNEF | 608 |
| PF-372 | C. jeikeium, C. jejuni | KKQDKRIEDKYKRMKKGD | 609 |
| PF-373 | S. epidermidis, E. coli, P. aeruginosa, | HFYLLFER | 610 |
| | C. albicans, MRSA, | | |
| | E. faecalis, C. jejuni | | |
| PF-374 | S. epidermidis, B. subtilis, B. fragilis, | HLFFVKGMFILCQKNQIN | 611 |
| | E. coli, P. aeruginosa, C. albicans, | DE | |
| | MRSA, S. pneumoniae, | | |
| | E. faecalis, C. jeikeium, C. jejuni | | |
| PF-375 | S. epidermidis, B. subtilis, B. fragilis, | MDSAKAQTMRTDWLAVS | 612 |
| | E. coli, P. aeruginosa, C. albicans, | CLVASAYLRSMLA | |
| | S. pneumoniae, E. faecalis, | | |
| | C. jeikeium, C. jejuni | | |
| PF-376 | S. epidermidis, B. subtilis, B. fragilis, | MTVFEALMLAIAFATLIV | 613 |
| | E. coli, P. aeruginosa, C. albicans, | KISNKNDKK | |
| | MRSA, S. pneumoniae, | | |
| | E. faecalis, C. jeikeium, C. jejuni | | |
| PF-378 | B. subtilis, B. fragilis, E. coli, P. aeruginosa, | ESAKSNLNFLMQEEWALF | 614 |
| | C. jeikeium | LLL | |
| PF-379 | S. epidermidis, B. subtilis, B. fragilis, | VFVVLFIIYLASKLLTKLFP | 615 |
| | E. coli, P. aeruginosa, C. albicans, | IKK | |
| | MRSA, S. pneumoniae, | | |
| | E. faecalis, C. jeikeium, C. jejuni | | |
| PF-380 | S. epidermidis, B. subtilis, B. fragilis, | KKIIPLITLFVVTLVG | 616 |
| | E. coli, P. aeruginosa, C. albicans, | | |
| | MRSA, S. pneumoniae, | | |
| | E. faecalis, C. jeikeium, C. jejuni | | |
| PF-381 | E. coli, P. aeruginosa, C. jejuni | QGANPCQQVGFTVNDPD | 617 |
| | | CRLAKTV | |
| PF-382 | S. epidermidis, B. subtilis, B. fragilis, | KYKCSWCKRVYTLRKDH | 618 |
| | E. coli, P. aeruginosa, E. faecalis, | KTAR | |
| | C. jeikeium, C. jejuni | | |
| PF-383 | S. epidermidis, B. subtilis, B. fragilis, | WSEIEINTKQSN | 619 |
| | E. coli, C. jejuni | | |
| PF-384 | E. faecalis, C. jeikeium, C. jejuni | HISKERFEAY | 620 |
| PF-385 | S. epidermidis, E. coli, P. aeruginosa, | MIKKSILKIKYYVPVLISLT | 621 |
| | C. albicans, E. faecalis | LILSA | |
| PF-386 | S. epidermidis, B. subtilis, B. fragilis, | FTLTLITTIVAILNYKDKKK | 622 |
| | E. coli, P. aeruginosa, C. albicans, | | |
| | S. pneumoniae, E. faecalis, | | |
| | C. jeikeium, C. jejuni | | |
| PF-387 | B. subtilis, E. coli, P. aeruginosa, | GAVGIAFFAGNMKQDKRI | 623 |
| | E. faecalis, C. jeikeium, C. jejuni | ADRQNKKSEKK | |
| PF-388 | E. faecalis, C. jeikeium, C. jejuni | ITPLLDEIGKVCIDKISK | 624 |
| PF-389 | S. epidermidis, C. albicans, | GLQFKEIAEEFHITTTALQ | 625 |
| | MRSA, S. pneumoniae, E. faecalis, | QWHKDNGYPIYNKNNRK | |
| | C. jeikeium | | |
| PF-390 | S. epidermidis, P. aeruginosa, C. albicans, | VVAYVITQVGAIRF | 626 |
| | MRSA | | |
| PF-392 | S. epidermidis, B. subtilis, S. pneumoniae, | DPAGCNDIVRKYCK | 627 |
| | C. jeikeium, C. jejuni | | |
| PF-393 | S. epidermidis, E. coli, C. albicans, | DLVQSILSEFKKSG | 628 |
| | MRSA, S. pneumoniae, | | |
| | C. jejuni | | |
| PF-394 | S. epidermidis, MRSA, C. jejuni | VLKEECYQKN | 629 |
| PF-395 | S. epidermidis, E. coli, P. aeruginosa, | YCVPLGNMGNMNNKIW | 630 |
| | S. pneumoniae, E. faecalis, | | |
| | C. jeikeium, C. jejuni | | |
| PF-396 | S. epidermidis, E. coli, P. aeruginosa, | LIYTILASLGVLTVLQAILG | 631 |
| | C. albicans, E. faecalis, | REPKAVKA | |
| | C. jeikeium | | |
| PF-397 | S. epidermidis, MRSA, S. pneumoniae, | VEDLMEDLNA | 632 |
| | E. faecalis, C. jejuni | | |
| PF-398 | S. epidermidis, B. subtilis, B. fragilis, | ILVVLAGILLVVLSYVGIS | 633 |
| | E. coli, P. aeruginosa, C. albicans, | KFKMNC | |
| | MRSA, S. pneumoniae, | | |
| | E. faecalis, C. jeikeium, C. jejuni | | |
| PF-399 | S. epidermidis, M. luteus, P. mirabilis, | FPIISALLGAIICIAIYSFIVN | 634 |
| | E. coli, P. aeruginosa, | RKA | |
| | C. albicans, MRSA, S. pneumoniae, | | |
| | E. faecalis, C. jejuni | | |
| PF-400 | S. epidermidis, E. coli, S. pneumoniae, | VIAWKFRNKFENSGV | 635 |
| | E. faecalis, C. jeikeium | | |
| PF-401 | S. epidermidis, E. coli, P. aeruginosa, | YWLSRVTTGHSFAFEKPV | 636 |
| | MRSA, E. faecalis, C. jejuni | PLSLTIK | |
| PF-402 | S. epidermidis, P. aeruginosa, E. faecalis, | FIDVLKSKINEFLN | 637 |
| | C. jejuni | | |
| PF-403 | E. coli, P. aeruginosa, S. pneumoniae, | LLSTEQLLKYYDGETFDG | 638 |
| | E. faecalis, C. jeikeium, | FQLPSNE | |
| | C. jejuni | | |
| PF-404 | S. epidermidis, E. coli, P. aeruginosa, | VLYFQATVV | 639 |
| | E. faecalis, C. jeikeium, | | |
| | C. jejuni | | |
| PF-405 | S. epidermidis, E. coli, E. faecalis | LVRIEVDDLEEWYERNFI | 640 |
| PF-406 | E. coli, C. jejuni | YLEMNADYLSNMDIFDEL | 641 |
| | | WEKYLENNK | |
| PF-407 | S. epidermidis, B. subtilis, E. coli, | KPKNKKEKTVISYEKLLS | 642 |
| | P. aeruginosa, MRSA, S. pneumoniae, | MY | |
| | E. faecalis, C. jeikeium, | | |
| | C. jejuni | | |
| PF-408 | S. epidermidis, E. coli, P. aeruginosa, | YCVPLGNMGNMNNKIW | 643 |
| | MRSA, E. faecalis, C. jeikeium, | | |
| | C. jejuni | | |
| PF-409 | S. epidermidis, MRSA, C. jeikeium, | DLVQSILSEFKKSG | 644 |
| | C. jejuni | | |
| PF-410 | S. epidermidis, M. luteus, B. fragilis, | FALELIALCRNLFIVYFP | 645 |
| | P. mirabilis, E. coli, P. aeruginosa, | | |
| | C. albicans, MRSA, | | |
| | S. pneumoniae, E. faecalis | | |
| PF-411 | M. luteus, B. subtilis, B. fragilis, | WVAVAILLNIALQTQLT | 646 |
| | P. mirabilis, P. aeruginosa, C. albicans, | | |
| | MRSA, S. pneumoniae, | | |
| | E. faecalis, C. jeikeium, C. jejuni | | |
| PF-412 | M. luteus, E. coli, C. albicans, C. jeikeium, | TSGWLGQLEQ | 647 |
| | C. jejuni | | |
| PF-413 | P. aeruginosa, C. albicans, C. jejuni | TFAGSIKIGVPDLVHVTFN | 648 |
| | | CKR | |
| PF-414 | E. coli, C. albicans, C. jeikeium | LLNKKLE | 649 |
| PF-416 | S. pneumoniae, C. jeikeium | SKAGLYGKIERSDKRE | 650 |
| PF-417 | S. epidermidis, C. jeikeium, C. jejuni | DSYFRS | 651 |
| PF-418 | S. epidermidis, M. luteus, P. mirabilis, | FFLVHFYIRKRKGKVSIFL | 652 |
| | E. coli, P. aeruginosa, | NYF | |
| | C. albicans, MRSA, S. pneumoniae, | | |
| | E. faecalis, C. jeikeium, | | |
| | C. jejuni | | |
| PF-421 | C. jeikeium | KHCFEITDKTDVV | 653 |
| PF-422 | C. albicans, MRSA, C. jeikeium | MSRKKYENDEKSQKKLKI | 654 |
| | | GRKSDVFYGIID | |
| PF-423 | S. epidermidis, M. luteus, E. coli, | AGKKERLLSFREQFLNKN | 655 |
| | S. pneumoniae, E. faecalis, C. jeikeium | KKK | |
| PF-424 | S. epidermidis, C. albicans, | IAAFVTSRAFSDTVSPI | 656 |
| | MRSA | | |
| PF-425 | S. epidermidis, M. luteus, E. coli, | MMELVLKTIIGPIVVGVVL | 657 |
| | P. aeruginosa, C. albicans, | RIVDKWLNKDK | |
| | MRSA, S. pneumoniae, C. jeikeium | | |
| PF-426 | S. epidermidis, E. coli, P. aeruginosa, | MLQKYTQMISVTKCIITKN | 658 |
| | C. albicans, MRSA, | KKTQENVDAYN | |
| | S. pneumoniae, E. faecalis, C. jeikeium | | |
| PF-427 | M. luteus, P. aeruginosa, C. albicans, | YVLEYHGLRATQDVDAF | 659 |
| | C. jejuni | MAL | |
| PF-428 | S. epidermidis, C. albicans, E. faecalis, | ENEESIF | 660 |
| | C. jeikeium | | |
| PF-429 | S. epidermidis, S. pneumoniae, C. jeikeium | AATLICVGSGIMSSL | 661 |
| PF-430 | S. epidermidis, M. luteus, E. coli, | AVVCGYLAYTATS | 662 |
| | S. pneumoniae, E. faecalis, C. jeikeium, | | |
| | C. jejuni | | |
| PF-431 | S. epidermidis, M. luteus, E. coli, | VAYAAICWW | 663 |
| | P. aeruginosa, MRSA, S. pneumoniae, | | |
| | E. faecalis, C. jeikeium, | | |
| | C. jejuni | | |
| PF-432 | S. epidermidis, M. luteus, E. coli, | FNGDSEFFLCIAF | 664 |
| | P. aeruginosa, C. albicans, | | |
| | MRSA, S. pneumoniae, E. faecalis, | | |
| | C. jeikeium, C. jejuni | | |
| PF-433 | S. epidermidis, E. coli, S. pneumoniae, | MRKEFHNVLSSGQLLADK | 665 |
| | C. jeikeium | RPARDYNRK | |
| PF-434 | S. epidermidis, M. luteus, S. pneumoniae, | GQLLADKRPARDYNRK | 666 |
| | C. jeikeium | | |
| PF-435 | C. jeikeium | MSRWDGHSDKGEAPAGK | 667 |
| | | PPMHGFGLNGENK | |
| PF-436 | C. jeikeium | KKHVLVGKQEKNG | 668 |
| PF-438 | S. epidermidis, E. coli, S. pneumoniae, | QPYFQNQFKKITGYTPLQ | 669 |
| | C. jeikeium, C. jejuni | YRKEKR | |
| PF-439 | S. epidermidis, M. luteus, B. fragilis, | RVLVLKKFHGIMDGNRN | 670 |
| | P. mirabilis, E. coli, P. aeruginosa, | VAVFFVGQ | |
| | C. albicans, MRSA, | | |
| | S. pneumoniae, E. faecalis, C. jeikeium, | | |
| | C. jejuni | | |
| PF-440 | S. epidermidis, M. luteus, P. mirabilis, | MFIISPDLFNIAVILYILFFI | 671 |
| | E. coli, P. aeruginosa, | HDILLLILS | |
| | C. albicans, MRSA, S. pneumoniae, | | |
| | E. faecalis, C. jeikeium, | | |
| | C. jejuni | | |
| PF-441 | C. jeikeium | TQVHKMARGIDPGPANGI | 672 |
| | | YR | |
| PF-442 | S. epidermidis, E. coli, C. albicans, | MQIFYIKTKIFLSFFLFLLIF | 673 |
| | S. pneumoniae, E. faecalis | SQCFYKIEE | |
| PF-443 | S. epidermidis, M. luteus, P. mirabilis, | KLLYFFNYFENLQQVHLL | 674 |
| | E. coli, P. aeruginosa, | VQL | |
| | C. albicans, S. pneumoniae, E. faecalis, | | |
| | C. jeikeium, C. jejuni | | |
| PF-444 | M. luteus, C. albicans, S. pneumoniae, | MAAKLWEEGKMVYASSA | 675 |
| | C. jeikeium | SMTKRLKLAMSKV | |
| PF-445 | M. luteus, S. pneumoniae, C. jeikeium | ASMTKRLKLAMSKV | 676 |
| PF-446 | M. luteus, C. jeikeium | SGNEKV | 677 |
| PF-447 | S. epidermidis, M. luteus, E. coli, | IDKSRNKDQFSHIFGLYNI | 678 |
| | S. pneumoniae | CSG | |
| PF-448 | S. epidermidis, M. luteus, P. mirabilis, | SLQSQLGPCLHDQRH | 679 |
| | E. coli, S. pneumoniae, | | |
| | E. faecalis, C. jeikeium, C. jejuni | | |
| PF-450 | S. epidermidis, M. luteus, E. coli, | HRNLIILQRTIFI | 680 |
| | P. aeruginosa, C. albicans, | | |
| | MRSA, S. pneumoniae, E. faecalis, | | |
| | C. jeikeium, C. jejuni | | |
| PF-451 | S. epidermidis, M. luteus, E. coli, | MVNYIIGSYMLYREQNNN | 681 |
| | P. aeruginosa, C. albicans, | EALRKFDITLAM | |
| | MRSA, S. pneumoniae, E. faecalis, | | |
| | C. jeikeium, C. jejuni, M. smegmatis | | |
| PF-452 | M. luteus, P. aeruginosa, C. albicans, | MNNWIKVAQISVTVINEVI | 682 |
| | S. pneumoniae, E. faecalis, | DIMKEKQNGGK | |
| | C. jeikeium, M. smegmatis | | |
| PF-453 | M. luteus, E. coli, P. aeruginosa, | IIQDIAHAFGY | 683 |
| | S. pneumoniae, E. faecalis, C. jeikeium, | | |
| | C. jejuni | | |
| PF-454 | S. epidermidis, M. luteus, E. coli, | MSVFVPVTNIFMFIMSPIF | 684 |
| | P. aeruginosa, C. albicans, | NVNLLHFKVYI | |
| | MRSA, S. pneumoniae, E. faecalis, | | |
| | C. jeikeium, C. jejuni, M. smegmatis | | |
| PF-456 | C. albicans, MRSA, E. faecalis, | TCVKPRTIN | 685 |
| | C. jeikeium, C. jejuni | | |
| PF-457 | C. albicans, S. pneumoniae, E. faecalis, | INKYHHIA | 686 |
| | C. jeikeium | | |
| PF-458 | S. epidermidis, M. luteus, E. coli, | ISLIIFIMLFVVALFKCITNY | 687 |
| | P. aeruginosa, C. albicans, | KHQS | |
| | MRSA, S. pneumoniae, E. faecalis, | | |
| | C. jeikeium, C. jejuni | | |
| PF-459 | P. aeruginosa | EKRMSFNENQSHRPLL | 688 |
| PF-460 | S. epidermidis, M. luteus, P. mirabilis, | MEHVLPFQNTPPNIVIIYK | 689 |
| | E. coli, P. aeruginosa, | DFTHLKSITFS | |
| | C. albicans, MRSA, S. pneumoniae, | | |
| | E. faecalis, C. jeikeium, | | |
| | C. jejuni, M. smegmatis | | |
| PF-461 | E. coli, S. pneumoniae | MTLAIKNCSVTKCLGFGD | 690 |
| | | FVNDDSDSYFDA | |
| PF-462 | E. faecalis, C. jeikeium | KNKTDTL | 691 |
| PF-463 | S. epidermidis, E. coli, P. aeruginosa, | MVILVFSLIFIFTDNYLVY | 692 |
| | C. albicans, S. pneumoniae, | QSKSIKEDVMI | |
| | E. faecalis, M. smegmatis | | |
| PF-464 | S. epidermidis, C. albicans, | VDMVNRFLGN | 693 |
| | MRSA, S. pneumoniae, E. faecalis, | | |
| | C. jeikeium, C. jejuni | | |
| PF-465 | S. epidermidis, M. luteus, E. coli, | KPVGKALEEIADGKIEPVV | 694 |
| | P. aeruginosa, C. albicans, | PKEYLG | |
| | MRSA, S. pneumoniae, E. faecalis, | | |
| | C. jeikeium, C. jejuni | | |
| PF-466 | MRSA, C. jeikeium, C. jejuni | VRKSDQ | 695 |
| PF-467 | MRSA, E. faecalis, C. jeikeium, | YYKDYFKEI | 696 |
| | C. jejuni | | |
| PF-469 | S. epidermidis, M. luteus, P. mirabilis, | YKVNYNNIDNHFNTLRH | 697 |
| | E. coli, C. albicans, | | |
| | MRSA, S. pneumoniae, E. faecalis, | | |
| | C. jeikeium, C. jejuni | | |
| PF-470 | M. luteus, E. coli, MRSA, E. faecalis, | PYSDSYATRPHWEQHRAR | 698 |
| | C. jeikeium, C. jejuni | | |
| PF-471 | S. epidermidis, M. luteus, E. coli, | MVGKIRGVTPRNDLLNAN | 699 |
| | P. aeruginosa, C. albicans, | ITGQLNLNYRLI | |
| | MRSA, S. pneumoniae, E. faecalis, | | |
| | C. jeikeium, C. jejuni | | |
| PF-472 | S. epidermidis, E. coli, P. aeruginosa, | MHISHLLDEVEQTEREKA | 700 |
| | C. albicans, MRSA, | VNVLENMNGNVI | |
| | S. pneumoniae, E. faecalis, C. jeikeium | | |
| PF-473 | S. epidermidis, E. coli, P. aeruginosa, | MAADIISTIGDLVKWIIDT | 701 |
| | C. albicans, MRSA, | VNKFKK | |
| | S. pneumoniae, E. faecalis | | |
| PF-474 | S. epidermidis, M. luteus, P. mirabilis, | MHRNLVLVKMEPIPHIMII | 702 |
| | E. coli, P. aeruginosa, | ANQIGIIIEKA | |
| | C. albicans, MRSA, S. pneumoniae, | | |
| | E. faecalis, C. jeikeium, | | |
| | C. jejuni, M. smegmatis | | |
| PF-475 | S. epidermidis, M. luteus, P. mirabilis, | MREKVRFTQAFKLFWTN | 703 |
| | E. coli, P. aeruginosa, | YFNFKGRSRRSEY | |
| | C. albicans, MRSA, S. pneumoniae, | | |
| | E. faecalis, C. jeikeium, | | |
| | C. jejuni | | |
| PF-476 | M. luteus, P. mirabilis, C. albicans, | WADAQYKLCENCSE | 704 |
| | S. pneumoniae, E. faecalis, | | |
| | C. jeikeium, C. jejuni | | |
| PF-477 | S. epidermidis, M. luteus, C. albicans, | HKNKLNIPHIKS | 705 |
| | S. pneumoniae, C. jeikeium, | | |
| | C. jejuni | | |
| PF-478 | S. epidermidis, M. luteus, P. mirabilis, | HLFILKSHLKPFPPFRYTYD | 706 |
| | E. coli, C. albicans, S. pneumoniae, | | |
| | E. faecalis, C. jeikeium, | | |
| | C. jejuni | | |
| PF-479 | S. epidermidis, M. luteus, P. mirabilis, | AYILKRREEKNK | 707 |
| | E. coli, C. albicans, S. pneumoniae, | | |
| | E. faecalis, C. jeikeium, | | |
| | C. jejuni | | |
| PF-480 | S. epidermidis, M. luteus, P. mirabilis, | MVEILVNTAISVYIVALYT | 708 |
| | E. coli, P. aeruginosa, | QWLSTRDNLKA | |
| | C. albicans, MRSA, S. pneumoniae, | | |
| | E. faecalis, C. jeikeium, | | |
| | C. jejuni, M. smegmatis | | |
| PF-481 | C. jeikeium | DELYEIMDKVIEEFNKDIE | 709 |
| | | QNNNNGNNEDLTENKIN | |
| PF-482 | S. epidermidis, M. luteus, P. mirabilis, | LVGYVRTSGTVRSYKIN | 710 |
| | E. coli, P. aeruginosa, | | |
| | C. albicans, MRSA, S. pneumoniae, | | |
| | E. faecalis, C. jeikeium, | | |
| | C. jejuni | | |
| PF-483 | P. mirabilis, C. jeikeium, C. jejuni | EDNKDKKDKKDK | 711 |
| PF-484 | S. epidermidis, M. luteus, P. mirabilis, | HKKDIRKQVFKN | 712 |
| | E. coli, E. faecalis, C. jeikeium, | | |
| | C. jejuni | | |
| PF-486 | S. mutans | MQKEGEEDY | 713 |
| PF-487 | S. mutans, E. coli | MYKAIAVLAMTIMAFFIF | 714 |
| | | VYPFFIVGLILG | |
| PF-488 | S. mutans, E. coli | YPNEQGHHKNNLKNIIIE | 715 |
| PF-489 | S. mutans | KVDRVSTTITEKIK | 716 |
| PF-490 | S. mutans, E. coli | RLILVSGNATVQK | 717 |
| PF-491 | S. mutans, E. coli | IHQYSSKPDIVGQEAKTVQ | 718 |
| | | QINS | |
| PF-492 | S. mutans, B. subtilis, E. coli | IQIDAASFYSISKSTIK | 719 |
| PF-493 | S. mutans, B. subtilis, E. coli | PGAFFFCRGRGCWCGIGW | 720 |
| PF-494 | S. mutans | FTEPLRPLQAKGQIISIKPS | 721 |
| | | TSSS | |
| PF-495 | S. mutans, E. coli | KGIYKKRTY | 722 |
| PF-496 | S. mutans, E. coli | EVTKRLVALAQQQLRG | 723 |
| PF-497 | S. mutans, B. subtilis, E. coli | LVLRICTDLFTFIKWTIKQ | 724 |
| | | RKS | |
| PF-498 | S. mutans, E. coli | MSEEEEVSEKVYNYLRRN | 725 |
| | | EFFEVRKEEFSA | |
| PF-499 | S. mutans, E. coli | VYSFLYVLVIVRKLLSMK | 726 |
| | | KRIERL | |
| PF-500 | S. mutans, E. coli | MGIFKEEKIKFIDCKGEEVI | 727 |
| | | LKIKIKDIKK | |
| PF-501 | S. mutans | GSTAHKSPIGSTNNQWGM | 728 |
| | | KKTPTD | |
| PF-502 | S. mutans | NKGKQMQDQTGKQPIVD | 729 |
| | | NG | |
| PF-503 | S. mutans | VVTLKDIVAVIEDQGYDVQ | 730 |
| PF-504 | S. mutans, E. coli | ILSVELSTKTSASGS | 731 |
| PF-505 | S. mutans | GYTKDPGTGI | 732 |
| PF-506 | S. mutans, E. coli | SGRGFALIVVLFILLIIVGA | 733 |
| | | ACIR | |
| PF-507 | S. mutans, E. coli | LALSIANLFKKKA | 734 |
| PF-508 | S. mutans | VSTFGKVVKVVDEK | 735 |
| PF-509 | S. mutans, B. subtilis, E. coli | EAKVQAKGEQIACNNY | 736 |
| PF-510 | S. mutans, E. coli | WYLYKKQSNQNDRGIPK | 737 |
| PF-511 | E. coli, P. aeruginosa, S. pneumoniae, | VMQSLYVKPPLILVTKLA | 738 |
| | C. jeikeium | QQN | |
| PF-512 | S. pneumoniae, C. jeikeium | SFMPEIQKNTIPTQMK | 739 |
| PF-513 | C. albicans | SNGVGLGVGIGSGIRF- | 740 |
| | | NH2 | |
| PF-514 | S. epidermidis, E. coli, P. aeruginosa, | QRFYKLFYHIDLTNEQAL | 741 |
| | C. albicans, MRSA, | KLFQVK | |
| | S. pneumoniae, E. faecalis, C. jeikeium | | |
| PF-515 | S. epidermidis, C. albicans, S. pneumoniae, | DKSTQDKDIKQAKLLAQE | 742 |
| | C. jeikeium | LGL-NH2 | |
| PF-517 | C. jejuni | VKPTMTASLISTVC | 743 |
| PF-518 | S. epidermidis, E. coli, P. aeruginosa, | SFYSKYSRYIDNLAGAIFL | 744 |
| | C. albicans, MRSA, | FF | |
| | S. pneumoniae, E. faecalis | | |
| PF-519 | M. luteus, E. faecalis, C. jeikeium | YLVYSGVLATAAAF-NH2 | 745 |
| PF-520 | S. epidermidis, M. luteus, E. coli, | LGLTAGVAYAAQPTNQPT | 746 |
| | C. albicans, MRSA, S. pneumoniae, | NQPTNQPTNQPTNQPTNQ | |
| | E. faecalis, C. jeikeium, | PRW-NH2 | |
| | C. jejuni | | |
| PF-521 | S. epidermidis, E. coli, P. aeruginosa, | CGKLLEQKNFFLKTR | 747 |
| | S. pneumoniae, E. faecalis | | |
| PF-522 | S. epidermidis, E. coli, P. aeruginosa, | FELVDWLETNLGKILKSK | 748 |
| | S. pneumoniae, E. faecalis | SA-NH2 | |
| PF-523 | S. epidermidis, M. luteus, C. albicans, | ASKQASKQASKQASKQAS | 749 |
| | S. pneumoniae, C. jeikeium, | KQASRSLKNHLL | |
| | C. jejuni | | |
| PF-524 | S. epidermidis, E. coli, P. aeruginosa, | PDAPRTCYHKPILAALSRI | 750 |
| | C. albicans, MRSA, | VVTDR | |
| | S. pneumoniae, E. faecalis, C. jeikeium | | |
| PF-526 | S. epidermidis, E. coli, P. aeruginosa, | VLLLFIFQPFQKQLL-NH2 | 751 |
| | C. albicans, MRSA, | | |
| | S. pneumoniae, E. faecalis, C. jeikeium, | | |
| | C. jejuni | | |
| PF-527 | S. epidermidis, M. luteus, P. mirabilis, | GSVIKKRRKRMAKKKHR | 752 |
| | E. coli, P. aeruginosa, | KLLKKTRIQRRRAGK | |
| | MRSA, S. pneumoniae, E. faecalis, | | |
| | C. jeikeium, C. jejuni | | |
| PF-528 | S. epidermidis, P. aeruginosa, C. albicans, | LVDVVVLIRRHLPKSCS- | 753 |
| | MRSA, S. pneumoniae, | NH2 | |
| | E. faecalis | | |
| PF-529 | S. epidermidis, E. coli, C. albicans, | LSEMERRRLRKRA-NH2 | 754 |
| | MRSA, S. pneumoniae, | | |
| | E. faecalis, C. jeikeium | | |
| PF-537 | S. epidermidis, M. luteus, P. mirabilis, | LANDYYKKTKKSW | 755 |
| | E. coli, C. albicans, | | |
| | MRSA, S. pneumoniae, E. faecalis, | | |
| | C. jeikeium | | |
| PF-539 | S. epidermidis, M. luteus, B. subtilis, | SIILTKKKRRKIPLSIDSQIY | 756 |
| | P. mirabilis, E. coli, P. aeruginosa, | KYTFKQ | |
| | C. albicans, MRSA, | | |
| | S. pneumoniae, E. faecalis, C. jeikeium | | |
| PF-540 | C. albicans | KSILILIKVIFIGQTTIIL | 757 |
| PF-542 | C. jeikeium | KKDNPSLNDQDKNAVLN | 758 |
| | | LLALAK | |
| PF-543 | S. epidermidis, M. luteus, B. subtilis, | NILFGIIGFVVAMTAAVIV | 759 |
| | P. mirabilis, E. coli, P. aeruginosa, | TAISIAK | |
| | C. albicans, MRSA, | | |
| | S. pneumoniae, E. faecalis, C. jeikeium | | |
| PF-544 | S. epidermidis, M. luteus, P. mirabilis, | FGEKQMRSWWKVHWFHP | 760 |
| | P. aeruginosa, MRSA, | | |
| | S. pneumoniae, E. faecalis, C. jeikeium, | | |
| | C. jejuni | | |
| PF-545 | S. epidermidis, E. coli, P. aeruginosa, | RESKLIAMADMIRRRI- | 761 |
| | S. pneumoniae, E. faecalis, | NH2 | |
| | C. jeikeium | | |
| PF-546 | S. epidermidis, E. coli, C. albicans, | PIIAPTIKTQIQ | 762 |
| | S. pneumoniae, E. faecalis, | | |
| | C. jeikeium | | |
| PF-547 | S. epidermidis, E. coli, P. aeruginosa, | WSRVPGHSDTGWKVWHR | 763 |
| | C. albicans, MRSA, | W-NH2 | |
| | S. pneumoniae, E. faecalis | | |
| PF-548 | M. luteus, P. mirabilis, E. coli, P. aeruginosa, | ARPIADLIHFNSTTVTASG | 764 |
| | C. albicans, S. pneumoniae, | DVYYGPG | |
| | E. faecalis, C. jeikeium, | | |
| | C. jejuni | | |
| PF-549 | E. coli, C. albicans, S. pneumoniae, | TGIGPIARPIEHGLDS | 765 |
| | C. jeikeium | | |
| PF-550 | S. pneumoniae | STENGWQEFESYADVGV | 766 |
| | | DPRRYVPL | |
| PF-551 | S. pneumoniae | QVKEKRREIELQFRDAEK | 767 |
| | | KLEASVQAE | |
| PF-552 | S. pneumoniae | ELDKADAALGPAKNLAPL | 768 |
| | | DVINRS | |
| PF-553 | S. epidermidis, E. coli, P. aeruginosa, | LTIVGNALQQKNQKLLLN | 769 |
| | C. albicans, MRSA, | QKKITSLG | |
| | S. pneumoniae, C. jeikeium | | |
| PF-554 | S. pneumoniae | AKNFLTRTAEEIGEQAVR | 770 |
| | | EGNINGP | |
| PF-555 | MRSA, S. pneumoniae, C. jeikeium | EAYMRFLDREMEGLTAA | 771 |
| | | YNVKLFTEAIS | |
| PF-556 | S. epidermidis, M. luteus, B. fragilis, | SLQIRMNTLTAAKASIEAA | 772 |
| | P. mirabilis, E. coli, P. aeruginosa, | | |
| | C. albicans, MRSA, | | |
| | S. pneumoniae, E. faecalis | | |
| PF-557 | S. pneumoniae | AANKAREQAAAEAKRKA | 773 |
| | | EEQAR | |
| PF-558 | S. epidermidis, E. coli, C. albicans, | ADAPPPLIVRYS | 774 |
| | C. jeikeium, C. jejuni | | |
| PF-559 | S. epidermidis, M. luteus, C. albicans, | SRPGKPGGVSIDVSRDRQ | 775 |
| | C. jeikeium, C. jejuni | DILSNYP | |
| PF-560 | S. epidermidis, M. luteus, E. coli, | FGNPFRGFTLAMEADFKK | 776 |
| | S. pneumoniae, C. jeikeium, C. jejuni | RK | |
| PF-562 | S. epidermidis, M. luteus, P. mirabilis, | TPEQWLERSTVVVTGLLN | 777 |
| | E. coli, P. aeruginosa, | RK | |
| | C. albicans, MRSA, S. pneumoniae, | | |
| | E. faecalis, C. jeikeium, | | |
| | C. jejuni | | |
| PF-563 | S. epidermidis, M. luteus, C. jeikeium | RPELDNELDVVQNSASLD | 778 |
| | | KLQASYN | |
| PF-564 | S. epidermidis, C. albicans, S. pneumoniae, | TIILNDQINSLQERLNKLN | 779 |
| | C. jeikeium | AETDRR | |
| PF-566 | P. mirabilis, S. pneumoniae | EAQQVTQQLGADFNAITT | 780 |
| | | PTATKV | |
| PF-567 | S. epidermidis, P. aeruginosa, C. albicans, | QQRVKAVDASLSQVSTQV | 781 |
| | MRSA, S. pneumoniae, | SGAVASA | |
| | C. jeikeium | | |
| PF-568 | S. epidermidis | TQAVQVKTAQAQQQ | 782 |
| PF-569 | M. luteus, P. mirabilis, S. pneumoniae, | KSKISEYTEKEFLEFVEDIY | 783 |
| | E. faecalis, C. jeikeium | TNNK | |
| PF-570 | S. pneumoniae, C. jeikeium | KKFPTEESHIQAVLEFKKL | 784 |
| | | TEHPSG | |
| PF-572 | S. epidermidis, M. luteus, E. coli, | WRASKGLPGFKAG | 785 |
| | S. pneumoniae, C. jeikeium | | |
| PF-573 | S. epidermidis, S. pneumoniae | EKKLIVKLIDSIGKSHEEIV | 786 |
| | | GAG | |
| PF-575 | M. luteus, E. coli, C. albicans, | LNFRAENKILEKIHISLIDT | 787 |
| | MRSA, S. pneumoniae, E. faecalis, | VEGSA | |
| | C. jeikeium | | |
| PF-576 | M. luteus, P. mirabilis, E. coli, P. aeruginosa, | AYSGELPEPLVRKMSKEQ | 788 |
| | C. albicans, S. pneumoniae | VRSVMGK | |
| PF-577 | S. epidermidis, M. luteus, P. mirabilis, | PFETRESFRVPVIGILGGW | 789 |
| | E. coli, P. aeruginosa, | DYFMHP | |
| | C. albicans, MRSA, S. pneumoniae, | | |
| | E. faecalis | | |
| PF-578 | S. epidermidis, M. luteus, P. mirabilis, | QKANLRIGFTYTSDSNVC | 790 |
| | P. aeruginosa, C. albicans, | NLTFALLGSK | |
| | MRSA, S. pneumoniae, | | |
| | E. faecalis, C. jeikeium | | |
| PF-579 | S. epidermidis, M. luteus, P. mirabilis, | MILVCAAVIWGRVLFILKF | 791 |
| | E. coli, P. aeruginosa, | PIYFSIRLAFL | |
| | C. albicans, MRSA, S. pneumoniae, | | |
| | E. faecalis, C. jeikeium, | | |
| | C. jejuni | | |
| PF-580 | S. epidermidis, M. luteus, E. coli, | EILNNNQVIKELTMKYKT | 792 |
| | P. aeruginosa, C. albicans, | QFESNLGGWTARARR | |
| | MRSA, S. pneumoniae, E. faecalis, | | |
| | C. jeikeium | | |
| PF-581 | S. epidermidis, M. luteus, E. coli, | WTARARR | 793 |
| | P. aeruginosa, C. albicans, | | |
| | MRSA, S. pneumoniae, E. faecalis, | | |
| | C. jeikeium | | |
| PF-583 | S. epidermidis, M. luteus, E. coli, | KFQGEFTNIGQSYIVSASH | 794 |
| | P. aeruginosa, C. albicans, | MSTSLNTGK | |
| | MRSA, S. pneumoniae, E. faecalis, | | |
| | C. jeikeium | | |
| PF-584 | S. epidermidis, M. luteus, P. mirabilis, | SYIKNLSNQKFLIAF | 795 |
| | C. albicans, MRSA, S. pneumoniae, | | |
| | E. faecalis, C. jeikeium | | |
| PF-585 | S. epidermidis, E. coli, C. albicans, | DYNHLLNVVQDWVNTN | 796 |
| | MRSA, S. pneumoniae, | | |
| | C. jeikeium | | |
| PF-586 | S. epidermidis, M. luteus, E. coli, | FFNQANYFFKEF | 797 |
| | P. aeruginosa, MRSA, S. pneumoniae, | | |
| | E. faecalis, C. jeikeium, | | |
| | C. jejuni | | |
| PF-587 | S. epidermidis, M. luteus, E. coli, | ASGKYQSYLLNVYVDSK | 798 |
| | MRSA, S. pneumoniae, E. faecalis, | KDRLDIFDKLKAKAKFVL | |
| | C. jeikeium | | |
| PF-588 | S. epidermidis, E. coli, C. albicans, | ESVEAIKAKAIK | 799 |
| | E. faecalis, C. jeikeium, | | |
| | C. jejuni | | |
| PF-589 | S. epidermidis, C. albicans, | APLRIDEIRNSNVIDEVLD | 800 |
| | MRSA, S. pneumoniae | CAPKKQEHFFVVPKIIE | |
| PF-590 | S. epidermidis, M. luteus, E. coli, | YYQAKLFPLL | 801 |
| | E. faecalis, C. jeikeium | | |
| PF-592 | S. epidermidis, M. luteus, P. mirabilis, | IMKNYKYFKLFIVKYALF | 802 |
| | E. coli, P. aeruginosa, | | |
| | C. albicans, MRSA, S. pneumoniae, | | |
| | E. faecalis, C. jeikeium, | | |
| | C. jejuni | | |
| PF-593 | C. jeikeium | MEISTLKKEKLHVKDELS | 803 |
| | | QYLANYKK | |
| PF-594 | C. jeikeium | IVSAIV | 804 |
| PF-595 | S. epidermidis, M. luteus, P. mirabilis, | LQNKIYELLYIKERSKLCS | 805 |
| | E. coli, P. aeruginosa, | | |
| | C. albicans, MRSA, S. pneumoniae, | | |
| | E. faecalis, C. jeikeium, | | |
| | C. jejuni | | |
| PF-596 | S. epidermidis, M. luteus, P. mirabilis, | SKMWDKILTILILILELIRE | 806 |
| | E. coli, P. aeruginosa, | LIKL | |
| | MRSA, E. faecalis, C. jeikeium, | | |
| | C. jejuni | | |
| PF-597 | P. mirabilis | DEIKVSDEEIEKFIKENNL | 807 |
| PF-598 | S. epidermidis, M. luteus, P. mirabilis, | MKFMLEVRNKAISAYKEI | 808 |
| | P. aeruginosa, C. albicans, | TRTQI | |
| | MRSA, S. pneumoniae, | | |
| | C. jeikeium | | |
| PF-599 | S. epidermidis, P. mirabilis, E. coli, | LFEIFKPKH | 809 |
| | C. albicans, MRSA, S. pneumoniae, | | |
| | C. jeikeium | | |
| PF-600 | S. epidermidis, M. luteus, B. subtilis, | TKKIELKRFVDAFVKKSY | 810 |
| | P. mirabilis, E. coli, P. aeruginosa, | ENYILERELKKLIKAINEEL | |
| | MRSA, S. pneumoniae, | PTK | |
| | E. faecalis, C. jeikeium | | |
| PF-601 | C. jeikeium | YRVTVKALE | 811 |
| PF-602 | P. mirabilis, C. jeikeium | LEKEKKEYIEKLFKTK | 812 |
| PF-603 | S. epidermidis, M. luteus, B. subtilis, | IDKLKKMNLQKLSYEVRI | 813 |
| | E. coli, P. aeruginosa, | SQDGKSIYARIK | |
| | MRSA, S. pneumoniae, E. faecalis, | | |
| | C. jeikeium | | |
| PF-604 | S. epidermidis, C. albicans, C. jeikeium | LMEQVEV | 814 |
| PF-605 | S. epidermidis, M. luteus, P. mirabilis, | HYRWNTQWWKY | 815 |
| | E. coli, P. aeruginosa, | | |
| | C. albicans, MRSA, S. pneumoniae, | | |
| | E. faecalis, C. jeikeium, | | |
| | C. jejuni | | |
| PF-607 | S. epidermidis, P. mirabilis, E. coli, | YIESDPRKFDYIFGAIRDH | 816 |
| | MRSA, S. pneumoniae, C. jeikeium | | |
| PF-609 | P. mirabilis, E. coli, S. pneumoniae | TEIKLDNNEYLVLNLDDIL | 817 |
| | | GILK | |
| PF-610 | S. epidermidis, M. luteus, P. mirabilis, | VFLKLKTSKIDLASIIFYP | 818 |
| | E. coli, P. aeruginosa, | | |
| | C. albicans, MRSA, S. pneumoniae, | | |
| | E. faecalis, C. jeikeium, | | |
| | C. jejuni | | |
| PF-612 | S. epidermidis, M. luteus, P. mirabilis, | GTTLKYGLERQLKIDIHPE | 819 |
| | E. coli, P. aeruginosa, | ITIINLNGGADEFAKL | |
| | C. albicans, MRSA, S. pneumoniae, | | |
| | E. faecalis, C. jeikeium | | |
| PF-613 | C. jeikeium | ADEFAKL | 820 |
| PF-614 | S. epidermidis, E. coli, C. jeikeium | GLDIYA | 821 |
| PF-615 | S. epidermidis, M. luteus, P. mirabilis, | FLNRFIFYIFTVKTKSALIK | 822 |
| | E. coli, P. aeruginosa, | NLFLD | |
| | C. albicans, MRSA, S. pneumoniae, | | |
| | C. jeikeium, C. jejuni | | |
| PF-616 | C. jeikeium | IVFVVTKEKK | 823 |
| PF-617 | P. aeruginosa, C. albicans | PMNAAEPE | 824 |
| PF-619 | S. epidermidis, M. luteus, B. subtilis, | WSRVPGHSDTGWKVWHRW | 825 |
| | P. mirabilis, E. coli, P. aeruginosa, | | |
| | C. albicans, MRSA, | | |
| | S. pneumoniae, E. faecalis, C. jeikeium | | |
| PF-621 | S. epidermidis, C. albicans | PPSSFLV | 826 |
| PF-622 | S. epidermidis, P. aeruginosa, C. albicans, | TREDVFSVRLINNIVNKQA | 827 |
| | S. pneumoniae, E. faecalis, | | |
| | C. jeikeium | | |
| PF-623 | S. epidermidis, P. aeruginosa, | VLFAVYLGALDWLFSWL | 828 |
| | MRSA, S. pneumoniae, E. faecalis, | TQKM | |
| | C. jeikeium | | |
| PF-625 | S. epidermidis, M. luteus, S. pneumoniae, | SDSTNNARTRKKARDVTT | 829 |
| | C. jeikeium | KDIDK | |
| PF-626 | S. epidermidis, M. luteus, E. coli, | KYDFDDFEPEEA | 830 |
| | C. albicans, MRSA, S. pneumoniae, | | |
| | E. faecalis, C. jeikeium | | |
| PF-627 | S. epidermidis, P. aeruginosa, | INDLLSYFTLHEK | 831 |
| | MRSA, S. pneumoniae, E. faecalis, | | |
| | C. jeikeium | | |
| PF-629 | S. epidermidis, P. aeruginosa, C. albicans, | GLAAIATVFALY | 832 |
| | MRSA, S. pneumoniae, | | |
| | E. faecalis, C. jeikeium | | |
| PF-630 | S. epidermidis, M. luteus, P. mirabilis, | IPATPIIHS | 833 |
| | P. aeruginosa, C. albicans, | | |
| | MRSA, S. pneumoniae, | | |
| | E. faecalis, C. jeikeium | | |
| PF-631 | S. epidermidis, P. aeruginosa, C. albicans, | LIIYFSKTGNTARATRQI | 834 |
| | MRSA, S. pneumoniae, | | |
| | E. faecalis, C. jeikeium | | |
| PF-632 | S. epidermidis, P. aeruginosa, C. albicans, | TTIQGVASLEKHGFRYTII | 835 |
| | MRSA, S. pneumoniae, | YPTRI | |
| | E. faecalis, C. jeikeium | | |
| PF-634 | S. epidermidis, M. luteus, P. mirabilis, | MPKARPVNHNKKKSKITI | 836 |
| | E. coli, P. aeruginosa, | KSNFTLFYMFNP | |
| | C. albicans, MRSA, S. pneumoniae, | | |
| | E. faecalis, C. jeikeium | | |
| PF-635 | S. epidermidis, P. aeruginosa, C. albicans, | MNAHGHSLIFQKMIVHAF | 837 |
| | MRSA, S. pneumoniae, | AFFSKQKNYLYF | |
| | E. faecalis, C. jeikeium | | |
| PF-636 | S. epidermidis, C. albicans, | LVRLA | 838 |
| | MRSA, S. pneumoniae, E. faecalis | | |
| PF-637 | S. epidermidis, P. aeruginosa, C. albicans, | SRIKQDARSVRKYDRIGIF | 839 |
| | MRSA, S. pneumoniae, | FYSFKSA | |
| | E. faecalis, C. jeikeium | | |
| PF-638 | S. epidermidis, C. albicans, | TFILPK | 840 |
| | MRSA, C. jeikeium | | |
| PF-639 | S. pneumoniae, C. jeikeium | QATQIKSWIDRLLVSED | 841 |
| PF-640 | C. albicans | MGDINRNF | 842 |
| PF-641 | S. epidermidis, M. luteus, E. coli, | SWKCHHLAIGGSWKCHH | 843 |
| | C. albicans, MRSA, E. faecalis, | LAI | |
| | C. jeikeium, C. jejuni | | |
| PF-642 | M. luteus, MRSA, C. jeikeium | FTTPMIGIPAGLLGGSYYL | 844 |
| | | KRREEKGK | |
| PF-643 | Mycobacteria spp | VRCRL | 845 |
| PF-644 | Mycobacteria spp | TSGLIIGENGLNGL | 846 |
| PF-645 | Mycobacteria spp | SNSVQQG | 847 |
| PF-646 | Mycobacteria spp | APASPGRRPG | 848 |
| PF-647 | Mycobacteria spp | GTFLGQKCAAATAS | 849 |
| PF-648 | S. mutans, E. coli | ARRYPAAGS | 850 |
| PF-649 | Mycobacteria spp | CPRYPFVDVGPAGPWRAR | 851 |
| | | WRVGS | |
| PF-650 | Mycobacteria spp | IRSDQPGRQSRSSPRWPTG | 852 |
| | | AGRHR | |
| PF-651 | Mycobacteria spp | PRWPTGAGRHR | 853 |
| PF-652 | Mycobacteria spp | FLAPARPDLQAQRQALAQ | 854 |
| PF-653 | Mycobacteria spp | QSVHPLPAETPVADVI | 855 |
| PF-654 | Mycobacteria spp | LSGRLAGRR | 856 |
| PF-655 | M. smegmatis | DAPCFDDQFGDLKCQMC | 857 |
| PF-656 | Mycobacteria spp | RGMFVPFHDVDCVQ | 858 |
| PF-657 | Mycobacteria spp | YVANYTITQFGRDFDDRL | 859 |
| | | AVAIHFA | |
| PF-658 | Mycobacteria spp | PTTPPPTTPPEIPTGGTVIST | 860 |
| PF-659 | Mycobacteria spp | TVIST | 861 |
| PF-660 | Mycobacteria spp | TDPQATAAPRRRTSPR | 862 |
| PF-661 | Mycobacteria spp | PDEDIRRRAILPPAGPCRP | 863 |
| | | MSPE | |
| PF-662 | Mycobacteria spp | GKQSRAHGPVASRREFRR | 864 |
| | | KSG | |
| PF-663 | Mycobacteria spp | ATLIPRKA | 865 |
| PF-664 | M. smegmatis | DQLCVEYPARVSTG | 866 |
| PF-665 | Mycobacteria spp | VLRVATAVGEVPTGL | 867 |
| PF-666 | Mycobacteria spp | PNRRSRPR | 868 |
| PF-667 | Mycobacteria spp | PAHQRLRIDQRLVADRDM | 869 |
| | | VQDYES | |
| PF-668 | Mycobacteria spp | TNAESMALAFRGRVHMS | 870 |
| | | VNIAGLT | |
| PF-669 | Mycobacteria spp | RADRIESYPADGDRVITL | 871 |
| | | WRNPYR | |
| PF670 | Mycobacteria spp | TVIVAPMHSGV | 872 |
| PF-671 | S. mutans, E. coli | TVSAFRTVH | 873 |
| PF-673 | S. mutans, E. coli | VRRLRM | 874 |
| PF-674 | S. mutans, E. coli | DGCDSEPALTYR | 875 |
| PF-675 | Mycobacteria spp | EIIPISPTRRCEMHTMSSAE | 876 |
| | | YRGL | |
| PF-676 | S. mutans, E. coli | AEYRGL | 877 |
| PF-677 | Mycobacteria spp | TCRGAGMH | 878 |
| PF-678 | Mycobacteria spp | RDRRWTRRDMYDWLESA | 879 |
| | | RV | |
| PF-679 | S. mutans, E. coli | CRARFIRR | 880 |
| PF-680 | Mycobacteria spp | ADPHPTTGI | 881 |
| PF-681 | M. smegmatis | TALTTVGVSGARLITYCV | 882 |
| | | GVEDI | |
| PF-682 | Mycobacteria spp | RRGKSEQGLSRR | 883 |
| PF-683 | Mycobacteria spp | LWPVA | 884 |
| PF-684 | Mycobacteria spp | RKLSLASGFALWRRSLV | 885 |
| PF-685 | Mycobacteria spp | PTLWLACL | 886 |
| PF-686 | M. smegmatis | LAVLMGYIGYRGWSGKR | 887 |
| | | HINRQ | |
| PF-687 | Mycobacteria spp | AKRVLSLAVAPHRRQPVQ | 888 |
| | | GT | |
| PF-688 | Mycobacteria spp | ARNHAVIPAG | 889 |
| PF-689 | S. mutans, E. coli | SAPSG | 890 |
| PF-690 | Mycobacteria spp | MIPLAGDPVSSHRTVEFG | 891 |
| | | VLGTYLVSGGSL | |
| PF-691 | Mycobacteria spp | HRTVEFGVLGTYLVSGGSL | 892 |
| PF-692 | Mycobacteria spp | GVAREDPLEPDPLAPIIDD | 893 |
| | | SR | |
| PF-693 | Mycobacteria spp | PDPAR | 894 |
| PF-694 | Mycobacteria spp | DLIRPLYSMSAPSVA | 895 |
| PF-695 | Mycobacteria spp | ALSVMLGNIPLVVPNANQL | 896 |
| PF-696 | Mycobacteria spp | IRSGISAAYARPLR | 897 |
| PF-697 | Mycobacteria spp | RADARAK | 898 |
| PF-698 | Mycobacteria spp | SSGRAGVKCRRPTGR | 899 |
| PF-699 | Mycobacteria spp | GRAGVKCRRPTGR | 900 |
| PF-700 | Mycobacteria spp | LNWPFTGR | 901 |
| PF-701 | S. mutans | PRGAQSGHG | 902 |
| PF-702 | Mycobacteria spp | LSGRLAGRR | 903 |
| PF-703 | Mycobacteria spp | MTTVDNIVGLVIAVALMA | 904 |
| | | FLFAALLFPEKF | |
| PF-704 | Mycobacteria spp | APAARAAL | 905 |
| PF-705 | S. mutans, E. coli | GEEEGTVAD | 906 |
| PF-706 | L. pneumophila | LGYGAWIGCGLGLNGFHR | 907 |
| | | ID | |
| PF-707 | S. mutans, E. coli | IDPESIVTTNNKQDNVDEQ | 908 |
| PF-709 | S. mutans | NKKHSPMD | 909 |
| PF-711 | S. mutans | KTAGPTGTIYKTN | 910 |
| PF-712 | S. mutans, E. coli | QIYRHVHKVQAKSANLRLY | 911 |
| PF-714 | L. pneumophila | FVVTQRMLRMYKK | 912 |
| PF-716 | S. mutans | HGENHHHKSDEKDNDSSE | 913 |
| | | KKD | |
| PF-717 | E. coli | PQSEVTFENIYAPKANGG | 914 |
| | | GLYGI | |
| PF-720 | S. mutans | SLDMGK | 915 |
| PF-724 | L. pneumophila | CYRFLTPKRPTRIS | 916 |
| PF-727 | S. mutans, E. coli | AYARCRHDYPFTLGQMQ | 917 |
| | | TH | |
| PF-728 | S. mutans, E. coli | AIGQEQDRREYYYYSGYP | 918 |
| | | YYY | |
| PF-731 | L. pneumophila | RHKLIRLPLSESVFCFLNN | 919 |
| | | PKI | |
| PF-732 | E. coli | DRPSQTTHHTLSSSRITGPS | 920 |
| PF-733 | S. mutans, E. coli | VISRQMGSEAVLELFIIM | 921 |
| PF-735 | S. mutans, E. coli | YDPLFPNDKN | 922 |
| PF-737 | S. epidermidis, S. pneumoniae | KSSGSSASASSTAGGSSSK | 923 |
| PF-738 | S. epidermidis, C. albicans, C. jeikeium | KSGATSAASGAKSGASS | 924 |
| PF-741 | S. epidermidis, M. luteus, P. mirabilis, | AKREDTVAAQIGANILNLIQ | 925 |
| | P. aeruginosa, C. albicans, | | |
| | MRSA, S. pneumoniae, | | |
| | E. faecalis | | |
| PF-744 | S. epidermidis, M. luteus, E. coli, | LGVGTFVGKVLIKNQQKQ | 926 |
| | MRSA, S. pneumoniae, E. faecalis, | KSKKKAQ | |
| | C. jeikeium | | |
| PF-745 | S. epidermidis, M. luteus, C. albicans | ANSQNSLFSNRSSFKSIFD | 927 |
| | | KKSNITTNATTPNSNIIIN | |
| PF-746 | S. epidermidis, M. luteus, E. coli, | FLGNSQYFTRK | 928 |
| | C. albicans, S. pneumoniae, E. faecalis, | | |
| | C. jeikeium | | |
| PF-748 | S. epidermidis, M. luteus, E. coli, | FQGFFDVAVNKWWEEHN | 929 |
| | P. aeruginosa, C. albicans, | KAKLWKNVKGKFLEGEG | |
| | MRSA, S. pneumoniae, E. faecalis, | EEEDDE | |
| | C. jeikeium | | |
| PF-749 | S. epidermidis, M. luteus, E. coli, | GVNKWWEEHNKAKLWK | 930 |
| | P. aeruginosa, C. albicans, S. pneumoniae, | NVKGKFLEGEGEEEDDE | |
| | C. jeikeium | | |
| PF-750 | M. luteus, C. jeikeium | AESSPAKTTA | 931 |
| PF-751 | S. epidermidis, E. coli, C. albicans, | AESSPAQETT | 932 |
| | C. jeikeium | | |
| PF-752 | S. epidermidis, E. coli, MRSA, S. pneumoniae, | LHVIRPRPELSELKFPITKI | 933 |
| | E. faecalis | LKVNKQGLKK | |
| PF-756 | S. epidermidis, M. luteus, C. albicans, | DALLRLA | 934 |
| | MRSA, C. jeikeium | | |
| PF-757 | M. luteus, C. albicans, MRSA | PQAISSVQQNA | 935 |
| PF-758 | S. epidermidis, M. luteus, E. coli, | PEIIKIVSGLL | 936 |
| | MRSA, S. pneumoniae, E. faecalis, | | |
| | C. jeikeium | | |
| PF-760 | S. epidermidis, M. luteus | DHITLDDYEIHDGFNFELY | 937 |
| | | YG | |
| PF-761 | S. epidermidis, M. luteus, P. mirabilis, | SKFELVNYASGCSCGADC | 938 |
| | P. aeruginosa, C. albicans, | KCASETECKCASKK | |
| | MRSA, S. pneumoniae, | | |
| | E. faecalis | | |
| PF-762 | M. luteus, C. albicans | PAPAPSAPAPAPEQPEQPA | 939 |
| PF-763 | S. epidermidis, M. luteus, E. coli, | GIWMARNYFHRSSIRKVY | 940 |
| | C. albicans, MRSA, S. pneumoniae, | VESDKEYERVHPMQKIQY | |
| | E. faecalis | EGNYKSQ | |
| PF-764 | S. epidermidis, M. luteus, C. albicans, | GYFEPGKRD | 941 |
| | MRSA, S. pneumoniae, | | |
| | E. faecalis, C. jeikeium | | |
| PF-765 | S. epidermidis, M. luteus, E. coli, | YLYWEVEHKPIIAKRDAY | 942 |
| | MRSA, E. faecalis, C. jeikeium | YAQLRKQKEIEEGA | |
| PF-766 | S. epidermidis, M. luteus, C. albicans, | DAYYAQLRKQKEIEEGA | 943 |
| | MRSA, E. faecalis, C. jeikeium | | |
| PF-767 | S. epidermidis, M. luteus, E. coli, | DGKQGEPVALKPTDN | 944 |
| | MRSA, S. pneumoniae, E. faecalis, | | |
| | C. jeikeium | | |
| PF-768 | S. epidermidis, S. pneumoniae, E. faecalis, | GFRGGKRGGARG | 945 |
| | C. jeikeium | | |
| PF-770 | S. epidermidis, M. luteus, P. mirabilis, | GVGIGFIMMGVVGYAVK | 946 |
| | E. coli, P. aeruginosa, | LVHIPIRYLIV | |
| | C. albicans, MRSA, S. pneumoniae, | | |
| | E. faecalis, C. jeikeium | | |
| PF-772 | S. epidermidis, C. albicans, | TKESSS | 947 |
| | MRSA, S. pneumoniae, C. jeikeium | | |
| PF-773 | S. epidermidis, C. albicans, C. jeikeium | TLKESK | 948 |
| PF-776 | S. epidermidis, M. luteus, P. mirabilis, | VSILLYLSATIILPNVLRLL | 949 |
| | C. albicans, MRSA, S. pneumoniae, | VARAIIVRV | |
| | E. faecalis | | |
| PF-777 | Mycobacteria spp. | PGADGKLAEASAAIARLV | 950 |
| | | RS | |
| PF-778 | Mycobacteria spp. | MNLILTAHGT | 951 |
| PF-779 | Mycobacteria spp. | IYGDFFNFYLCDISLKVNG | 952 |
| | | LQPGGPVRTVKLFGQPTG | |
| | | RCTPQ | |
| PF-780 | Mycobacteria spp. | AVYDALVALAAAEHRAE | 953 |
| | | LATRDARAKDTYEKIGVH | |
| | | VVVAA | |
| PF-781 | Mycobacteria spp. | PLVVVNHRRAERSRG | 954 |
| PF-782 | Mycobacteria spp. | TGPRRGIDLTSNRALSEVL | 955 |
| | | DEGLELNSRK | |
| PF-783 | Mycobacteria spp. | FTSEVRGVFTYRVNKAGL | 956 |
| | | ITNMRGYWNLDMMTFGN | |
| | | QE | |
| PF-784 | Mycobacteria spp. | MAMTTVDNIVGLVIAVAL | 957 |
| | | MAFLFAALLFPEKF | |
| PF-785 | Mycobacteria spp. | MRPQHSPAGKAFVVKKIT | 958 |
| | | HEQS | |
| PF-786 | Mycobacteria spp. | LSERERRRLKRGII | 959 |
| PF-787 | Mycobacteria spp. | MTERQRRALLKQHPEVVS | 960 |
| | | WSDYLEKRKRRTGTAG | |
| PF-788 | Mycobacteria spp. | GLITVFAGTARILQLRRAA | 961 |
| | | KKTHAAALR | |
| PF-789 | Mycobacteria spp. | PRGAQSGHG | 962 |
| PF-790 | Mycobacteria spp. | PAGPDHLDQRDHR | 963 |
| PF-791 | S. mutans | IFLTTQNTDYSEHNAA | 964 |
| PF-792 | S. mutans | ALHASGIQAI | 965 |
| PF-793 | S. mutans | YTQUNNASAYAMLLTNK | 966 |
| | | DTVP | |
| PF-794 | S. mutans | NLYFENQGN | 967 |
| PF-795 | S. mutans | ALHKSGIQVIADWVPDQI | 968 |
| | | YN | |
| PF-796 | S. mutans | YTQSNIPTAYALMLSNKD | 969 |
| | | SI | |
| PF-797 | S. mutans | WYYFDNNGYM | 970 |
| PF-798 | S. mutans | ALHSKGIKVMADWVPDQ | 971 |
| | | MYA | |
| PF-799 | S. mutans | YTHYNTALSYALLLTNKS | 972 |
| | | SVP | |
| PF-800 | S. mutans | WYYFDNNGYM | 973 |
| PF-C003 | A. naeslundii, P. gingivalis, S. mutans | FCSVDHDVITIAADHVKQ | 974 |
| | | GAEA | |
| PF-C008 | A. naeslundii | AQPRRTWLVNFGEVPSPG | 975 |
| | | LTNDGMPDH | |
| PF-C034 | S. mutans, E. coli | HPMPITVRSRKPGPLTAPS | 976 |
| | | EH | |
| PF-C045 | A. naeslundii, T. denticola | FREGMGWPLSNEGSPTAP | 977 |
| | | LPKHRNQV | |
| PF-C050 | A. naeslundii, S. mutans | QGLARPVLRRIPL | 978 |
| PF-C052 | A. naeslundii, F. nucleatum, S. mutans | SRFRNGV | 979 |
| PF-C055 | A. naeslundii, F. nucleatum, P. gingivalis, | YNLSIYIYFLHTITIAGLITL | 980 |
| | S. mutans | PFII | |
| PF-C057 | A. naeslundii, F. nucleatum, P. gingivalis, | YFWWYWVQDCIPYKNNE | 981 |
| | S. mutans | VWLELSNNMK | |
| PF-C058 | A. naeslundii, F. nucleatum, P. gingivalis, | FETGFGDGYYMSLWGLN | 982 |
| | S. mutans | EKDEVCKVVIPFINPELID | |
| PF-C061 | A. naeslundii, F. nucleatum, P. gingivalis, | TLNYKKMFFSVIFLLGLN | 983 |
| | S. mutans, T. denticola | YLICNSPLFFKQIEF | |
| PF-C062 | A. naeslundii, F. nucleatum, P. gingivalis, | PLARATEVVATLFIICSLLL | 984 |
| | S. mutans, T. denticola | YLTR | |
| PF-C063 | A. naeslundii, F. nucleatum, S. mutans | SHFRKGD | 985 |
| PF-C064 | A. naeslundii, F. nucleatum, P. gingivalis, | DEEALEMGANLYAQFAID | 986 |
| | S. mutans, T. denticola | FLNSKK | |
| PF-C065 | A. naeslundii, F. nucleatum, P. gingivalis, | DEERYSDSYFLKEKVFYLI | 987 |
| | S. mutans | LALFLILFHQKYLYFLEIITI | |
| PF-C068 | A. naeslundii, F. nucleatum, S. mutans | LNLFASI | 988 |
| PF-C069 | A. naeslundii, F. nucleatum, P. gingivalis, | NALMLREMQLAKNIKVE | 989 |
| | S. mutans, T. denticola | VTDVLSNKKYC | |
| PF-C071 | A. naeslundii, F. nucleatum, S. mutans | QVIVKIL | 990 |
| PF-C072 | A. naeslundii, F. nucleatum, P. gingivalis, | KKMFSLIRKVNWIFFILFIV | 991 |
| | S. mutans, T. denticola | LDLTNVFPLIRTILFAILSRQ | |
| PF-C075 | A. naeslundii, F. nucleatum, P. gingivalis, | KALVISVFAIVFSIIFVKFF | 992 |
| | S. mutans, T. denticola | YWRDKK | |
| PF-C080 | A. naeslundii, F. nucleatum, S. mutans | INIPGLF | 993 |
| PF-C084 | A. naeslundii, F. nucleatum, P. gingivalis, | FFSVIFLFGLNYLICNSPLF | 994 |
| | S. mutans | NILR | |
| PF-C085 | A. naeslundii, F. nucleatum, P. gingivalis, | KKFKIFVIINWFYHKYIILN | 995 |
| | S. mutans, T. denticola | FEENF | |
| PF-C086 | A. naeslundii, F. nucleatum, P. gingivalis, | ELFFTILSDCNELFLLHLLQ | 996 |
| | S. mutans, T. denticola | QPLFYIKKGK | |
| PF-C088 | A. naeslundii, F. nucleatum, P. gingivalis, | DIANNILNSVSERLIIA | 997 |
| | S. mutans, T. denticola | | |
| PF-C091 | A. naeslundii, F. nucleatum, P. gingivalis, | ASNTPRFVRLTLFNFYSKI | 998 |
| | S. mutans, T. denticola | WNVTHLFLFNNL | |
| PF-C093 | A. naeslundii, F. nucleatum, S. mutans | EKLGTMV | 999 |
| PF-C095 | A. naeslundii, F. nucleatum, P. gingivalis, | LLALNMNEDTYYFELFFIF | 1000 |
| | S. mutans | DNQNKKWLIFDLKERG | |
| PF-C098 | A. naeslundii, F. nucleatum, P. gingivalis, | PETKGKVSAFVFGIVVAN | 1001 |
| | S. mutans, T. denticola | VIAVVYILYMLREIGIIQ | |
| PF-C120 | A. naeslundii, F. nucleatum, P. gingivalis, | ASLSTMTFKVMELKELIIL | 1002 |
| | S. mutans, T. denticola | LCGLTMLMIQTEFV | |
| PF-C131 | A. naeslundii, F. nucleatum, P. gingivalis, | QWIVAKREIRMHIYCHISV | 1003 |
| | S. mutans | IHVIIFFG | |
| PF-C134 | A. naeslundii, F. nucleatum, P. gingivalis, | NELMKYPATLTATATTPG | 1004 |
| | S. mutans, T. denticola | IKYSHLCSVCL | |
| PF-C135 | A. naeslundii, F. nucleatum, P. gingivalis, | KNTHAYLRVLRLSSLILSY | 1005 |
| | S. mutans | QASVYPLFAYLCQQKDY | |
| PF-C136 | A. naeslundii, F. nucleatum, P. gingivalis, | LILSYQASVYPLFAYLCQQ | 1006 |
| | S. mutans, T. denticola | KDY | |
| PF-C137 | A. naeslundii, F. nucleatum, P. gingivalis, | QRMYWFKRGFETGDFSA | 1007 |
| | S. mutans | GDTFAELK | |
| PF-C139 | A. naeslundii, F. nucleatum, P. gingivalis, | LLASHPERLSLGVFFVYRV | 1008 |
| | S. mutans, T. denticola | LHLLLENT | |
| PF-C142 | A. naeslundii, F. nucleatum, P. gingivalis, | DFPPLSFFRRRFHAYTAPI | 1009 |
| | S. mutans, T. denticola | DNFFGANPF | |
| PF-C143 | A. naeslundii, F. nucleatum, P. gingivalis, | VVFGGGDRLV | 1010 |
| | S. mutans, T. denticola | | |
| PF-C145 | A. naeslundii, F. nucleatum, P. gingivalis, | YGKESDP | 1011 |
| | S. mutans, T. denticola | | |
| PF-C160 | F. nucleatum | AASGFTYCASNGVWHPY | 1012 |
| PF-C180 | F. nucleatum, P. gingivalis, S. mutans, | TVEELDKAFTWGAAAAL | 1013 |
| | T. denticola | AIGVIAINVGLAAGYCYN | |
| | | NNDVF | |
| PF-C181 | P. gingivalis | KMRAGQVVFIYKLILVLL | 1014 |
| | | FYVLQKLFDLKKGCF | |
| PF-C194 | A. naeslundii, F. nucleatum, P. gingivalis, | NTNDLLQAFELMGLGMA | 1015 |
| | S. mutans, T. denticola | GVFIVLGILYIVAELLIKIFP | |
| | | VNN | |
| PF-C259 | F. nucleatum, S. mutans | AEIQPHCLSVL | 1016 |
| PF-C271 | A. naeslundii, F. nucleatum, P. gingivalis, | FFPSYYSIIITYF | 1017 |
| | S. mutans, T. denticola | | |
| PF-C273 | A. naeslundii, P. gingivalis, S. mutans, | KNMLKRRMKQKRLFDEE | 1018 |
| | T. denticola | DRLRVLSKYTKSYY | |
| PF-C281 | A. naeslundii, F. nucleatum, P. gingivalis, | KKEKLLTAIRLQHRAEIRG | 1019 |
| | S. mutans, T. denticola | YFTIFFLFFRI | |
| PF-C285 | A. naeslundii, F. nucleatum, P. gingivalis, | FTIIELKKQKIKHGENNKK | 1020 |
| | S. mutans, T. denticola | TAHPLNEPFCARA | |
| PF-C290 | A. naeslundii, F. nucleatum, P. gingivalis, | GNVHPESDFHNLIQFIKTF | 1021 |
| | S. mutans, T. denticola | LYFTIFFKYFL | |
| PF-C291 | A. naeslundii, F. nucleatum, P. gingivalis, | HPFLTGTGCPLFLIFRLFFV | 1022 |
| | S. mutans | KAYFSFTVF | |
| PF-S003 | S. epidermidis, M. luteus, P. mirabilis, | ALALLKQDLLNFEGRGRII | 1023 |
| | E. coli, P. aeruginosa, | TSTYLQFNEGCVP | |
| | C. albicans, MRSA, S. pneumoniae, | | |
| | E. faecalis, C. jeikeium, | | |
| | C. jejuni, M. smegmatis | | |
| PF-S004 | S. epidermidis, MRSA, C. jeikeium | VLLNIFRTLLEFFSPSNAPG | 1024 |
| | | AEDVPLPDTQA | |
| PF-S007 | S. epidermidis, MRSA | VVAGVVLLTALAVGSKR | 1025 |
| | | KEKKQIKEIQRLLAATR | |
| PF-S015 | S. epidermidis, MRSA, C. jeikeium | IENLERGARRPP | 1026 |
| PF-S018 | S. epidermidis, M. luteus, C. albicans, | GMPQIPRLRI | 1027 |
| | MRSA, E. faecalis, C. jeikeium, | | |
| | C. jejuni | | |
| PF-S023 | S. epidermidis, MRSA | MAEDERRALKRRTNRGR | 1028 |
| | | TRTRKRITV | |
| PF-S026 | S. epidermidis, MRSA, C. jeikeium | TELKYNGEEYLLLTQRDIL | 1029 |
| | | AVIEK | |
| PF-S029 | M. luteus, P. mirabilis, E. coli, C. albicans, | TSDTQSQSPWLFDNADIV | 1030 |
| | C. jeikeium, C. jejuni | NIYPVQLMHSSDND | |
| PF-U | S. mutans | TFFRLFNRSFTQALGK | 1031 |
| PF-V | S. mutans | KFINGVLSQFVLERK | 1032 |
| PF-W | S. mutans | FIDSFIRSF | 1033 |
| PF-X | S. mutans | TFFRLFNRSFTQALGK | 1034 |
| PF-Y | S. mutans | FIKHFIHRF | 1035 |
| PF-Z | | KKHRKHRKHRKH | 1036 |

Additional illustrative suitable targeting peptides include, but are not limited to the peptides shown in Table 10 of copending PCT Patent Application No: PCT/US2010/020242, and Table 3 of copending U.S. Patent Application No. 61/334,511, both of which are incorporated herein by reference. Additional suitable targeting peptides include, but are not limited to, bacterial and/or fungal pheromones such as those shown in Table 12 of PCT Patent Application No: PCT/US2010/020242, which is incorporated herein by reference.

In certain embodiments the targeting moieties can comprise one or more antibodies that bind specifically or preferentially a microorganism or group of microorganisms (e.g., bacteria, fungi, yeasts, protozoa, molds, viruses, algae, etc.). The antibodies are selected to bind an epitope characteristic or the particular target microorganism(s). In various embodiments such epitopes or antigens are typically gram-positive or gram-negative specific, or genus-specific, or species-specific, or strain specific and located on the surface of a target microbial organism. The antibody that binds the epitope or antigen can direct the permeabilizing moiety to the site.

An illustrative, but not limiting list of antibodies that bind various microorganisms is shown in Table 3.

TABLE 3
Illustrative antibodies that bind target microorganisms.

| Source | Antibody |
|---|---|
| U.S. Pat. No. 7,195,763 | Polyclonal/monoclonal binds specific Gram(+) cell wall repeats |
| U.S. Pat. No. 6,939,543 | Antibodies against G(+) LTA |
| U.S. Pat. No. 7,169,903 | Antibodies against G(+) peptidoglycan |
| U.S. Pat. No. 6,231,857 | Antibody against S. mutans (Shi) |
| U.S. Pat. No. 5,484,591 | Gram(−) binding antibodies |
| US 2007/0231321 | Diabody binding to Streptococcus surface antigen I/II |
| US 2003/0124635 | Antibody against S. mutans |
| US 2006/0127372 | Antibodies to Actinomyces naeslundii, Lactobacillus casei |
| US 2003/0092086 | Antibody to S. sobrinus |
| U.S. Pat. No. 7,364,738 | Monoclonal antibodies to the ClfA protein in S. aureus |
| U.S. Pat. No. 7,632,502 | Antibodies against C. albicans |
| U.S. Pat. No. 7,608,265 | Monoclonal against C. difficile |
| U.S. Pat. No. 4,777,136 | Monoclonal Antibodies against Pseudomonas aeruginosa |
| see, e.g., ab20429, ab20560, | Antibody against S. pneumoniae |
| ab79522, ab35165, ab65602 | |
| from AbCAMm Cambridge | |
| Science Park, U.K. | |

Permeabilizing/Lytic Moieties.

In various embodiments the targeting moiety (e.g., targeting antibody or peptide) is attached (directly or indirectly) to a permeabilizing or lytic moiety to produce a selective permeabilizing reagent (i.e., a reagent that selectively permeabilizes a target microorganism, a target group of microorganisms, a target cell, etc.).

Suitable permeabilizing or lytic moieties include, but are not limited to, antimicrobial peptides, surfactants, lytic proteins, cationic colic acid, steroid antibiotics, nanotubes or nanoparticles (e.g., tubes 40 to 400 nm in diameter or particles with a characteristic dimension of typically <500 nm), tubular microtubes (e.g., tubes >400 nm in diameter), carrier proteins or peptides, carrier molecules such as ionophores, lipid flipases, lipases, lysozyme, phage injector assemblies, and the like.

In certain embodiments the permeabilizing or lytic moieties comprise one or more antimicrobial peptides. Illustrative suitable antimicrobial peptides are shown in Table 4.

TABLE 4
Novel antimicrobial peptides,
target microorganisms and MIC values.

| | | | |
|---|---|---|---|
| | Organism MIC | | SEQ |
| ID | (μM) | Structure/sequence | ID NO |

| | | | |
|---|---|---|---|
| K-1 | S. mutans, 25 | GLGRVIGRLIKQIIWRR | 1037 |
| K-2 | S. mutans, 12.5 | VYRKRKSILKIYAKLKGWH | 1038 |
| K-7 | S. mutans, 12.5 | NYRLVNAIFSKIFKKKFIKF | 1039 |
| K-8 | S. mutans, 4 | KILKFLFKKVF | 1040 |
| K-9 | S. mutans, 4 | FIRKFLKKWLL | 1041 |
| K-10 | S. mutans, 4 | KLFKFLRKHLL | 1042 |
| K-11 | S. mutans, 4 | KILKFLFKQVF | 1043 |
| K-12 | S. mutans, 8 | KILKKLFKFVF | 1044 |
| K-13 | S. mutans, 16 | GILKKLFTKVF | 1045 |
| K-14 | S. mutans, 8 | LRKFLHKLF | 1046 |
| K-15 | S. mutans, 4 | LRKNLRWLF | 1047 |
| K-16 | S. mutans, 8 | FIRKFLQKLHL | 1048 |
| | P. aeruginosa, 12.5 | | |
| | MRSA, 25 | | |
| K-17 | S. mutans, 8 | FTRKFLKFLHL | 1049 |
| K-18 | S. mutans, 16 | KKFKKFKVLKIL | 1050 |
| K-19 | S. mutans, 16 | LLKLLKLKKLKF | 1051 |
| K-20 | S. mutans, 8 | FLKFLKKFFKKLKY | 1052 |
| K-21 | S. mutans, 8 | GWLKMFKKIIGKFGKF | 1053 |
| K-22 | S. mutans, 8 | GIFKKFVKILYKVQKL | 1054 |
| 1T-88 | | GRLVLEITADEVKALGEALANAKI | 1055 |
| PF-531 | A. baumannii, 25 | YIQFHLNQQPRPKVKKIKIFL-NH2 | 1056 |
| | P. aeruginosa, 50 | | |
| | T. rubrum, 50 | | |
| | A. niger, 25 | | |
| | B. subtilis, 25 | | |
| | C. difficile, 12.5 | | |
| | C. jeikeium, 6.25 | | |
| | S. epidermidis, 50 | | |
| | S. mutans, 12.5 | | |
| PF-527 | P. aeruginosa, 50 | GSVIKKRRKRMAKKKHRKLLKKTRIQR | 1057 |
| | T. rubrum, 25 | RRAGK | |
| | A. niger, 50 | | |
| | B. subtilis, 12.5 | | |
| | C. jeikeium, 6.25 | | |
| | MRSA, 50 | | |
| | S. epidermidis, 25 | | |
| PF-672 | C. albicans, 1.56 | MRFGSLALVAYDSAIKHSWPRPSSVRR | 1058 |
| | T. rubrum, 0.78 | LRM | |
| | A. niger, 3 | | |
| | B. subtilis, 0.78 | | |
| | E. faecalis, 3.13 | | |
| | MRSA, 1.56 | | |
| | S. epidermidis, 0.39 | | |
| PF-606 | E. coli, 50 | FESKILNASKELDKEKKVNTALSFNSHQ | 1059 |
| | MRSA, 50 | DFAKAYQNGKI | |
| | S. epidermidis, 50 | | |
| | S. mutans, 50 | | |
| | S. pneumoniae, 50 | | |
| PF-547 | T. rubrum, 25 | WSRVPGHSDTGWKVWHRW-NH2 | 1060 |
| | B. subtilis, 25 | | |
| | S. mutans, 12.5 | | |
| PF-006 | A. baumannii, 50 | MGIIAGIIKFIKGLIEKFTGK | 1061 |
| | B. subtilis, 25 | | |
| | MRSA, 50 | | |
| PF-545 | A. niger, 50 | RESKLIAMADMIRRRI-NH2 | 1062 |
| | B. subtilis, 25 | | |
| | MRSA, 50 | | |
| PF-278 | C. albicans, 50 | LSLATFAKIFMTRSNWSLKRFNRL | 1063 |
| | T. rubrum, 50 | | |
| | S. epidermidis, 50 | | |
| PF-283 | T. rubrum, 50 | MIRIRSPTKKKLNRNSISDWKSNTSGRF | 1064 |
| | B. subtilis, 50 | FY | |
| | S. epidermidis, 50 | | |
| PF-307 | C. albicans, 50 | MKRRRCNWCGKLFYLEEKSKEAYCCK | 1065 |
| | T. rubrum, 50 | ECRKKAKKVKK | |
| | B. subtilis, 50 | | |
| PF-168 | T. rubrum, 50 | VLPFPAIPLSRRRACVAAPRPRSRQRAS | 1066 |
| | A. niger, 50 | | |
| | MRSA, 50 | | |
| PF-538 | A. baumannii, 25 | KNKKQTDILEKVKEILDKKKKTKSVGQ | 1067 |
| | C. difficile, 25 | KLY | |
| PF-448 | A. niger, 25 | SLQSQLGPCLHDQRH | 1068 |
| | S. pneumoniae, 50 | | |
| PF-583 | MRSA, 50 | KFQGEFTNIGQSYIVSASHMSTSLNTGK | 1069 |
| | S. epidermidis, 50 | | |
| PF-600 | E. coli, 50 | TKKIELKRFVDAFVKKSYENYILERELK | 1070 |
| | S. pneumoniae, 50 | KLIKAINEELPTK | |
| PF-525 | A. niger, 50 | KFSDQIDKGQDALKDKLGDL | 1071 |
| | S. pneumoniae, 50 | | |
| PF-529 | A. niger, 50 | LSEMERRRLRKRA-NH2 | 1072 |
| | S. pneumoniae, 50 | | |
| PF-148 | A. niger, 50 | RRGCTERLRRMARRNAWDLYAEHFY | 1073 |
| | B. subtilis, 50 | | |
| PF-530 | A. baumannii, 25 | SKFKVLRKIIIKEYKGELMLSIQKQR | 1074 |
| PF-522 | C. difficile, 25 | FELVDWLETNLGKILKSKSA-NH2 | 1075 |
| PF-497 | B. subtilis, 50 | LVLRICTDLFTFIKWTIKQRKS | 1076 |
| PF-499 | B. subtilis, 50 | VYSFLYVLVIVRKLLSMKKRIERL | 1077 |
| PF-322 | B. subtilis, 50 | GIVLIGLKLIPLLANVLR | 1078 |
| PF-511 | S. pneumoniae, 50 | VMQSLYVKPPLILVTKLAQQN | 1079 |
| PF-512 | S. pneumoniae, 50 | SFMPEIQKNTIPTQMK | 1080 |
| PF-520 | S. pneumoniae, 50 | LGLTAGVAYAAQPTNQPTNQPTNQPTN | 1081 |
| | | QPTNQPTNQPRW-NH2 | |
| PF-521 | S. pneumoniae, 50 | CGKLLEQKNFFLKTR | 1082 |
| PF-523 | S. pneumoniae, 50 | ASKQASKQASKQASKQASKQASRSLKN | 1083 |
| | | HLL | |
| PF-524 | S. pneumoniae, 50 | PDAPRTCYHKPILAALSRIVVTDR | 1084 |
| PF-209 | MRSA, 50 | NYAVVSHT | 1085 |
| PF-437 | S. pneumoniae, 50 | FQKPFTGEEVEDFQDDDEIPTII | 1086 |
| V | | KNLRIIRKGIHIIKKY | 1087 |
| W | | KLFKFLRKHLL | 1088 |
| X | | FLKFLKKFFKKLKY | 1089 |
| Y | | KNLRIIRKGIHIIKKY | 1090 |
| Z | | KNLRRIIRKGIHIIKKYG | 1091 |

Suitable antimicrobial peptides can include other known antimicrobial peptide sequences. For example, in certain embodiments, the antimicrobial peptides comprise one or more amino acid sequences described in the “Collection of Anti-Microbial Peptides” (CAMP) an online database developed for advancement the understanding of antimicrobial peptides (see, e.g., Thomas et al. (2009) Nucleic Acids Research, 2009, 1-7.doi:10.1093/nar/gkp1021) available at www.bicnirrh.res.in/antimicrobial. Numerous antimicrobial peptides can be found in the antimicrobial peptide database (http://aps.unmc.edu/AP/main.php).

A number of antimicrobial peptides are also disclosed in U.S. Pat. Nos. 7,271,239, 7,223,840, 7,176,276, 6,809,181, 6,699,689, 6,420,116, 6,358,921, 6,316,594, 6,235,973, 6,183,992, 6,143,498, 6,042,848, 6,040,291, 5,936,063, 5,830,993, 5,428,016, 5,424,396, 5,032,574, 4,623,733, which are incorporated herein by reference for the disclosure of particular antimicrobial peptides. In certain embodiments the antimicrobial peptides include any one or more of the peptides disclosed as having antimicrobial activity in PCT Application No: PCT/US2010/020242, which is incorporated herein by reference for the peptides listed therein.

Joining the Lytic/Permeabilizing Moiety to the Targeting Moiety.

The targeting moiety (e.g., targeting peptide, antibody, etc.) can be attached directly to the permeabilizing/lytic moiety or it can be attached by means of one or more linkers. For example, the targeting moiety and the permeabilizing/lytic moiety can be conjugated via a single multifunctional (e.g., bi-, tri-, or tetra-) linking agent or a pair of complementary linking agents. In another embodiment, the targeting moiety and the effector are conjugated via two, three, or more linking agents.

A “linker” or “linking agent” as used herein, is a molecule that is used to join two or more molecules. In certain embodiments the linker is typically capable of forming covalent bonds to both molecule(s) (e.g., the targeting moiety and the effector). Suitable linkers are well known to those of skill in the art and include, but are not limited to, straight or branched-chain carbon linkers, heterocyclic carbon linkers, or peptide linkers.

A bifunctional linker having one functional group reactive with a group on one molecule (e.g., a targeting peptide), and another group reactive on the other molecule (e.g., an antimicrobial peptide), can be used to form the desired conjugate. Alternatively, derivatization can be performed to provide functional groups. Thus, for example, procedures for the generation of free sulfhydryl groups on peptides are also known (see, e.g., U.S. Pat. No. 4,659,839).

In certain embodiments the linking agent is or comprises a functional group. Functional groups include monofunctional linkers comprising a reactive group as well as multifunctional crosslinkers comprising two or more reactive groups capable of forming a bond with two or more different functional targets (e.g., labels, proteins, macromolecules, semiconductor nanocrystals, or substrate). In some embodiments, the multifunctional crosslinkers are heterobifunctional crosslinkers comprising two or more different reactive groups.

Suitable reactive groups include, but are not limited to thiol (—SH), carboxylate (COOH), carboxyl (—COOH), carbonyl, amine (NH₂), hydroxyl (—OH), aldehyde (—CHO), alcohol (ROH), ketone (R₂CO), active hydrogen, ester, sulfhydryl (SH), phosphate (—PO₃), or photoreactive moieties. Amine reactive groups include, but are not limited to e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, but are not limited to e.g., haloacetyl and alkyl halide derivates, maleimides, aziridines, acryloyl derivatives, arylating agents, and thiol-disulfides exchange reagents. Carboxylate reactive groups include, but are not limited to e.g., diazoalkanes and diazoacetyl compounds, such as carbonyldiimidazoles and carbodiimides. Hydroxyl reactive groups include, but are not limited to e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, but are not limited to e.g., hydrazine derivatives for Schiff base formation or reduction amination. Active hydrogen reactive groups include, but are not limited to e.g., diazonium derivatives for mannich condensation and iodination reactions. Photoreactive groups include, but are not limited to e.g., aryl azides and halogenated aryl azides, benzophenones, diazo compounds, and diazirine derivatives.

Other suitable reactive groups and classes of reactions useful in forming chimeric moieties include those that are well known in the art of bioconjugate chemistry. Currently favored classes of reactions available with reactive chelates are those which proceed under relatively mild conditions. These include, but are not limited to, nucleophilic substitutions (e.g., reactions of amines and alcohols with acyl halides, active esters), electrophilic substitutions (e.g., enamine reactions), and additions to carbon-carbon and carbon-heteroatom multiple bonds (e.g., Michael reaction, Diels-Alder addition). These and other useful reactions are discussed in, for example, March (1985) Advanced Organic Chemistry, 3rd Ed., John Wiley & Sons, New York, Hermanson (1996) Bioconjugate Techniques, Academic Press, San Diego; and Feeney et al. (1982) Modification of Proteins; Advances in Chemistry Series, Vol. 198, American Chemical Society, Washington, D.C.

Many procedures and linker molecules for attachment of various molecules to peptides or proteins are known (see, e.g., European Patent Application No. 188,256; U.S. Pat. Nos. 4,671,958, 4,659,839, 4,414,148, 4,699,784; 4,680,338; 4,569,789; and 4,589,071; and Borlinghaus et al. (1987) Cancer Res. 47: 4071-4075).

Where the targeting moiety (e.g., targeting peptide, antibody, etc.) and permeabilizing/lytic moiety are both peptides, the selective permeabilizing reagent can be a fusion protein.

In certain embodiments the chimeric fusion proteins are synthesized using recombinant DNA methodology. Generally this involves creating a DNA sequence that encodes the fusion protein, placing the DNA in an expression cassette under the control of a particular promoter, expressing the protein in a host, isolating the expressed protein and, if required, renaturing the protein.

In other embodiments, the fusion protein can be chemically synthesized.

In certain embodiments a peptide linker/spacer is used to join the one or more targeting moieties the permeabilizing/lytic moiety. In various embodiments the peptide linker is relatively short, typically less than about 10 amino acids, preferably less than about 8 amino acids and more preferably about 3 to about 5 amino acids. Suitable illustrative linkers include, but are not limited to PSGSP ((SEQ ID NO:1092), ASASA (SEQ ID NO: 1093), or GGG (SEQ ID NO: 1094). In certain embodiments longer linkers such as (GGGGS)₃ (SEQ ID NO:1095) can be used. Illustrative peptide linkers and other linkers are shown in Table 5.

TABLE 5
Illustrative peptide and non-peptide linkers.

| | SEQ |
|---|---|
| Linker | ID NO: |
| AAA | |
| GGG | |
| SGG | |
| SAT | |
| PYP | |
| ASA | |
| GGSGGS | 1096 |
| GGGG | 1097 |
| PSPSP | 1098 |
| ASASA | 1099 |
| PSPSP | 1100 |
| KKKK | 1101 |
| RRRR | 1102 |
| GGGGS | 1103 |
| GGGGS GGGGS | 1104 |
| GGGGS GGGGS GGGGS | 1105 |
| GGGGS GGGGS GGGGS GGGGS | 1106 |
| GGGGS GGGGS GGGGS GGGGS GGGGS | 1107 |
| GGGGS GGGGS GGGGS GGGGS GGGGS GGGGS | 1108 |
| polyethylene glycol (PEG) | |
| 2-nitrobenzene or O-nitrobenzyl | |
| Nitropyridyl disulfide | |
| Dioleoylphosphatidylethanolamine (DOPE) | |
| S-acetylmercaptosuccinic acid | |
| 1,4,7,10-tetraazacyclododecane-1,4,7,10- | |
| tetracetic acid (DOTA) | |
| β-glucuronide and β-glucuronide variants | |
| Poly(alkylacrylic acid) | |
| Benzene-based linkers (for example: | |
| 2,5-Bis(hexyloxy)-1,4-bis[2,5- | |
| bis(hexyloxy)-4-formyl- | |
| phenylenevinylene]benzene) and like | |
| molecules | |
| Disulfide linkages | |
| Poly(amidoamine) or like dendrimers linking | |
| multiple target and killing peptides in one | |
| molecule | |
| Carbon nanotubes | |
| Hydrazone and hydrazone variant linkers | |
| PEG of any chain length | |
| Succinate, formate, acetate butyrate, | |
| other like organic acids | |
| Aldols, alcohols, or enols | |
| Peroxides | |
| alkane or alkene groups of any chain length | |
| One or more porphyrin or dye molecules | |
| containing free amide and | |
| carboxylic acid groups | |
| One or more DNA or RNA nucleotides, | |
| including polyamine and | |
| polycarboxyl-containing variants | |
| Inulin, sucrose, glucose, or other single, | |
| di or polysaccharides | |
| Linoleic acid or other polyunsaturated fatty | |
| acids | |
| Variants of any of the above linkers | |
| containing halogen or thiol groups | |

(All amino-acid-based linkers could be L, D, combinations of L and D forms, β-form, and the like)

STAMPs as Selective Permeabilization Reagents.

In various embodiments the selective permeabilization reagent is a STAMP (a Specifically Targeted Anti-Microbial Peptide). Typical STAMPs comprise one or more targeting peptides attached to one or more antimicrobial peptides. In certain embodiments the STAMPs are fusion proteins, while in other embodiments, the STAMPs are chemical conjugates.

In certain embodiments suitable STAMPs are contemplated comprising any one or more of the targeting peptides described herein attached directly or through a peptide or non-peptide linker to any one or more of the antimicrobial peptides described herein.

One suitable STAMP for selectively permeabilizing S. mutans is the C16G2 STAMP (SEQ ID NO:1111) which comprises an S. mutans binding peptide TFFRLFNRSFTQALGK (SEQ ID NO:1109) attached to an antimicrobial peptide KNLRIIRKGIHIIKKY (SEQ ID NO:1110).

TABLE 6
Illustrative STAMPs
for use as selective permeabilization reagents.

| | | C-term | SEQ ID |
|---|---|---|---|
| Name | Amino Acid Sequence | modification | NO |
| Sm-STAMPs | | | |
| C16G2 | TFFRLFNRSFTQALGKGGGKN | optionally | 1111 |
| | LRIIRKGIHIIKKY | amidated | |
| 1845L621 | KFINGVLSQFVLERKPYPKLF | optionally | 1112 |
| | KFLRKHLL | amidated | |
| b43BD2.21 | FIDSFIRSFGGGKLFKFLRKH | optionally | 1113 |
| | LL | amidated | |
| C16AF5 | TFFRLFNRSFTQALGKGGGFL | optionally | 1114 |
| | KFLKKFFKKLKY | amidated | |
| 2_1G2 | FIKHFIHRFGGGKNLRIIRKG | optionally | 1115 |
| | IHIIKKY | amidated | |
| Pa-STAMPs | | | |
| G10KHc | KKHRKHRKHRKHGGSGGSKNL | optionally | 1116 |
| | RRIIRKGIHIIKKYG | amidated | |

These STAMPs are intended to be illustrative and not limiting. Using the teachings provided herein methods utilizing numerous other STAMPs that are selectively permeabilizing to S. mutans or other microorganisms will be available to one of skill in the art.

### Detection Reagents.

Selection of detection reagent will vary with the format of the assay and/or the metabolite/enzyme (cellular component) that is to be detected. In certain embodiments the detection reagent comprises one or more reagents for the detection of components of a cell (e.g., an enzyme, a metabolite, an ionic species, another intracellular component). Such components include, but are not limited to, ATP, DNA, calcium, beta-galactosidase (beta-gal), beta-glucuronidase, alcohol dehydrogenase or other NAD oxidoreductase, a transferase, an alkaline phosphatase or other hydrolase, a lyase, an isomerase, an oxidase, a gyrase, a nuclease (DNases and RNases), a restriction enzyme, and the like. In certain embodiments, the detection reagent comprises one or more impermeant labels.

Substrates for Enzyme/Metabolite Activity.

Reagents for the detection of cellular components (e.g., enzymes and metabolites), as described above, are well known to those of skill in the art. In certain embodiments, effective detection of permeabilization or lysis of a microorganism can readily be achieved by detecting released ATP. Assays for ATP are well known to those of skill in the art.

A useful assay for detecting ATP released by the selectively permeabilized target microorganism or cell is a luciferase assay. Luciferase assays are based on the use of luciferase in the presence of a luciferase substrate (e.g., luciferin) to produce light (bioluminescence) in the presence of ATP. The light production thus provides a measure of the amount of ATP present in the sample. The luminescence generated by a luciferase reaction is typically detected with a luminometer although other detection means may be used. The presence of light greater than background level indicates the presence of ATP in the sample. The background level of luminescence is typically measured in the same matrix in which the sample exists, but in the absence of the sample. Suitable control reactions are readily designed by one of skill in the art. Luciferase assays for ATP are well known to those of skill in the art are commercially available.

Another method of detecting ATP involves the use of a target-responsive electrochemical aptamer switch (TREAS). In a typical TREAS design for ATP detection, an aptamer oligonucleotide dually labeled with thiol and ferrocene groups is hybridized with its complementary strand, and the thiolated duplex is self-assembled on a an electrode (e.g., a gold electrode). This duplex is responsive to the target ATP, which liberates the complementary strand and forms the aptamer—target complex. The electroactive ferrocene moiety, which is distal to the electrode surface in the absence of ATP, is moved to the proximal position during the binding-induced structural transition. This binding turns on the electron transfer and leads to measurable electrochemical signals for quantification of ATP. Such detection schemes can readily detect ATP at nanomolar levels (see, e.g., Zuo et al. (2007) J. Am. Chem. Soc., 129(5): 1042-1043). Another aptamer-based approach for the detection of ATP utilizes a signaling DNA aptamer attached to silica nanoparticles (see, e.g., Wang et al., (2008) Nanotechnology 19: 415605).

Still another approach to the detection of ATP utilizes a ligase-based ATP electrochemical assay using molecular beacon-like DNA. In this method, biotin-tagged molecular beacon (MB)-like DNA is self-assembled onto an electrode (e.g., a gold electrode) to form a stem-loop structure by means of gold-thiol chemistry, which results in blockage of electronic transmission producing an eT OFF state. In the presence of ATP, two nucleotide fragments which were complementary to the loop of the MB-like DNA can be ligated by ATP-dependent T4 DNA ligase. Hybridization of the ligated DNA with the MB-like DNA induces a significant conformational change in this surface-confined DNA structure, which in turn releases the biotin from the surface allowing free exchange of electrons with the electrode generating a measurable electrochemical signal (eT ON). The resulting change in electron transfer efficiency is readily measured, e.g., by differential pulse voltammetry at target ATP concentrations as low as 0.05 nM and with a linear response range from 0.1 to 1000 nM. This method had been successfully applied to the determination of ATP in the Escherichia coli O157:H7 extracts of water samples, and the linear response was found between the concentrations of 10³ and 10⁷ cfu/mL (see, e.g., Wang et al. (2009) Biosensors and Bioelectronics, 25(9): 2101-2106).

These approaches to the detection of ATP are illustrative and not intended to be limiting. Numerous other ATP detection schemes are known to those of skill and are commercially available.

In certain embodiments Ca²⁺ ion released by the permeabilized cell/microorganism is detected. Numerous fluorogenic or chromogenic indicators for calcium ions are well known to those of skill in the art. Such indicators include, but are not limited to Bis-fura, BTC, Calcium Green-1, Calcium Green-2, Calcium Green-5N, Calcium Orange, Calcium Crimson, Fluo-3, Fluo-4, Fluo-5F, Fluo-4FF, Fluo-5N, Fura-2, Fura-4F, Fura-6F, Fura-FF, Fura Red, Indo-1, Mag-fluo-4, Mag-fura-2, Mag-indo-1, Magnesium Green, Oregon Green 488 BAPTA-1, Oregon Green 488 BAPTA-2, Oregon Green 488 BAPTA-6F, Oregon Green 488 BAPTA-5N, Quin-2, Rhod-2, Rhod-3, Rhod-FF, Rhod-5N, X-rhod-1, and X-rhod-5F.

Similarly, assays for other metabolites, enzymes, and intracellular components (e.g., kinases, phosphatases, lipases, cellulases, etc.) are well known to those of skill in the art. For example, Table 7 lists a few indicators for various enzymatic activities.

TABLE 7
Illustrative indicators for enzyme/metabolite activity.

| Detection reagent | Activity Detected |
|---|---|
| 1-Methyl-3-indolyl-β-D-galactopyranoside | Chromogenic substrate for β-galactosidase |
| | that produces a green insoluble product. |
| 2-Acetamido-2-deoxy-3-O-(β-D- | An acceptor for the α1,2-fucosyltransferase |
| galactopyranosyl)-D-glucopyranose | enzyme from Helicobacter pylori. |
| 2-Ketobutyric acid, sodium salt | Substrate for the determination of lactate |
| | dehydrogenase isoenzymes. |
| 2-Methoxy-4-(2-nitrovinyl)phenyl β-D- | A chromogenic substrate (505 nm) for β- |
| glucopyranoside | glucosidase. |
| 2-Nitrophenyl b-D-galactopyranoside | A β-galactosidase substrate for colorimetric |
| | and EIA applications; counterpart of widely |
| | employed pNPP/alkaline phosphatase substrate. |
| | o-Nitrophenol is produced as the end product |
| | and is monitored at 405 nm. |
| 3-Indoxyl phosphate, disodium salt | This compound is a histochemical substrate |
| | for alkaline phosphatase. |
| 4-Chloro-1-naphthol | Chromogenic peroxidase substrate that is |
| | useful in enzyme-linked detection |
| | procedures. |
| 4-Methylumbelliferyl butyrate | Suitable to use as a fluorogenic substrate for |
| | esterases/lipases, such as butyrate esterase. |
| 4-Methylumbelliferyl Oleate | Suitable as fluorogenic substrate for lipases. |
| 4-Nitrophenyl acetate | A chromogenic esterase substrate. |
| 4-Nitrophenyl myristate | Suitable as a substrate for lipase. |
| 4-Nitrophenyl phenylphosphat | A 5′-Nucleotide Phosphodiesterase |
| | substrate. |
| 4-Nitrophenyl α-D-maltohexaoside | A substrate used in the determination of α- |
| | amylase activity. |
| 4-Nitrophenyl α-D-xylopyranoside | A chromogenic substrate for α-xylosidase. |
| 5-Bromo-4-chloro-3-indolyl alpha-L- | Employed as a chromogenic substrate for α- |
| fucopyranoside | D-Fucosidase, producing a blue precipitate. |
| 5-Bromo-4-chloro-3-indoxyl-3-acetate | A histochemical substrate for esterase. |
| 6-Chloro-3-indolyl β-D-glucopyranoside | A substrate used as a chromogenic medium |
| | for the detection of yeasts with β- |
| | glucosidase activity. |
| 8-Hydroxyquinoline-beta-D-glucuronic | A substrate for the histochemical |
| acid | demonstration of β-glucuronidase and for |
| | quantitative assay systems |
| Cellotetraose | A substrate for many cellulases and for 1,4- |
| | β-D-glucan glucohydrolases. |
| Naphthol AS-BI-phosphate, disodium salt | Substrate for the histochemical |
| hydrate | demonstration of acid and alkaline |
| | phosphatase. |
| o-Nitropheny1-β-D-xylobioside | A substrate for measuring xylanase activity. |
| | Xylanases are enzymes which hydrolyze |
| | xylan to xylooligosaccharides and have |
| | many applications in the food and feed |
| | industries. |
| Resorufin acetate | A fluorogenic substrate for hydrolytic |
| | enzymes (cellulases, chymotrypsin). |
| XGLUC Cyclohexylammonium Salt | Chromogenic substrate for β-glucuronidase |
| | (GUS) gene detection. |

Other suitable indicators include, but are not limited to coumarin-4-acetic acid 7-O-caprylate, coumarin-4-acetic acid 7-O-beta-D-glucuronide, and coumarin-4-acetic acid 7-O-beta-D-galactopyranoside.

Reagents for the Detection of Nucleic Acids.

In certain embodiments, selective permeabilization of the target microorganism releases a nucleic acid (e.g., RNA, DNA) which is then detected using a reagent suitable for the detection of nucleic acids. Labeled nucleic acid probes can also introduce another level of specificity and/or selectivity into the assay.

In certain embodiments, nucleic acid(s) released by the permeabilized microorganism is detected using, for example molecular beacons. Molecular beacons are single stranded hairpin shaped oligonucleotide probes labeled with a fluorophore and a quencher (e.g., a fluorescence resonance energy transfer (FRET) system). In the presence of the target sequence, they unfold, bind, the quencher is displaced from the fluorescent moiety, and the beacon fluoresces. In certain embodiments the use of “sloppy molecular beacons” is contemplated (see, e.g., Chakravorty et al. (2010) J. Clin. Microbiol., 48(1): 258-267, for a description of sloppy molecular beacons and their use to detect bacteria).

Released nucleic acids can also be detected using standard well-known PCR methods (e.g., lab-on-a-chip PCR amplification, standard PCR, etc.) with probes designed to amplify nucleic acid(s) from the target organism of interest.

A number of non-PCR based methods can also be used to detect the released nucleic acid(s). Illustrative methods include, but are not limited to the use of strand-displacing polymerases at a constant temperature (e.g., Loop-mediated Isothermal Amplification (LAMP) and Reaction Displacement Chimeric (RDC), or the use of transcription-mediated amplification (e.g., Nucleic acid sequence based amplification (NASBA)). All these methods do not require temperature cycling, operate at a constant temperature, and offer potential advantages including cost, speed, portability and reduced sensitivity to inhibitors over PCR.

Loop-mediated Isothermal Amplification (LAMP), developed by the Eiken Chemical Company is a simple, rapid, specific and cost-effective nucleic acid amplification technology. Details of the method are well known to those of skill in the art (see, e.g., //loopamp.eiken.co.jp/e/lamp/index.html). It is characterized by the use of 4 different primers, specifically designed to recognize 6 distinct regions on the target DNA template, in a process that proceeds at a constant temperature driven by a strand displacement reaction. Amplification and detection of target genes can be completed in a single step, by incubating the mixture of DNA template, primers and a strand displacement DNA polymerase, at a constant temperature. It provides high amplification efficiency, with replication of the original template copy, occurring 10⁹-10¹⁰ times during a 15-60 min reaction.

RDC (Reaction déplacement chimeric) is an isothermal DNA amplification procedure developed by Biomerieux, and is based on the use of chimeric primers consisting of an RNA stretch embedded within flanking DNA sequences. Cleavage of the hybrid duplex between the RNA region formed when the primer is hybridized to its DNA target provides the initiation for a strand-displacing polymerase (for details see, e.g., U.S. Pat. No. 5,824,517).

In various embodiments these various technologies can be can be interfaced with a unique reporter system known as BART (bioluminescent assay for real-time). BART is a bioluminescence real time assay developed by Lumora (www.lumora.co.uk) that allows the quantitative analysis of DNA amplification in real time. In BART, PPi produced during DNA amplification is converted to ATP by the action of ATP sulphurylase. This ATP is then used in a coupled simultaneous reaction by thermsotable firefly luciferase and luciferin to produce a light output permitting real-time analysis of amplification kinetics. A unique feature of BART is an initial burst of light, associated with the onset of exponential amplification, followed by a rapid decrease, as pyrophosphate reaches a critical threshold. The time to reach this light peak is therefore a function of the amount of target DNA in the sample at the beginning of the reaction (time to maximum; Tₘₐₓ), and a unique feature of the BART reporter. Quantification of BART is based on time to peak and not absolute light intensity, making it less prone to inhibition simplifying data interpretation and the hardware requirements.

NASBA is an isothermal nucleic acid amplification method that mimics retroviral replication and was originally applied to detection and quantification of RNA targets, but has also been adapted for DNA detection. Amplification occurs because the target is transcribed into RNA, which is then reverse-transcribed back into DNA, thereby providing more template copies for RNA transcription. The transcription is carried out by T7 RNA polymerase and requires the incorporation of the appropriate promoter sequence onto the template, which is achieved by appropriate primer design. This method was modified to allow DNA amplification using a two step procedure: first step with tailed primers, second step with universal primers. NASBA was developed well with performance characteristics similar to PCR, and adaptation to real-time detection using Molecular Beacons has been reported.

Other methods of detecting nucleic acids besides the molecular beacons, labeled probes, PCR, and various alternative amplification strategies described above are known to those of skill in the art.

Moreover, because target specificity is provided by the permeabilization reagent (e.g., STAMP) there is no need for the use of a sequence-specific detection reagent. Thus, for example, nucleic acid(s) can be detected by using labels known to preferentially bind DNA or RNA.

The foregoing detection methods and reagents for the detection of cellular components are meant to be illustrative and not limiting. Using the teachings provided herein detection schemes for other cellular components are readily available to one of skill in the art.

Impermeant Indicator Detection Reagents.

In certain embodiments the uptake of impermeant labels (detection reagents) into the selectively permeabilized target microorganisms, cells, etc. is detected. Impermeant labels are well known to those of skill in the art. Illustrative impermeant labels include, but are not limited to labels such as propidium iodide, SYTOX Green, SYBR®-14, YoYo®-1, YO-PROTM-1, BO-PRO-1, PO-PRO-1, YO-PRO-1, TO-PRO-1, TO-PRO-3, BO-PRO-3, YO-PRO-3, TO-PRO-#, POPO-1, BOBO-1, YOYO-1, TOTO-1, POPO-3, BOBO-2, YOYO-3, TOTO-3, ethidium homodimers-1, ethidium homodimers-2, ethidium bromide, ethidium monoazide, and Trypan blue. In this regard, it is noted that BO stains are benzothiazolium-4-pyridinium dyes, YO stains are benzoxazolium-4-quinolinium dyes, and TO stains are benzothiazolium-4-quinolinium dyes. Such impermeant labels are commercially available (see, e.g., Molecular Probes, Inc., and Invitrogen, Inc.).

It will be appreciated that this list of impermeant labels is not intended to be limiting. Numerous other impermeant labels are known to those of skill in the art an in view of the teachings provided herein it will be recognized that they are suitable in the methods described herein.

### Assay Formats and Devices.

The assays described herein can be performed in any of a wide variety of formats that permit detection of one target microorganism, or a plurality of different microorganism and/or evaluation of a single sample, or evaluation of a plurality of different samples. For example, in certain embodiments, different selective permeabilization reagents are located in different reaction chambers (e.g., in a microfluidic device), in different wells (e.g., in a microtiter plate), on different regions of a surface, e.g., in an array format, and the like. In such embodiments, different reaction chambers, wells, regions can be used to assay for different target microorganisms/cells, and the like.

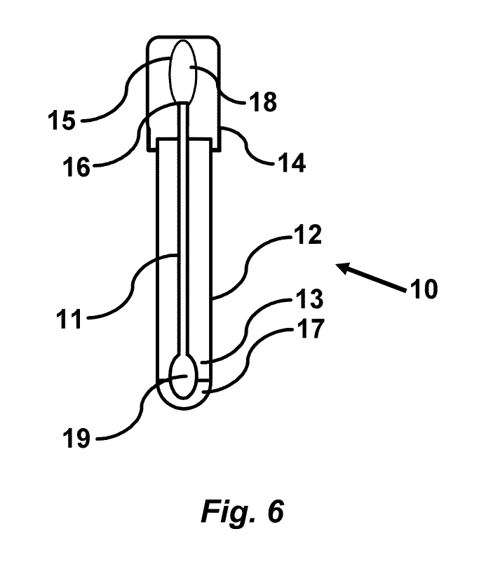

In certain embodiments the assay is provided as a diagnostic test unit. One illustrative diagnostic test is shown in FIG. 6. As schematically shown in this figure the device comprises a swab member 11 carried by a housing base 12 defining a sample chamber 13. The swab member 11 can further comprise a housing cap 14 comprising a first reagent chamber 15 where said housing cap interfits with said housing base 12 to cooperatively form a capped sample chamber 13 with the swab disposed in the sample chamber. The swab member additionally comprises a break-off nib, channel, or port 16 that communicates between the first reagent chamber and the sample chamber. A permeabilization reagent (e.g., a STAMP) 17 that selectively permeabilizes or lyses a target microorganism is disposed within the first reagent chamber 15 or within said sample chamber 13. An optional detection reagent or impermeant label 18 can be disposed within the first reagent chamber 15 when the permeabilization reagent is disposed within the sample chamber 13 or disposed within the sample chamber 13 when the permeabilization reagent is disposed within the first reagent chamber 15. Alternatively, the detection reagent and permeabilization reagent can be disposed within the same chamber, e.g., within the first reagent chamber 15, within the sample chamber 13, or within a second reagent chamber disposed in the housing cap or housing base.

In one usage (as illustrated in FIG. 2), the swab member (e.g., the swab tip is contacted with the sample of interest (e.g., the oral mucosa) to collect a sample. The swab member 11 is then inserted into the housing base 12 where the housing cap interfits with the housing base forming a closed sample chamber 13 with the swab tip 19 disposed therein. The swab can then be allowed to incubate with the permeabilization reagent 17 disposed within the sample chamber 13 to selectively permeabilize target microorganisms that may be present in the sample obtained on the swab tip. After incubation, the housing cap 14 is compressed delivering the detection reagent or impermeant label 18 past the break off nib or through the port or channel 16 into the sample chamber. The detection reagent or impermeant label is allowed to react, with optional mixing of the reaction chamber, to produce a detectable signal, and the signal is read in a test reader.

In certain embodiments the assay comprises a test strip based assay for use in a colorimetric, fluorescent or electrochemical meter. In certain embodiments the test strip is for use in an electrochemical meter.

In general, existing test strips for use in electrochemical meters comprise a substrate, working and reference electrodes formed on the surface of the substrate, and a means for making connection between the electrodes and the meter. The working electrode is coated with an enzyme and/or an enzyme substrate, e.g., as described herein and typically a mediator compound that transfers electrons from the enzyme to the electrode resulting in a measurable current when the target analyte is present. Representative mediator compounds include, but are not limited to a ferricyanide, metallocene compounds such as ferrocene, quinones, phenazinium salts, redox indicator DCPIP, and imidazole-substituted osmium compounds.

In this regard, commercial glucose meters (glucometers) are widely available and easily adapted for use with the test strip assays described herein. A typical glucometer utilizes a test strip comprising an enzyme electrode containing glucose oxidase. The glucose oxidase catalyzes the oxidation of glucose to hydrogen peroxide and D-glucono-δ-lactone in the presence of a cofactor flavin adenine dinucleotide (FAD) which is reduced to FADH₂. Then FADH₂ is oxidized by the final electron acceptor, molecular oxygen. The enzyme is reoxidized with an excess of phenol or ferrocyanide ion. The total charge passing through the electrode is measured and is proportional to the concentration of glucose in the blood. The coulometric method is a technique used to define a reaction where the amount of charge measured over a fixed time is measured. The amperometric method is used by some meters that allows the reaction to go to completion and where the total charge transfer is measured.

In one illustrative embodiment, the detection reagent (optionally in and/or on a test strip) an enzyme and a substrate for that enzyme and the detecting involves detecting the reaction between the enzyme and the substrate in the presence of a cofactor or a coenzyme (e.g., FAD, NAD, NADP, ATP, etc.) that is released from the microorganism.

In one illustrative embodiment, the “test strip” comprises glucose or another substrate for glucose oxidase, and glucose oxidase. When the target microorganism is present in the sample, it is lysed/permeabilized by the selective permeabilization reagent releasing one or more coenzymes (e.g., NAD, FAD, NADP). In the presence of the coenzyme, the glucose is oxidized with the corresponding reduction of the coenzyme. The coenzyme is subsequently oxidized and releases electrons (with or without a mediator (e.g. ferrocene, hexacyanoferrate III/hexacyanoferrate II, oxygen/hydrogen peroxide, phenanthroline quinine, nitrosalines, or organic salts such as N-methylphenazinium cation with tetracyanoquinodimethane radical anion). This reaction can be detected directly using a redox color change reagent, a redox fluorescent reagent, or electrochemically. Additional enzymes/enzymatic reactions can be utilized to couple the redox reaction with the detection means.

In another illustrative embodiment, the solid support (test strip) contains hexokinase, a hexose, glucose-6-phosphate dehydrogenase, and NAD. ATP released by the selectively lysed/permeabilized microorganism provides energy to permit the hexokinase to phorphorylate a hexose (e.g. glucose) with the corresponding reduction of NAD to NADH which is then detected directly or with the use of a mediator.

In another illustrative embodiment, the solid support (test strip) comprises glucose-6-phosphate dehydrogenase which in the presence of glucose-6-phosphate reduces NAD to NADH.

In still another illustrative embodiment, the “test strip” contains an alcohol dehydrogenase and an alcohol, and, optionally, a mediator. A coenzyme (e.g., NAD) released from the selectively permeabilized cells permits the reaction between the alcohol and alcohol dehydrogenase to proceed and the reduced NAD is detected.

In a number of embodiments, as indicated above, the detecting comprises detecting released NAD by detecting the reduction of said NAD to NADH.

In certain embodiments wherein the detection of the reduction of NAD, FAD, or NADP e.g., by detection of a colorimetric reagent that changes color when oxidized or reduced, by use of a fluorometric reagent, and/or by electrochemical means (e.g., measurement of impedence, voltage, conductance, current, or charge).

In various embodiments the test strip will carry a calibration code that can be entered into the reading meter, or that can be read directly by the meter. The calibration code can identify the assay chemistry and/or provide a meter calibration.

It will be appreciated that, in various embodiments, the selective permeabilization of the target “cells” can be performed on the test strip, in a sample chamber affixed to the test strip, in a sample collection device, or in a separate reaction chamber.

The materials used in, and the fabrication of enzymatic test strips are well known to those of skill in the art. Examples of glucometers and enzyme test strips are disclosed in European Patent No. 0 127 958, and U.S. Pat. Nos. 5,141,868, 5,286,362, 5,288,636, and 5,437,999 which are incorporated herein by reference.

It will be appreciated that this diagnostic test unit and method of use is intended to be illustrative and not limiting. Using the teachings provided herein numerous diagnostic test units comprising various permeabilization reagents (e.g., STAMPs) as described herein and various detection reagents will be available to one of skill in the art. In addition, using the teachings provided herein, numerous test units known to those of skill and commercially available can readily be adapted to perform the assays described herein (see, e.g., U.S. Pat. No. 5,078,968, U.S. Pat. No. 4,978,504, U.S. Pat. No. 4,707,450, U.S. Pat. No. 5,879,635, U.S. Pat. No. 5,266,266, U.S. Pat. No. 5,238,649, U.S. Pat. No. 5,869,003, U.S. Pat. No. 6,248,294; and U.S. Pat. Nos. 7,892,849, 7,824,344, 7,718,439, 7,556,723, 7,407,811, 7,390,667, 7,115,362, 6,951,728, 6,939,685, 6,923,764, 6,881,578, 6,818,180, 6,800,488, 6,780,651, 6,759,190, 6,638,772, 6,586,199, and 6,572,822, describing test strip, test strip indicators, and test strip meters, and the like).

### EXAMPLES

The following examples are offered to illustrate, but not to limit the claimed invention.

### Example 1

### Detection of S. Mutans Using the Selective Permeability Reagent C16G2 Stamp

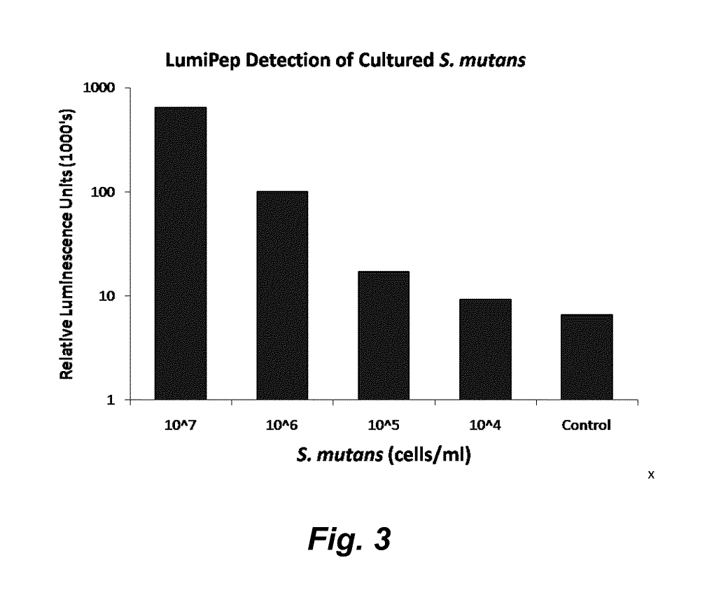

A first experiment was performed to determine the detection level of the assay. S. mutans was grown overnight in media and serially diluted to known concentrations in growth media. A 250 μl aliquot of each dilution was mixed with the STAMP (C16G2, SEQ ID NO:1111) and incubated for 10 minutes at room temperature. After incubation the luciferase reagent was added to the dilution, mixed briefly and luminescence measured. The control sample was fresh growth media. As shown in FIG. 3 the assay is capable of quantitatively detecting as little as 10⁴ cells/ml of cultured S. mutans grown in the lab. STAMP utilized, C 16G2.

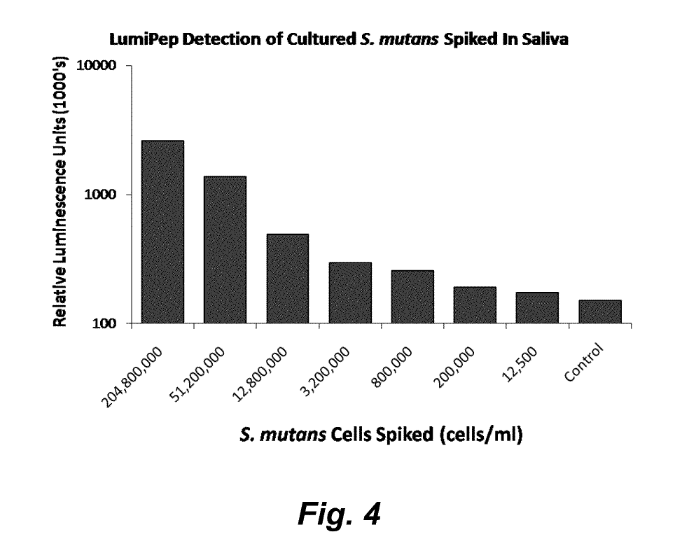

The ability of the assay to detect S. mutans in an unstimulated saliva sample was then determined. The saliva sample came from a volunteer who demonstrated low background levels of native S. mutans. S. mutans was grown overnight in media and serially diluted to known concentrations in the freshly collected unfiltered saliva sample. A 250 μl aliquot of each dilution was mixed with the C16G2 STAMP and incubated for 10 minutes at room temperature. After incubation the luciferase reagent was added to the saliva sample, mixed briefly and luminescence measured. The control sample was fresh saliva. FIG. 4 shows that the assay is capable of quantifying S. mutans spiked in a fresh unfiltered saliva sample. STAMP utilized, C16G2.

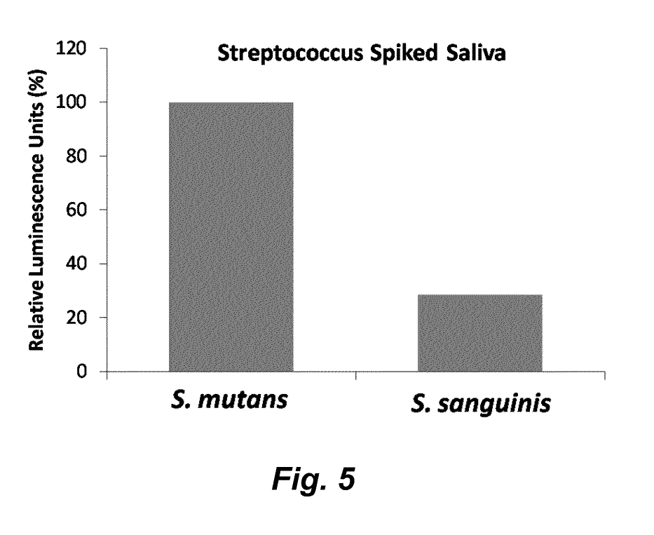

An experiment was performed to determine the difference in detection of S. mutans and the non-targeted streptococci, S. sanguinis. The saliva sample came from a volunteer who demonstrated low background levels of native S. mutans. S. mutans and S. sanguinis were grown overnight in media and serially diluted to 10⁷ cells/ml in the freshly collected unfiltered saliva sample. A 250 μl aliquot of each dilution was mixed with the Cl₆G2 STAMP and incubated for 10 minutes at room temperature. After incubation the luciferase reagent was added to the saliva sample, mixed briefly and luminescence measured. As shown in FIG. 5, FIG. 3 the assay demonstrates targeted permeabilization of spiked S. mutans in fresh saliva samples. In contrast, the non-target organism (S. sangu9nis) was note permeabilized.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of detecting or quantifying a target microorganism in a sample, said method comprising:
contacting said target microorganism with a selective permeabilization reagent that selectively permeabilizes or lyses said microorganism;
contacting the selectively permeabilized microorganism with a detection reagent that is taken into said selectively permeabilized organism or that contacts metabolites or enzymes released by said selectively permeabilized microorganism, wherein said detection reagent produces a signal in the presence of said metabolites or enzymes; and
detecting a signal produced by said detection reagent in the presence of said metabolites or enzymes wherein the strength of said signal indicates the presence and/or amount of said target microorganism in said sample.

2. The method of claim 1, wherein said metabolites or enzymes comprise a metabolite or enzyme selected from the group consisting of ATP, DNA, RNA, calcium, beta-galactosidase (beta-gal), beta-glucuronidase, alcohol dehydrogenase or other NAD oxidoreductase, a transferase, an alkaline phosphatase or other hydrolase, a lyase, an isomerase, an oxidase, a gyrase, a DNA nuclease (DNases), and 1RNA nuclease (RNase), and a restriction enzyme.

3. The method of claim 2, wherein said metabolites or enzymes comprise ATP.

4. The method of claim 3, wherein said detection reagent comprises a luciferase and said signal comprises a luminescence signal.

5. The method of claim 3, wherein said detection reagent comprises a target responsive electrochemical aptamer switch (TREAS) for ATP detection and said signal comprises an electrochemical signal.

6. The method of claim 3, wherein said detection reagent comprises a molecular beacon (MB)-like DNA for the detection of ATP and said signal comprises a fluorescent signal.

7. The method of claim 3, wherein said detection reagent comprises an enzyme substrate and said detecting comprises detecting a reaction between the released enzyme and said enzyme substrate.

8. The method of claim 7, wherein said enzyme substrate is a substrate for an enzyme selected from the group consisting of beta-galactosidase (beta-gal), beta-glucuronidase, alcohol dehydrogenase or other NAD oxidoreductases, transferases, alkaline phosphatases or other hydrolases, lyases, isomerases, oxidases, gyrases, a DNA nuclease (DNases), and 1RNA nuclease (RNase), and a restriction enzyme.

9. The method of claim 8, wherein said substrate is selected from the group consisting of coumarin-4-acetic acid 7-O-caprylate, coumarin-4-acetic acid 7-O-beta-D-glucuronide, and coumarin-4-acetic acid 7-O-beta-D-galactopyranoside.

10. The method of claim 3, wherein said detection reagent comprises an enzyme and a substrate for that enzyme and said detecting comprise detecting the reaction between the enzyme and the substrate in the presence of a cofactor or a coenzyme that is released from said microorganism.

11. The method of claim 10, wherein said detection reagent comprises an enzyme that uses NAD, NADP, or FAD as a cofactor.

12. The method of claim 7, wherein said enzyme substrate and/or said enzyme is provided on and/or in a solid support.

13. The method of claim 12, wherein said substrate comprises glucose or another substrate for glucose oxidase, and glucose dehydrogenase.

14. The method of claim 13, wherein said detecting comprises detecting the reduction of one or more coenzymes selected from the group consisting of NAD, NADP, and FAD.

15. The method of claim 12, wherein said substrate comprises hexokinase, a hexose, glucose-6-phosphate dehydrogenase, and NAD.

16. The method of claim 15, wherein said detecting comprises detecting released ATP by detecting the reduction of said NAD to NADH.

17. The method of claim 12, wherein said substrate comprises glucose-6-phosphate dehydrogenase.

18. The method of claim 17, wherein said detecting comprises detecting released NAD by detecting the reduction of said NAD to NADH.

19. The method of claim 12, wherein the detection of the reduction of NAD NADP, or FAD comprises detection of a colorimetric reagent that changes color when oxidized or reduced.

20. The method of claim 12, wherein the detection of the reduction of NAD NADP or FAD comprises electrochemical detection of a reagent that is oxidized or reduced.

21. The method of claim 12, wherein said substrate comprises a test strip compatible with a glucometer readout device.

22. (canceled)

23. The method of claim 12, wherein said contacting the target microorganism with a selective permeabilization reagent occurs on and/or in said substrate.

24. The method of claim 12, wherein said contacting the target microorganism with a selective permeabilization reagent occurs in a sample collection device before application to said substrate.

25. The method of claim 1, wherein said permeabilization reagent comprises a reagent that disrupts or permeabilizes a microorganism or cell attached to a targeting peptide or antibody that preferentially or specifically binds to said target microorganism.

26. (canceled)

27. The method of claim 25, wherein said targeting peptide is a targeting peptide selected from the targeting peptides listed in Table 2.

28. The method of claim 25, wherein said targeting peptide is attached directly or indirectly to an antimicrobial peptide.

29. The method of claim 28, wherein said antimicrobial peptide is an antimicrobial peptide selected from the antimicrobial peptides listed in Table 4.

30. The method of claim 25, wherein said target microorganism is S. mutans, and said targeting peptide attached to an antimicrobial peptide comprises an amino acid sequence selected from the group consisting of TFFRLFNRSFTQALGKGGGKNLRIIRKGIHIIKKY (C16G2, SEQ ID NO:1110), KFINGVLSQFVLERKPYPKLFKFLRKHLL (1845L621, SEQ ID NO:1111), FIDSFIRSFGGGKLFKFLRKHLL (b43BD2.21, (SEQ ID NO:1112), TFFRLFNRSFTQALGKGGGFLKFLKKFFKKLKY (C16AF5, (SEQ ID NO:1113), FIKHFIHRFGGGKNLRIIRKGIHIIKKY (2_{—}1G2, (SEQ ID NO:1115), and KKHRKHRKHRKH GGSGGS KNLRRIIRKGIHIIKKYG (G10KHc, (SEQ ID NO:1115).

31-33. (canceled)

34. The method of claim 1, wherein said sample comprises a sample from saliva, plaque, urine, feces, cerebrospinal fluid, blood, vaginal secretions, soil, a surface swab, an agricultural product, a meat product, a poultry product, and a fish product.

35. A method of detecting or quantifying a target microorganism in a sample, said method comprising:
contacting said target microorganism with a permeabilization reagent that selectively permeabilizes said microorganism;
contacting the selectively permeabilized microorganism with a cell-impermeant label; and
detecting said label in said cell where the presence or amount of said label associated with a microorganism indicates the presence or amount of said target microorganism in said sample.

36. The method of claim 35, wherein said detecting comprises a method selected from the group consisting of microscopy, flow cytometry, solid phase cytometry, luminometry, and spectroscopy.

37. The method of claim 35, wherein said impermeant label comprises a label selected from the group consisting of propidium iodide, SYTOX Green, SYBR®-14, YoYo®-1, YO-PRO™-1, BO-PRO-1, PO-PRO-1, YO-PRO-1, TO-PRO-1, TO-PRO-3, BO-PRO-3, YO-PRO-3, TO-PRO-#, POPO-1, BOBO-1, YOYO-1, TOTO-1, POPO-3, BOBO-2, YOYO-3, TOTO-3, ethidium homodimers-1, ethidium homodimers-2, ethidium bromide, ethidium monoazide, and Trypan blue.

38-39. (canceled)

40. The method of claim 35, wherein said permeabilization reagent comprises a reagent that disrupts or permeabilizes a microorganism attached to a targeting peptide that preferentially or specifically binds to said target microorganism.

41-44. (canceled)

45. The method of claim 40, wherein said target microorganism is S. mutans, and said permeabilization reagent comprises an amino acid sequence selected from the group consisting of TFFRLFNRSFTQALGK GGG KNLRIIRKGIHIIKKY (C16G2, SEQ ID NO:1110), KFINGVLSQFVLERK PYP KLFKFLRKHLL (1845L621, SEQ ID NO:1111), FIDSFIRSF GGG KLFKFLRKHLL (b43BD2.21, (SEQ ID NO:1112), TFFRLFNRSFTQALGK GGG FLKFLKKFFKKLKY (C16AF5, (SEQ ID NO:1113), FIKHFIHRF GGG KNLRIIRKGIHIIKKY (2_{—}1G2, (SEQ ID NO:1114), and KKHRKHRKHRKH GGSGGS KNLRRIIRKGIHIIKKYG (G10KHc, (SEQ ID NO:1115).

46-49. (canceled)

50. A diagnostic test device, said device comprising:
a substrate test strip comprising a selective permeabilization reagent;
an enzyme substrate; and
a detection reagent that detects a change in oxidation state of a coenzyme.

51. The device of claim 50, wherein said substrate comprises glucose or another substrate for glucose oxidase, and glucose dehydrogenase.

52. The device of claim 51, wherein said substrate comprises one or more coenzymes selected from the group consisting of NAD and FAD.

53. The device of claim 50, wherein said substrate comprises hexokinase, a hexose, glucose-6-phosphate dehydrogenase, and NAD.

54-55. (canceled)

56. The device of claim 50, wherein the detection reagent that is detectable using an electrochemical detection device.

57-58. (canceled)

59. A diagnostic test unit comprising:
a swab member carried by a housing base defining a sample chamber;
a housing cap comprising a first reagent chamber wherein said housing cap interfits with said housing base to cooperatively form a capped sample chamber with said swab disposed therein and a break-off nib, channel, or port that communicates between said first reagent chamber and said sample chamber; and
a permeabilization reagent that selectively permeabilizes or lyses a target microorganism wherein said permeabilization reagent is disposed within said first reagent chamber or within said sample chamber.

60-68. (canceled)

69. The diagnostic test unit of claim 59, wherein said target microorganism is S. mutans, and said targeting peptide attached to an antimicrobial peptide comprises an amino acid sequence selected from the group consisting of TFFRLFNRSFTQALGKGGGKNLRIIRKGIHIIKKY (C16G2, SEQ ID NO:1110), KFINGVLSQFVLERKPYPKLFKFLRKHLL (1845L621, SEQ ID NO:1111), FIDSFIRSFGGGKLFKFLRKHLL (b43BD2.21, (SEQ ID NO:1112), TFFRLFNRSFTQALGKGGGFLKFLKKFFKKLKY (C16AF5, (SEQ ID NO:1113), and FIKHFIHRFGGGKNLRIIRKGIHIIKKY (2_{—}1G2, (SEQ ID NO:1114), and KKHRKHRKHRKH GGSGGS KNLRRIIRKGIHIIKKYG (G10KHc, (SEQ ID NO:1115).

70. (canceled)
